(19) United States
(12) Patent Application Publication
(10) Pub. No.: US 2026/0184692 A1
(43) Pub. Date: Jul. 2, 2026

(54) HETEROCYCLIC COMPOUNDS, PREPARATION METHODS AND USES THEREOF

(71) Applicants: InventisBio Co., Ltd., Shanghai (CN); InventisBio LLC, Short Hills, NJ (US)

(72) Inventors: Xing DAI, Shanghai (CN); Younong YU, Short Hills, NJ (US); Hong YANG, Shanghai (CN); Yong LUO, Shanghai (CN); Jifang WENG, Shanghai (CN); Rongfeng LIU, Shanghai (CN); Yaolin WANG, Shanghai (CN)

(21) Appl. No.: 19/131,661

(22) PCT Filed: Nov. 24, 2023
(86) PCT No.: PCT/CN2023/133935

§ 371 Date: May 21, 2025

(30) Foreign Application Priority Data
Nov. 24, 2022 (WO) PCT/CN2022/134025
Feb. 15, 2023 (WO) PCT/CN2023/076151
Oct. 3, 2023 (WO) PCT/CN2023/123074

(51) Int. Cl. C07D 401/14; A61K 31/438; A61K 31/4545; A61K 31/4747; A61K 31/496; A61K 31/497; A61K 31/501; A61K 31/506; A61K 31/5377; A61K 31/5383; A61K 31/5386; A61K 31/55; A61K 45/06; A61P 35/00; C07D 221/20; C07D 401/04; C07D 401/12; C07D 405/14; C07D 413/14; C07D 417/14; C07D 471/04; C07D 471/10; C07D 498/04; C07D 498/08; C07D 498/14

(52) U.S. Cl. CPC C07D 401/14; A61K 31/438; A61K 31/4545; A61K 31/4747; A61K 31/496; A61K 31/497; A61K 31/501; A61K 31/506; A61K 31/5377; A61K 31/5383; A61K 31/5386; A61K 31/55; A61K 45/06; A61P 35/00; C07D 221/20; C07D 401/04; C07D 401/12; C07D 405/14; C07D 413/14; C07D 417/14; C07D 471/04; C07D 471/10; C07D 498/04; C07D 498/08; C07D 498/14

(57) ABSTRACT

Provided herein are novel compounds, for example, compounds having a Formula (I), a prodrug thereof, or a pharmaceutically acceptable salt thereof. Also provided herein are pharmaceutical compositions comprising a compound having a Formula (I), a prodrug thereof, or a pharmaceutically acceptable salt thereof, methods of preparing the compounds and methods of using the compounds, for example, in inhibiting KIF18A in a cell, and/or in treating various cancers associated with KIF18A protein.

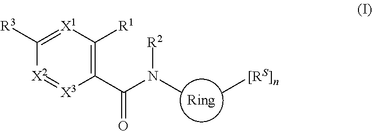

### CROSS REFERENCE TO RELATED APPLICATION

This application claims priority to International Application Nos. PCT/CN2022/134025, filed on Nov. 24, 2022; PCT/CN2023/076151, filed on Feb. 15, 2023; and PCT/CN2023/123074, filed on Oct. 3, 2023, the contents of each of which are incorporated herein by reference in their entireties.

### BACKGROUND OF THE INVENTION

### Field of the Invention

In various embodiments, the present invention generally relates to novel heterocyclic compounds, compositions of the same, methods of preparing and methods of using the same, e.g., for inhibiting KIF18A and/or for treating a number of diseases or disorders, such as cancers associated with KIF18A protein.

### Background

KIF18A, a member of the kinesin-8 family, moves towards the plus-end direction of microtubules powered by the energy derived from ATP hydrolysis in cells. KIF18A is positioned at the plus-end of the microtubule to regulate the dynamic instability of the microtubule and exert the activity of microtubule depolymerase essential for chromosome segregation. During mitosis, KIF18A can regulate spindle microtubule dynamics and chromosome amplitude, and plays a key role in the timely completion of chromosome alignment, genomic stability and successful completion of mitosis. In addition, KIF18A is also involved in various other cellular processes, including cell cycle, cell migration, and the organization of the cytoplasm. Alterations in the expression or activity of KIF18A can result in abnormal cell division and contribute to the development of cancer.

Thus, there is an unmet medical need for therapeutic treatments of cancer associated with KIF18A protein.

### BRIEF SUMMARY OF THE INVENTION

In various embodiments, the present disclosure provides novel compounds, pharmaceutical compositions, methods of preparing and using the same. Typically, the compounds herein are KIF18A inhibitors. The compounds and compositions herein are useful for treating various diseases or disorders, such as cancers associated with KIF18A protein.

In various embodiments, the present disclosure provides a compound of Formula I, or a pharmaceutically acceptable salt thereof:

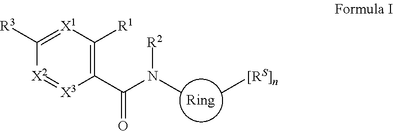

wherein the variables are defined herein. In some embodiments, the compound of Formula I can have a subformula of Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, 1-2-C, I-2-D, or I-2-E, as defined herein. In some embodiments, the present disclosure provides a compound selected from those as shown in Table A, or a pharmaceutically acceptable salt thereof. In some embodiments, when applicable, the compound can exist as a mixture of atropisomers in any ratio. In some embodiments, when applicable, the compound can exist as an isolated individual atropisomer substantially free (e.g., with less than 20%, less than 10%, less than 5%, less than 1%, by weight, by HPLC area, or both, or with a non-detectable amount) of the other atropisomer(s).

Certain embodiments are directed to a pharmaceutical composition comprising one or more of the compounds of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of those shown in Table A, or a pharmaceutically acceptable salt thereof) and optionally a pharmaceutically acceptable excipient. The pharmaceutical composition described herein can be formulated for different routes of administration, such as oral administration, parenteral administration, or inhalation etc.

Certain embodiments are directed to a method of treating a disease or disorder associated with KIF18A protein. In some embodiments, the method comprises administering to a subject in need thereof a therapeutically effective amount of a compound of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof) or a therapeutically effective amount of a pharmaceutical composition described herein. In some embodiments, a method of treating cancer is provided. In some embodiments, the method comprises administering to a subject in need thereof a therapeutically effective amount of a compound of the present disclosure or a therapeutically effective amount of a pharmaceutical composition described herein. In various embodiments, the cancer can be selected from breast, bladder, colon, cervix, lung, pancreas, prostate, and ovarian cancers. The administering is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. The compounds of the present disclosure can be used as a monotherapy or in a combination therapy. In some embodiments, the combination therapy includes treating the subject with a chemotherapeutic agent, therapeutic antibody, radiation, cell therapy, or immunotherapy.

It is to be understood that both the foregoing summary and the following detailed description are exemplary and explanatory only, and are not restrictive of the invention herein.

### BRIEF DESCRIPTION OF THE FIGURES

FIG. 1a and FIG. 1b show inhibition of tumor growth of exemplary compound tested in OVCAR3 in vivo mouse xenograft model study.

### DETAILED DESCRIPTION OF THE INVENTION

In various embodiments, provided herein are novel compounds, pharmaceutical compositions, methods of preparation and methods of use.

### Compounds

Some embodiments of the present disclosure are directed to novel compounds. The compounds herein typically can be an inhibitor of KIF18A.

In some embodiments, the present disclosure provides a compound of Formula I, or a pharmaceutically acceptable salt thereof:

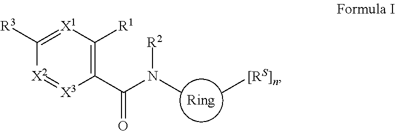

wherein:
R¹ are an optionally substituted C₃₋₁₀ carbocyclyl, an optionally substituted 4-10 membered heterocyclyl, an optionally substituted aryl, or an optionally substituted heteroaryl;
R² is hydrogen, an optionally substituted C₁₋₆ alkyl, an optionally substituted C₁₋₆ heteroalkyl, or a nitrogen protecting group;
or R¹ and R², together with the intervening C, C, C, and N atoms, are joined to form a 5-14 membered heterocyclyl, which is optionally substituted;
R³ is R^{A}, OR^{A}, SR^{A}, S(O)R^{A}, S(O)₂R^{A}, COR^{A}, COOR^{A}, CN, NHR^{A}, CONHR^{A}, S(O)₂NHR^{A}, S(O)(NH)R^{A}, NHCOR^{A}, NHS(O)₂R^{A}, or NO₂, wherein R^{A} is independently hydrogen, halogen, CN, OH, an optionally substituted C₁₋₆ alkyl, an optionally substituted C₂₋₆ alkenyl, an optionally substituted C₂₋₆ alkynyl, an optionally substituted C₁₋₆ heteroalkyl, an optionally substituted C₁₋₆ haloalkyl, an optionally substituted C₃₋₁₀ carbocyclyl, an optionally substituted 4-10 membered heterocyclyl, an optionally substituted aryl, or an optionally substituted heteroaryl;
X¹ is N or CR⁴, wherein R⁴ is H, F, Cl, OH, NH₂, CN, CD₃, CF₃, an optionally substituted C₁₋₄ alkyl, an optionally substituted C₁₋₄ heteroalkyl, or an optionally substituted C₃₋₆ cycloalkyl;
X² is N or CR⁵, wherein R⁵ is H, F, Cl, OH, NH₂, CN, CD₃, CF₃, an optionally substituted C₁₋₄ alkyl, an optionally substituted C₁₋₄ heteroalkyl, or an optionally substituted C₃₋₆ cycloalkyl;
X³ is N or CR⁶, wherein R⁶ is H, F, Cl, OH, NH₂, CN, CD₃, CF₃, an optionally substituted C₁₋₄ alkyl, an optionally substituted C₁₋₄ heteroalkyl, or an optionally substituted C₃₋₆ cycloalkyl;
or
R⁵ and R⁶, together with the intervening C and C atoms, are joined to form a 5-8 membered heteroaryl, which is optionally substituted;

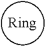

is an optionally substituted C₃₋₁₀ carbocyclic ring, an optionally substituted 4-10 membered heterocyclic ring, an optionally substituted aromatic ring containing 0, 1, 2, or 3 heteroatoms independently selected from N, O, and S;
R^{S} at each occurrence is independently R^{T}, OR^{T}, SR^{T}, NHR^{T}, COR^{T}, COOR^{T}, CONHR^{T}, NHCOR^{T}, CN, or NO₂, wherein R^{T} is independently hydrogen, halogen (e.g. F, Cl, or Br), CN, OH, an optionally substituted C₁₋₆ alkyl, an optionally substituted C₂₋₆ alkenyl, an optionally substituted C₂₋₆ alkynyl, an optionally substituted C₁₋₆ heteroalkyl, an optionally substituted C₁₋₆ haloalkyl, an optionally substituted C₃₋₁₀ carbocyclyl, an optionally substituted 4-10 membered heterocyclyl, an optionally substituted aryl, or an optionally substituted heteroaryl; and
n is 0, 1, 2, or 3.

In some embodiments, the present disclosure also provides a prodrug of the compound of Formula I (e.g., any of the subformulae herein), or a pharmaceutically acceptable salt thereof. As understood in the art, a prodrug of an active ingredient generally refers to a compound that can be converted into the active ingredient upon administration to a subject, such as a mammal, preferably, a human. A prodrug is typically stable such that it can be prepared and/or formulated prior to administration to a subject. In some embodiments, the prodrug is an ester prodrug, such as those derived from an OH group of the compound of Formula I and a carboxylic acid having 1-20 carbons, wherein one or more carbons can have optional substituents, such as OH, NH₂, monoalkyl amine, dialkyl amine, etc. In some embodiments, the prodrug is an amino ester prodrug, e.g., a prodrug derived from an OH group of the compound of Formula I and an amino acid, such as a nature amino acid (e.g., L-valine) or a non-natural amino acid, or a peptide such as dipeptide, tripeptide, or tetrapeptide. Other types of prodrugs are also suitable.

In some embodiments, X¹ is N or CR⁴, wherein R⁴ is H, F, or Cl. In some embodiments, X² is N or CR⁵, wherein R⁵ is H, F, or Cl. In some embodiments, X³ is N or CR⁶, wherein R⁶ is H, F, or Cl. In some embodiments, X¹ is N, and X² and X³ is each independently CH, CF, or CCH₃. In some embodiments, X² is N, and X¹ and X³ is each independently CH, CF, or CCH₃. In some embodiments, X³ is N, and X¹ and X² is each independently CH, CF, or CCH₃. In some embodiments, both X¹ and X² are N, and X³ is CH or CF. In some embodiments, both X² and X³ are N, and X¹ is CH or CF. In some embodiments, X¹, X², and X³ are CH. In some embodiments, one of X¹, X², and X³ is CF, and the other two of X¹, X², and X³ are CH.

In some embodiments, the compound of Formula I can be characterized by having Formula I-1 and I-2:

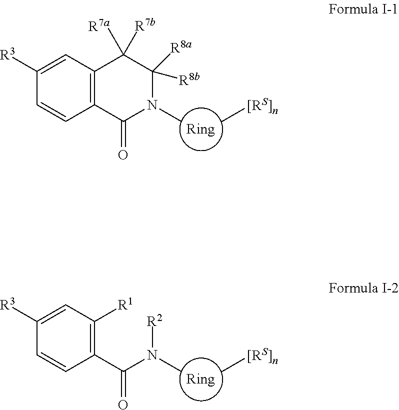

wherein the variables R¹, R², R³,

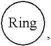

R^{S}, and n include any of those described herein in any combination;
wherein R^{7a} and R^{7b} in Formula I-1 are each independently hydrogen, halogen, CN, OH, an optionally substituted C₁₋₆ alkyl, an optionally substituted C₂₋₆ alkenyl, an optionally substituted C₂₋₆ alkynyl, an optionally substituted C₁₋₆heteroalkyl, or an optionally substituted C₁₋₆ haloalkyl; or
R^{7a} and R^{7b}, together with the intervening C atom, are joined to form an optionally substituted C₃₋₁₀ carbocyclyl, or an optionally substituted 4-10 membered heterocyclyl;
wherein R^{8a} and R^{8b} in Formula I-1 are each independently H, F, Cl, CN, OH, C₁₋₄ alkyl (e.g., methyl, ethyl, n-propyl, isopropyl), C₁₋₄haloalkyl (e.g., fluoromethyl, difluoromethyl, trifluoromethyl, etc.), C₁₋₄ alkoxy (e.g., methoxy, ethoxy, isopropoxy, etc.), or C₁₋₄ haloalkoxy (e.g., CF₃O—, CF₃CH₂O—, etc.);
wherein R¹ and R² in Formula I-2 are not joined to form a 5-14 membered heterocyclyl, which is optionally substituted.

In some embodiments, in Formula I-1, R^{7a} and R^{7b}, together with the intervening C atom, are joined to form an optionally substituted C₃₋₁₀ carbocyclyl. For example, R^{7a} and R^{7b}, together with the intervening C atom, are joined to form an optionally substituted C₄ carbocyclyl, an optionally substituted C₅ carbocyclyl, an optionally substituted C₆ carbocyclyl, an optionally substituted C₇ carbocyclyl, or an optionally substituted C₈ carbocyclyl.

In some embodiments, in Formula I-1, both R^{8a} and R^{8b} are hydrogen. In some embodiments, in Formula I-1, R^{8a} is hydrogen, and R^{8b} is F or Cl.

In some embodiments, the compound of Formula I-1 can be characterized by having Formula I-1-A:

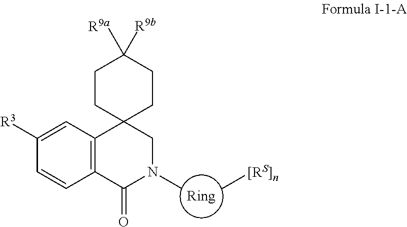

wherein the variables R³,

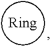

R^{S}, and n include any of those described herein in any combination;
wherein R^{9a} and R^{9b} are each independently hydrogen, halogen, an optionally substituted C₁₋₆ alkyl, or an optionally substituted C₁₋₆ heteroalkyl; or
R^{9a} and R^{9b}, together with the intervening C atom, are joined to form an optionally substituted C₃₋₆ carbocyclyl, or an optionally substituted 4-6 membered heterocyclyl, or
R^{9a} and R^{9b} are joined to form ═CF₂, ═CCl₂, or ═C(CH₃)₂.

In some embodiments, R^{9a} and R^{9b} are each independently H, F, Cl, methyl, ethyl, fluoromethyl, difluoromethyl, trifluoromethyl, methoxy, ethoxy, CF₃O—, or CF₃CH₂O—. In some embodiments, R^{9a} and R^{9b} are each independently H, F, Cl, methyl, ethyl, CD₃, trifluoromethyl, or CF₃O—. In some embodiments, R^{9a} and R^{9b}, together with the intervening C atom, are joined to form an optionally substituted cyclopropyl or cyclobutyl. In some embodiments, R^{9a} and R^{9b}, together with the intervening C atom, are joined to form an optionally substituted cyclopropyl.

In some embodiments,

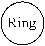

is a 5- or 6-membered heteroaryl ring containing one or two ring N atoms, such as imidazolyl, pyridinyl, pyrimidinyl, pyrazinyl, or pyridazinyl. In some embodiments,

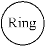

is a 9-membered heteroaryl ring containing one, two or three ring N atoms, such as benzoimidazolyl, pyrrolopyridinyl, or imidazopyridinyl. In some embodiments, R^{S} at each occurrence can be any one as defined below in R^{B}, R^{C}, R^{D}, R^{E}, R^{F}, R^{G1}, or R^{G2}. In some embodiments, n is 1, 2, or 3.

In some embodiments,

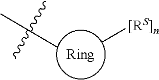

is selected from the following groups which are further optionally substituted:

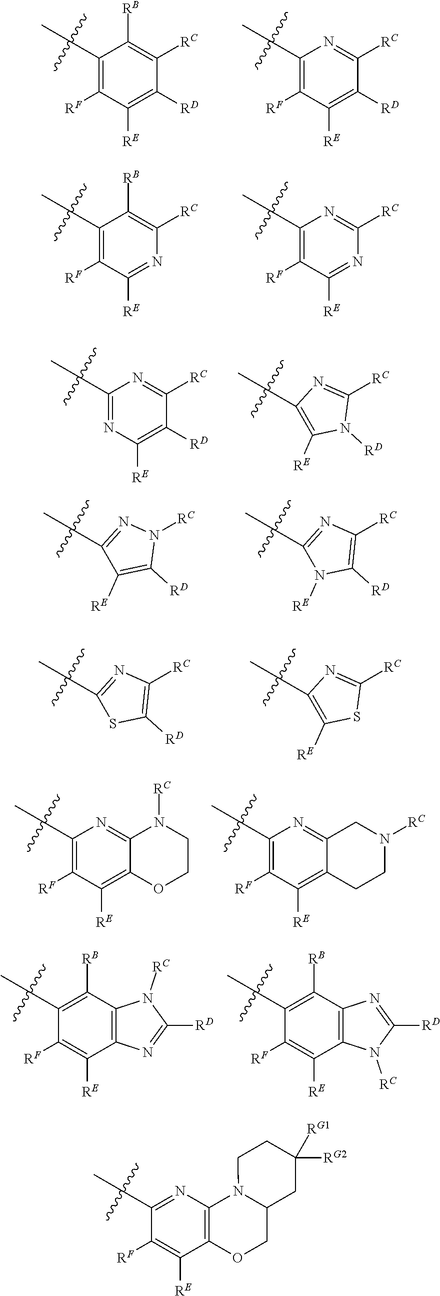

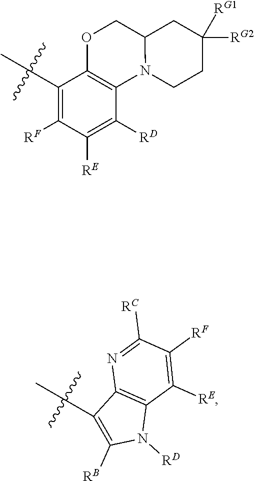

wherein:
each of R^{B}, R^{D}, R^{E}, R^{F}, R^{G1}, and R^{G2} are independently hydrogen, halogen, CN, OH, NH₂, an optionally substituted C₁₋₆ alkyl, an optionally substituted C₁₋₄ heteroalkyl, or an optionally substituted C₃₋₆ cycloalkyl;
R^{C} is -L₁-L₂, wherein Li is null, —O—, —NH—, —N(C₁₋₆ alkyl)-, —CH₂—, —CH(C₁₋₆ alkyl)-, —CH(OH)—, —C(O)—, —S—, —S(O)₂—, or —S(O)₂NH—, and L₂ is an optionally substituted C₁₋₆ alkyl, an optionally substituted C₁₋₆heteroalkyl, an optionally substituted C₁₋₆ haloalkyl, an optionally substituted C₃₋₁₀ carbocyclyl, or an optionally substituted 4-10 membered heterocyclyl, preferably L₂ is C₄₋₈ carbocyclic which is monocyclic, or includes a spiro, bridged, and/or fused ring, and is unsubstituted or substituted with one or more groups independently selected from F, OH, and C₁₋₆ alkyl, or C₄₋₁₀ heterocyclyl, which is monocyclic, or includes a spiro, bridged, and/or fused ring, and is unsubstituted or substituted with one or more groups independently selected from F, OH, and C₁₋₆ alkyl and containing one, two or three heteroatoms independently selected from N, O and S.

In some embodiments,

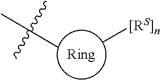

is selected from the following groups which are further optionally substituted:

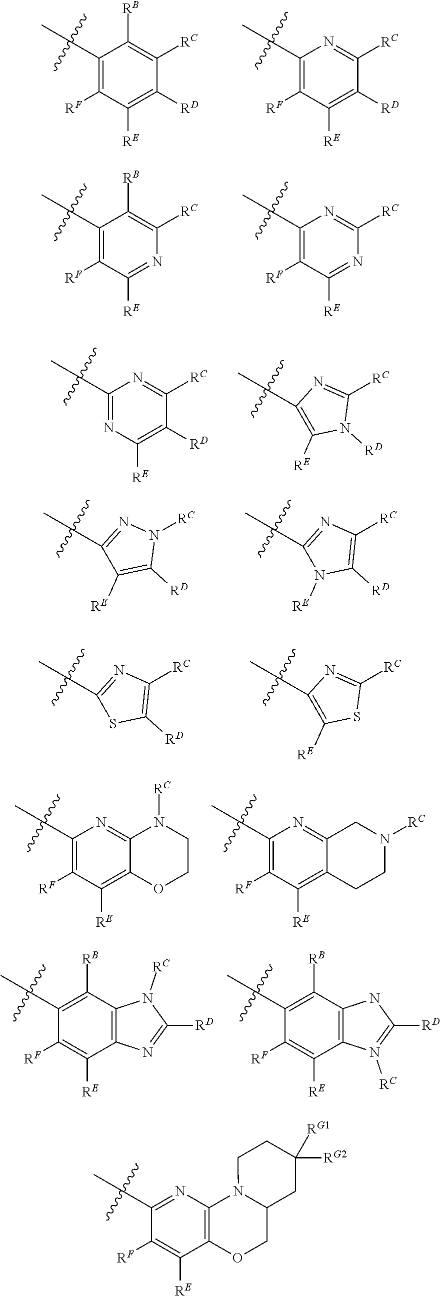

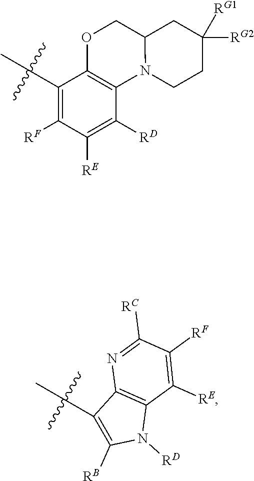

wherein:
each of R^{B}, R^{D}, R^{E}, R^{F}, R^{G1}, and R^{G2} are independently hydrogen, halogen, CN, OH, NH₂, an optionally substituted C₁₋₆ alkyl, an optionally substituted C₁₋₄ heteroalkyl, or an optionally substituted C₃₋₆ cycloalkyl;
R^{C} is an optionally substituted C₁₋₆ alkyl, an optionally substituted C₁₋₆ heteroalkyl, or an optionally substituted C₆ haloalkyl; or
R^{C} is selected from the following groups which are further optionally substituted:

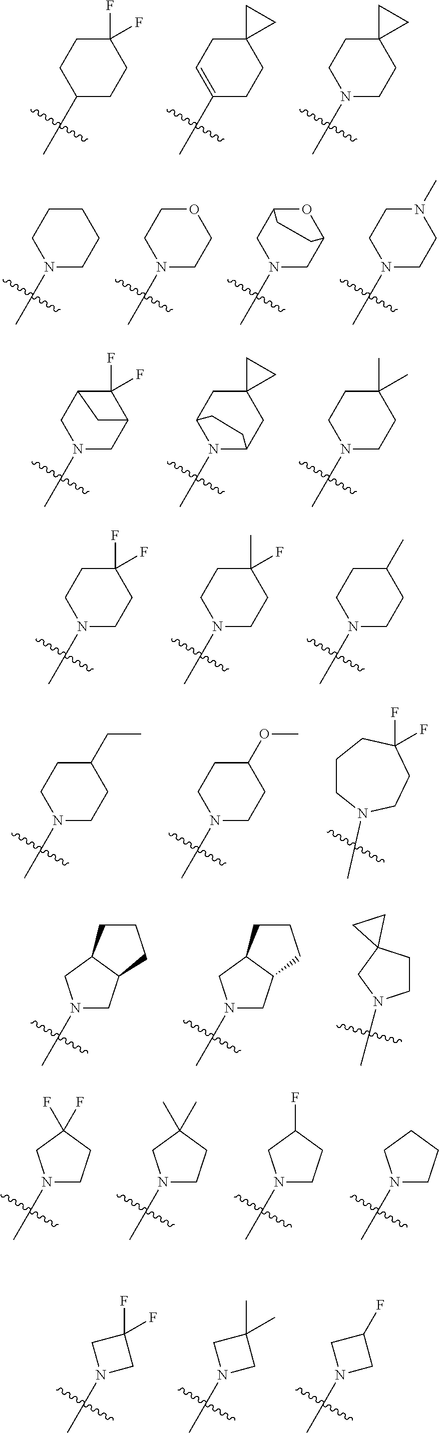

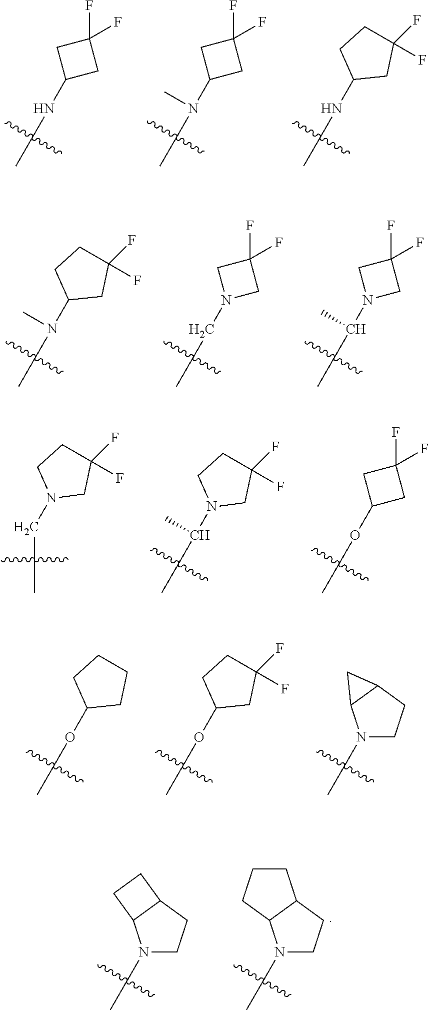

In some embodiments,

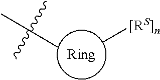

is selected from the following groups which are further optionally substituted:

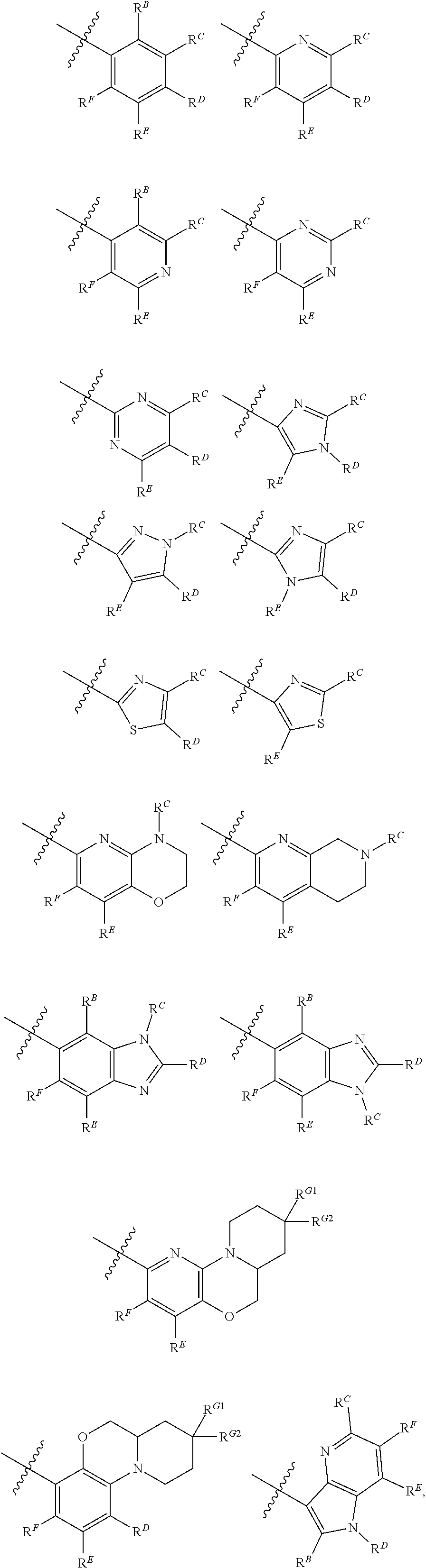

wherein:
each of R^{B}, R^{D}, R^{E}, R^{F}, R^{G1}, and R^{G2} are independently hydrogen, halogen, CN, OH, NH₂, an optionally substituted C₁₋₆ alkyl, an optionally substituted C₁₋₄heteroalkyl, or an optionally substituted C₃₋₆ cycloalkyl;
R^{C} is selected from the following groups which are further optionally substituted:

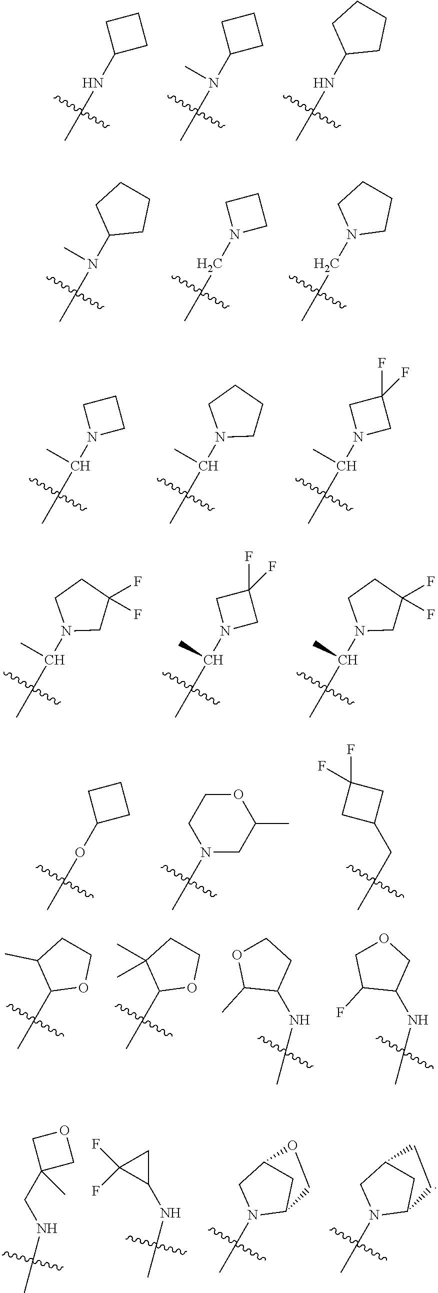

In some embodiments,

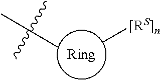

is

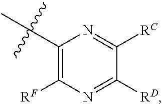

wherein the variables R^{C}, R^{D}, and R^{F} include any of those described herein in any combination. In some embodiments,

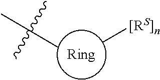

is

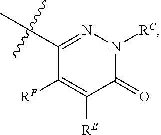

wherein the variables R^{C}, R^{E}, and R^{F} include any of those described herein in any combination.

In some embodiments, R^{B}, R^{D}, R^{E}, and R^{F} are each independently H, F, Cl, CN, CH₃, CH₂CH₃, CHF₂, CF₃, CH₂CF₃, OCH₃, OCH₂CH₃, O—CH(CH₃)₂, OCHF₂, OCF₃, OCH₂CF₃, OCH₂CH₂CF₃, or SCF₃. In some embodiments, R^{B} is hydrogen. In some embodiments, both R^{E} and R^{F} are hydrogen. In some embodiments, both R^{G1} and R^{G2} are Cl or F. In some embodiments, R^{C} is CH₃, CH₂CF₃, CH₂CH₂CF₃, OCF₃, OCH₂CF₃, OCH₂CH₂CF₃, or CF₃; or R^{C} is selected from:

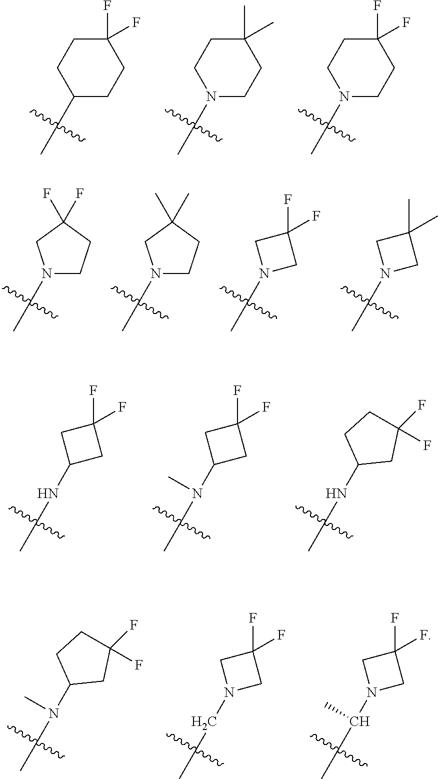

In some embodiments, R^{C} is selected from:

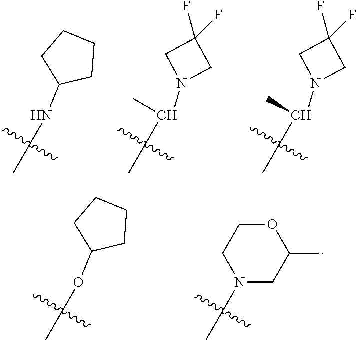

In some embodiments, R^{C} is NHCH₂CH₂CF₃, or selected from:

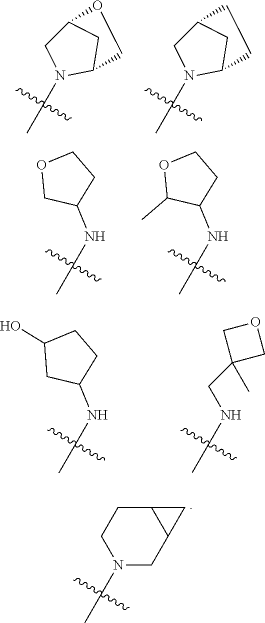

In some embodiments, R^{C} is selected from:

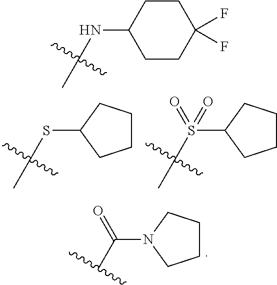

In some embodiments, R^{C} is selected from:

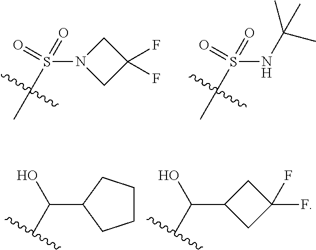

In some embodiments, the compound of Formula I-1-A can be characterized by having Formula I-1-A-a, I-1-A-b, I-1-A-c, I-1-A-d, I-1-A-e, I-1-A-f, I-1-A-g, or I-1-A-h:

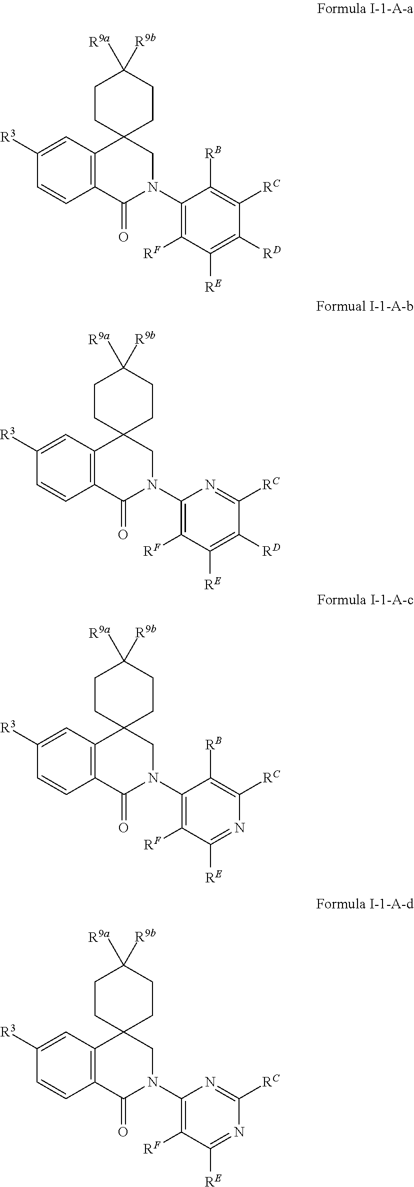

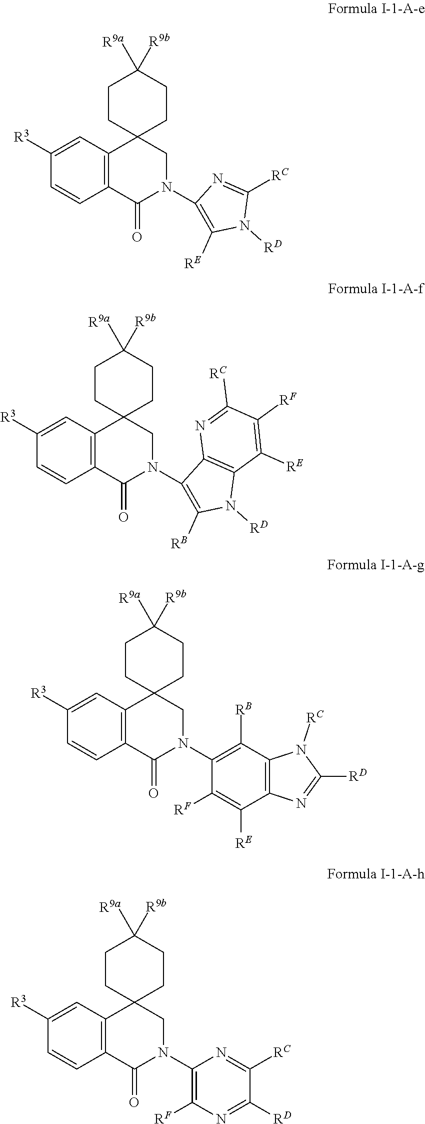

wherein the variables R³, R^{9a}, R^{9b}, R^{B}, R^{C}, R^{D}, R^{E}, and R^{F} include any of those described herein in any combination.

In Formula I-1-A (e.g., Formula I-1-A-a, I-1-A-b, I-1-A-c, I-1-A-d, I-1-A-e, I-1-A-f, I-1-A-g, or I-1-A-h), R^{9a} and R^{9b} can be selected as follows. In some embodiments, R^{9a} and R^{9b} are each independently hydrogen, halogen, an optionally substituted C₁₋₆ alkyl, or an optionally substituted C₁₋₆ heteroalkyl. In some embodiments, R^{9a} and R^{9b} are each independently H, F, Cl, methyl, ethyl, fluoromethyl, difluoromethyl, trifluoromethyl, methoxy, ethoxy, CF₃O—, or CF₃CH₂O—. In some embodiments, R^{9a} and R^{9b} are each independently H, F, Cl, methyl, ethyl, CD₃, trifluoromethyl, or CF₃O—. In some embodiments, both R^{9a} and R^{9b} are F or methyl, or R^{9a} is F and R^{9b} is methyl. In some embodiments, R^{9a} and R^{9b}, together with the intervening C atom, are joined to form an optionally substituted C₃₋₆ carbocyclyl, or an optionally substituted 4-6 membered heterocyclyl. In some embodiments, R^{9a} and R^{9b}, together with the intervening C atom, are joined to form an optionally substituted cyclopropyl or cyclobutyl, preferably cyclopropyl. In some embodiments, R^{9a} and R^{9b} are joined to form ═CF₂, ═CCl₂, or ═C(CH₃)₂. In some embodiments, R^{9a} and R^{9b} are joined to form ═CF₂.

In Formula I-1-A (e.g., Formula I-1-A-a, I-1-A-b, I-1-A-c, I-1-A-d, I-1-A-e, I-1-A-f, I-1-A-g, or I-1-A-h), R³ can be selected as follows. In some embodiments, R³ is —CONHR^{A}, —S(O)₂NHR^{A}, —NHCOR^{A}, or —NHS(O)₂R^{A}, wherein R^{A} is a C₂₋₄ alkyl optionally substituted with F, OH, or NH₂; or R³ is selected from:

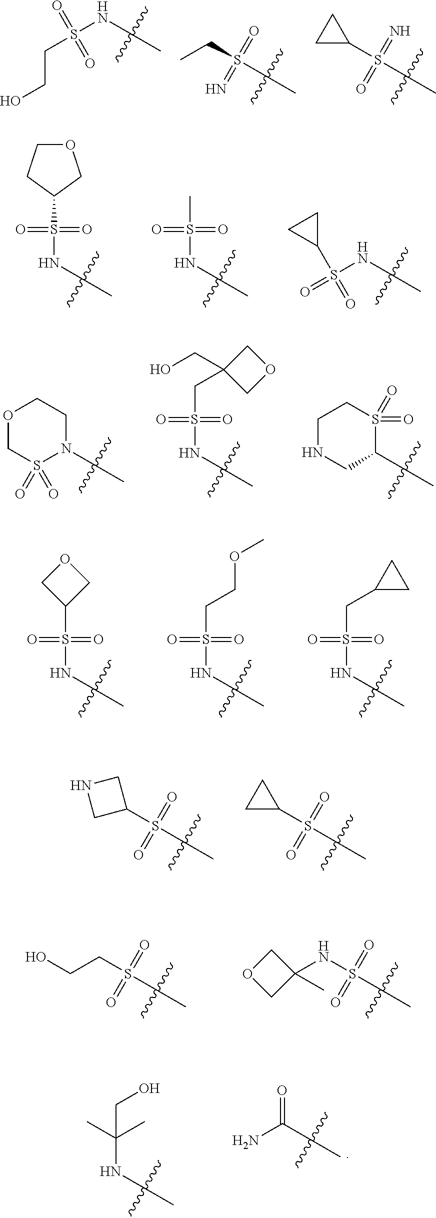

In some embodiments, R³ is selected from:

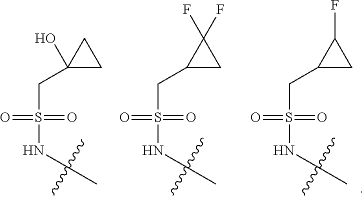 [SMILES: *NS(=O)(=O)CC1(O)CC1 ; *NS(=O)(=O)CC1CC1(F)F ; *NS(=O)(=O)CC1CC1F]

In more preferred embodiments, R³ is —NHS(O)₂R^{A}, wherein R^{A} is —CH₂CH₂OH or —CH₂CH₂CH₂OH. In more preferred embodiments, R³ is

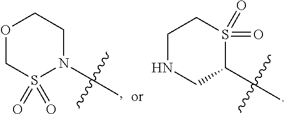

In some embodiments, R³ is —NHS(O)₂R^{A}, wherein R^{A} is —CH₂CH₃. In some embodiments, R³ is —NHS(O)₂R^{A}, wherein R^{A} is —CH₂CH₂N(CH₃)₂. In some embodiments, R³ is —NHS(O)₂R^{A}, wherein R^{A} is —CH₂CH₂OH. In some embodiments, R³ is —NHS(O)₂R^{A}, wherein R^{A} is —CH₂CH₂O-G^{X}, wherein G^{X} represents a group such that —CH₂CH₂O-G^{X} can be converted into —CH₂CH₂OH in vivo when the compound having the —CH₂CH₂O-G^{X} as R^{A} is administered to a subject, such as a mammal, preferably a human. In some embodiments, G^{X} is an acyl group as defined herein, for example, an acyl group represented by —C(O)-G^{X1}, wherein G^{X1} is a C₁₋₂₀ aliphatic group (e.g., alkyl, alkenyl, carbocyclic, etc.), an aromatic ring, or a 4-14 membered heterocyclic structure, each of which is optionally substituted. In some embodiments, G^{X} is an amino acyl group, which broadly refers to an acyl group herein that is substituted with one or more amino groups (e.g., one or more groups selected from NH₂, monoalkyl amino group, or dialkylamino group). For example, in some embodiments, the moiety of —CH₂CH₂O-G^{X} may be viewed as an ester of —CH₂CH₂OH and an amino acid, such as a natural amino acid (e.g., L-valine) or a non-natural amino acid, or a peptide such as a dipeptide, tripeptide, or tetrapeptide. In some embodiments, R³ is —NHS(O)₂R^{A}, wherein R^{A} is —CH₂CH₂OC(O)R″, wherein R″ is an optionally substituted C₁₋₆ alkyl, an optionally substituted C₂₋₆ alkenyl, an optionally substituted C₂₋₆ alkynyl, an optionally substituted C₁₋₆ heteroalkyl, or an optionally substituted C₃₋₆ cycloalkyl. For example, in some embodiments, R³ is —NHS(O)₂R^{A}, wherein R^{A} is —CH₂CH₂OC(O)CH(NH₂)CH(CH₃)₂. In some embodiments, R³ is —NHS(O)₂R^{A}, wherein R^{A} is a C₁₋₄ alkyl optionally substituted with F, such as CF₃ or CH₂CF₃. In some preferred embodiments, R³ is

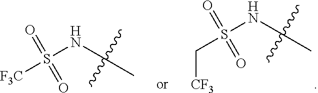 [SMILES: *NS(=O)(=O)C(F)(F)F ; *NS(=O)(=O)CC(F)(F)F]

In some embodiments, R³ is —NHS(O)₂R^{A}, wherein R^{A} is NH—C₁₋₄ alkyl or 4-membered heterocyclyl, which is optionally substituted with F and/or OH, such as NH—CH₃ or

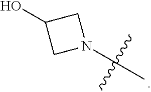 [SMILES: *N1CC(O)C1]

In some preferred embodiments, R³ is

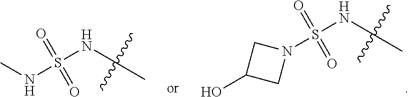

In some embodiments, R³ is S(O)₂R^{A}, wherein R^{A} is C₁₋₄ alkyl optionally substituted with F and/or OH, such as CH₃. In some preferred embodiments, R³ is

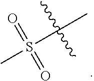 [SMILES: *S(C)(=O)=O]

In some embodiments, R³ is NHR^{A}, wherein R^{A} is C₁₋₆ alkyl optionally substituted with F and/or OH, such as C(CH₃)₂CH₂OH. In some preferred embodiments, R³ is

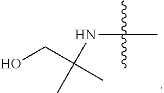 [SMILES: *NC(C)(C)CO]

In Formula I-1-A-a, I-1-A-c, I-1-A-f, or I-1-A-g, R^{B} can be selected as follows. In some embodiments, R^{B} is hydrogen, halogen, CN, OH, NH₂, an optionally substituted C₁₋₆ alkyl, an optionally substituted C₁₋₄ heteroalkyl, or an optionally substituted C₃₋₆ cycloalkyl. In some embodiments, R^{B} is selected from H, halogen, CN, C₁₋₃ alkyl, or C₁₋₃ alkoxy; preferably H, F, Cl, CN, CH₃, or OCH₃. In some embodiments, R^{B} is hydrogen. In some embodiments, R^{B} is F.

In Formula I-1-A-a, I-1-A-b, I-1-A-c, I-1-A-d, I-1-A-e, I-1-A-f, I-1-A-g, or I-1-A-h, R^{C} can be selected as follows. In some embodiments, R^{C} is -L₁-L₂, wherein Li is null, —O—, —NH—, —N(C₁₋₆ alkyl)-, —CH₂—, —CH(C₁₋₆ alkyl)-, —CH(OH)—, —C(O)—, —S—, —S(O)₂—, or —S(O)₂NH—, and L₂ is an optionally substituted C₁₋₆ alkyl, an optionally substituted C₁₋₆ heteroalkyl, an optionally substituted C₁₋₆ haloalkyl, an optionally substituted C₃₋₁₀ carbocyclyl, or an optionally substituted 4-10 membered heterocyclyl, preferably L₂ is C₄₈ carbocyclyl, which is monocyclic, or includes a spiro, bridged, and/or fused ring, and is unsubstituted or substituted with one or more groups independently selected from F, OH, and C₁₋₆ alkyl, or C₄₋₁₀ heterocyclyl which is monocyclic, or includes a spiro, bridged, and/or fused ring, and is unsubstituted or substituted with one or more groups independently selected from F, OH, and C₁₋₆ alkyl and containing one, two or three heteroatoms independently selected from N, O and S. In some embodiments, R^{C} is an optionally substituted C₁₋₆ alkyl, an optionally substituted C₁₋₆ heteroalkyl, or an optionally substituted C₁₋₆ haloalkyl. In some embodiments, R^{c} is selected from the following groups which are further optionally substituted:

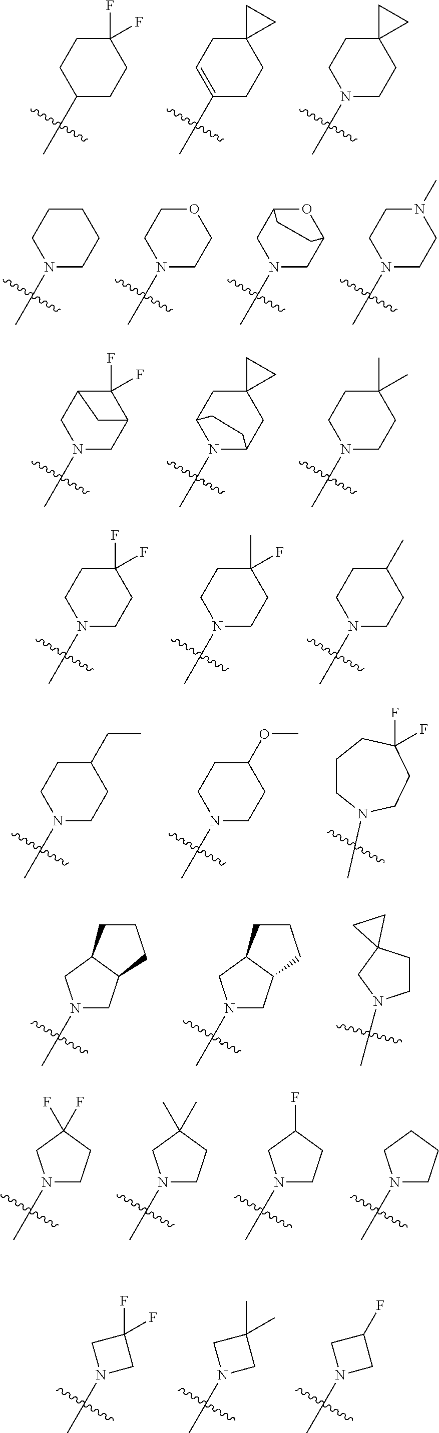

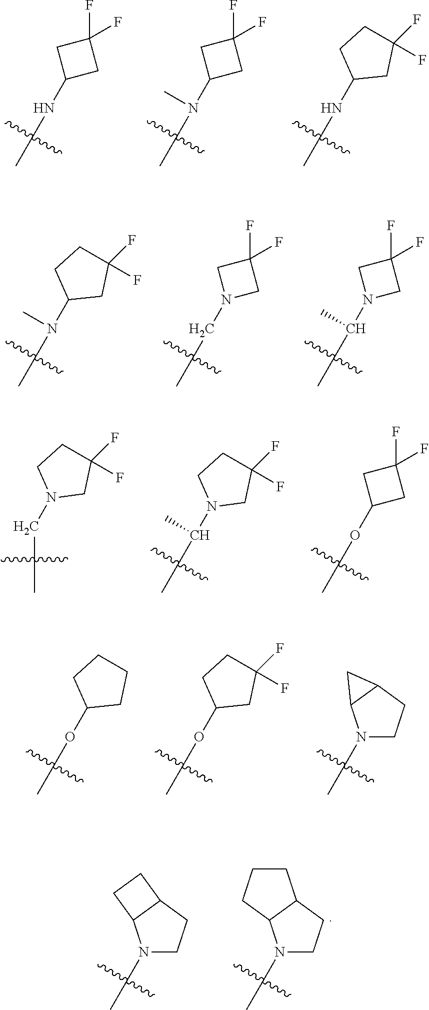

In some embodiments, R^{C} is selected from the following groups which are further optionally

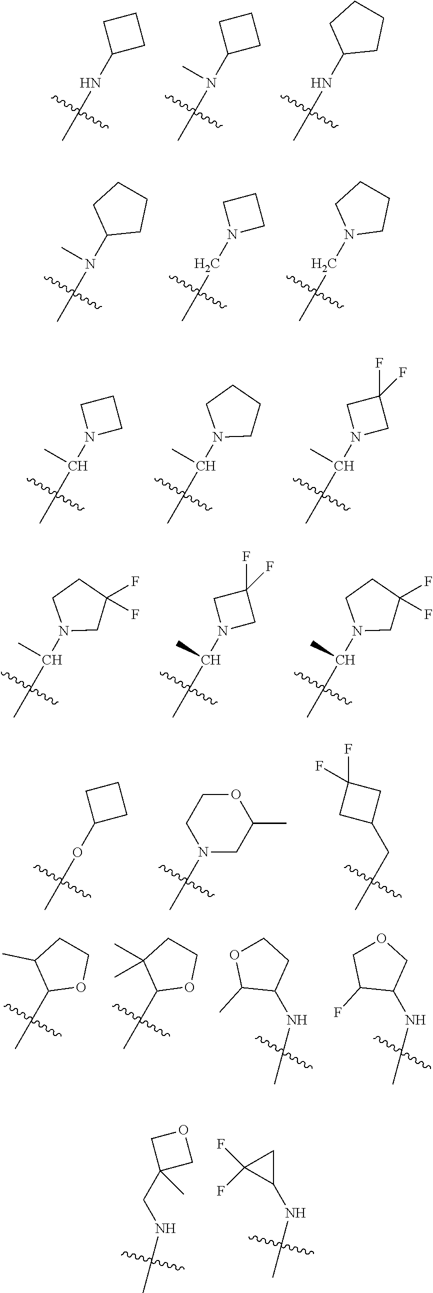

substituted:

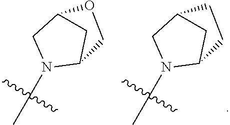 [SMILES: *N1C[C@H]2C[C@@H]1CO2 ; *N1C[C@@H]2CC[C@H]1C2]

In some embodiments, R^{c} is CH₃, CH₂CF₃, CH₂CH₂CF₃, OCF₃, OCH₂CF₃, OCH₂CH₂CF₃, or CF₃. In some embodiments, R^{C} is selected from the following groups which are further optionally substituted:

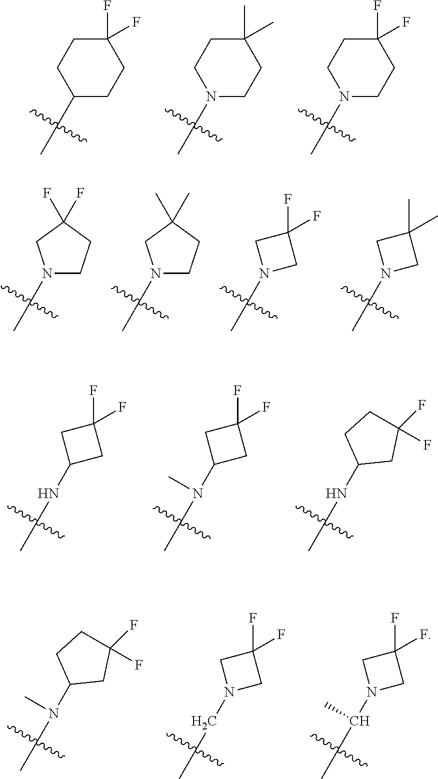

In some embodiments, R^{C} is selected from the following groups which are further optionally substituted:

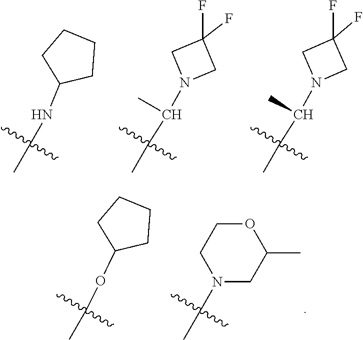

In some embodiments, R^{C} is NHCH₂CH₂CF₃, or selected from the following groups which are further optionally substituted:

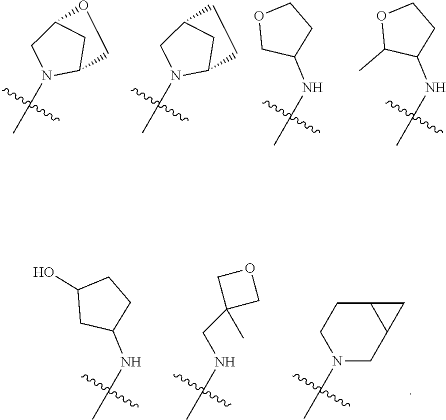

In some embodiments, R^{C} is selected from the following groups which are further optionally substituted:

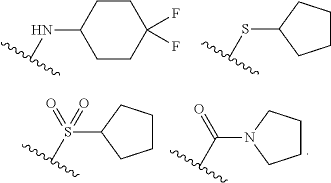

In some embodiments, R^{C} is selected from the following groups which are further optionally substituted:

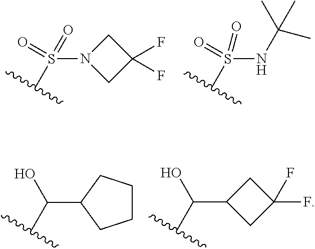

In some embodiments, R^{C} is

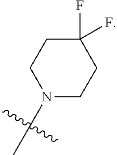 [SMILES: *N1CCC(F)(F)CC1]

In Formula I-1-A-a, I-1-A-b, I-1-A-e, I-1-A-f, I-1-A-g, or I-1-A-h, R^{D} can be selected as follows. In some embodiments, R^{D} is hydrogen, halogen, CN, OH, NH₂, C₁₋₆ alkyl, O(C₁₋₆ alkyl), O(CH₂)₁₋₃O(C₁₋₆ alkyl), O(CH₂)₁₋₃NH(C₁₋₆ alkyl), O(CH₂)₁₋₃N(C₁₋₆ alkyl)(C₁₋₆ alkyl), NH(C₁₋₆ alkyl), N(C₁₋₆ alkyl)(C₁₋₆ alkyl), C(O)NH₂, C(O)NH(C₁₋₆ alkyl), C(O)N(C₁₋₆ alkyl)(C₁₋₆ alkyl), an optionally substituted C₃₋₆ cycloalkyl, an optionally substituted 4-6 membered heterocyclyl, an optionally substituted phenyl, or an optionally substituted 5-6 membered heteroaryl, wherein C₁₋₆ alkyl is optionally substituted. In some embodiments, R^{D} is hydrogen, F, Cl, CN, OH, NH₂, C₁₋₆ alkyl, O(C₁₋₆ alkyl), O(CH₂)₁₋₃O(C₁₋₆ alkyl), O(CH₂)₁₋₃ NH(C₁₋₆ alkyl), O(CH₂)₁₋₃N(C₁₋₆ alkyl)(C₁₋₆ alkyl), NH(C₁₋₆ alkyl), N(C₁₋₆ alkyl)(C₁₋₆ alkyl), C(O)NH₂, C(O)NH(C₁₋₆ alkyl), C(O)N(C₁₋₆ alkyl)(C₁₋₆ alkyl), C₃₋₅ cycloalkyl, 4-6 membered heterocyclyl, phenyl, or 5-6 membered heteroaryl, wherein cycloalkyl, heterocyclyl, phenyl, or heteroaryl is unsubstituted or substituted with one or more groups independently selected from halogen (such as F or Cl) and C₁₋₆ alkyl, and C₁₋₆ alkyl is unsubstituted or substituted with F. In some embodiments, R^{D} is hydrogen, F, Cl, CN, OH, NH₂, C₁₋₆ alkyl, O(C₁₋₆ alkyl), O(CH₂)₁₋₃O(C₁₋₆ alkyl), O(CH₂)₁₋₃NH(C₁₋₆ alkyl), O(CH₂)₁₋₃N(C₁₋₆ alkyl)(C₁₋₆ alkyl), NH(C₁₋₆ alkyl), N(C₁₋₆ alkyl)(C₁₋₆ alkyl), C(O)NH₂, C(O)NH(C₁₋₆alkyl), C(O)N(C₁₋₆ alkyl)(C₁₋₆ alkyl), C₃₋₅ cycloalkyl, or 5-6 membered heteroaryl containing one, two or three ring nitrogen atoms, wherein cycloalkyl or heteroaryl is unsubstituted or substituted with one or more groups independently selected from halogen (such as F or Cl) and C₁₋₆ alkyl, and C₁₋₆ alkyl is unsubstituted or substituted with F. In some embodiments, R^{D} is hydrogen, halogen, CN, OH, NH₂, an optionally substituted C₁₋₆ alkyl, an optionally substituted C₁₋₄ heteroalkyl, or an optionally substituted C₃₋₆ cycloalkyl. In some embodiments, R^{D} is selected from H, halogen, CN, C₁₋₃ alkyl, or C₁₋₃ alkoxy; preferably H, F, Cl, CN, CH₃, or OCH₃. In some preferred embodiments, R^{D} is H, F, Cl, OH, CN, CH₃, OCH₃, CHF₂, CF₃, OCHF₂, CH₂CH₃, N(CH₃)₂, C(O)NH₂, C(O)NHCH₃, C(O)NHCH₂CH₃, OCH₂CH₂OCH₃, OCH₂CH₂N(CH₃)₂, or cyclopropyl. In some embodiments, R^{D} is 5-membered heteroaryl containing one, two or three ring nitrogen atoms and optionally substituted with C₁₋₂ alkyl, such as pyrazolyl optionally substituted with methyl, imidazolyl optionally substituted with methyl, or triazolyl optionally substituted with methyl. In some preferred embodiments, R^{D} is selected from

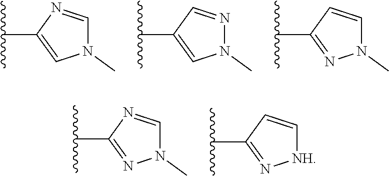

In some embodiments, R^{D} is 6-membered heteroaryl containing one or two ring nitrogen atoms and optionally substituted with C₁₋₂ alkyl, such as pyridinyl optionally substituted with methyl or pyrimidinyl optionally substituted with methyl. In some preferred embodiments, R^{D} is selected from

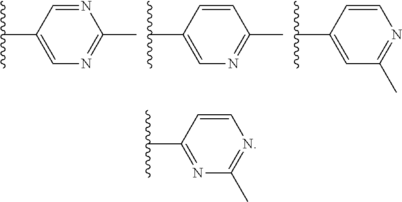

In some embodiments, R^{D} is 5-6 membered heterocyclyl containing one or two ring nitrogen atoms and optionally substituted with oxo and/or C₁₋₂ alkyl, such as pyrrolidinyl optionally substituted with oxo, piperidinyl optionally substituted with methyl, piperazinyl optionally substituted with oxo and methyl, or tetrahydropyridinyl optionally substituted with methyl. In some preferred embodiments, R^{D} is selected from

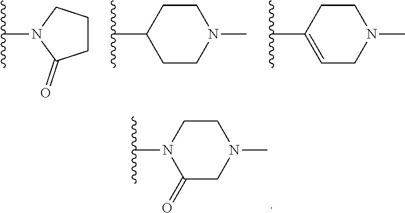

In some embodiments, R^{D} is C(O)-(5-6 membered heterocyclyl), wherein the heterocyclyl contains one or two ring nitrogen atoms and optionally substituted with C₁₋₂ alkyl, such as C(O)-piperazinyl optionally substituted with methyl. In some preferred embodiments, R^{D} is

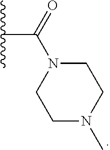 [SMILES: *C(=O)N1CCN(C)CC1]

In Formula I-1-A-a, I-1-A-b, I-1-A-c, I-1-A-d, I-1-A-e, I-1-A-f, or I-1-A-g, R^{E} can be selected as follows. In some embodiments, R^{E} is hydrogen, halogen, CN, OH, NH₂, an optionally substituted C₁₋₆ alkyl, an optionally substituted C₁₋₄ heteroalkyl, or an optionally substituted C₃₋₆ cycloalkyl. In some embodiments, R^{E} is hydrogen.

In Formula I-1-A-a, I-1-A-b, I-1-A-c, I-1-A-d, I-1-A-f, I-1-A-g, or I-1-A-h, R^{E} can be selected as follows. In some embodiments, R^{F} is hydrogen, halogen, CN, OH, NH₂, an optionally substituted C₁₋₆ alkyl, an optionally substituted C₁₋₄ heteroalkyl, or an optionally substituted C₃₋₆ cycloalkyl. In some embodiments, R^{E} is hydrogen.

In some embodiments, the compound of Formula I-2 can be characterized by having Formula T-2-A, T-2-B, T-2-C, I-2-D, or T-2-E:

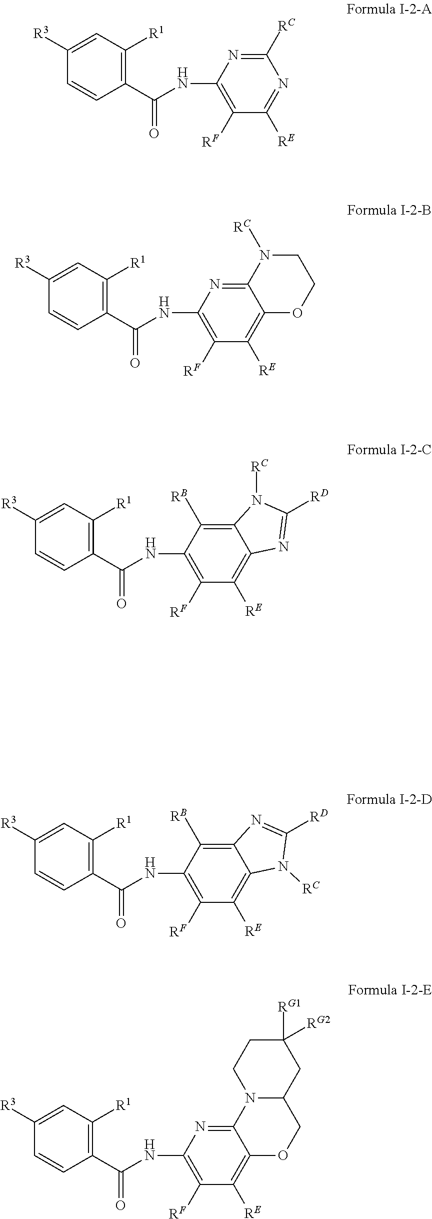

wherein the variables R¹, R³, R^{B}, R^{C}, R^{D}, R^{F}, R^{F}, R^{G1}, and R^{G2} include any of those described herein in any combination.

In some embodiments, in Formula I, specifically in Formula I-2 (e.g. in Formula I-2-A, I-2-B, I-2-C, I-2-D, or I-2-E), R¹ is selected from the following groups which are further optionally substituted:

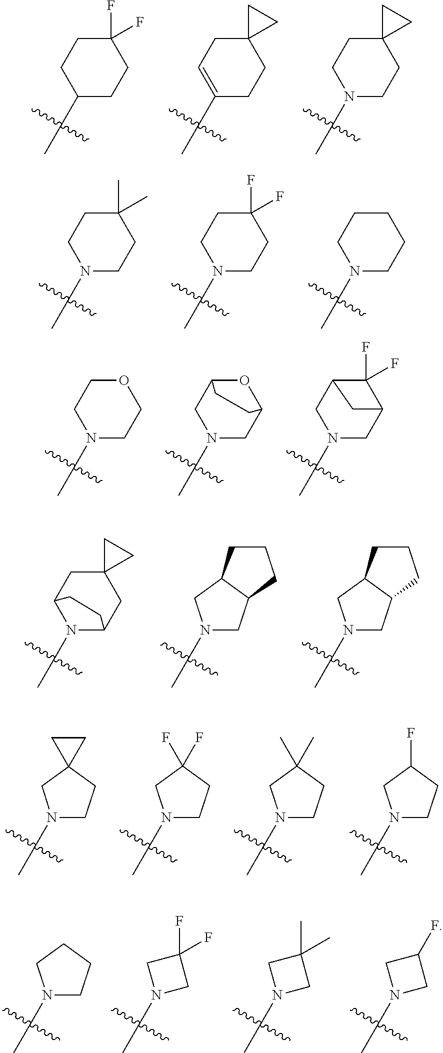

In some preferred embodiments, R¹ is selected from:

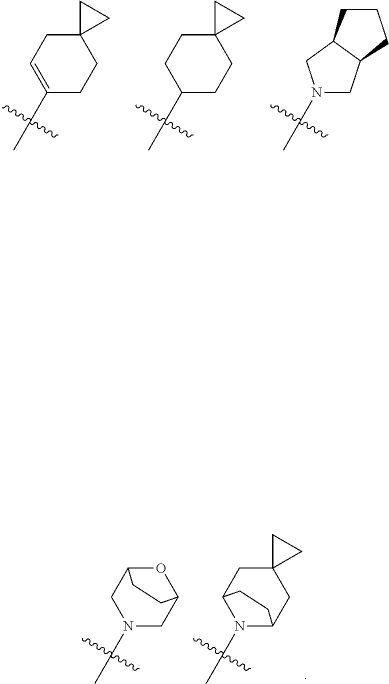

In some embodiments, R³ is —CONHR^{A}, —S(O)₂NHR^{A}, —NHCOR^{A}, or —NHS(O)₂R^{A}, wherein R^{A} is a C₂₋₄ alkyl optionally substituted with F, OH, or NH₂; or R³ is selected from:

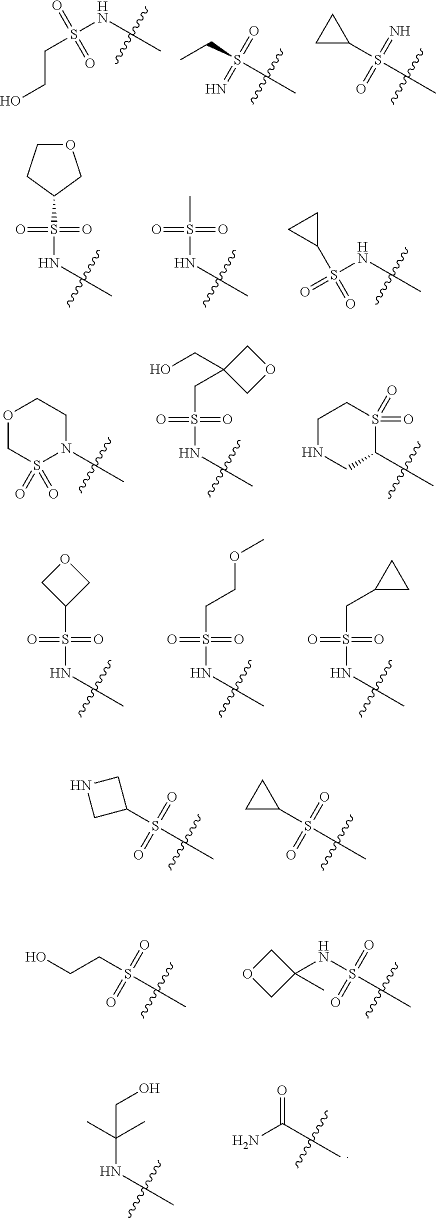

In more preferred embodiments, R³ is —NHS(O)₂R^{A}, wherein R^{A} is —CH₂CH₂OH or —CH₂CH₂CH₂OH. In more preferred embodiments, R³ is

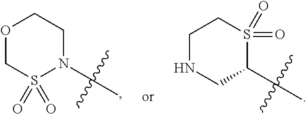

In some embodiments, R³ is —NHS(O)₂R^{A}, wherein R^{A} is —CH₂CH₃. In some embodiments, R³ is —NHS(O)₂R^{A}, wherein R^{A} is —CH₂CH₂N(CH₃)₂. In some embodiments, R³ is —NHS(O)₂R^{A}, wherein R^{A} is —CH₂CH₂OH. In some embodiments, R³ is —NHS(O)₂R^{A}, wherein R^{A} is —CH₂CH₂O-G^{X}, wherein G^{X} represents a group such that —CH₂CH₂O-G^{X} can be converted into —CH₂CH₂OH in vivo when the compound having the —CH₂CH₂O-G^{X} as R^{A} is administered to a subject, such as a mammal, preferably a human. In some embodiments, G^{X} is an acyl group as defined herein, for example, an acyl group represented by —C(O)-G^{X1}, wherein G^{X1} is a C₁₋₂₀ aliphatic group (e.g., alkyl, alkenyl, carbocyclic, etc.), an aromatic ring, or a 4-14 membered heterocyclic structure, each of which is optionally substituted. In some embodiments, G^{X} is an amino acyl group, which broadly refers to an acyl group herein that is substituted with one or more amino groups (e.g., one or more groups selected from NH₂, monoalkyl amino group, or dialkylamino group). For example, in some embodiments, the moiety of —CH₂CH₂O-G^{X} may be viewed as an ester of —CH₂CH₂OH and an amino acid, such as a natural amino acid (e.g., L-valine) or a non-natural amino acid, or a peptide such as a dipeptide, tripeptide, or tetrapeptide. In some embodiments, R³ is —NHS(O)₂R^{A}, wherein R^{A} is —CH₂CH₂OC(O)R″, wherein R″ is an optionally substituted C₁₋₆ alkyl, an optionally substituted C₂₋₆ alkenyl, an optionally substituted C₂₋₆ alkynyl, an optionally substituted C₁₋₆ heteroalkyl, or an optionally substituted C₃₋₆ cycloalkyl. For example, in some embodiments, R³ is —NHS(O)₂R^{A}, wherein R^{A} is —CH₂CH₂OC(O)CH(NH₂)CH(CH₃)₂.

In some embodiments, in Formula I-1-A-a, I-1-A-c, I-1-A-g, I-2-C, or I-2-D, R^{B} is selected from H, halogen, CN, C₁₋₃ alkyl, or C₁₋₃ alkoxy; preferably H, F, Cl, CN, CH₃, or OCH₃.

In some embodiments, in Formula I-1-A-a, I-1-A-b, I-1-A-g, or I-1-A-h, R^{D} is selected from H, halogen, CN, C₁₋₃ alkyl, or C₁₋₃ alkoxy; preferably H, F, Cl, CN, CH₃, or OCH₃.

In some embodiments, the present disclosure also provides a compound selected from those as shown in Table A, or a pharmaceutically acceptable salt thereof. In some embodiments, the present disclosure also provides a prodrug of a compound selected from those as shown in Table A, or a pharmaceutically acceptable salt thereof, e.g., an ester prodrug or an amino ester prodrug as described herein.

TABLE A
Exemplary compounds of the present disclosure

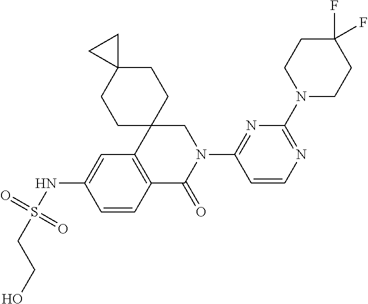 [SMILES: O=C1c2ccc(NS(=O)(=O)CCO)cc2C2(CCC3(CC3)CC2)CN1c1ccnc(N2CCC(F)(F)CC2)n1]

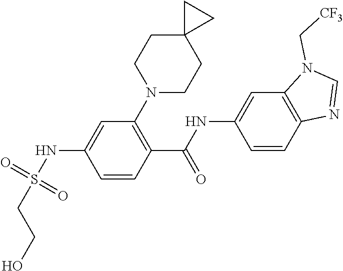 [SMILES: O=C(Nc1ccc2ncn(CC(F)(F)F)c2c1)c1ccc(NS(=O)(=O)CCO)cc1N1CCC2(CC1)CC2]

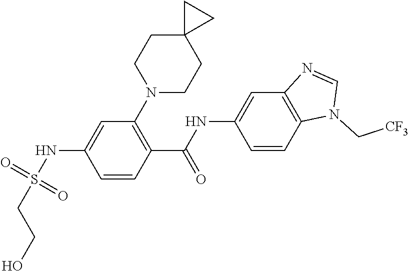 [SMILES: O=C(Nc1ccc2c(c1)ncn2CC(F)(F)F)c1ccc(NS(=O)(=O)CCO)cc1N1CCC2(CC1)CC2]

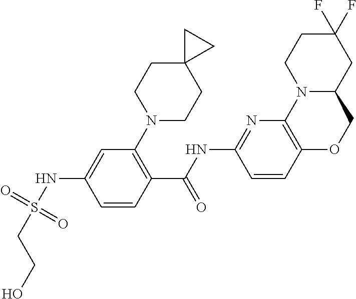 [SMILES: O=C(Nc1ccc2c(n1)N1CCC(F)(F)C[C@H]1CO2)c1ccc(NS(=O)(=O)CCO)cc1N1CCC2(CC1)CC2]

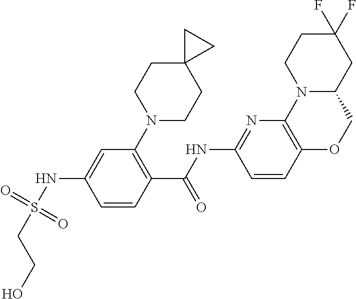 [SMILES: O=C(Nc1ccc2c(n1)N1CCC(F)(F)C[C@@H]1CO2)c1ccc(NS(=O)(=O)CCO)cc1N1CCC2(CC1)CC2]

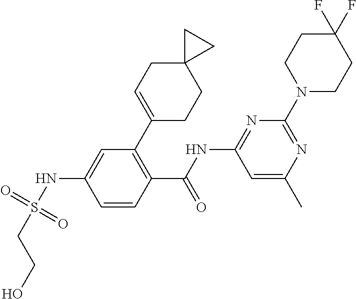 [SMILES: Cc1cc(NC(=O)c2ccc(NS(=O)(=O)CCO)cc2C2=CCC3(CC2)CC3)nc(N2CCC(F)(F)CC2)n1]

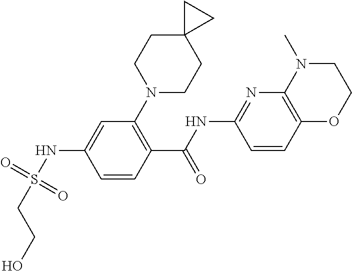 [SMILES: CN1CCOc2ccc(NC(=O)c3ccc(NS(=O)(=O)CCO)cc3N3CCC4(CC3)CC4)nc21]

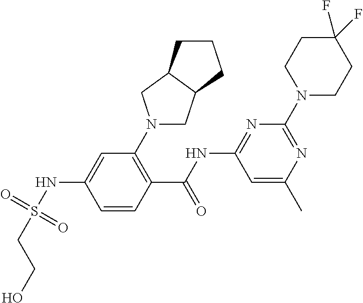 [SMILES: Cc1cc(NC(=O)c2ccc(NS(=O)(=O)CCO)cc2N2C[C@H]3CCC[C@H]3C2)nc(N2CCC(F)(F)CC2)n1]

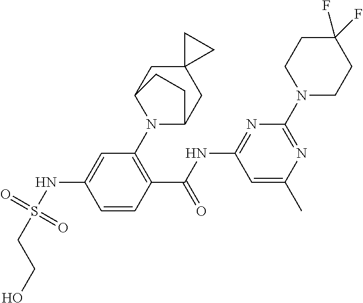 [SMILES: Cc1cc(NC(=O)c2ccc(NS(=O)(=O)CCO)cc2N2C3CCC2CC2(CC2)C3)nc(N2CCC(F)(F)CC2)n1]

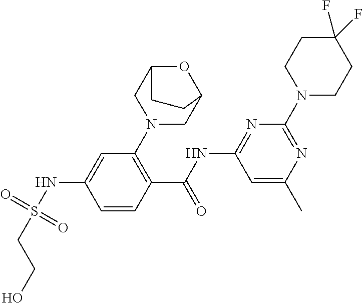 [SMILES: Cc1cc(NC(=O)c2ccc(NS(=O)(=O)CCO)cc2N2CC3CCC(C2)O3)nc(N2CCC(F)(F)CC2)n1]

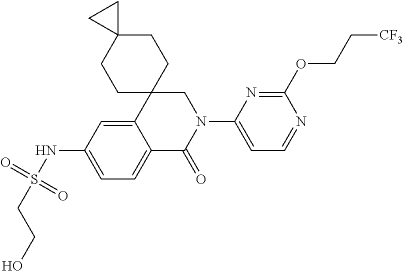 [SMILES: O=C1c2ccc(NS(=O)(=O)CCO)cc2C2(CCC3(CC3)CC2)CN1c1ccnc(OCCC(F)(F)F)n1]

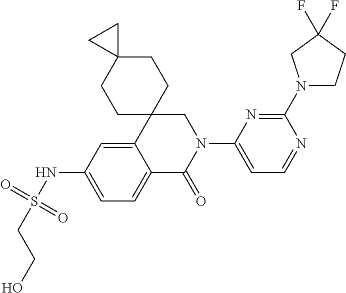 [SMILES: O=C1c2ccc(NS(=O)(=O)CCO)cc2C2(CCC3(CC3)CC2)CN1c1ccnc(N2CCC(F)(F)C2)n1]

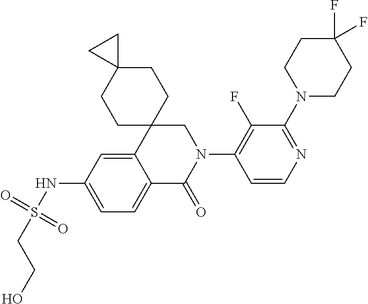 [SMILES: O=C1c2ccc(NS(=O)(=O)CCO)cc2C2(CCC3(CC3)CC2)CN1c1ccnc(N2CCC(F)(F)CC2)c1F]

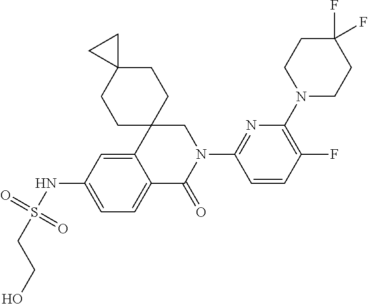 [SMILES: O=C1c2ccc(NS(=O)(=O)CCO)cc2C2(CCC3(CC3)CC2)CN1c1ccc(F)c(N2CCC(F)(F)CC2)n1]

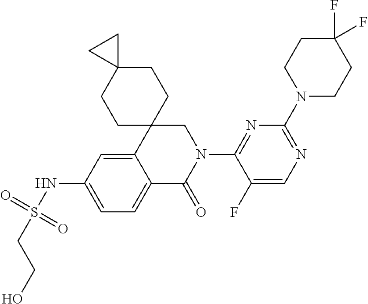 [SMILES: O=C1c2ccc(NS(=O)(=O)CCO)cc2C2(CCC3(CC3)CC2)CN1c1nc(N2CCC(F)(F)CC2)ncc1F]

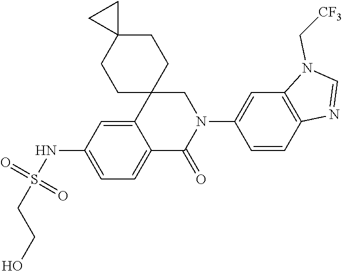 [SMILES: O=C1c2ccc(NS(=O)(=O)CCO)cc2C2(CCC3(CC3)CC2)CN1c1ccc2ncn(CC(F)(F)F)c2c1]

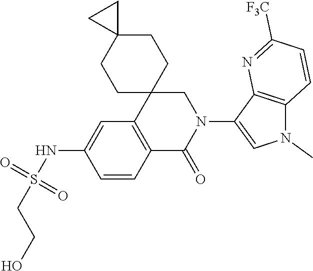 [SMILES: Cn1cc(N2CC3(CCC4(CC4)CC3)c3cc(NS(=O)(=O)CCO)ccc3C2=O)c2nc(C(F)(F)F)ccc21]

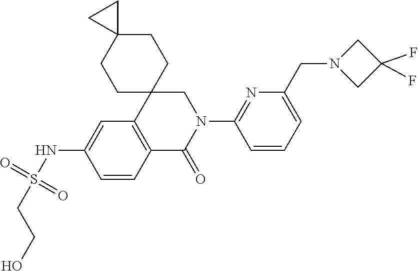 [SMILES: O=C1c2ccc(NS(=O)(=O)CCO)cc2C2(CCC3(CC3)CC2)CN1c1cccc(CN2CC(F)(F)C2)n1]

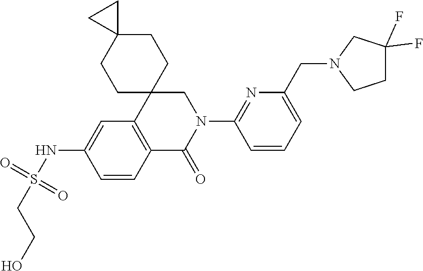 [SMILES: O=C1c2ccc(NS(=O)(=O)CCO)cc2C2(CCC3(CC3)CC2)CN1c1cccc(CN2CCC(F)(F)C2)n1]

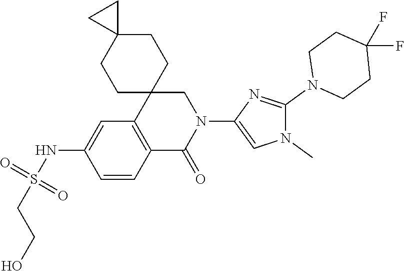 [SMILES: Cn1cc(N2CC3(CCC4(CC4)CC3)c3cc(NS(=O)(=O)CCO)ccc3C2=O)nc1N1CCC(F)(F)CC1]

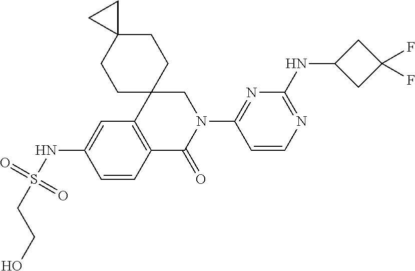 [SMILES: O=C1c2ccc(NS(=O)(=O)CCO)cc2C2(CCC3(CC3)CC2)CN1c1ccnc(NC2CC(F)(F)C2)n1]

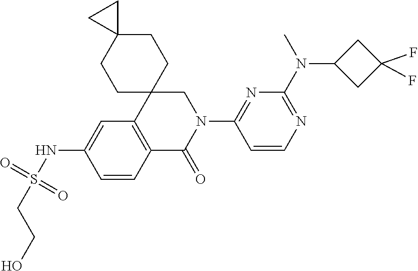 [SMILES: CN(c1nccc(N2CC3(CCC4(CC4)CC3)c3cc(NS(=O)(=O)CCO)ccc3C2=O)n1)C1CC(F)(F)C1]

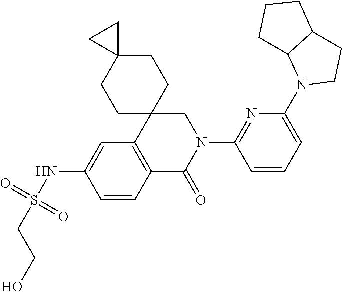 [SMILES: O=C1c2ccc(NS(=O)(=O)CCO)cc2C2(CCC3(CC3)CC2)CN1c1cccc(N2CCC3CCCC32)n1]

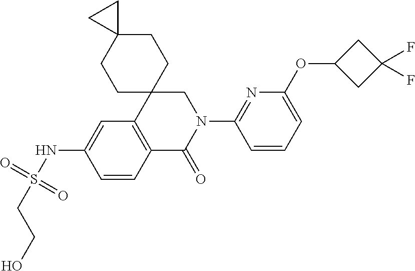 [SMILES: O=C1c2ccc(NS(=O)(=O)CCO)cc2C2(CCC3(CC3)CC2)CN1c1cccc(OC2CC(F)(F)C2)n1]

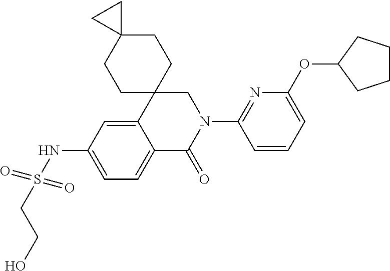 [SMILES: O=C1c2ccc(NS(=O)(=O)CCO)cc2C2(CCC3(CC3)CC2)CN1c1cccc(OC2CCCC2)n1]

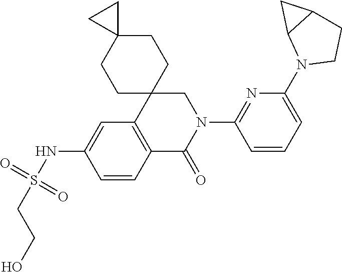 [SMILES: O=C1c2ccc(NS(=O)(=O)CCO)cc2C2(CCC3(CC3)CC2)CN1c1cccc(N2CCC3CC32)n1]

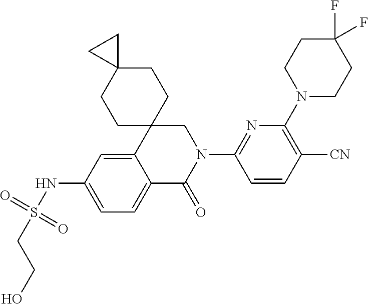 [SMILES: N#Cc1ccc(N2CC3(CCC4(CC4)CC3)c3cc(NS(=O)(=O)CCO)ccc3C2=O)nc1N1CCC(F)(F)CC1]

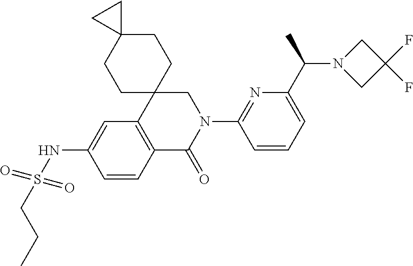 [SMILES: CCCS(=O)(=O)Nc1ccc2c(c1)C1(CCC3(CC3)CC1)CN(c1cccc([C@@H](C)N3CC(F)(F)C3)n1)C2=O]

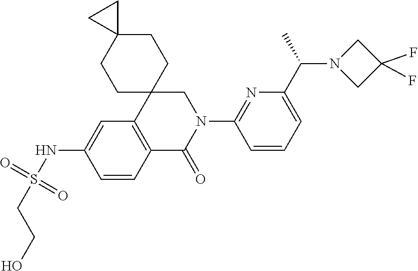 [SMILES: C[C@@H](c1cccc(N2CC3(CCC4(CC4)CC3)c3cc(NS(=O)(=O)CCO)ccc3C2=O)n1)N1CC(F)(F)C1]

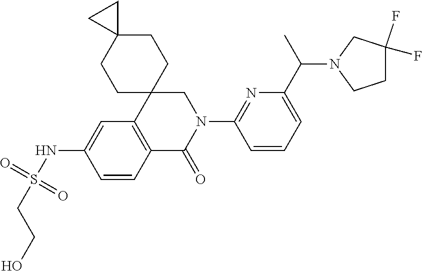 [SMILES: CC(c1cccc(N2CC3(CCC4(CC4)CC3)c3cc(NS(=O)(=O)CCO)ccc3C2=O)n1)N1CCC(F)(F)C1]

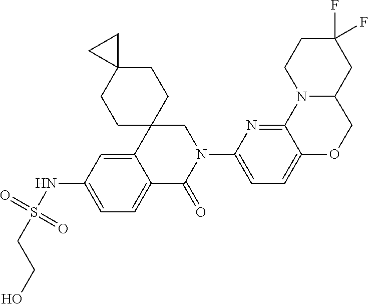 [SMILES: O=C1c2ccc(NS(=O)(=O)CCO)cc2C2(CCC3(CC3)CC2)CN1c1ccc2c(n1)N1CCC(F)(F)CC1CO2]

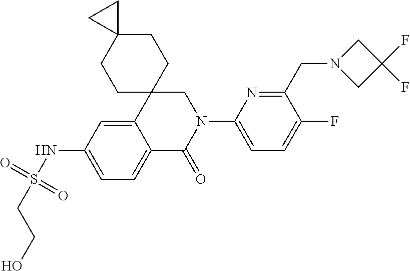 [SMILES: O=C1c2ccc(NS(=O)(=O)CCO)cc2C2(CCC3(CC3)CC2)CN1c1ccc(F)c(CN2CC(F)(F)C2)n1]

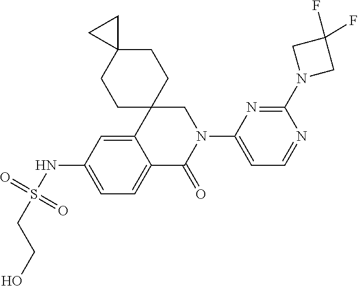 [SMILES: O=C1c2ccc(NS(=O)(=O)CCO)cc2C2(CCC3(CC3)CC2)CN1c1ccnc(N2CC(F)(F)C2)n1]

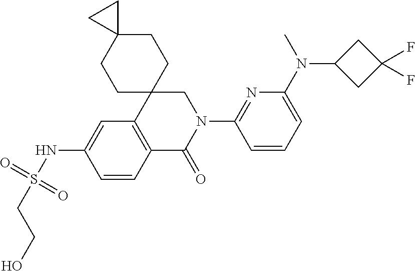 [SMILES: CN(c1cccc(N2CC3(CCC4(CC4)CC3)c3cc(NS(=O)(=O)CCO)ccc3C2=O)n1)C1CC(F)(F)C1]

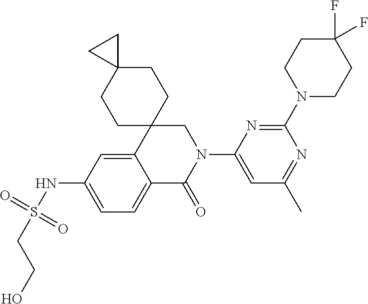 [SMILES: Cc1cc(N2CC3(CCC4(CC4)CC3)c3cc(NS(=O)(=O)CCO)ccc3C2=O)nc(N2CCC(F)(F)CC2)n1]

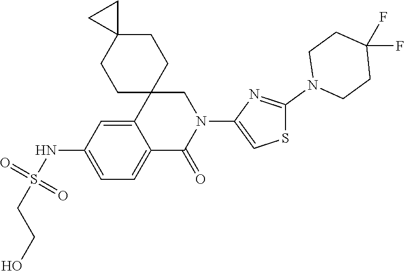 [SMILES: O=C1c2ccc(NS(=O)(=O)CCO)cc2C2(CCC3(CC3)CC2)CN1c1csc(N2CCC(F)(F)CC2)n1]

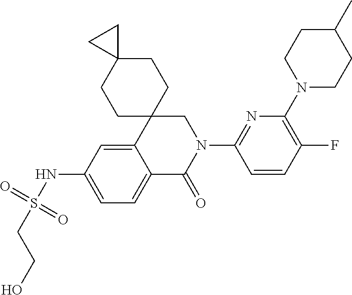 [SMILES: CC1CCN(c2nc(N3CC4(CCC5(CC5)CC4)c4cc(NS(=O)(=O)CCO)ccc4C3=O)ccc2F)CC1]

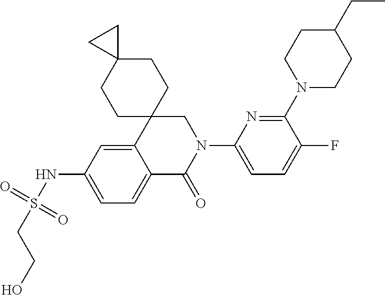 [SMILES: CCC1CCN(c2nc(N3CC4(CCC5(CC5)CC4)c4cc(NS(=O)(=O)CCO)ccc4C3=O)ccc2F)CC1]

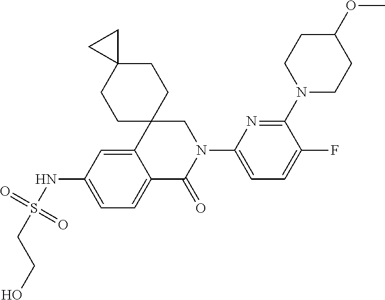 [SMILES: COC1CCN(c2nc(N3CC4(CCC5(CC5)CC4)c4cc(NS(=O)(=O)CCO)ccc4C3=O)ccc2F)CC1]

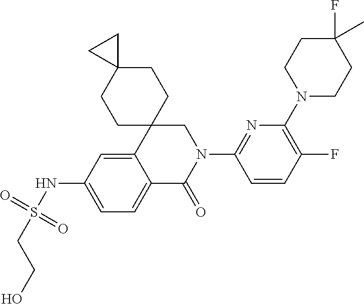 [SMILES: CC1(F)CCN(c2nc(N3CC4(CCC5(CC5)CC4)c4cc(NS(=O)(=O)CCO)ccc4C3=O)ccc2F)CC1]

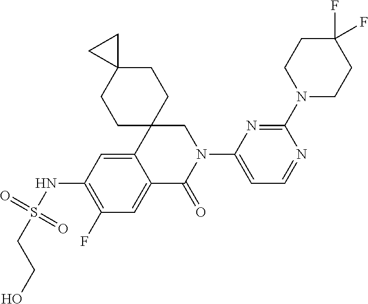 [SMILES: O=C1c2cc(F)c(NS(=O)(=O)CCO)cc2C2(CCC3(CC3)CC2)CN1c1ccnc(N2CCC(F)(F)CC2)n1]

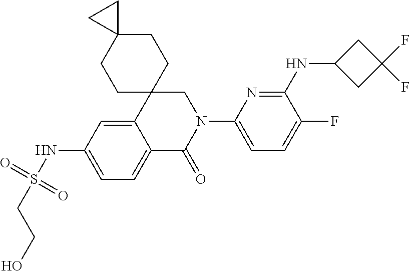 [SMILES: O=C1c2ccc(NS(=O)(=O)CCO)cc2C2(CCC3(CC3)CC2)CN1c1ccc(F)c(NC2CC(F)(F)C2)n1]

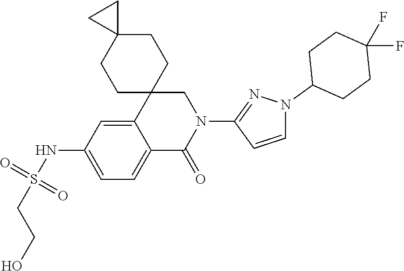 [SMILES: O=C1c2ccc(NS(=O)(=O)CCO)cc2C2(CCC3(CC3)CC2)CN1c1ccn(C2CCC(F)(F)CC2)n1]

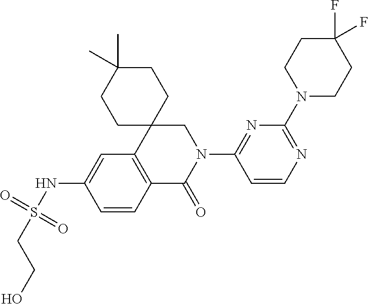 [SMILES: CC1(C)CCC2(CC1)CN(c1ccnc(N3CCC(F)(F)CC3)n1)C(=O)c1ccc(NS(=O)(=O)CCO)cc12]

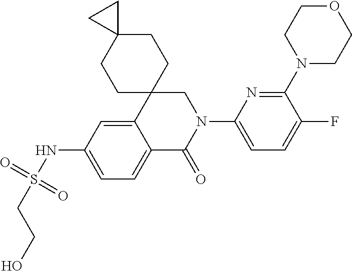 [SMILES: O=C1c2ccc(NS(=O)(=O)CCO)cc2C2(CCC3(CC3)CC2)CN1c1ccc(F)c(N2CCOCC2)n1]

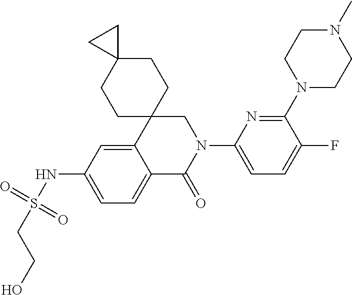 [SMILES: CN1CCN(c2nc(N3CC4(CCC5(CC5)CC4)c4cc(NS(=O)(=O)CCO)ccc4C3=O)ccc2F)CC1]

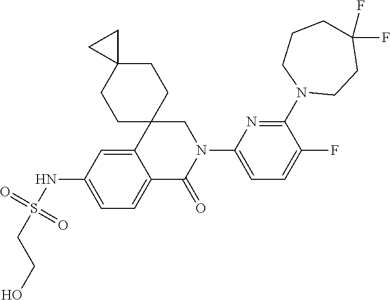 [SMILES: O=C1c2ccc(NS(=O)(=O)CCO)cc2C2(CCC3(CC3)CC2)CN1c1ccc(F)c(N2CCCC(F)(F)CC2)n1]

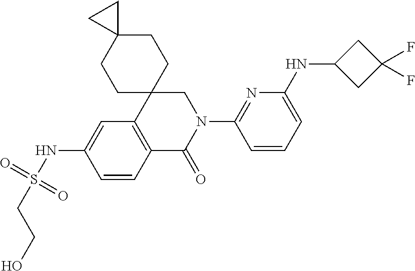 [SMILES: O=C1c2ccc(NS(=O)(=O)CCO)cc2C2(CCC3(CC3)CC2)CN1c1cccc(NC2CC(F)(F)C2)n1]

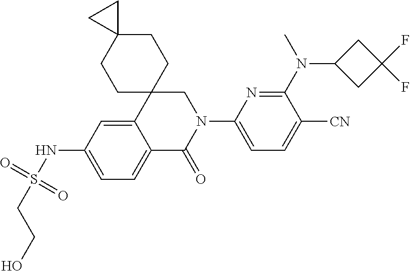 [SMILES: CN(c1nc(N2CC3(CCC4(CC4)CC3)c3cc(NS(=O)(=O)CCO)ccc3C2=O)ccc1C#N)C1CC(F)(F)C1]

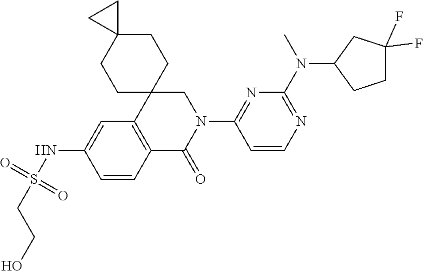 [SMILES: CN(c1nccc(N2CC3(CCC4(CC4)CC3)c3cc(NS(=O)(=O)CCO)ccc3C2=O)n1)C1CCC(F)(F)C1]

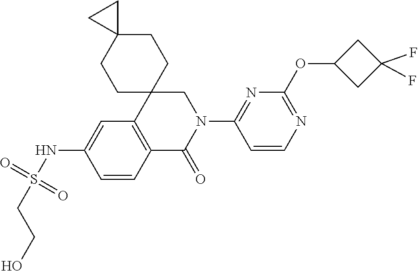 [SMILES: O=C1c2ccc(NS(=O)(=O)CCO)cc2C2(CCC3(CC3)CC2)CN1c1ccnc(OC2CC(F)(F)C2)n1]

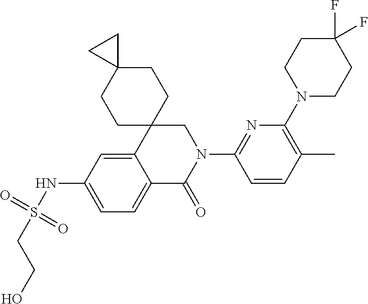 [SMILES: Cc1ccc(N2CC3(CCC4(CC4)CC3)c3cc(NS(=O)(=O)CCO)ccc3C2=O)nc1N1CCC(F)(F)CC1]

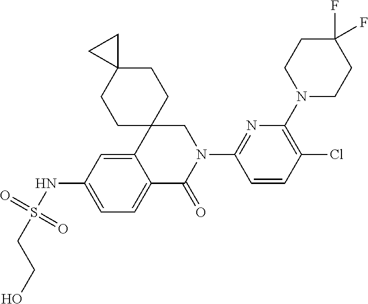 [SMILES: O=C1c2ccc(NS(=O)(=O)CCO)cc2C2(CCC3(CC3)CC2)CN1c1ccc(Cl)c(N2CCC(F)(F)CC2)n1]

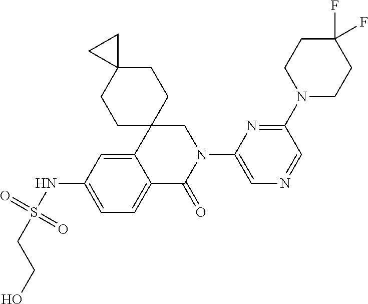 [SMILES: O=C1c2ccc(NS(=O)(=O)CCO)cc2C2(CCC3(CC3)CC2)CN1c1cncc(N2CCC(F)(F)CC2)n1]

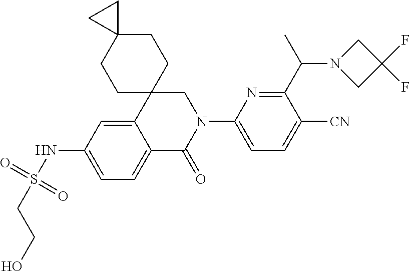 [SMILES: CC(c1nc(N2CC3(CCC4(CC4)CC3)c3cc(NS(=O)(=O)CCO)ccc3C2=O)ccc1C#N)N1CC(F)(F)C1]

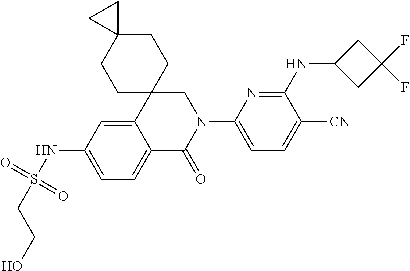 [SMILES: N#Cc1ccc(N2CC3(CCC4(CC4)CC3)c3cc(NS(=O)(=O)CCO)ccc3C2=O)nc1NC1CC(F)(F)C1]

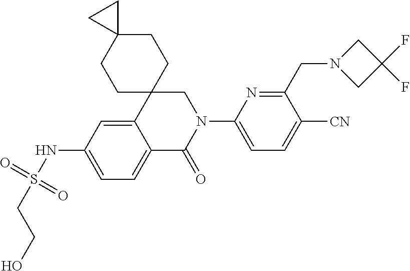 [SMILES: N#Cc1ccc(N2CC3(CCC4(CC4)CC3)c3cc(NS(=O)(=O)CCO)ccc3C2=O)nc1CN1CC(F)(F)C1]

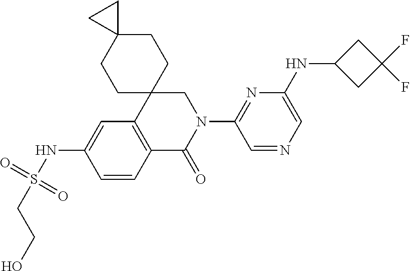 [SMILES: O=C1c2ccc(NS(=O)(=O)CCO)cc2C2(CCC3(CC3)CC2)CN1c1cncc(NC2CC(F)(F)C2)n1]

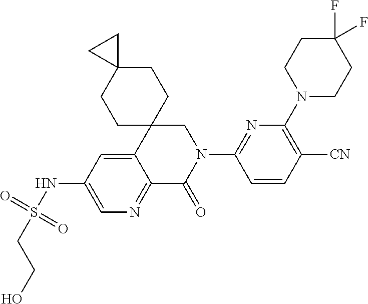 [SMILES: N#Cc1ccc(N2CC3(CCC4(CC4)CC3)c3cc(NS(=O)(=O)CCO)cnc3C2=O)nc1N1CCC(F)(F)CC1]

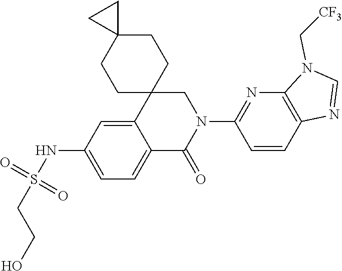 [SMILES: O=C1c2ccc(NS(=O)(=O)CCO)cc2C2(CCC3(CC3)CC2)CN1c1ccc2ncn(CC(F)(F)F)c2n1]

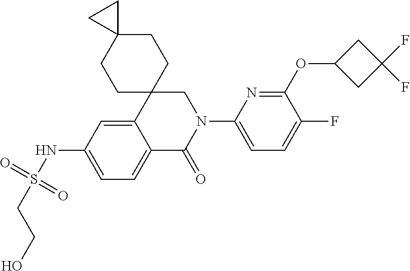 [SMILES: O=C1c2ccc(NS(=O)(=O)CCO)cc2C2(CCC3(CC3)CC2)CN1c1ccc(F)c(OC2CC(F)(F)C2)n1]

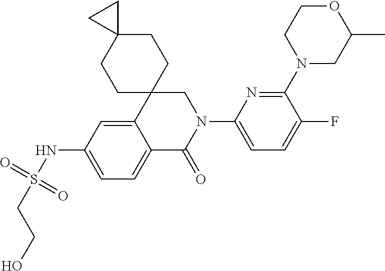 [SMILES: CC1CN(c2nc(N3CC4(CCC5(CC5)CC4)c4cc(NS(=O)(=O)CCO)ccc4C3=O)ccc2F)CCO1]

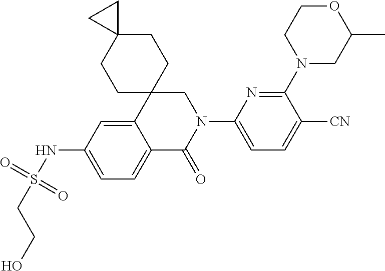 [SMILES: CC1CN(c2nc(N3CC4(CCC5(CC5)CC4)c4cc(NS(=O)(=O)CCO)ccc4C3=O)ccc2C#N)CCO1]

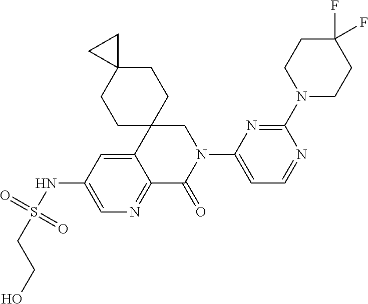 [SMILES: O=C1c2ncc(NS(=O)(=O)CCO)cc2C2(CCC3(CC3)CC2)CN1c1ccnc(N2CCC(F)(F)CC2)n1]

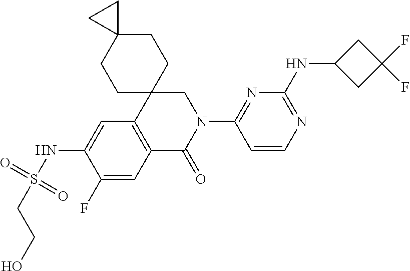 [SMILES: O=C1c2cc(F)c(NS(=O)(=O)CCO)cc2C2(CCC3(CC3)CC2)CN1c1ccnc(NC2CC(F)(F)C2)n1]

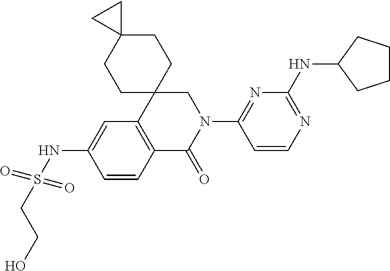 [SMILES: O=C1c2ccc(NS(=O)(=O)CCO)cc2C2(CCC3(CC3)CC2)CN1c1ccnc(NC2CCCC2)n1]

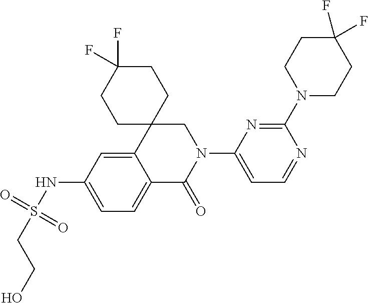 [SMILES: O=C1c2ccc(NS(=O)(=O)CCO)cc2C2(CCC(F)(F)CC2)CN1c1ccnc(N2CCC(F)(F)CC2)n1]

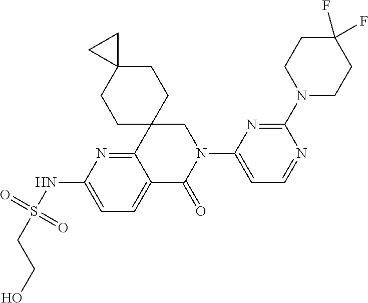 [SMILES: O=C1c2ccc(NS(=O)(=O)CCO)nc2C2(CCC3(CC3)CC2)CN1c1ccnc(N2CCC(F)(F)CC2)n1]

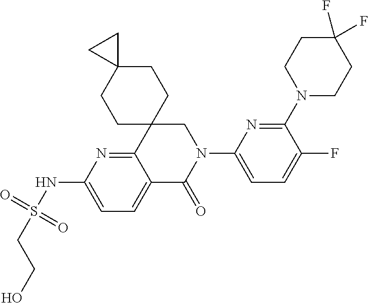 [SMILES: O=C1c2ccc(NS(=O)(=O)CCO)nc2C2(CCC3(CC3)CC2)CN1c1ccc(F)c(N2CCC(F)(F)CC2)n1]

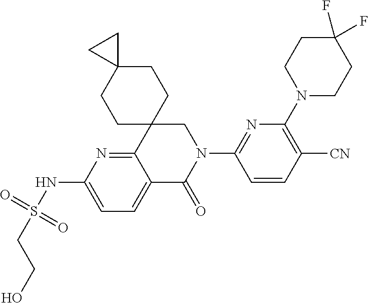 [SMILES: N#Cc1ccc(N2CC3(CCC4(CC4)CC3)c3nc(NS(=O)(=O)CCO)ccc3C2=O)nc1N1CCC(F)(F)CC1]

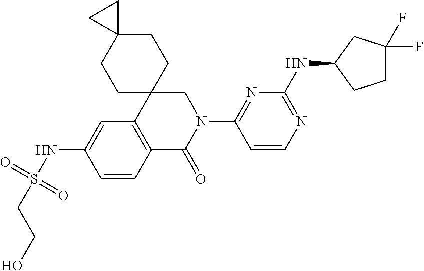 [SMILES: O=C1c2ccc(NS(=O)(=O)CCO)cc2C2(CCC3(CC3)CC2)CN1c1ccnc(N[C@@H]2CCC(F)(F)C2)n1]

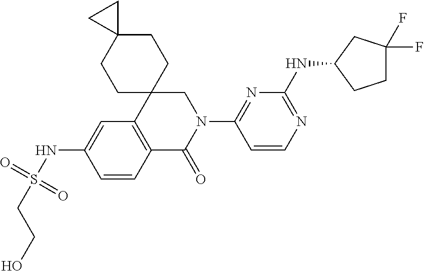 [SMILES: O=C1c2ccc(NS(=O)(=O)CCO)cc2C2(CCC3(CC3)CC2)CN1c1ccnc(N[C@H]2CCC(F)(F)C2)n1]

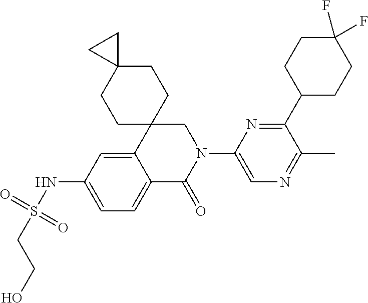 [SMILES: Cc1ncc(N2CC3(CCC4(CC4)CC3)c3cc(NS(=O)(=O)CCO)ccc3C2=O)nc1C1CCC(F)(F)CC1]

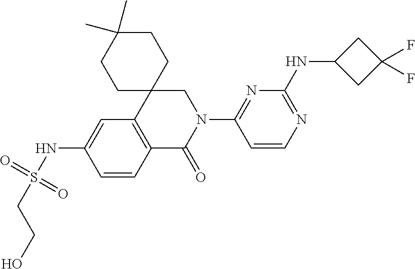 [SMILES: CC1(C)CCC2(CC1)CN(c1ccnc(NC3CC(F)(F)C3)n1)C(=O)c1ccc(NS(=O)(=O)CCO)cc12]

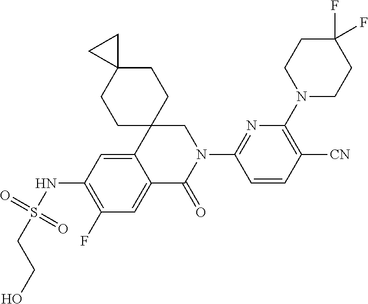 [SMILES: N#Cc1ccc(N2CC3(CCC4(CC4)CC3)c3cc(NS(=O)(=O)CCO)c(F)cc3C2=O)nc1N1CCC(F)(F)CC1]

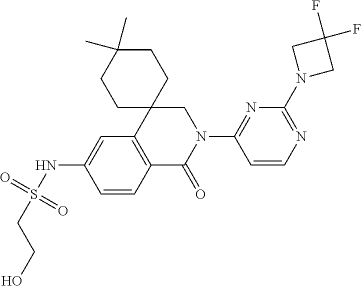 [SMILES: CC1(C)CCC2(CC1)CN(c1ccnc(N3CC(F)(F)C3)n1)C(=O)c1ccc(NS(=O)(=O)CCO)cc12]

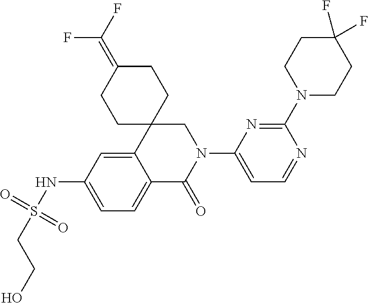 [SMILES: O=C1c2ccc(NS(=O)(=O)CCO)cc2C2(CCC(=C(F)F)CC2)CN1c1ccnc(N2CCC(F)(F)CC2)n1]

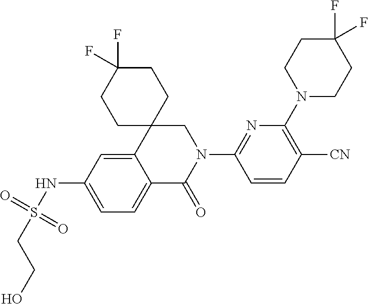 [SMILES: N#Cc1ccc(N2CC3(CCC(F)(F)CC3)c3cc(NS(=O)(=O)CCO)ccc3C2=O)nc1N1CCC(F)(F)CC1]

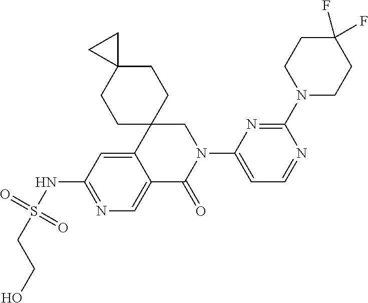 [SMILES: O=C1c2cnc(NS(=O)(=O)CCO)cc2C2(CCC3(CC3)CC2)CN1c1ccnc(N2CCC(F)(F)CC2)n1]

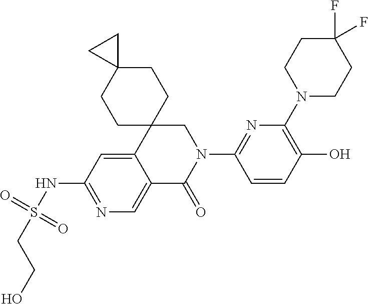 [SMILES: O=C1c2cnc(NS(=O)(=O)CCO)cc2C2(CCC3(CC3)CC2)CN1c1ccc(O)c(N2CCC(F)(F)CC2)n1]

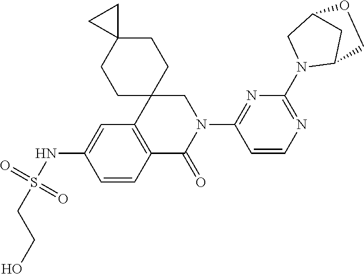 [SMILES: O=C1c2ccc(NS(=O)(=O)CCO)cc2C2(CCC3(CC3)CC2)CN1c1ccnc(N2C[C@H]3C[C@@H]2CO3)n1]

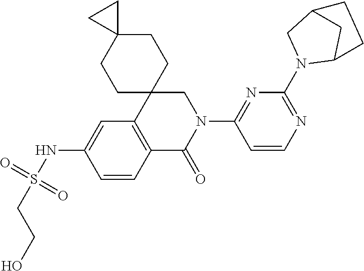 [SMILES: O=C1c2ccc(NS(=O)(=O)CCO)cc2C2(CCC3(CC3)CC2)CN1c1ccnc(N2CC3CCC2C3)n1]

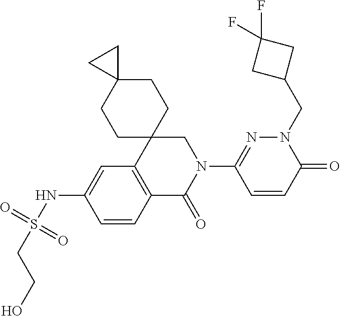 [SMILES: O=C1c2ccc(NS(=O)(=O)CCO)cc2C2(CCC3(CC3)CC2)CN1c1ccc(=O)n(CC2CC(F)(F)C2)n1]

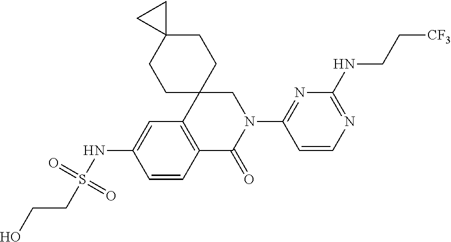 [SMILES: O=C1c2ccc(NS(=O)(=O)CCO)cc2C2(CCC3(CC3)CC2)CN1c1ccnc(NCCC(F)(F)F)n1]

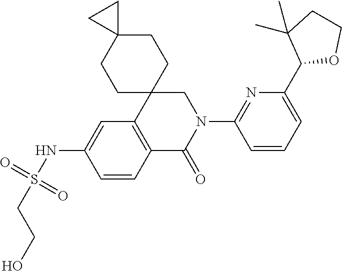 [SMILES: CC1(C)CCO[C@@H]1c1cccc(N2CC3(CCC4(CC4)CC3)c3cc(NS(=O)(=O)CCO)ccc3C2=O)n1]

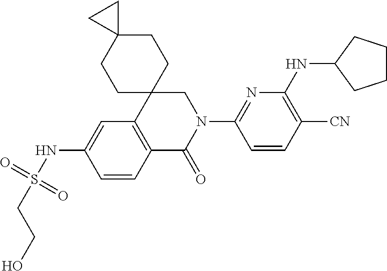 [SMILES: N#Cc1ccc(N2CC3(CCC4(CC4)CC3)c3cc(NS(=O)(=O)CCO)ccc3C2=O)nc1NC1CCCC1]

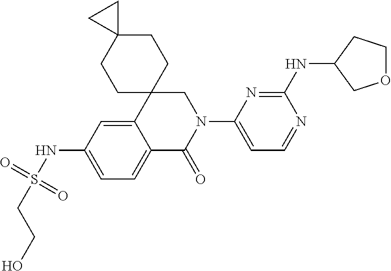 [SMILES: O=C1c2ccc(NS(=O)(=O)CCO)cc2C2(CCC3(CC3)CC2)CN1c1ccnc(NC2CCOC2)n1]

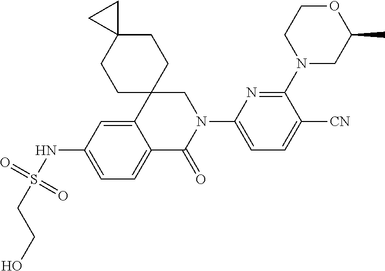 [SMILES: C[C@H]1CN(c2nc(N3CC4(CCC5(CC5)CC4)c4cc(NS(=O)(=O)CCO)ccc4C3=O)ccc2C#N)CCO1]

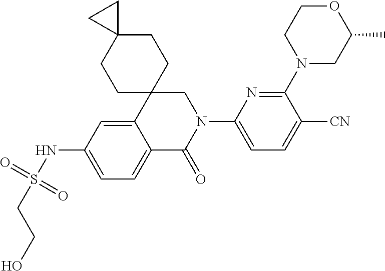 [SMILES: C[C@@H]1CN(c2nc(N3CC4(CCC5(CC5)CC4)c4cc(NS(=O)(=O)CCO)ccc4C3=O)ccc2C#N)CCO1]

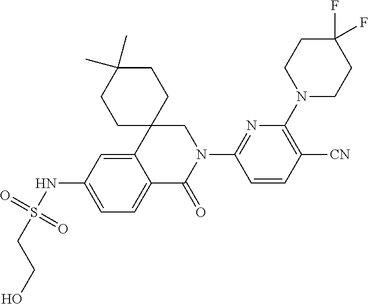 [SMILES: CC1(C)CCC2(CC1)CN(c1ccc(C#N)c(N3CCC(F)(F)CC3)n1)C(=O)c1ccc(NS(=O)(=O)CCO)cc12]

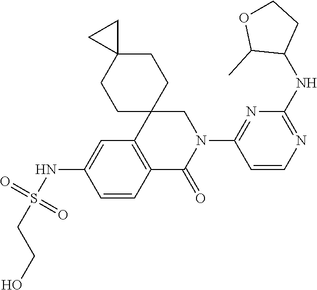 [SMILES: CC1OCCC1Nc1nccc(N2CC3(CCC4(CC4)CC3)c3cc(NS(=O)(=O)CCO)ccc3C2=O)n1]

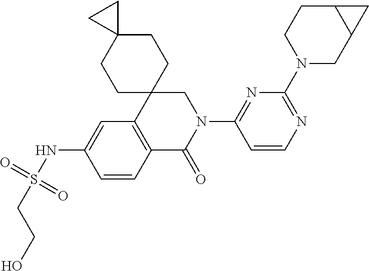 [SMILES: O=C1c2ccc(NS(=O)(=O)CCO)cc2C2(CCC3(CC3)CC2)CN1c1ccnc(N2CCC3CC3C2)n1]

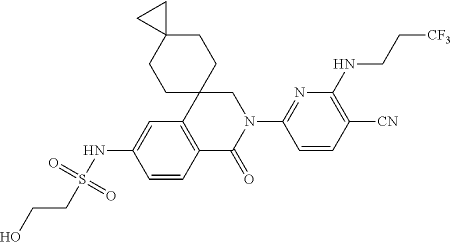 [SMILES: N#Cc1ccc(N2CC3(CCC4(CC4)CC3)c3cc(NS(=O)(=O)CCO)ccc3C2=O)nc1NCCC(F)(F)F]

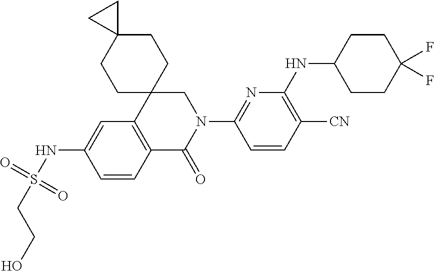 [SMILES: N#Cc1ccc(N2CC3(CCC4(CC4)CC3)c3cc(NS(=O)(=O)CCO)ccc3C2=O)nc1NC1CCC(F)(F)CC1]

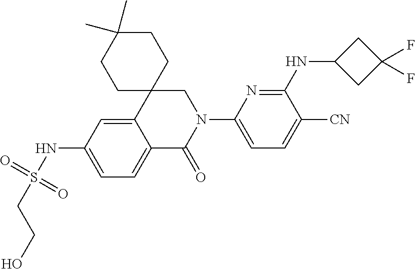 [SMILES: CC1(C)CCC2(CC1)CN(c1ccc(C#N)c(NC3CC(F)(F)C3)n1)C(=O)c1ccc(NS(=O)(=O)CCO)cc12]

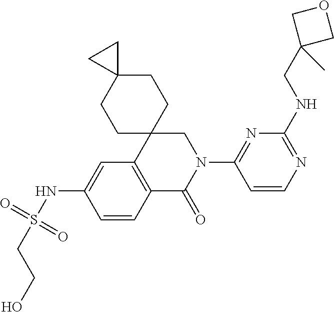 [SMILES: CC1(CNc2nccc(N3CC4(CCC5(CC5)CC4)c4cc(NS(=O)(=O)CCO)ccc4C3=O)n2)COC1]

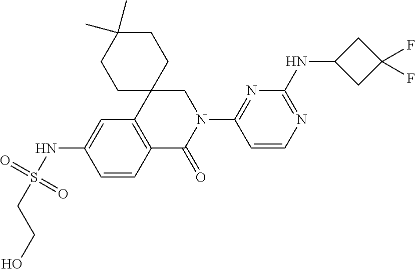 [SMILES: CC1(C)CCC2(CC1)CN(c1ccnc(NC3CC(F)(F)C3)n1)C(=O)c1ccc(NS(=O)(=O)CCO)cc12]

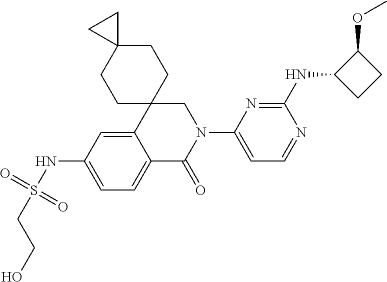 [SMILES: CO[C@H]1CC[C@@H]1Nc1nccc(N2CC3(CCC4(CC4)CC3)c3cc(NS(=O)(=O)CCO)ccc3C2=O)n1]

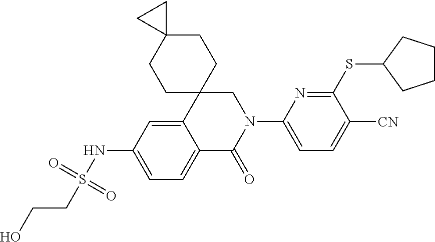 [SMILES: N#Cc1ccc(N2CC3(CCC4(CC4)CC3)c3cc(NS(=O)(=O)CCO)ccc3C2=O)nc1SC1CCCC1]

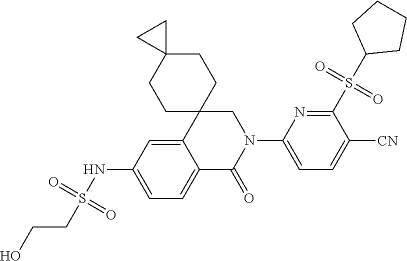 [SMILES: N#Cc1ccc(N2CC3(CCC4(CC4)CC3)c3cc(NS(=O)(=O)CCO)ccc3C2=O)nc1S(=O)(=O)C1CCCC1]

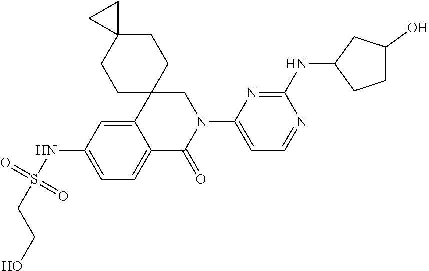 [SMILES: O=C1c2ccc(NS(=O)(=O)CCO)cc2C2(CCC3(CC3)CC2)CN1c1ccnc(NC2CCC(O)C2)n1]

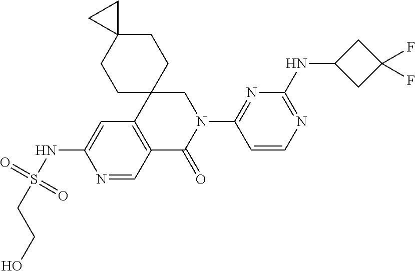 [SMILES: O=C1c2cnc(NS(=O)(=O)CCO)cc2C2(CCC3(CC3)CC2)CN1c1ccnc(NC2CC(F)(F)C2)n1]

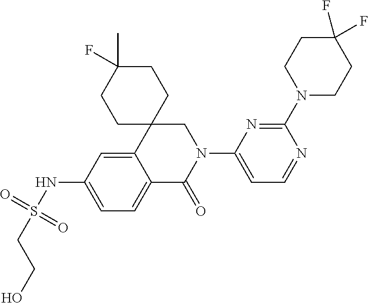 [SMILES: CC1(F)CCC2(CC1)CN(c1ccnc(N3CCC(F)(F)CC3)n1)C(=O)c1ccc(NS(=O)(=O)CCO)cc12]

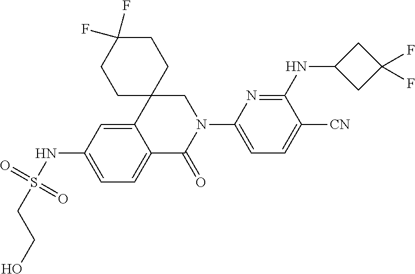 [SMILES: N#Cc1ccc(N2CC3(CCC(F)(F)CC3)c3cc(NS(=O)(=O)CCO)ccc3C2=O)nc1NC1CC(F)(F)C1]

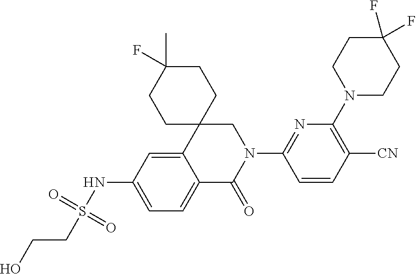 [SMILES: CC1(F)CCC2(CC1)CN(c1ccc(C#N)c(N3CCC(F)(F)CC3)n1)C(=O)c1ccc(NS(=O)(=O)CCO)cc12]

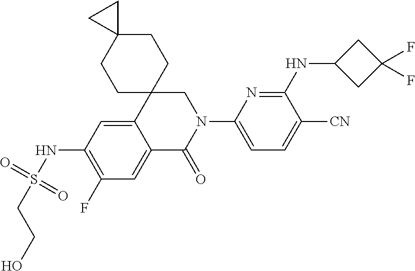 [SMILES: N#Cc1ccc(N2CC3(CCC4(CC4)CC3)c3cc(NS(=O)(=O)CCO)c(F)cc3C2=O)nc1NC1CC(F)(F)C1]

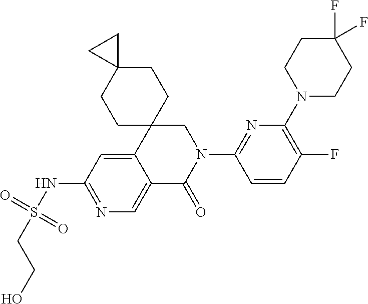 [SMILES: O=C1c2cnc(NS(=O)(=O)CCO)cc2C2(CCC3(CC3)CC2)CN1c1ccc(F)c(N2CCC(F)(F)CC2)n1]

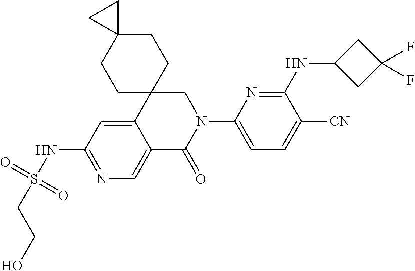 [SMILES: N#Cc1ccc(N2CC3(CCC4(CC4)CC3)c3cc(NS(=O)(=O)CCO)ncc3C2=O)nc1NC1CC(F)(F)C1]

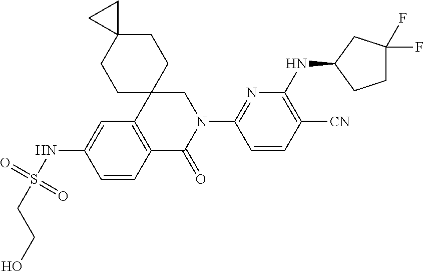 [SMILES: N#Cc1ccc(N2CC3(CCC4(CC4)CC3)c3cc(NS(=O)(=O)CCO)ccc3C2=O)nc1N[C@@H]1CCC(F)(F)C1]

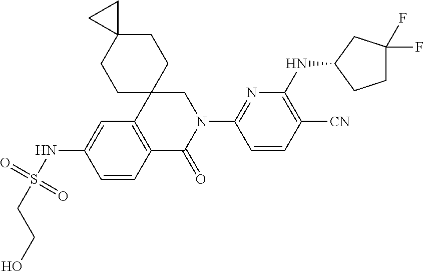 [SMILES: N#Cc1ccc(N2CC3(CCC4(CC4)CC3)c3cc(NS(=O)(=O)CCO)ccc3C2=O)nc1N[C@H]1CCC(F)(F)C1]

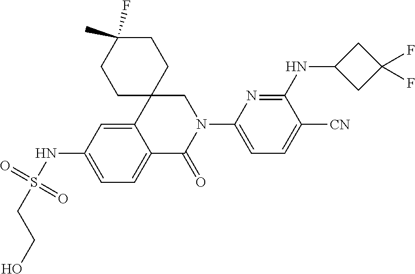

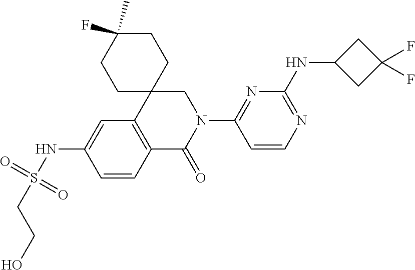

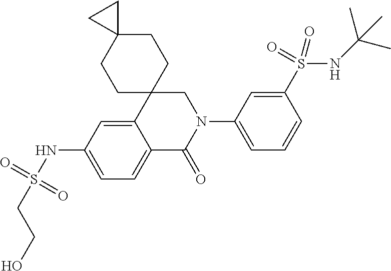 [SMILES: CC(C)(C)NS(=O)(=O)c1cccc(N2CC3(CCC4(CC4)CC3)c3cc(NS(=O)(=O)CCO)ccc3C2=O)c1]

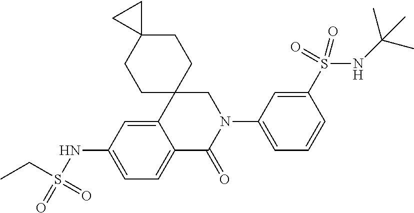 [SMILES: CCS(=O)(=O)Nc1ccc2c(c1)C1(CCC3(CC3)CC1)CN(c1cccc(S(=O)(=O)NC(C)(C)C)c1)C2=O]

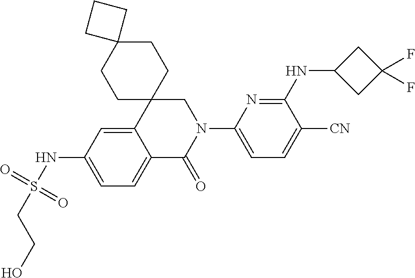 [SMILES: N#Cc1ccc(N2CC3(CCC4(CCC4)CC3)c3cc(NS(=O)(=O)CCO)ccc3C2=O)nc1NC1CC(F)(F)C1]

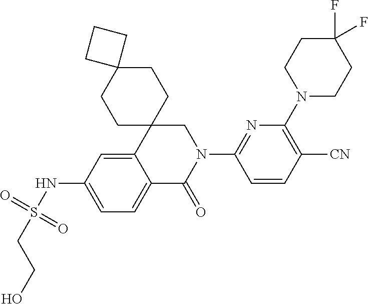 [SMILES: N#Cc1ccc(N2CC3(CCC4(CCC4)CC3)c3cc(NS(=O)(=O)CCO)ccc3C2=O)nc1N1CCC(F)(F)CC1]

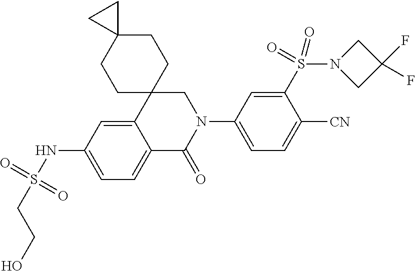 [SMILES: N#Cc1ccc(N2CC3(CCC4(CC4)CC3)c3cc(NS(=O)(=O)CCO)ccc3C2=O)cc1S(=O)(=O)N1CC(F)(F)C1]

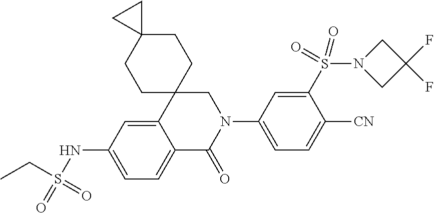 [SMILES: CCS(=O)(=O)Nc1ccc2c(c1)C1(CCC3(CC3)CC1)CN(c1ccc(C#N)c(S(=O)(=O)N3CC(F)(F)C3)c1)C2=O]

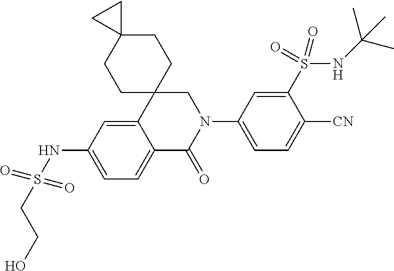 [SMILES: CC(C)(C)NS(=O)(=O)c1cc(N2CC3(CCC4(CC4)CC3)c3cc(NS(=O)(=O)CCO)ccc3C2=O)ccc1C#N]

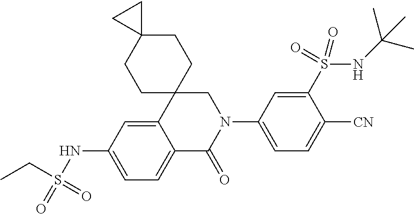 [SMILES: CCS(=O)(=O)Nc1ccc2c(c1)C1(CCC3(CC3)CC1)CN(c1ccc(C#N)c(S(=O)(=O)NC(C)(C)C)c1)C2=O]

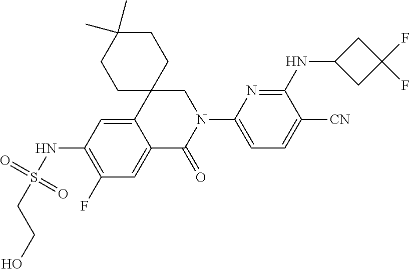 [SMILES: CC1(C)CCC2(CC1)CN(c1ccc(C#N)c(NC3CC(F)(F)C3)n1)C(=O)c1cc(F)c(NS(=O)(=O)CCO)cc12]

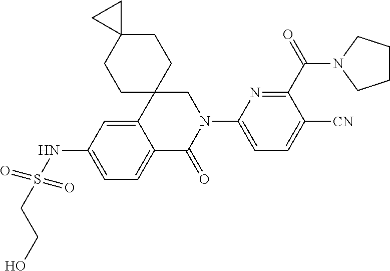 [SMILES: N#Cc1ccc(N2CC3(CCC4(CC4)CC3)c3cc(NS(=O)(=O)CCO)ccc3C2=O)nc1C(=O)N1CCCC1]

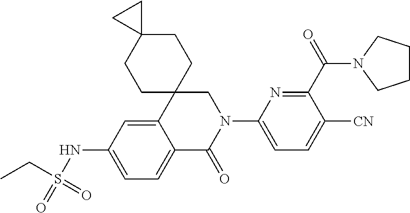 [SMILES: CCS(=O)(=O)Nc1ccc2c(c1)C1(CCC3(CC3)CC1)CN(c1ccc(C#N)c(C(=O)N3CCCC3)n1)C2=O]

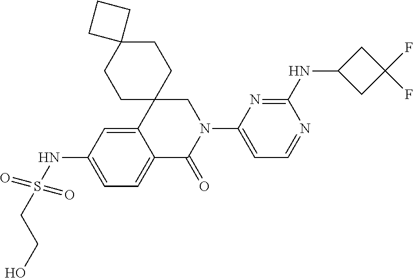 [SMILES: O=C1c2ccc(NS(=O)(=O)CCO)cc2C2(CCC3(CCC3)CC2)CN1c1ccnc(NC2CC(F)(F)C2)n1]

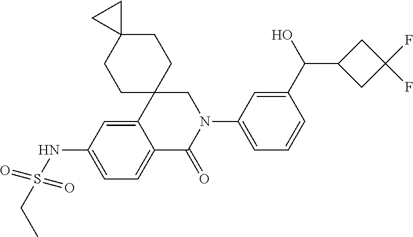 [SMILES: CCS(=O)(=O)Nc1ccc2c(c1)C1(CCC3(CC3)CC1)CN(c1cccc(C(O)C3CC(F)(F)C3)c1)C2=O]

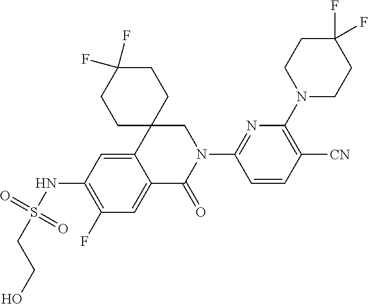 [SMILES: N#Cc1ccc(N2CC3(CCC(F)(F)CC3)c3cc(NS(=O)(=O)CCO)c(F)cc3C2=O)nc1N1CCC(F)(F)CC1]

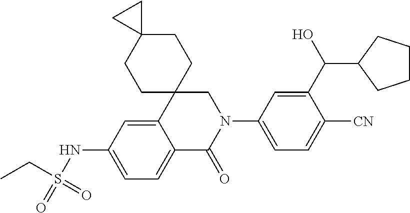 [SMILES: CCS(=O)(=O)Nc1ccc2c(c1)C1(CCC3(CC3)CC1)CN(c1ccc(C#N)c(C(O)C3CCCC3)c1)C2=O]

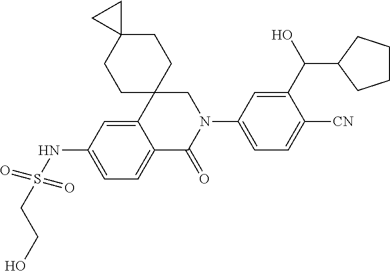 [SMILES: N#Cc1ccc(N2CC3(CCC4(CC4)CC3)c3cc(NS(=O)(=O)CCO)ccc3C2=O)cc1C(O)C1CCCC1]

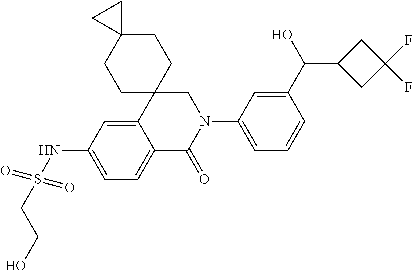 [SMILES: O=C1c2ccc(NS(=O)(=O)CCO)cc2C2(CCC3(CC3)CC2)CN1c1cccc(C(O)C2CC(F)(F)C2)c1]

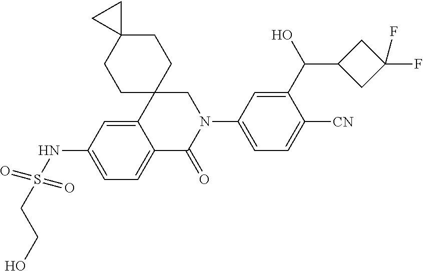 [SMILES: N#Cc1ccc(N2CC3(CCC4(CC4)CC3)c3cc(NS(=O)(=O)CCO)ccc3C2=O)cc1C(O)C1CC(F)(F)C1]

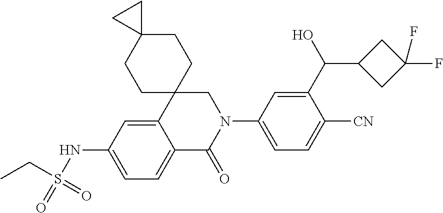 [SMILES: CCS(=O)(=O)Nc1ccc2c(c1)C1(CCC3(CC3)CC1)CN(c1ccc(C#N)c(C(O)C3CC(F)(F)C3)c1)C2=O]

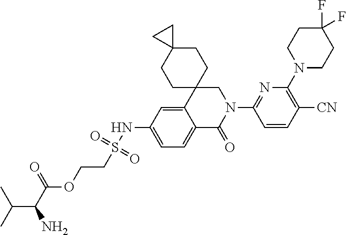 [SMILES: CC(C)[C@H](N)C(=O)OCCS(=O)(=O)Nc1ccc2c(c1)C1(CCC3(CC3)CC1)CN(c1ccc(C#N)c(N3CCC(F)(F)CC3)n1)C2=O]

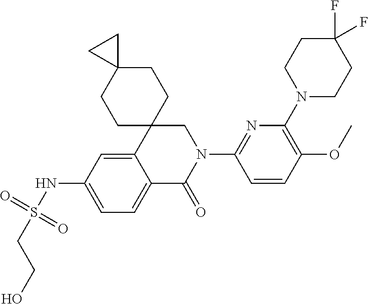 [SMILES: COc1ccc(N2CC3(CCC4(CC4)CC3)c3cc(NS(=O)(=O)CCO)ccc3C2=O)nc1N1CCC(F)(F)CC1]

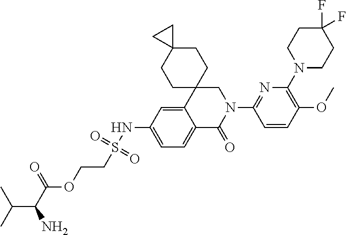 [SMILES: COc1ccc(N2CC3(CCC4(CC4)CC3)c3cc(NS(=O)(=O)CCOC(=O)[C@@H](N)C(C)C)ccc3C2=O)nc1N1CCC(F)(F)CC1]

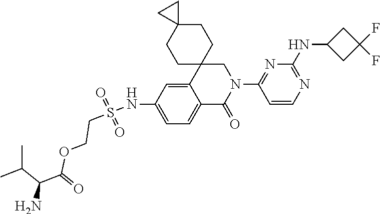 [SMILES: CC(C)[C@H](N)C(=O)OCCS(=O)(=O)Nc1ccc2c(c1)C1(CCC3(CC3)CC1)CN(c1ccnc(NC3CC(F)(F)C3)n1)C2=O]

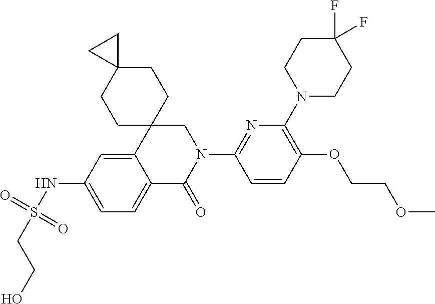 [SMILES: COCCOc1ccc(N2CC3(CCC4(CC4)CC3)c3cc(NS(=O)(=O)CCO)ccc3C2=O)nc1N1CCC(F)(F)CC1]

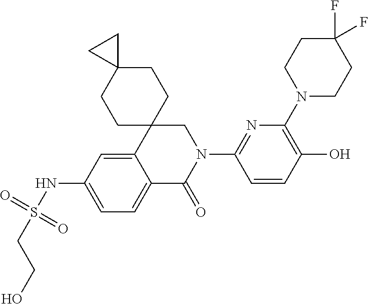 [SMILES: O=C1c2ccc(NS(=O)(=O)CCO)cc2C2(CCC3(CC3)CC2)CN1c1ccc(O)c(N2CCC(F)(F)CC2)n1]

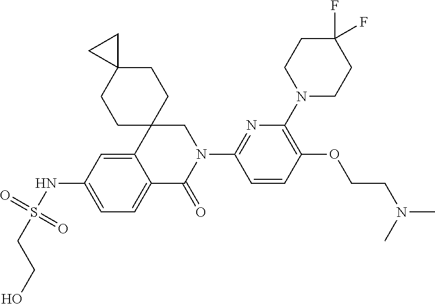 [SMILES: CN(C)CCOc1ccc(N2CC3(CCC4(CC4)CC3)c3cc(NS(=O)(=O)CCO)ccc3C2=O)nc1N1CCC(F)(F)CC1]

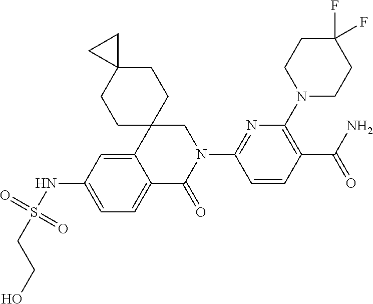 [SMILES: NC(=O)c1ccc(N2CC3(CCC4(CC4)CC3)c3cc(NS(=O)(=O)CCO)ccc3C2=O)nc1N1CCC(F)(F)CC1]

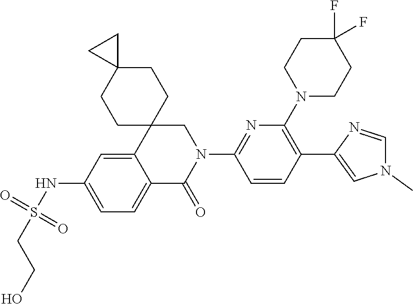 [SMILES: Cn1cnc(-c2ccc(N3CC4(CCC5(CC5)CC4)c4cc(NS(=O)(=O)CCO)ccc4C3=O)nc2N2CCC(F)(F)CC2)c1]

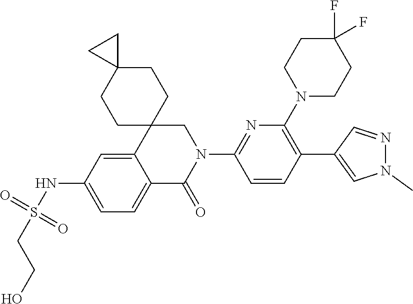 [SMILES: Cn1cc(-c2ccc(N3CC4(CCC5(CC5)CC4)c4cc(NS(=O)(=O)CCO)ccc4C3=O)nc2N2CCC(F)(F)CC2)cn1]

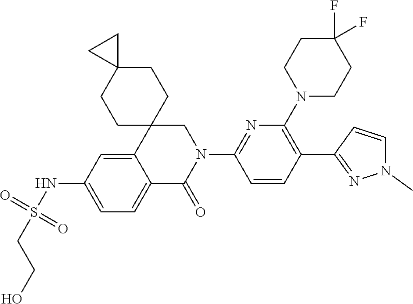 [SMILES: Cn1ccc(-c2ccc(N3CC4(CCC5(CC5)CC4)c4cc(NS(=O)(=O)CCO)ccc4C3=O)nc2N2CCC(F)(F)CC2)n1]

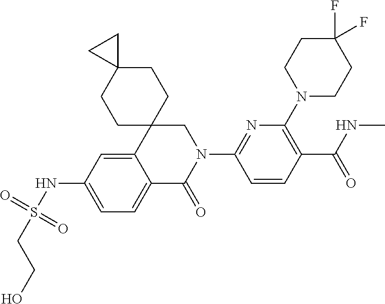 [SMILES: CNC(=O)c1ccc(N2CC3(CCC4(CC4)CC3)c3cc(NS(=O)(=O)CCO)ccc3C2=O)nc1N1CCC(F)(F)CC1]

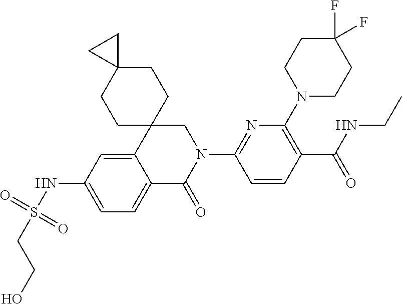 [SMILES: CCNC(=O)c1ccc(N2CC3(CCC4(CC4)CC3)c3cc(NS(=O)(=O)CCO)ccc3C2=O)nc1N1CCC(F)(F)CC1]

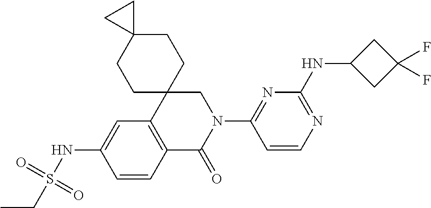 [SMILES: CCS(=O)(=O)Nc1ccc2c(c1)C1(CCC3(CC3)CC1)CN(c1ccnc(NC3CC(F)(F)C3)n1)C2=O]

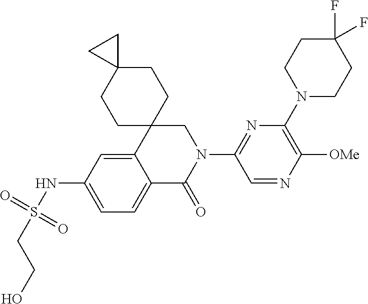 [SMILES: COc1ncc(N2CC3(CCC4(CC4)CC3)c3cc(NS(=O)(=O)CCO)ccc3C2=O)nc1N1CCC(F)(F)CC1]

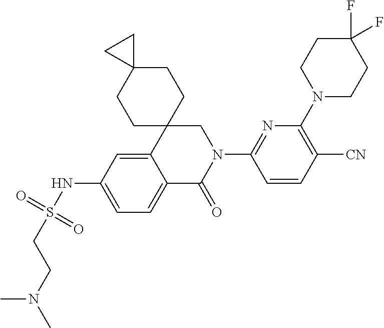 [SMILES: CN(C)CCS(=O)(=O)Nc1ccc2c(c1)C1(CCC3(CC3)CC1)CN(c1ccc(C#N)c(N3CCC(F)(F)CC3)n1)C2=O]

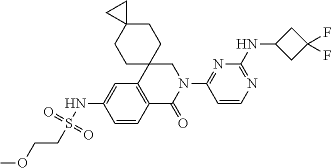 [SMILES: COCCS(=O)(=O)Nc1ccc2c(c1)C1(CCC3(CC3)CC1)CN(c1ccnc(NC3CC(F)(F)C3)n1)C2=O]

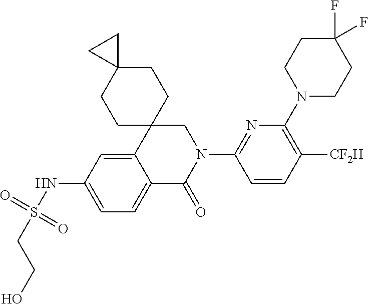 [SMILES: O=C1c2ccc(NS(=O)(=O)CCO)cc2C2(CCC3(CC3)CC2)CN1c1ccc(C(F)F)c(N2CCC(F)(F)CC2)n1]

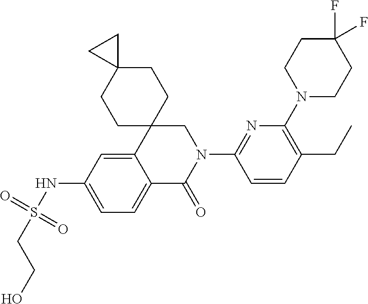 [SMILES: CCc1ccc(N2CC3(CCC4(CC4)CC3)c3cc(NS(=O)(=O)CCO)ccc3C2=O)nc1N1CCC(F)(F)CC1]

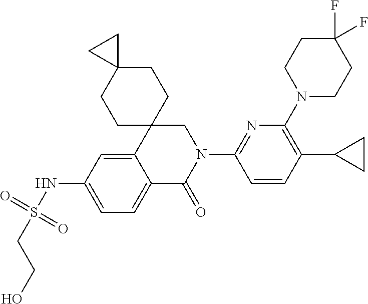 [SMILES: O=C1c2ccc(NS(=O)(=O)CCO)cc2C2(CCC3(CC3)CC2)CN1c1ccc(C2CC2)c(N2CCC(F)(F)CC2)n1]

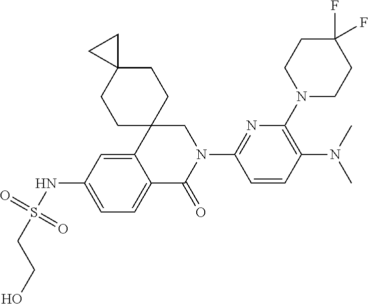 [SMILES: CN(C)c1ccc(N2CC3(CCC4(CC4)CC3)c3cc(NS(=O)(=O)CCO)ccc3C2=O)nc1N1CCC(F)(F)CC1]

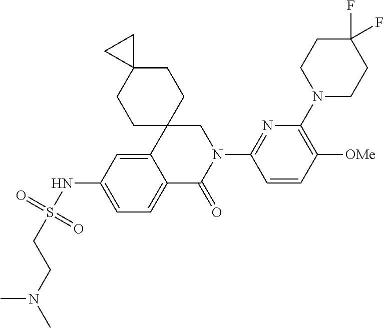 [SMILES: COc1ccc(N2CC3(CCC4(CC4)CC3)c3cc(NS(=O)(=O)CCN(C)C)ccc3C2=O)nc1N1CCC(F)(F)CC1]

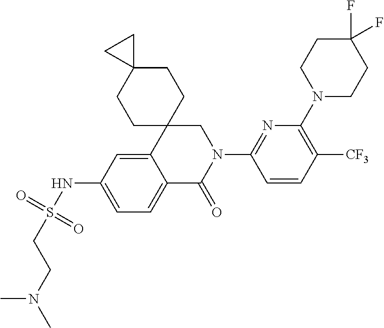 [SMILES: CN(C)CCS(=O)(=O)Nc1ccc2c(c1)C1(CCC3(CC3)CC1)CN(c1ccc(C(F)(F)F)c(N3CCC(F)(F)CC3)n1)C2=O]

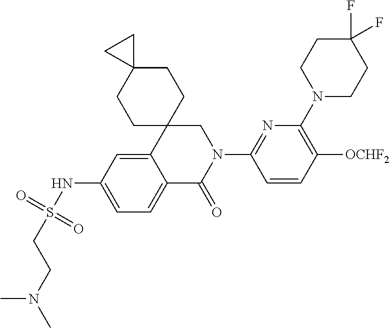 [SMILES: CN(C)CCS(=O)(=O)Nc1ccc2c(c1)C1(CCC3(CC3)CC1)CN(c1ccc(OC(F)F)c(N3CCC(F)(F)CC3)n1)C2=O]

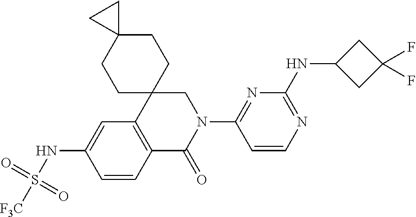 [SMILES: O=C1c2ccc(NS(=O)(=O)C(F)(F)F)cc2C2(CCC3(CC3)CC2)CN1c1ccnc(NC2CC(F)(F)C2)n1]

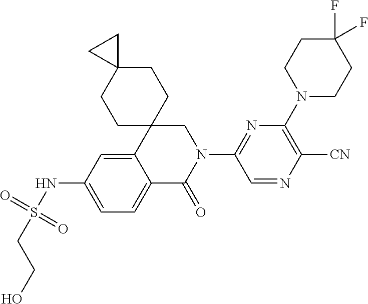 [SMILES: N#Cc1ncc(N2CC3(CCC4(CC4)CC3)c3cc(NS(=O)(=O)CCO)ccc3C2=O)nc1N1CCC(F)(F)CC1]

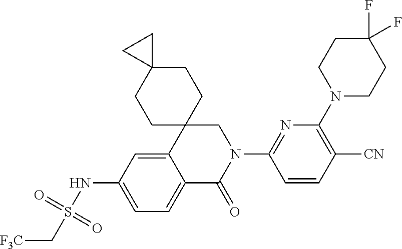 [SMILES: N#Cc1ccc(N2CC3(CCC4(CC4)CC3)c3cc(NS(=O)(=O)CC(F)(F)F)ccc3C2=O)nc1N1CCC(F)(F)CC1]

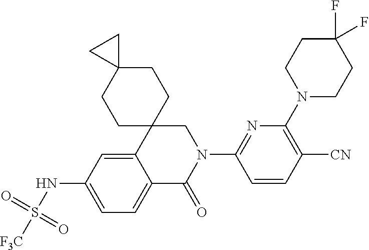 [SMILES: N#Cc1ccc(N2CC3(CCC4(CC4)CC3)c3cc(NS(=O)(=O)C(F)(F)F)ccc3C2=O)nc1N1CCC(F)(F)CC1]

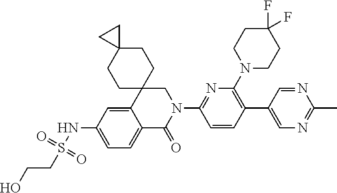 [SMILES: Cc1ncc(-c2ccc(N3CC4(CCC5(CC5)CC4)c4cc(NS(=O)(=O)CCO)ccc4C3=O)nc2N2CCC(F)(F)CC2)cn1]

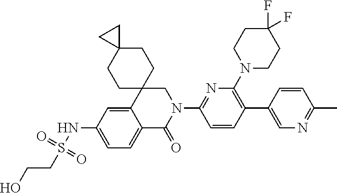 [SMILES: Cc1ccc(-c2ccc(N3CC4(CCC5(CC5)CC4)c4cc(NS(=O)(=O)CCO)ccc4C3=O)nc2N2CCC(F)(F)CC2)cn1]

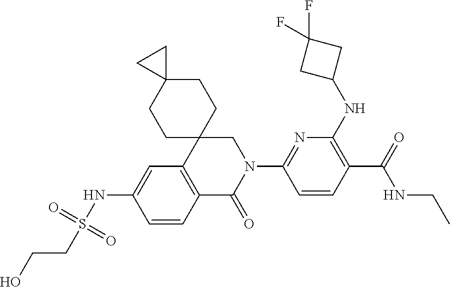 [SMILES: CCNC(=O)c1ccc(N2CC3(CCC4(CC4)CC3)c3cc(NS(=O)(=O)CCO)ccc3C2=O)nc1NC1CC(F)(F)C1]

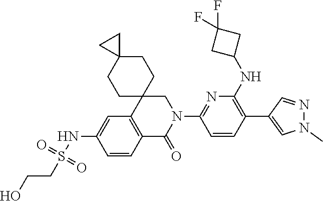 [SMILES: Cn1cc(-c2ccc(N3CC4(CCC5(CC5)CC4)c4cc(NS(=O)(=O)CCO)ccc4C3=O)nc2NC2CC(F)(F)C2)cn1]

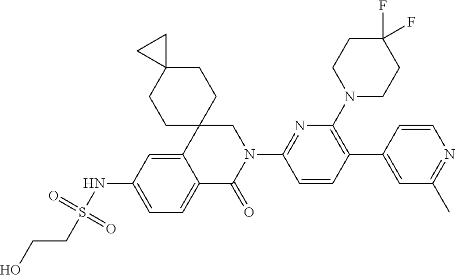 [SMILES: Cc1cc(-c2ccc(N3CC4(CCC5(CC5)CC4)c4cc(NS(=O)(=O)CCO)ccc4C3=O)nc2N2CCC(F)(F)CC2)ccn1]

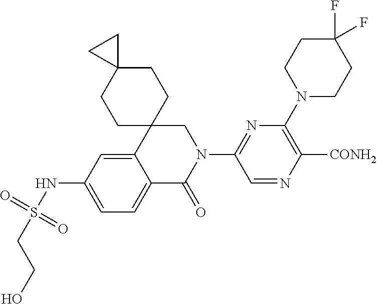 [SMILES: NC(=O)c1ncc(N2CC3(CCC4(CC4)CC3)c3cc(NS(=O)(=O)CCO)ccc3C2=O)nc1N1CCC(F)(F)CC1]

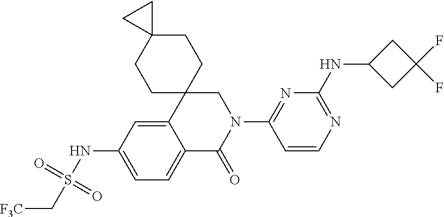 [SMILES: O=C1c2ccc(NS(=O)(=O)CC(F)(F)F)cc2C2(CCC3(CC3)CC2)CN1c1ccnc(NC2CC(F)(F)C2)n1]

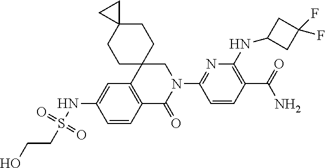 [SMILES: NC(=O)c1ccc(N2CC3(CCC4(CC4)CC3)c3cc(NS(=O)(=O)CCO)ccc3C2=O)nc1NC1CC(F)(F)C1]

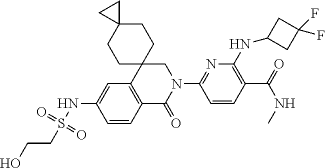 [SMILES: CNC(=O)c1ccc(N2CC3(CCC4(CC4)CC3)c3cc(NS(=O)(=O)CCO)ccc3C2=O)nc1NC1CC(F)(F)C1]

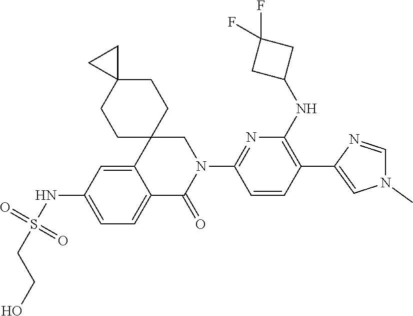 [SMILES: Cn1cnc(-c2ccc(N3CC4(CCC5(CC5)CC4)c4cc(NS(=O)(=O)CCO)ccc4C3=O)nc2NC2CC(F)(F)C2)c1]

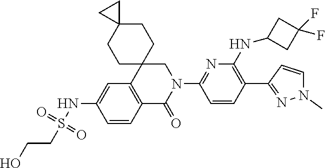 [SMILES: Cn1ccc(-c2ccc(N3CC4(CCC5(CC5)CC4)c4cc(NS(=O)(=O)CCO)ccc4C3=O)nc2NC2CC(F)(F)C2)n1]

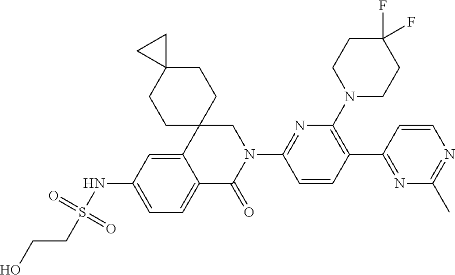 [SMILES: Cc1nccc(-c2ccc(N3CC4(CCC5(CC5)CC4)c4cc(NS(=O)(=O)CCO)ccc4C3=O)nc2N2CCC(F)(F)CC2)n1]

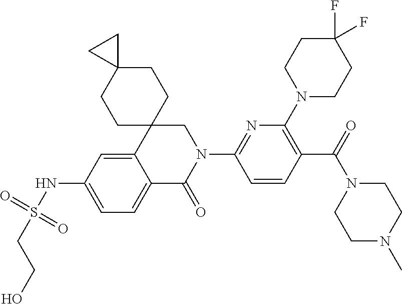 [SMILES: CN1CCN(C(=O)c2ccc(N3CC4(CCC5(CC5)CC4)c4cc(NS(=O)(=O)CCO)ccc4C3=O)nc2N2CCC(F)(F)CC2)CC1]

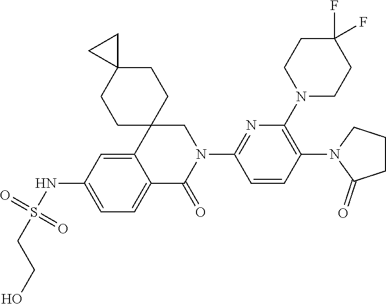 [SMILES: O=C1c2ccc(NS(=O)(=O)CCO)cc2C2(CCC3(CC3)CC2)CN1c1ccc(N2CCCC2=O)c(N2CCC(F)(F)CC2)n1]

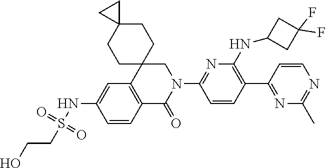 [SMILES: Cc1nccc(-c2ccc(N3CC4(CCC5(CC5)CC4)c4cc(NS(=O)(=O)CCO)ccc4C3=O)nc2NC2CC(F)(F)C2)n1]

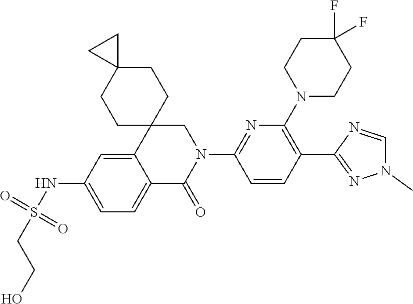 [SMILES: Cn1cnc(-c2ccc(N3CC4(CCC5(CC5)CC4)c4cc(NS(=O)(=O)CCO)ccc4C3=O)nc2N2CCC(F)(F)CC2)n1]

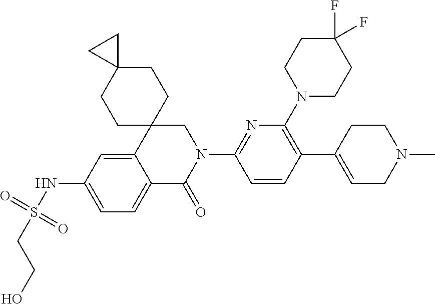 [SMILES: CN1CC=C(c2ccc(N3CC4(CCC5(CC5)CC4)c4cc(NS(=O)(=O)CCO)ccc4C3=O)nc2N2CCC(F)(F)CC2)CC1]

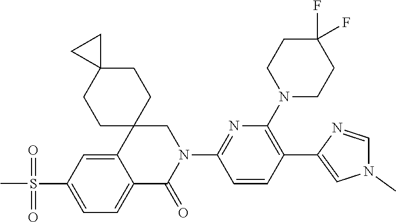

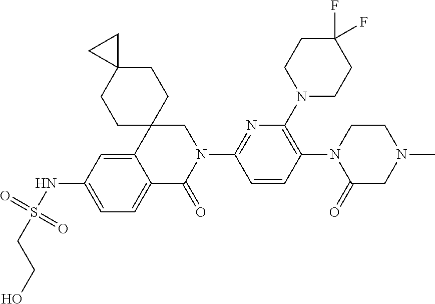 [SMILES: CN1CCN(c2ccc(N3CC4(CCC5(CC5)CC4)c4cc(NS(=O)(=O)CCO)ccc4C3=O)nc2N2CCC(F)(F)CC2)C(=O)C1]

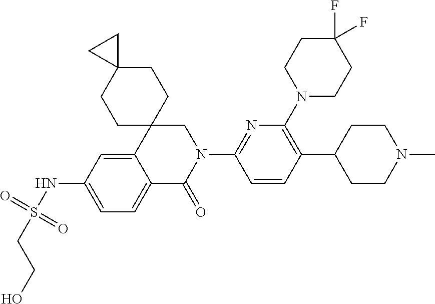 [SMILES: CN1CCC(c2ccc(N3CC4(CCC5(CC5)CC4)c4cc(NS(=O)(=O)CCO)ccc4C3=O)nc2N2CCC(F)(F)CC2)CC1]

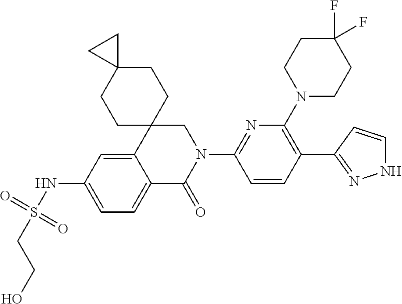 [SMILES: O=C1c2ccc(NS(=O)(=O)CCO)cc2C2(CCC3(CC3)CC2)CN1c1ccc(-c2cc[nH]n2)c(N2CCC(F)(F)CC2)n1]

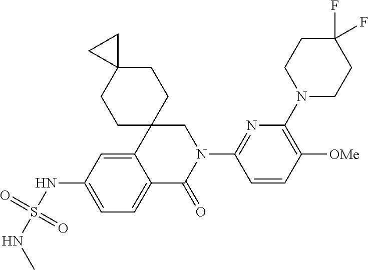 [SMILES: CNS(=O)(=O)Nc1ccc2c(c1)C1(CCC3(CC3)CC1)CN(c1ccc(OC)c(N3CCC(F)(F)CC3)n1)C2=O]

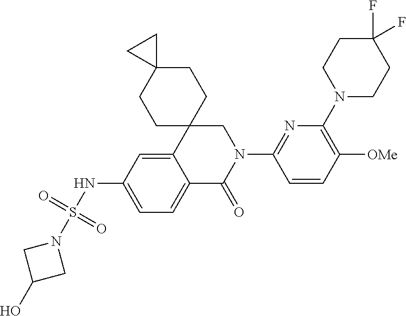 [SMILES: COc1ccc(N2CC3(CCC4(CC4)CC3)c3cc(NS(=O)(=O)N4CC(O)C4)ccc3C2=O)nc1N1CCC(F)(F)CC1]

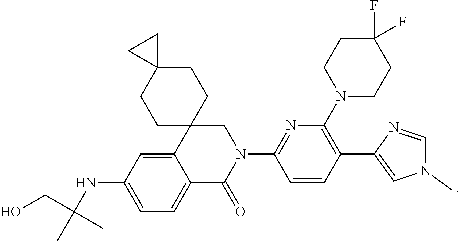 [SMILES: Cn1cnc(-c2ccc(N3CC4(CCC5(CC5)CC4)c4cc(NC(C)(C)CO)ccc4C3=O)nc2N2CCC(F)(F)CC2)c1]

### Method of Synthesis

The compounds of the present disclosure can be readily synthesized by those skilled in the art in view of the present disclosure. Exemplified synthesis is also shown in the Examples section.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wilev-VCH. 1999), and any of available undates as of this filing.

### Pharmaceutical Compositions

Certain embodiments are directed to a pharmaceutical composition comprising one or more of the compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof) and a pharmaceutically acceptable excipient. Pharmaceutically acceptable excipients are known in the art. Non-limiting suitable excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof), e.g., in a therapeutically effective amount. In any of the embodiments described herein, the pharmaceutical composition can comprise a therapeutically effective amount of a compound shown in Table A or in the Examples section, or a pharmaceutically acceptable salt thereof.

The pharmaceutical composition can also be formulated for delivery via any of the known routes of delivery, which include but are not limited to oral, parenteral, inhalation, etc.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

In some embodiments, the pharmaceutical composition is formulated for inhalation. The inhalable formulations can be, for example, formulated as a nasal spray, dry powder, or an aerosol administrable through a metered-dose inhaler. Excipients for preparing formulations for inhalation are known in the art. Non-limiting suitable excipients include, for example, lactose, talc, silicic acid, aluminum hydroxide, calcium silicates and polyamide powder, and mixtures of these substances. Sprays can additionally contain propellants, such as chlorofluorohydrocarbons and volatile unsubstituted hydrocarbons, such as butane and propane.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof). In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, which can depend on the recipient of the treatment, the disease or disorder being treated and the severity thereof, the composition containing the compound, the time of administration, the route of administration, the duration of treatment, the compound potency (e.g., for inhibiting KIF18A), its rate of clearance and whether or not another drug is co-administered.

For veterinary use, a compound of the present disclosure can be administered as a suitably acceptable formulation in accordance with normal veterinary practice. The veterinarian can readily determine the dosing regimen and route of administration that is most appropriate for a particular animal.

In some embodiments, all the necessary components for the treatment of KIF18A-related disorder using a compound of the present disclosure either alone or in combination with another agent or intervention traditionally used for the treatment of such disease can be packaged into a kit. Specifically, in some embodiments, the present invention provides a kit for use in the therapeutic intervention of the disease comprising a packaged set of medicaments that include the compound disclosed herein as well as buffers and other components for preparing deliverable forms of said medicaments, and/or devices for delivering such medicaments, and/or any agents that are used in combination therapy with the compound of the present disclosure, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the internet.

### Method of Treatment

Compounds of the present disclosure are useful as therapeutic active substances for the treatment and/or prophylaxis of diseases or disorders that are associated with KIF18A protein.

In some embodiments, the present disclosure provides a method of inhibiting KIF18A in a cell, the method comprising contacting the cell with an effective amount of one or more compounds of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof).

In some embodiments, the present disclosure provides a method of treating a disease or disorder, e.g., a cancer associated with KIF18A protein, in a subject in need thereof. In some embodiments, the method comprises administering to the subject a therapeutically effective amount of a compound of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof) or a therapeutically effective amount of a pharmaceutical composition described herein.

In some embodiments, a method for treatment of cancer is provided, the method comprising administering to a subject in need thereof an effective amount of any of the compound of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof) or a pharmaceutical composition comprising the compound of the present disclosure. In some embodiments, the cancer comprises a KIF18A protein. In various embodiments, the cancer can be a solid or hematologically derived tumor selected from cancer of the cancer of the bladder, endometrial, lung squamous cell, breast, colon, kidney, liver, lung, small cell lung cancer, esophagus, gall-bladder, brain, head and neck, ovary, pancreas, stomach, cervix, thyroid, prostate and skin. In some embodiments, the cancer is a hematopoietic tumor of lymphoid lineage selected from leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma. In some embodiments, the cancer is a hematopoietic tumor of myeloid lineage selected from acute and chronic myelogenous leukemias, myelodysplastic syndrome and promyelocytic leukemia. In some embodiments, the cancer is a tumor of mesenchymal origin selected from fibrosarcoma and rhabdomyosarcoma. In some embodiments, the cancer is a tumor of the central and peripheral nervous system selected from astrocytoma, neuroblastoma, glioma and schwannoma. In some embodiments, the cancer is a melanoma, seminoma, teratocarcinoma, osteosarcoma, xenoderoma pigmentosum, keratoctanthoma, thyroid follicular cancer or Kaposi's sarcoma.

In some embodiments the present disclosure provides a method of treating a disease or disorder (e.g., a cancer described herein) in a subject in need thereof, wherein the method comprises determining if the subject has KIF18A protein, and if the subject is determined to have the KIF18A protein, then administering to the subject a therapeutically effective dose of at least one compound of the present disclosure (e.g., a compound of Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A, or a pharmaceutically acceptable salt thereof) or a pharmaceutical composition comprising the at least one compound of the present disclosure.

Compounds of the present disclosure can be used as a monotherapy or in a combination therapy. In some embodiments, the combination therapy includes treating the subject with a chemotherapeutic agent, therapeutic antibody, radiation, cell therapy, or immunotherapy. In some embodiments, compounds of the present disclosure can also be co-administered with an additional pharmaceutically active compound, either concurrently or sequentially in any order, to a subject in need thereof (e.g., a subject having a cancer associated with KIF18A protein as described herein). In some embodiments, the additional pharmaceutically active compound can be a chemotherapeutic agent, a therapeutic antibody, etc. Any of the known chemotherapeutics can be used in combination with the compounds of the present disclosure. In some embodiments, compounds of the present disclosure can also be used in combination with a radiation therapy, hormone therapy, cell therapy, surgery and immunotherapy, which therapies are well known to those skilled in the art.

The administering herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally.

Dosing regimen including doses can vary and can be adjusted, which can depend on the recipient of the treatment, the disease or disorder being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable atoms or groups for the variables herein are independently selected. The definitions of the variables can be combined. Using Formula I as an example, any of the definitions of one of R¹, R², R³,

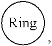

X¹, X², X³, R⁴, R⁵, R⁶, R^{7a}, R^{7b}, R⁸, R^{8b}, R^{9a}, R^{9b}, R^{A}, R^{B}, R^{C}, R^{D}, R^{E}, R^{F}, R^{G1}, R^{G2}, R^{S}, R^{T} and n in Formula I can be combined with any of the definitions of the others of R¹, R², R³,

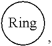

X¹, X², X³, R⁴, R⁵, R⁶, R^{7a}, R^{7b}, R^{8a}, R^{8b}, R^{9a}, R^{9b}, R^{A}, R^{B}, R^{C}, R^{D}, R^{E}, R^{F}, R^{G1}, R^{G2}, R^{S}, R^{T} and n in Formula I. Such combination is contemplated and within the scope of the present invention.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds of the present disclosure can comprise one or more asymmetric centers and/or axial chirality, and thus can exist in various isomeric forms, e.g., enantiomers and/or diastereomers. For example, the compounds described herein can be in the form of an individual enantiomer, diastereomer, atropisomer, or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, it should be understood that with respect to that particular chiral center or axial chirality, the compound exists predominantly as the as-drawn stereoisomer, such as with less than 20%, less than 10%, less than 5%, less than 1%, by weight, by HPLC area, or both, or with a non-detectable amount of the other stereoisomer(s). The presence and/or amounts of stereoisomers can be determined by those skilled in the art in view of the present disclosure, including through the use of chiral HPLC.

Compounds of the present disclosure can have atropisomers. In any of the embodiments described herein, when applicable, the compound of the present disclosure can exist as a mixture of atropisomers in any ratio. In some embodiments, when applicable, the compound can exist as an isolated individual atropisomer substantially free (e.g., with less than 20%, less than 10%, less than 5%, less than 1%, by weight, by HPLC area, or both, or with a non-detectable amount) of the other atropisomer(s). The Examples section shows some exemplary isolated atropisomers of compounds of the present disclosure. As understood by those skilled in the art, when the rotation is restricted around a single bond, e.g., a biaryl single bond, a compound may exist in a mixture of atropisomers with each individual atropisomer isolable.

When a range of values is listed, it is intended to encompass each value and sub-range within the range. For example, “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” or “compound(s) of the present invention” refers to any of the compounds described herein according to Formula I (e.g., Formula I-1, I-1-A, I-1-A-a, I-1-A-b, I-1-A-c, I-1-A-d, I-1-A-e, I-1-A-f, I-1-A-g, I-1-A-h, I-2, I-2-A, I-2-B, I-2-C, I-2-D, or I-2-E), any of compounds shown in Table A and in the Examples section, isotopically labeled compound(s) thereof (such as a deuterated analog wherein one of the hydrogen atoms is substituted with a deuterium atom with an abundance above its natural abundance), possible stereoisomers thereof (including diastereoisomers, enantiomers, and racemic mixtures), geometric isomers thereof, atropisomers thereof, tautomers thereof, conformational isomers thereof, and/or pharmaceutically acceptable salts thereof (e.g., acid addition salt such as HCl salt or base addition salt such as Na salt). Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₁₂ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group as used herein refers to a group selected from methyl, ethyl, propyl (n-propyl), isopropyl, butyl (n-butyl), sec-butyl, tert-butyl, and iso-butyl. An optionally substituted C₁₋₄ alkyl group refers to the C₁₋₄ alkyl group as defined, optionally substituted with one or more permissible substituents as described herein. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “heteroalkyl” refers to an alkyl group as defined above, with one or more carbon being replaced with a heteroatom, such as O or N. A heteroalkyl can be designated by its number of carbons. For example, a C₁₋₄ heteroalkyl refers to a heteroalkyl group containing 1-4 carbons. When optionally substituted, either the heteroatom or the carbon atom of the heteroalkyl group can be substituted with a permissible substituent. As used herein, the term “heteroalkylene” as used by itself or as part of another group refers to a divalent radical derived from a heteroalkyl group.

As used herein, the term “alkenyl” as used by itself or as part of another group refers to an alkyl group as defined above containing one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to an alkyl group as defined above containing one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆haloalkyl group. In one embodiment, the haloalkyl group is a C₁₋₄ haloalkyl group.

“Carbocyclyl” or “carbocyclic” as used by itself or as part of another group refers to a radical of a non-aromatic cyclic hydrocarbon group having from 3 to 10 ring carbon atoms (“C₃₋₁₀ carbocyclyl”) and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbomyl, decalin, adamantyl, cyclopentenyl, and cyclohexenyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”).

“Heterocyclyl” or “heterocyclic” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

As commonly understood by those skilled in the art, alkylene, alkenylene, alkynylene, carbocyclylene, heterocyclylene, arylene, and heteroarylene refer to the corresponding divalent radicals of alkyl, alkenyl, alkynyl, carbocyclyl, heterocyclyl, aryl, and heteroaryl groups, respectively.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” non-aromatic group herein can be unsubstituted or substituted with 1, 2, or 3 substituents or even 4 or 5 substituents independently selected from F, Cl, —OH, oxo (as applicable), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1 or 2 ring heteroatoms or even 3 or 4 ring heteroatoms independently selected from O, S, and N, 4-7 membered heterocyclyl containing 1 or 2 ring heteroatoms or even 3 or 4 ring heteroatoms independently selected from O, S, and N, or independently selected from Br, I, —NH₂ and —CN, wherein each of the alkyl, alkenyl, alkynyl, alkoxy, cycloalkyl, cycloalkoxy phenyl, heteroaryl, and heterocyclyl, is optionally substituted with 1, 2, or 3 substituents or even 4 or 5 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, or independently selected from Cl, Br, I, —NH₂ and —CN. In some embodiments, the “optionally substituted” aromatic group (including aryl and heteroaryl groups) herein can be unsubstituted or substituted with 1, 2, or 3 substituents or even 4 or 5 substituents independently selected from F, Cl, —OH, —CN, C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1 or 2 ring heteroatoms or even 3 or 4 ring heteroatoms independently selected from O, S, and N, 4-7 membered heterocyclyl containing 1 or 2 ring heteroatoms or even 3 or 4 ring heteroatoms independently selected from O, S, and N, or independently selected from Br, I and —NH₂, wherein each of the alkyl, alkenyl, alkynyl, alkoxy, cycloalkyl, cycloalkoxy, phenyl, heteroaryl, and heterocyclyl, is optionally substituted with 1, 2, or 3 substituents or even 4 or 5 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy, or independently selected from C₁, Br, —NH₂ and —CN.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa} SC(═O)OR^{aa}, —SC(═O)R^{aa}, —P(═O)(R^{aa})₂, —P(═O)(OR^{cc})₂, —OP(═O)(R^{aa})₂, —OP(═O)(OR″)₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄—P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₃₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₄₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR″, —N(R^{cc})₂, —CN, —C(═O)R^{aa}, —C(═O)N(R^{cc})₂, —CO₂R^{aa}, —SO₂R^{aa}, —C(═NR″)OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R″)₂, —P(═O)(OR^{cc})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, OC(═NR^{ff})R^{ee}, —OC(═NR″)OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R″, —SO₂OR^{ee}, —OSO₂R, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₁₋₁₀ aryl, 3-10 membered heterocyclyl, 5-10 membered heteroaryl; or two geminal R⁹ substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, BPh₄⁻, Al(OC(CF₃)₃)₄⁻, and a carborane anion (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R″)₂, —C(═O)R^{aa}, —C(═O)N(R″)₂, —CO₂R^{aa}, SO₂R^{aa}, —C(═NR″)R^{aa}, —C(═NR^{cc})OR^{aa}, —C(═NR^{cc})N(R^{cc})₂, —SO₂N(R^{cc})₂, —SO₂R^{cc}, SO₂OR^{cc}, —SOR^{aa}, —C(═S)N(R^{cc})₂, —C(═O)SR^{cc}, —C(═S)SR^{cc}, C₁₋₁₀ alkyl, ar-C₁₋₁₀ alkyl, heteroar-C₁₋₁₀ alkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein.

Exemplary oxygen atom substituents include, but are not limited to, —R^{aa}, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, alkyl ethers or substituted alkyl ethers such as methyl, allyl, benzyl, substituted benzyls such as 4-methoxybenzyl, methoxylmethyl (MOM), benzyloxymethyl (BOM), 2-methoxyethoxymethyl (MEM), etc., silyl ethers such as trymethylsilyl (TMS), triethylsilyl (TES), triisopropylsilyl (TIPS), t-butyldimethylsilyl (TBDMS), etc., acetals or ketals, such as tetrahydropyranyl (THP), esters such as formate, acetate, chloroacetate, dichloroacetate, trichloroacetate, trifluoroacetate, methoxyacetate, etc., carbonates, sulfonates such as methanesulfonate (mesylate), benzylsulfonate, and tosylate (Ts), etc.

The term “leaving group” is given its ordinary meaning in the art of synthetic organic chemistry, for example, it can refer to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat”, “treating”, “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the phrase “administration” of a compound, “administering” a compound, or other variants thereof means providing the compound or a prodrug of the compound to the individual in need of treatment.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra.

The abbreviations used in the present disclosure are described as follows:

| Abbreviation | Chemical Name |
|---|---|
| AcOH | Acetic Acid |
| AIBN | 2,2′-Azobis(2-methylpropionitrile) |
| BINAP | racemic-2,2′-Bis(diphenylphosphino)-1,1′- |
| | binaphthyl |
| Bu^{t}OK | Potassium tert-butoxide |
| DABCO | 1,4-Diazabicyclo[2.2.2]octane |
| DAST | Diethylaminosulphur trifluoride |
| DCE | Dichloroethane |
| DCM | dichloromethane |
| DEA | Diethylamine |
| DEAD | DiethylAzodicarboxylate |
| DIAD | Diisopropyl azodicarboxylate |
| DIPEA | N,N-Diisopropylethylamine |
| DMA | N,N-Dimethylacetamide |
| DMAP | 4-Dimethylaminopyridine |
| DMF | N,N-dimethylformamide |
| DMSO | Dimethyl sulfoxide |
| EtOAc | Ethyl acetate |
| EtOH | Ethanol |
| FA | Formic acid |
| HATU | O-(7-Azabenzotriazol-1-yl)-N,N,N′,N′- |
| | tetramethyluronium hexafluorophosphate |
| HPLC | High Performance Liquid Chromatography |
| KHMDS | Potassium bis(trimethylsilyl)amide |
| KOAc | Potassium acetate |
| LDA | Lithium diisopropylamide |
| LiHMDS | Lithium bis(trimethylsilyl)amide |
| MeCN | Acetonitrile |
| MeOH | Methanol |
| NaOBu^{t} | Sodium tert Butoxide |
| NBS | N-Bromosuccinimide |
| NIS | N-Iodosuccinimide |
| NMP | N-methylpyrrolidin-2-one |
| Pd(dppf)Cl₂•CH₂Cl₂ | (1,1′-Bis(diphenylphosphino)ferrocene- |
| | palladium(II)dichloride dichloromethane |
| | complex) |
| Pd(OAc)₂ | Palladium (II) acetate |
| Pd(OH)₂ | Palladium hydroxide |
| Pd₂(dba)₃ | Tris(dibenzylideneacetone)dipalladium(0) |
| Pd(PPh₃)₄ | Tetrakis(triphenylphosphine)palladium |
| PPh₃ | triphenylphosphine |
| Ruphos | 2-Dicyclohexylphosphino-2′,6′- |
| | diisopropoxybiphenyl |
| S-BuLi | sec-Butyllithium |
| SFC | Supercritical Fluid Chromatography |
| TBAF | Tetra-n-butylammonium fluoride |
| TCFH | N,N,N′,N′-Tetramethylchloroformamidinium |
| | hexafluorophosphate |
| TEA | Triethylamine |
| TFA | Trifluoroacetic acid |
| TFAA | Trifluoroacetic anhydride |
| THF | Tetrahydrofuran |
| TMEDA | N,N,N′,N′-Tetramethylethylenediamine |
| TMSCN | Trimethylsilyl cyanide |
| Xant phos Pd G2 | Chloro[(4,5-bis(diphenylphosphino)-9,9- |
| | diMethylxanthene)-2-(2′-aMino-1,1′- |
| | biphenyl)]palladiuM(II) |
| Xantphos | 9,9-Dimethyl-4,5-bis(diphenylphosphino) |
| | xanthene |

### Example 1 Synthesis of Compound 1

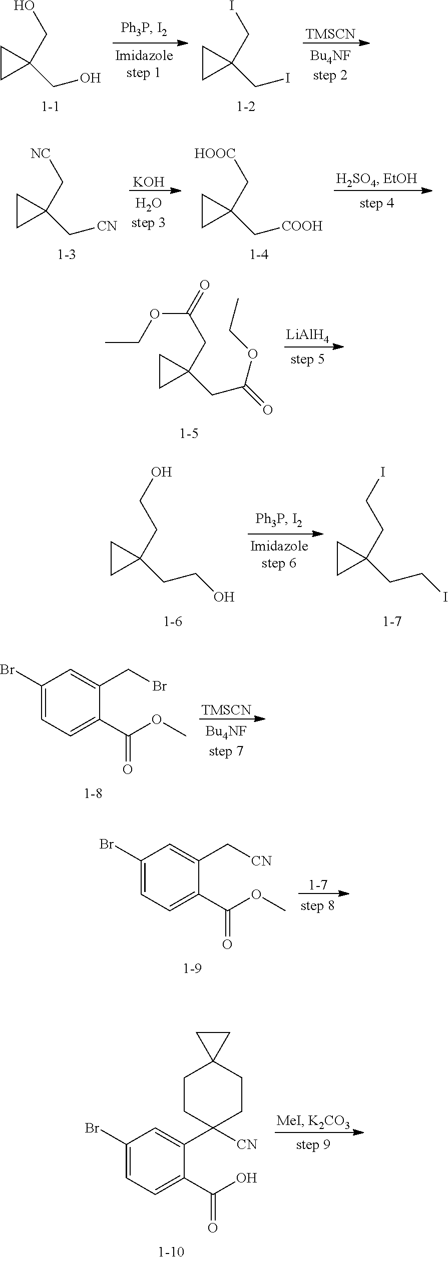

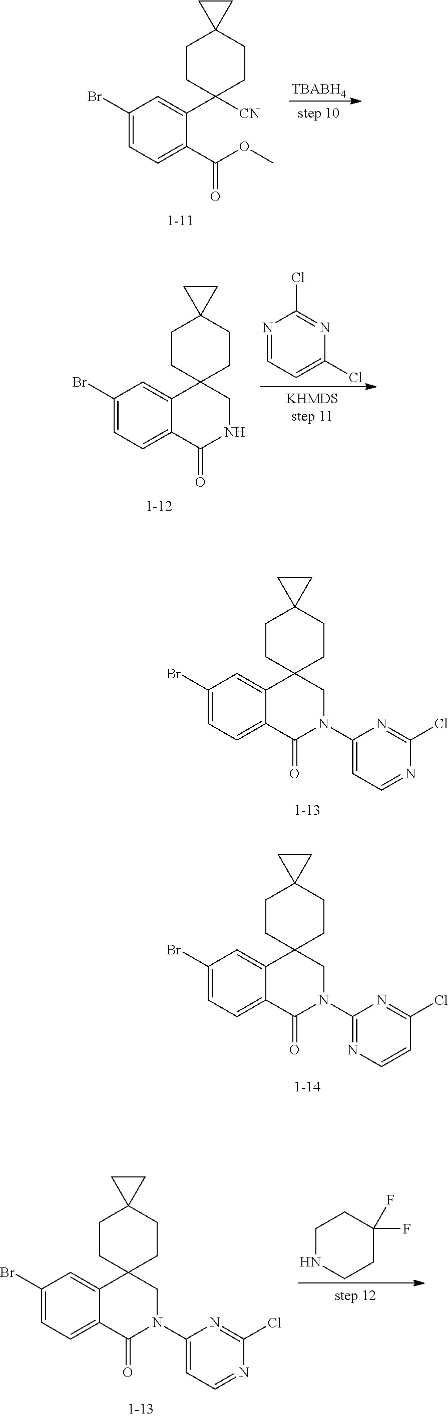

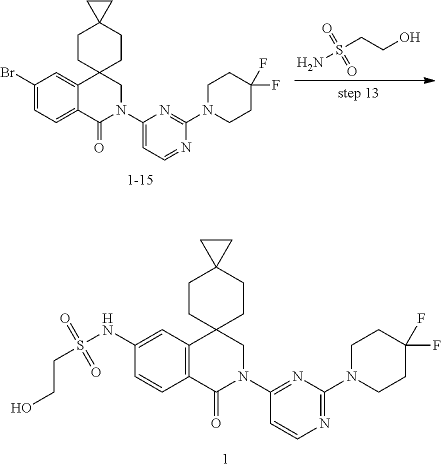 [SMILES: O=C1c2ccc(Br)cc2C2(CCC3(CC3)CC2)CN1c1ccnc(N2CCC(F)(F)CC2)n1 ; NS(=O)(=O)CCO ; O=C1c2ccc(NS(=O)(=O)CCO)cc2C2(CCC3(CC3)CC2)CN1c1ccnc(N2CCC(F)(F)CC2)n1]

Step 1: To a solution of PPh₃ (32.1 g, 122.4 mmol) and 1H-imidazole (16.7 g, 244.8 mmol) in DCM (150 mL) was treated with iodine (31.1 g, 122.4 mmol) at 0° C. After iodine was completely dissolved, a solution of 1-1 (5 g, 49.0 mmol) was added to the reaction mixture. The mixture was stirred at 0° C. for 1 h and at 25° C. for overnight. The reaction mixture was poured into water and extracted with DCM. The combined organic layers were washed with Na₂SO₃, dried over Na₂SO₄, filtered and concentrated in vacuo at 25° C. The residue was purified by column chromatography on silica gel (petroleum ether/ethyl acetate=1/0 to 100/1) to afford 1-2.

Step 2: To a solution of 1-2 (8.7 g, 27.0 mmol) and TMSCN (7.10 mL, 56.8 mmol) in THF (150 mL) was added TBAF (56.8 mL, 56.8 mmol) at 0° C. The mixture was stirred at room temperature for overnight. H₂O was added to the mixture and the mixture was extracted with EtOAc. The combined organic layers were dried over Na₂SO₄, filtered and the filtrate was concentrated under vacuo. The residue was purified by column chromatography on silica gel (petroleum ether/ethyl acetate=1/0 to 5/1) to afford 1-3.

Step 3: To a solution of 1-3 (2.0 g, 16.6 mmol) in H₂O (20 mL) was added KOH (4.67 g, 83.2 mmol) at room temperature. Then the mixture was stirred at 100° C. for 12 h. The mixture was cooled to room temperature and acidified with concentrated HCl until pH to 2. The mixture was extracted with EtOAc. The combined organic layers were dried over Na₂SO₄, filtered and the filtrate was concentrated to afford 1-4 which was used for the next step directly without further purification.

Step 4: To a solution of 1-4 (2.47 g, crude) in EtOH (20 mL) was added concentrated H₂SO₄ (2 mL) at room temperature. Then the mixture was stirred at 80° C. for 3 h. The mixture was cooled to room temperature and H₂O was added. The mixture was extracted with EtOAc. The combined organic layers were dried over Na₂SO₄, filtered and the filtrate was concentrated under vacuo to afford 1-5 which was used for the next step directly without further purification.

Step 5: To a solution of 1-5 (3.3 g, crude) in THF (30 mL) was added LiAlH₄ (12.3 mL, 30.8 mmol) at 0° C. slowly. Then the mixture was stirred at 0° C. for 1 h. The reaction was quenched with Na₂SO₄-10H₂O and the mixture was stirred at room temperature for 15 minutes. The mixture was filtered and the filtrate was concentrated under vacuo to afford 1-6 which was used for the next step directly without further purification.

Step 6: To a mixture of imidazole (9.2 g, 135.2 mmol) and PPh₃ (17.7 g, 67.6 mmol) in DCM (300 mL) was added iodine (17.2 g, 67.6 mmol) at 0° C. After the mixture was stirred at 0° C. for 15 minutes, a solution of 1-6 (2.2 g, crude) in DCM (10 mL) was added into the mixture. Then the mixture was stirred at room temperature for 1 h. H₂O was added to the mixture and the mixture was extracted with DCM. The combined organic layers were dried over Na₂SO₄, filtered and the filtrate was concentrated under vacuo. The residue was purified by column chromatography on silica gel (100% of petroleum ether) to afford 1-7.

Step 7: To a solution of 1-8 (10 g, 32.45 mmol) and TMSCN (4.53 mL, 35.7 mmol) in THF (100 mL) was added TBAF (35.7 mL, 35.7 mmol) at 0° C. The mixture was stirred for overnight at room temperature. H₂O was added to the mixture and the mixture was extracted with EtOAc. The combined organic layers were dried over Na₂SO₄, filtered and the filtrate was concentrated under vacuo. The residue was purified by column chromatography on silica gel (petroleum ether/ethyl acetate=1/0 to 6/1) to afford 1-9.

Step 8: To a solution of 1-9 (1.4 g, 5.5 mmol) in DMF (25 mL) was added NaH (661.2 mg, 16.5 mmol) at 0° C. After the mixture was stirred at 0° C. for 0.5 h, 1-7 (2.12 g, 6.1 mmol) was added to the mixture. Then the mixture was stirred at room temperature for 1 h. The reaction was quenched with H₂O and extracted with EtOAc. The combined organic layers were dried over Na₂SO₄, filtered and the filtrate was concentrated under vacuo to afford 1-10 which was used for the next step directly without further purification.

Step 9: To a solution of 1-10 (1.8 g, crude) in DMF (25 mL) were added K₂CO₃ (1.52 g, 11.0 mmol) and CH₃I (0.7 mL, 11.0 mmol) at room temperature. Then the mixture was stirred at room temperature for 1 h. The mixture was filtered and the filtrate was concentrated under vacuo. The residue was purified by column chromatography on silica gel (petroleum ether/ethyl acetate=1/0 to 6/1) to afford 1-11.

Step 10: To a solution of 1-11 (1.3 g, 3.7 mmol) in DCM (20 mL) was added tetrabutylammonium borohydride (2.88 g, 11.2 mmol) at room temperature, then the mixture was stirred at 50° C. for 4 h. The reaction was quenched with HCl (1 N) and H₂O and extracted with DCM. The combined organic layers were dried over Na₂SO₄, filtered and the filtrate was concentrated under vacuo. The residue was purified by column chromatography on silica gel (petroleum ether/ethyl acetate=1/0 to 1/1) to afford 1-12.

Step 11: To a suspension of 1-12 (100 mg, 0.31 mmol) in THF (3 mL) was added KHMDS (0.47 mL, 0.47 mmol) at 0° C. After the mixture was stirred at 0° C. for 10 minutes, 2,4-dichloropyrimidine (69.8 mg, 0.47 mmol) was added. Then the mixture was stirred at 60° C. for 5 h. The reaction was quenched with H₂O. The mixture was concentrated under vacuo, the residue was purified by column chromatography on silica gel (petroleum ether/ethyl acetate=1/0 to 0/1) to afford 1-13 and 1-14.

Step 12: A mixture of 1-13 (40 mg, 0.092 mmol), 4,4-difluorohexahydropyridine (22.3 mg, 0.18 mmol) and DIPEA (0.06 mL, 0.37 mmol) in DMF (2 mL) was heated at 100° C. for 3 h. The mixture was concentrated under vacuo, the residue was purified by column chromatography on silica gel (petroleum ether/ethyl acetate=1/0 to 6/1) to afford 1-15.

Step 13: A mixture of 1-15 (32 mg, 0.062 mmol), 2-hydroxyethane-1-sulfonamide (11.6 mg, 0.093 mmol), CuI (11.8 mg, 0.062 mmol), K₃PO₄ (39.4 mg, 0.19 mmol) and methyl[(1R,2R)-2-(methylamino)cyclohexyl]amine (4.4 mg, 0.031 mmol) in DMF (1 mL) was heated at 90′ for 2 h. The mixture was concentrated under vacuo, the residue was purified by column chromatography on silica gel (DCM/MeOH=1/0 to 20/1) to afford 1. LCMS (ESI, m/z): [M+H]⁺=562.2; ¹H NMR (400 MHz, CDCl₃, ppm) δ 8.32-8.30 (d, J=5.7 Hz, 1H), 8.19-8.17 (d, J=8.4 Hz, 1H), 7.72-7.71 (d, J=5.7 Hz, 1H), 7.39 (s, 1H), 7.24-7.19 (m, 1H), 6.94 (s, 1H), 4.46 (s, 2H), 4.21-4.14 (m, 2H), 4.08-3.97 (m, 4H), 3.42-3.34 (m, 2H), 2.46-2.40 (m, 1H), 2.12-2.01 (m, 6H), 1.98-1.87 (m, 2H), 1.79-1.70 (m, 2H), 0.93-0.84 (m, 2H), 0.42-0.28 (m, 4H). ¹⁹F NMR (376 MHz, CDCl₃, ppm) δ−96.72 (2F).

### Example 2 Synthesis of Compounds 2 and 3 PGP-268 C₃

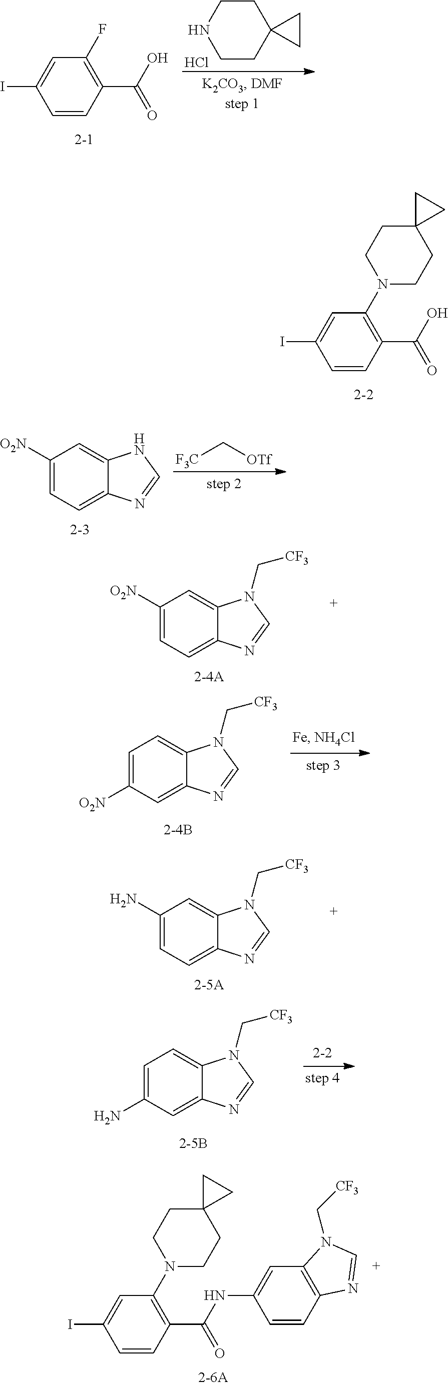

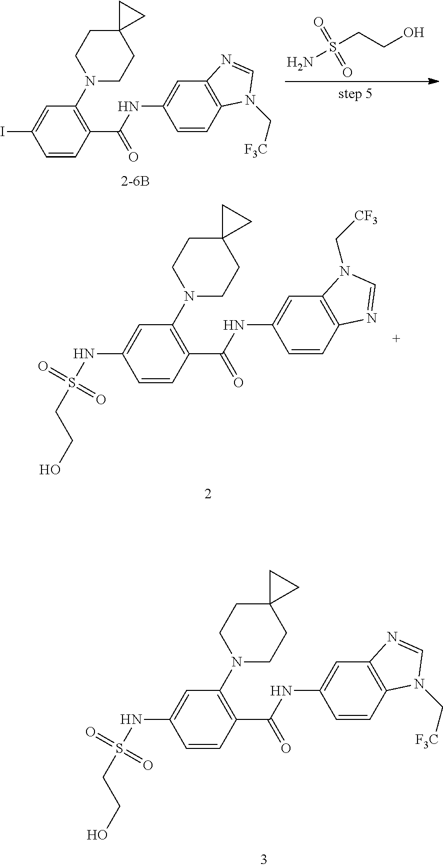 [SMILES: O=C(Nc1ccc2c(c1)ncn2CC(F)(F)F)c1ccc(I)cc1N1CCC2(CC1)CC2 ; NS(=O)(=O)CCO ; O=C(Nc1ccc2ncn(CC(F)(F)F)c2c1)c1ccc(NS(=O)(=O)CCO)cc1N1CCC2(CC1)CC2 ; O=C(Nc1ccc2c(c1)ncn2CC(F)(F)F)c1ccc(NS(=O)(=O)CCO)cc1N1CCC2(CC1)CC2]

Step 1: To a solution of 2-1 (10 g, 37.59 mmol) in DMSO (65 mL) was 6-azaspiro[2.5]octane hydrochloride (7.22 g, 48.9 mmol) at 20° C. Then K₂CO₃ (15.6 g, 112.8 mmol) was added and the reaction mixture was stirred at 140° C. for 48 h under N₂. The reaction mixture was slowly poured into ice water, then extracted with hexanes. The water phase was adjusted to pH=6 with HCl (2N). The solid was filtered and washed with water, collected and dried to afford 2-2.

Step 2: To a mixture of 2-3 (1 g, 6.13 mmol) and Cs₂CO₃ (4.0 g, 12.26 mmol) in DMF (10 mL) was added 2,2,2-trifluoroethyl trifluoromethanesulfonate (1.56 g, 6.74 mmol). The mixture was stirred at 25° C. for 16 h. The reaction mixture was diluted with water and extracted with EtOAc. The combined organic layers were dried over Na₂SO₄, filtered and concentrated under vacuo. The residue was purified by column chromatography on silica gel (petroleum ether/ethyl acetate=1/0 to 0/1) to afford a mixture of 2-4A and 2-4B.

Step 3: To a mixture of 2-4A and 2-4B (1 g, 4.08 mmol) in EtOH (10 mL) and H₂O (1 mL) were added Fe (0.7 g, 12.24 mmol) and NH₄Cl (1.1 g, 20.4 mmol). The reaction mixture was stirred at 80° C. for 4 h. The reaction mixture was diluted with EtOAc and filtered. The filtrated was concentrated to afford a mixture of 2-5A and 2-5B which was used for the next step directly without further purification.

Step 4: A mixture of 2-2 (415.1 mg, 1.16 mmol), HATU (485.9 mg, 1.28 mmol) and DIEA (0.19 mL, 1.16 mmol) in DMF (5 mL) was stirred at room temperature for 15 minutes. Then the mixture of 2-5A and 2-5B (250 mg, crude) was added to the mixture. The mixture was stirred at room temperature for 16 h. The solvent was removed under vacuo. The residue was purified by column chromatography on silica gel (petroleum ether/ethyl acetate=1/0 to 0/1) to afford a mixture of 2-6A and 2-6B.

Step 5: A mixture of 2-hydroxyethane-1-sulfonamide (169.3 mg, 1.35 mmol), CuI (103.0 mg, 0.54 mmol), Sarcosine (96.4 mg, 1.08 mmol) and K₃PO₄ (344.6 mg, 1.62 mmol) in DMF (10 mL) was heated to stirred at 50° C. for 5 minutes under N₂, then the mixture of 2-6A and 2-6B (300 mg, 0.54 mmol) was added and the resulting mixture was stirred at 100° C. for 4 h under N₂. The solvent was removed under vacuo. The residue was purified by column chromatography on silica gel (DCM/MeOH=1/0 to 10/1) to afford compounds 2 and 3. 2: LCMS (ESI, m/z): [M+H]⁺=552.2; ¹H NMR (400 MHz, DMSO-d₆, ppm) δ 11.74 (s, 1H), 10.19-10.02 (m, 1H), 8.61-8.22 (m, 2H), 7.88-7.86 (m, 1H), 7.81-7.69 (m, 1H), 7.47-7.38 (m, 1H), 7.19-7.18 (m, 1H), 7.06-7.04 (m, 1H), 5.35-5.33 (m, 2H), 5.02-4.90 (m, 1H), 3.79-3.73 (m, 2H), 3.37-3.33 (m, 2H), 3.01-2.98 (m, 4H), 1.61-1.44 (m, 4H), 0.35 (s, 4H). ¹⁹F NMR (376 MHz, DMSO-d₆, ppm) δ−69.98 (3F). 3: LCMS (ESI, m/z): [M+H]⁺=552.2; ¹H NMR (400 MHz, DMSO-d₆, ppm) δ 11.74 (s, 1H), 10.10 (s, 1H), 8.29-8.23 (m, 2H), 7.85-7.82 (m, 1H), 7.72-7.70 (m, 1H), 7.63-7.61 (m, 1H), 7.17-7.16 (m, 1H), 7.04-7.02 (m, 1H), 5.37-5.31 (m, 2H), 4.98-4.95 (m, 1H), 3.79-3.74 (m, 2H), 3.37-3.33 (m, 2H), 3.00-2.98 (m, 4H), 1.58-1.45 (m, 4H), 0.35 (s, 4H). ¹⁹F NMR (376 MHz, DMSO-d₆, ppm) δ−70.28 (3F).

### Example 3 Synthesis of Compounds 4 and 5

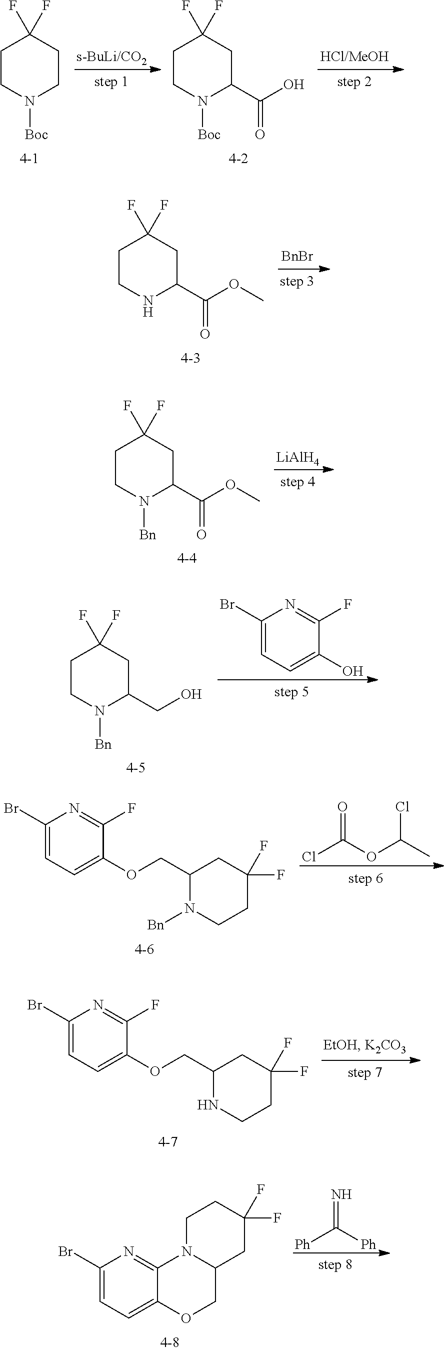

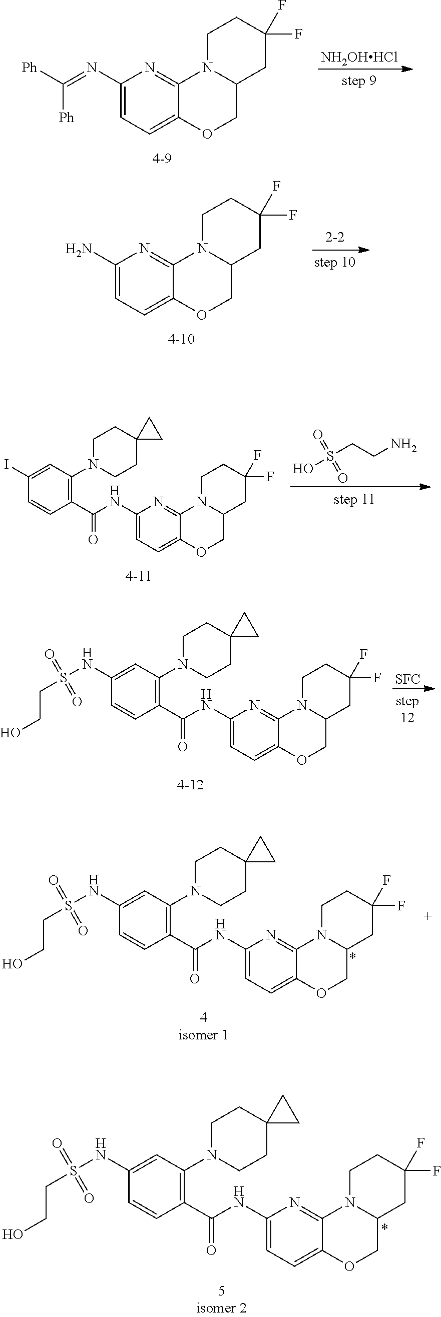

Step 1: To a solution of 4-1 (6.0 g, 27.12 mmol) in THF (300 mL) was added N, N, N′, N′-tetramethylethylenediamine (5.67 g, 48.81 mmol) at −78° C. under N₂, then S-BuLi (39.64 mL, 51.53 mmol) was added in 15 minutes and the reaction mixture was stirred at −78° C. for 1.5 h. Dry Ice (11.9 g, 271.19 mmol) was added into the reaction mixture and stirred at −78° C. for 10 minutes. The reaction was quenched with NH₄Cl aqueous, then HCl (1N) was added to the mixture to adjust the pH to 5. The mixture was extracted with EtOAc, the organic layer was dried over Na₂SO₄, filtered and concentrated to afford 4-2 which was used for the next step directly without further purification.

Step 2: A solution of 4-2 (4.0 g, crude) in HCl/MeOH (40.00 mL) was stirred at 60° C. for 36 h. The reaction mixture was concentrated in vacuo. EtOAc and NaHCO₃ was added to the residue. The organic layer was separated and washed with brine, dried over Na₂SO₄, filtered and concentrated in vacuo to afford 4-3 which was used for the next step directly without further purification.

Step 3: To a solution of 4-3 (1.46 g, 8.14 mmol) in MeCN (20 mL) was added Na₂CO₃ (1.73 g, 16.29 mmol) and benzyl bromide (1.45 mL, 12.22 mmol), the reaction mixture was stirred at 80° C. for 3 h. The mixture was concentrated in vacuo, the residue was diluted with EtOAc and water. The organic layer was separated and washed with brine, dried over Na₂SO₄, filtered and concentrated under vacuo. The residue was purified by column chromatography on silica gel (petroleum ether/ethyl acetate=1/0 to 5/1) to afford 4-4.

Step 4: A solution of 4-4 (1.37 g, 5.09 mmol) in THF (5 mL) was added to a solution of LiAlH₄ (2.04 mL, 5.1 mmol) in THF (15 mL) at 0° C., the reaction was stirred at 0° C. for 10 minutes. The reaction was quenched with Na₂SO₄·10H₂O and diluted with EtOAc. The mixture was filtered and the filtrate was concentrated in vacuo. The residue was purified by column chromatography on silica gel (DCM/MeOH=1/0 to 10/1) to afford 4-5.

Step 5: To a solution of 4-5 (226.2 mg, 0.94 mmol) in THF (5 mL) was added 6-bromo-2-fluoropyridin-3-ol, PPh₃ (614.8 mg, 2.34 mmol) and DEAD (614.8 mg, 2.34 mmol) at 0° C., the mixture was stirred at room temperature for 1 h. The reaction mixture was concentrated in vacuo, the residue was diluted with EtOAc and water. The organic layer was separated and washed with brine, dried over Na₂SO₄, filtered and concentrated under vacuo. The residue was purified by column chromatography on silica gel (petroleum ether/ethyl acetate=1/0 to 4/1) to afford 4-6.

Step 6: To a solution of 4-6 (322 mg, 0.78 mmol) in 1,2-dichloroethane (6 mL) was added 1-chloroethyl chloromethanoate (443.4 mg, 3.10 mmol), the mixture was stirred at 110° C. for 18 h. The mixture was concentrated in vacuo, MeOH (8 mL) was added to the residue and the mixture was stirred at 80° C. for 1 h. The reaction mixture was concentrated under vacuo to afford 4-7 which was used for the next step directly without further purification.

Step 7: To a solution of 4-7 (280 mg, crude) in EtOH (10 mL) was added K₂CO₃ (428.1 mg, 3.1 mmol) and the mixture was stirred at 80° C. for 3 h. The reaction mixture was concentrated under vacuo. The residue was diluted with EtOAc and water. The organic layer was separated and washed with brine, dried over Na₂SO₄, filtered and concentrated under vacuo. The residue was purified by column chromatography on silica gel (petroleum ether/ethyl acetate=1/0 to 4/1) to afford 4-8.

Step 8: To a solution of 4-8 (142 mg, 0.47 mmol) in toluene (5 mL) was added BINAP (58.0 mg, 0.093 mmol), NaOBu^{t} (89.4 mg, 0.93 mmol), diphenylmethanimine (97.0 mg, 0.54 mmol) and Pd₂(dba)₃ (85.2 mg, 0.093 mmol) under N₂, the mixture was stirred at 100° C. for 18 h. The mixture was diluted with EtOAc and water, the organic layer was separated and washed with brine, dried over Na₂SO₄, filtered and concentrated under vacuo to afford 4-9 which was used for the next step directly without further purification.

Step 9: To a solution of 4-9 (186.5 mg, crude) in MeOH (5 mL) was added Hydroxylamine hydrochloride (159.8 mg, 2.30 mmol), the mixture was stirred at room temperature for 30 minutes. The mixture was concentrated in vacuo. The residue was diluted with EtOAc and water, the organic layer was separated and washed with brine, dried over Na₂SO₄, filtered and concentrated under vacuo. The residue was purified by column chromatography on silica gel (petroleum ether/ethyl acetate=1/0 to 4/1) to afford 4-10.

Step 10: To a solution of 2-2 (139.7 mg, 0.39 mmol) in DMF (3 mL) was added 4-10 (78.6 mg, 0.33 mmol), DIPEA (0.14 mL, 0.82 mmol) and HATU (185.8 mg, 0.49 mmol), the mixture was stirred at 0° C. for 30 minutes. The mixture was diluted with EtOAc and water. The organic layer was separated and washed with brine, dried over Na₂SO₄, filtered and concentrated under vacuo. The residue was purified by column chromatography on silica gel (petroleum ether/ethyl acetate=1/0 to 4/1) to afford 4-11.

Step 11: To a solution of 2-hydroxyethane-1-sulfonamide (53.9 mg, 0.43 mmol) in DMF (2 mL) was added CuI (20.5 mg, 0.11 mmol), N-methylglycine (19.2 mg, 0.22 mmol), and K₃PO₄ (228.6 mg, 1.08 mmol), the reaction was stirred at 50° C. for 5 minutes under N₂. Then 4-11 (125 mg, 0.22 mmol) in DMF (0.5 mL) was added into the reaction mixture, the reaction mixture was heated at 100° C. for 3 h. The mixture was diluted with EtOAc and water. The organic layer was separated and washed with brine, dried over Na₂SO₄, filtered and concentrated under vacuo. The residue was purified by column chromatography on silica gel (DCM/MeOH=1/0 to 5/1) to afford 4-12.

Step 12: 4-12 (100 mg) was purified by SFC (column: ChiralCel OJ, 250×30 mm I.D., 10 μm (0.1% NH₃·H₂O in methanol)/Supercritical CO2=25/75) to afford 4 (48 mg) and 5 (33 mg) respectively. 4: SFC analysis: 98.88% ee; retention time: 4.358 min; column: ChiralCel OJ, 150×4.6 mm I.D., 3 μm, methanol (0.05% DEA) in CO2, 5% to 40%; pressure: 100 bar; flow rate: 2.5 mL/min. LCMS (ESI, m/z): [M+H]⁺=578.4; ¹H NMR (400 MHz, CDCl₃, ppm): 6 12.99 (s, 1H), 8.22-8.19 (m, 1H), 7.71-7.69 (m, 1H), 7.32 (s, 114), 7.20 (s, 1H), 7.06-7.04 (m, 2H), 4.90-4.73 (m, 114), 4.34-4.23 (m, 114), 4.18-4.09 (m, 2H), 4.05-3.97 (m, 1H), 3.62-3.51 (m, 1H), 3.39-3.29 (m, 2H), 3.13-3.00 (m, 4H), 2.99-2.84 (m, 2H), 2.23-1.62 (m, 8H), 0.39 (s, 4H). ¹⁹F NMR (376 MHz, CDCl₃, ppm) δ−89.49 (1F), −102.64 (1F). 5: SFC analysis: 99.5% ee; retention time: 4.571 min; column: ChiralCel OJ, 150×4.6 mm I.D., 3 μm, methanol (0.05% DEA) in CO2, 5% to 40%; pressure: 100 bar; flow rate: 2.5 mL/min. LCMS (ESI, m/z): [M+H]⁺=578.4; ¹H NMR (400 MHz, CDCl₃, ppm): δ 12.92 (s, 1H), 8.15-8.13 (m, 1H), 7.64-7.62 (m, 1H), 7.25 (s, 1H), 7.09 (s, 1H), 6.99-6.97 (m, 2H), 4.83-4.71 (m, 1H), 4.27-4.19 (m, 1H), 4.11-4.02 (m, 2H), 3.98-3.90 (m, 1H), 3.54-3.45 (m, 1H), 3.32-3.23 (m, 2H), 3.06-2.92 (m, 4H), 2.92-2.73 (m, 2H), 2.15-1.55 (m, 8H), 0.33 (s, 4H). ¹⁹F NMR (376 MHz, CDCl₃, ppm) δ−89.48 (1F), −102.63 (1F).

### Example 4 Synthesis of Compound 6

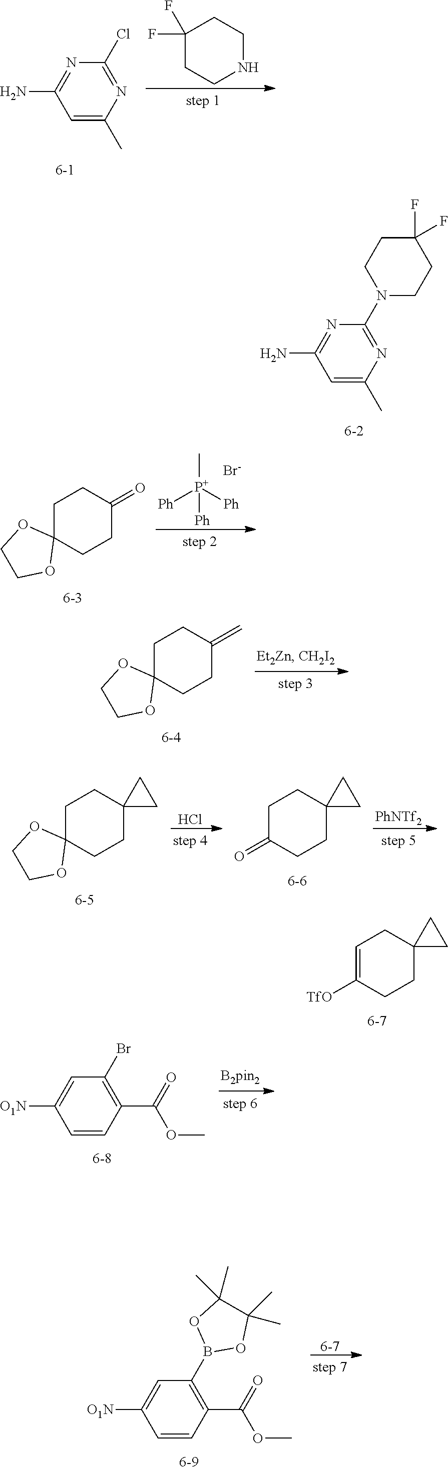

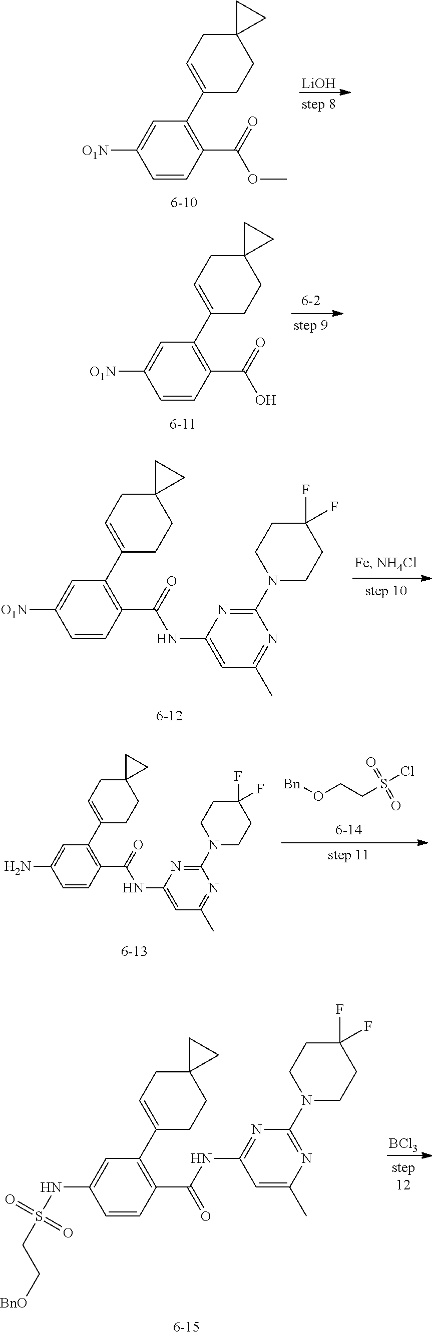

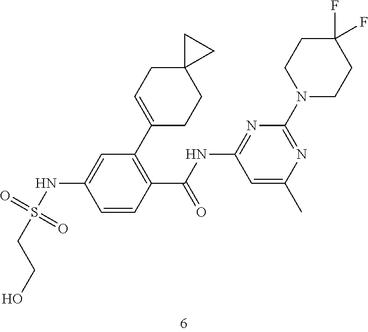 [SMILES: Cc1cc(NC(=O)c2ccc(NS(=O)(=O)CCO)cc2C2=CCC3(CC2)CC3)nc(N2CCC(F)(F)CC2)n1]

Step 1: A mixture of 6-1 (2.0 g, 13.93 mmol), 4,4-difluorohexahydropyridine (2.53 g, 20.9 mmol) and DIPEA (6.91 mL, 41.79 mmol) in NMP (20 mL) was stirred at 170° C. for 28 h. The mixture was diluted with EtOAc and water. The organic layer was separated and washed with brine, dried over Na₂SO₄, filtered and concentrated under vacuo. The residue was purified by column chromatography on silica gel (petroleum ether/ethyl acetate=1/0 to 1/1) to afford 6-2.

Step 2: To a suspension of Methyltriphenylphosphonium bromide (54.9 g, 153.67 mmol) in THF (60 mL) was added n-Butyllithium (2.5M, 61.47 mL, 153.67 mmol) at −78° C. slowly. After addition, the mixture was stirred at −78° C. for 15 minutes and 0° C. for 1 h. This mixture was cooled to −78° C., and a solution of 6-3 (20 g, 128.06 mmol) in THF (60 mL) was added to the mixture slowly. Then the mixture was stirred at room temperature for 1 h. The reaction was quenched with saturated NH₄Cl. This mixture was extracted with EtOAc. The combined organic layers were dried over Na₂SO₄, filtered and the filtrate was concentrated under vacuo. The residue was purified by column chromatography on silica gel (petroleum ether/ethyl acetate=1/0 to 10/1) to afford 6-4.

Step 3: To a solution of 8 6-4 (11 g, 71.33 mmol) in toluene (110 mL) was added diethylzinc (1M, 178.33 mL, 178.33 mmol) at −60° C. The mixture was stirred for 15 minutes at this temperature. Diiodomethane (28.73 mL, 356.66 mmol) was added dropwise to the mixture over a period of 30 minutes. The solution warmed to room temperature and stirred for 16 h. The reaction was quenched by saturated NH₄Cl. The mixture was extracted with diethyl ether. The organic phase was washed with water and dried over Na₂SO₄, filtered and the filtrate was concentrated under vacuo. The residue was purified by column chromatography on silica gel (petroleum ether/ethyl acetate=1/0 to 10/1) to afford 6-5.

Step 4: To the solution of 6-5 (9.2 g, 54.68 mmol) in THF (40 mL) was added HCl (1M, 40 mL, 40.0 mmol) at room temperature, the mixture was stirred at room temperature for 16 h. The reaction mixture was extracted with EtOAc. The organic phase was dried by Na₂SO₄, filtered and concentrated, the residue was purified by column chromatography on silica gel (petroleum ether/ethyl acetate=1/0 to 10/1) to afford 6-6.

Step 5: To the stirred solution of 6-6 (4.5 g, 36.24 mmol) in dry THF (60 mL) was added dropwise LiHMDS (1M, 39.86 mL, 39.86 mmol) at −78° C. and stirred for 1 h under N₂. A solution of N, N-Bis(trifluoromethylsulfonyl)aniline (15.5 g, 43.49 mmol) in THF (40 mL) was added dropwise to the mixture at −78° C. The mixture was stirred at room temperature for another 2 h. The reaction was quenched with water and extracted with EtOAc. The combined organic phase was washed with brine, dried over anhydrous Na₂SO₄ and concentrated in vacuo. The residue was purified by column chromatography on silica gel (petroleum ether/ethyl acetate=10/1 to 5/1) to afford 6-7.

Step 6: The mixture of 6-8 (4.2 g, 16.15 mmol), Bis(pinacolato)diboron (6.2 g, 24.23 mmol), KOAc (3.2 g, 32.30 mmol) and Pd(dppf)Cl₂·CH₂Cl₂ (1.3 g, 1.62 mmol) in dioxane (50 mL) was stirred at 90° C. under N₂ for 16 h. The reaction was quenched by water and extracted with EtOAc. The organic phase was dried over anhydrous Na₂SO₄, filtered and concentrated in vacuo, the residue was purified by column chromatography on silica gel (petroleum ether/ethyl acetate=5/1 to 3/1) to afford 6-9.

Step 7: The mixture of 6-7 (1.88 g, 7.33 mmol), 6-9 (2.70 g, 8.79 mmol), K₂CO₃ (3.0 g, 21.98 mmol) and Pd(dppf)Cl₂·CH₂Cl₂ (0.6 g, 0.73 mmol) in dioxane (35 mL) and H₂O (7 mL) was stirred at 90° C. under N₂ for 2 h. The reaction mixture was extracted with EtOAc. The organic layer was dried over anhydrous Na₂SO₄, filtered and concentrated in vacuo, the residue was purified by column chromatography on silica gel (petroleum ether/ethyl acetate=20/1 to 10/1) to afford 6-10.

Step 8: To the solution of 6-10 (1.85 g, 6.44 mmol) in THF (15 mL) and MeOH (5 mL) was added LiOH (1 M, 30 mL, 30 mmol) at room temperature, the mixture was stirred at room temperature for 3 h. HCl was added to the mixture to adjust the pH to 7, the mixture was extracted with EtOAc. The organic layer was dried over Na₂SO₄, filtered and concentrated in vacuo to afford 6-11 which was used for the next step directly without further purification.

Step 9: To a solution of 6-11 (1.68 g, crude) in DMF (20 mL) was added DIPEA (3.55 mL, 21.47 mmol) and TCFH (2.1 g, 7.36 mmol) at room temperature, the mixture was stirred at room temperature for 1 h. Then 6-2 (1.4 g, 6.13 mmol) was added to the reaction mixture, then the mixture was stirred at 100° C. for 16 h. The reaction was quenched with water and extracted with EtOAc. The organic layer was washed with water, dried over anhydrous Na₂SO₄, filtered and concentrated in vacuo. The residue was purified by column chromatography on silica gel (petroleum ether/ethyl acetate=10/1 to 3/1) to afford 6-12.

Step 10: To the solution of 6-12 (520 mg, 1.08 mmol) in EtOH (15 mL) and H₂O (15 mL) was added HCl (0.5 mL), NH₄Cl (460.2 mg, 8.60 mmol) and Fe (240 mg, 4.30 mmol) at room temperature, the mixture was stirred at 70° C. for 2 h. The mixture was extracted with EtOAc, the organic layer was dried over anhydrous Na₂SO₄, filtered and concentrated in vacuo to afford 6-13 which was used for the next step directly without further purification.

Step 11: To a solution of 6-13 (200 mg, 0.44 mmol) in THF (8 mL) was added triethylamine (0.18 mL, 1.32 mmol) and DMAP (16.2 mg, 0.13 mmol), the mixture was cooled to 0° C., then 6-14 (310.5 mg, crude) in THF (5 mL) was added into the reaction mixture and stirred for 30 min. The mixture was concentrated in vacuo. The residue was diluted with EtOAc and water. The organic layer was separated and washed with saturated brine. The organic layer was dried over anhydrous Na₂SO₄, filtered and concentrated in vacuo. The residue was purified by column chromatography on silica gel (petroleum ether/ethyl acetate=1/0 to 3/1) to afford 6-15.

Step 12: To a solution of 6-15 (100 mg, 0.15 mmol) in DCM (2 mL) was added BCl₃ at −78° C. under N₂. The reaction mixture was stirred at −78° C. for 1 h. The reaction was quenched with MeOH (3 mL) and concentrated in vacuo. The residue was diluted with EtOAc and water. The organic layer was separated and washed with saturated brine. The organic layer was dried over anhydrous Na₂SO₄, filtered and concentrated in vacuo. The residue was purified by column chromatography on silica gel (DCM/MeOH=1/0 to 5/1) and then by reverse phase HPLC (MeCN/water: 0-70%) to afford 6. LCMS (ESI, m/z): [M+H]⁺=562.2; ¹H NMR (400 MHz, DMSO-d₆, ppm) δ 10.21-9.77 (m, 2H), 7.56-7.44 (m, 1H), 7.28-7.11 (m, 2H), 7.10-7.02 (m, 1H), 5.79-5.67 (m, 1H), 5.28-4.60 (m, 1H), 3.97-3.66 (m, 6H), 3.32-3.27 (m, 2H), 2.34-2.23 (m, 5H), 2.04-1.84 (m, 6H), 1.48-1.35 (m, 2H), 0.32 0.17 (m, 4H). ¹⁹F NMR (376 MHz, DMSO-d₆, ppm) 8-94.87 (2F).

### Example 5 Synthesis of Compound 7

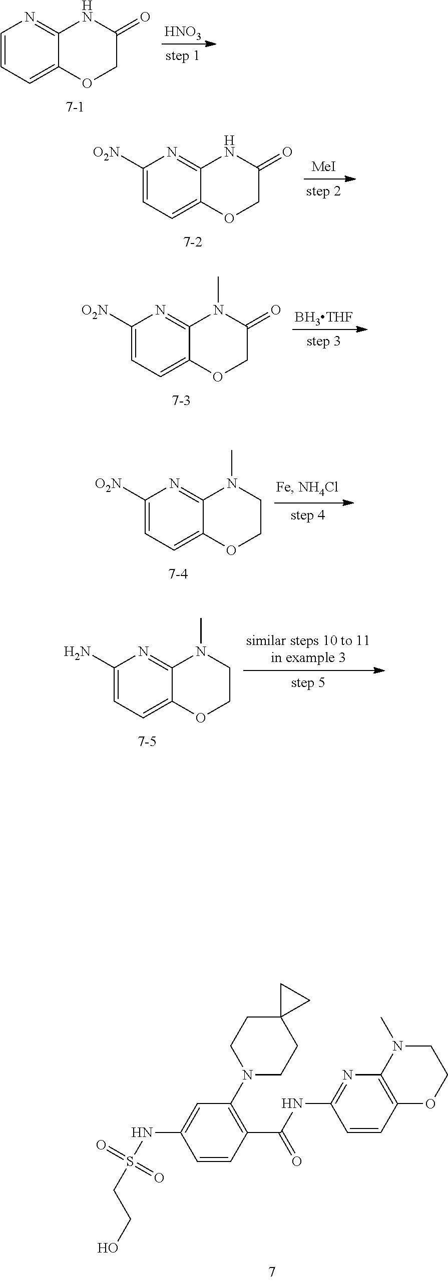

Step 1: To a solution of 7-1 (1 g, 6.66 mmol) in concentrated H₂SO₄ (3.5 mL) was added a mixture of concentrated H₂SO₄ (1.5 mL) and concentrated nitric acid (1.5 mL, 6.66 mmol) at 0° C. for 5 minutes, and the reaction was stirred at room temperature for 1 h. The reaction solution was poured into ice. The precipitated powder was filtered, washed with water, and then dried to afford 7-2.

Step 2: To a solution of 7-2 (1 g, 5.13 mmol) in DMF (10 mL) was added Mel (1.5 g, 10.25 mmol) and K₂CO₃ (2.1 g, 15.37 mmol), the reaction mixture was stirred at room temperature for 16 h. The mixture was diluted with ice-water. The precipitated powder was filtered, washed with water, and then dried to afford 7-3.

Step 3: To a solution of 7-3 (1 g, 4.78 mmol) in THF (10 mL) was added BH₃·THF (9.56 mL, 9.56 mmol), the reaction mixture was stirred at 80° C. for 2 h. The reaction was quenched with MeOH at 70° C. The mixture was concentrated in vacuo to afford 7-4.

Step 4: To a solution of 7-4 (120 mg, 0.62 mmol) in H₂O (0.3 mL) and EtOH (2 mL) was added Fe (171.7 mg, 3.08 mmol) and NH₄Cl (164.5 mg, 3.08 mmol), the mixture was stirred at 75° C. for 1 h. The reaction mixture was filtered, the filtrate was concentrated in vacuo to afford 7-5 which was used for the next step directly without further purification.

Step 5: Compound 7 was prepared from compound 7-5 following the procedure for the synthesis of compound 4-12 in example 3. LCMS (ESI, m/z): [M+H]⁺=502.4; ¹H NMR (400 MHz, DMSO-d₆, ppm) δ12.88 (s, 1H), 10.18 (s, 1H), 8.07-8.05 (m, 1H), 7.43-7.41 (m, 1H), 7.27-7.26 (m, 1H), 7.15-7.05 (m, 1H), 6.98-6.96 (m, 1H), 5.10-4.85 (m, 1H), 4.26-4.14 (m, 2H), 3.78-3.75 (m, 2H), 3.50-3.40 (m, 2H), 3.40-3.31 (m, 2H), 3.06 (s, 3H), 3.10-2.85 (m, 4H), 2.05-1.50 (m, 4H), 0.38 (s, 4H).

### Example 6 Synthesis of Compound 9

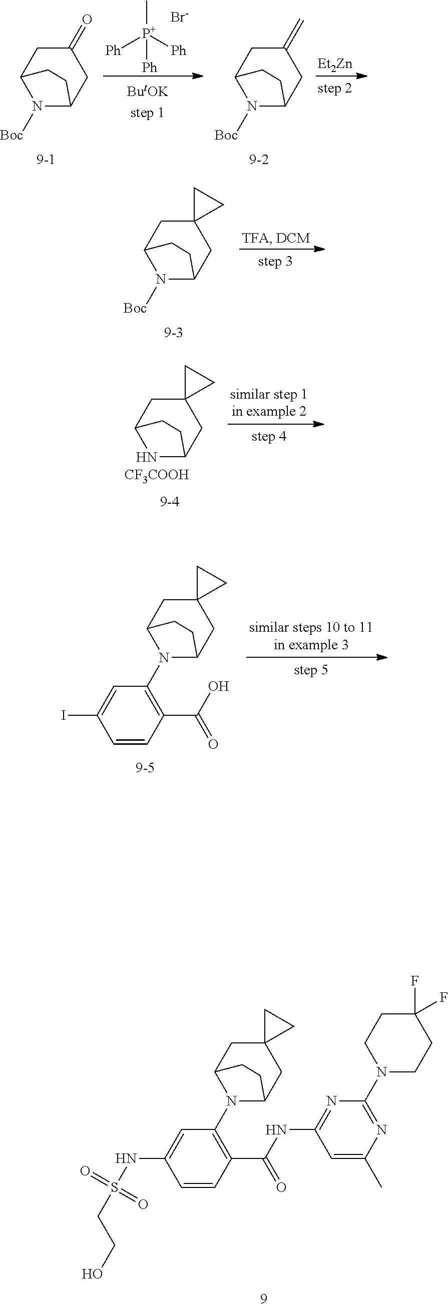

Step 1: To a solution of Methyltriphenylphosphonium bromide (15.9 g, 44.39 mmol) in THF (50 mL) was added Bu^{t}OK (5.0 g, 44.39 mmol) at 15° C. The mixture was stirred at 55° C. under N₂ for 2 h. Then 9-1 (5.0 g, 22.19 mmol) was added to the mixture and stirred at 55° C. for 16 h. The mixture was concentrated under vacuo. The residue was purified by column chromatography on silica gel (petroleum ether/ethyl acetate=1/0 to 10/1) to afford 9-2.

Step 2: Diethylzinc (34.70 mL, 34.70 mmol) was added to a solution of 9-2 (3.1 g, 13.88 mmol) in toluene (10 mL) at −60 to −50° C. The mixture was stirred for 15 minutes, then diiodomethane (5.59 mL, 69.41 mmol) was added dropwise to the mixture over 30 minutes. The mixture was warmed to room temperature and stirred at room temperature for 16 h. The reaction mixture was poured into ice-cooled saturated aqueous NH₄C₁. The reaction mixture was extracted with diethyl ether. The organic phase was washed with water and dried over Na₂SO₄, filtered and concentrated under vacuo. The residue was purified by column chromatography on silica gel (petroleum ether/ethyl acetate=1/0 to 10/1) to afford 9-3.

Step 3: To a solution of 9-3 (600 mg, 2.53 mmol) in DCM (10 mL) was added TFA (2.88 g, 25.28 mmol) at 0° C. The mixture was stirred at room temperature for 2 h. The mixture was concentrated under reduced pressure to afford 9-4.

Step 4: Compound 9-5 was prepared from compound 9-4 following the procedure for the synthesis of compound 2-2 in example 2.

Step 5: Compound 9 was prepared from compound 9-5 following the procedure for the synthesis of compound 4-12 in example 3. LCMS (ESI, m/z): [M+H]⁺=591.2; ¹H NMR (400 MHz, DMSO-d₆, ppm) δ 11.66 (s, 1H), 7.72-7.70 (m, 1H), 7.28 (s, 1H), 6.93 (s, 1H), 6.82-6.79 (m, 1H), 3.76-3.72 (m, 4H), 3.61-3.58 (m, 4H), 3.21-3.16 (m, 2H), 2.41-2.37 (m, 2H), 2.17 (s, 3H), 1.86-1.78 (m, 8H), 0.87-0.84 (m, 2H), 0.55-0.38 (m, 2H), 0.09-0.08 (m, 2H). ¹⁹F NMR (376 MHz, DMSO-d₆, ppm) δ−95.17 (2F).

### Example 7 Synthesis of Compound 11

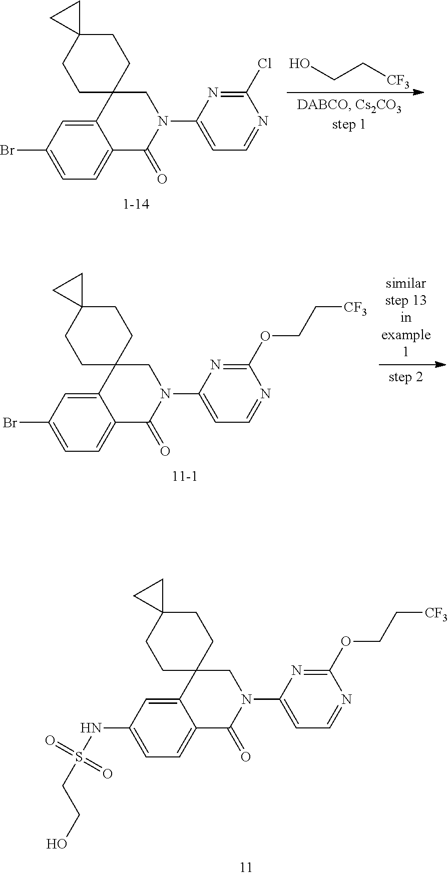

Step 1: A mixture of 1-14 (40 mg, 0.092 mmol), 3,3,3-trifluoropropan-1-ol (21.1 mg, 0.19 mmol), DABCO (5.2 mg, 0.046 mmol) and Cs₂CO₃ (90.3 mg, 0.28 mmol) in THF (1 mL) and DMF (1 mL) was stirred at room temperature for 12 h. H₂O was added to the mixture. The mixture was extracted with DCM. The combined organic layers were dried over Na₂SO₄, filtered and the filtrate was concentrated under vacuo. The residue was purified by column chromatography on silica gel (DCM/MeOH=1/0 to 25/1) to afford 11-1.

Step 2: Compound 11 was prepared from compound 11-1 following the procedure for the synthesis of compound 1 in example 1. LCMS (ESI, m/z): [M+H]⁺=555.4; ¹H NMR (400 MHz, CDCl₃, ppm) δ 8.36 (s, 1H), 8.16-7.94 (m, 2H), 7.31 (s, 1H), 7.19-7.13 (m, 1H), 6.85 (s, 1H), 5.31-5.21 (m, 1H), 4.63-4.53 (m, 2H), 4.37 (s, 2H), 4.14-4.04 (m, 2H), 3.34-3.24 (m, 2H), 2.74-2.58 (m, 2H), 2.01-1.81 (m, 4H), 1.71-1.63 (m, 2H), 0.84-0.78 (m, 2H), 0.34-0.19 (m, 4H). ¹⁹F NMR (376 MHz, CDCl₃, ppm) δ−64.77 (3F).

### Example 8 Synthesis of Compound 13

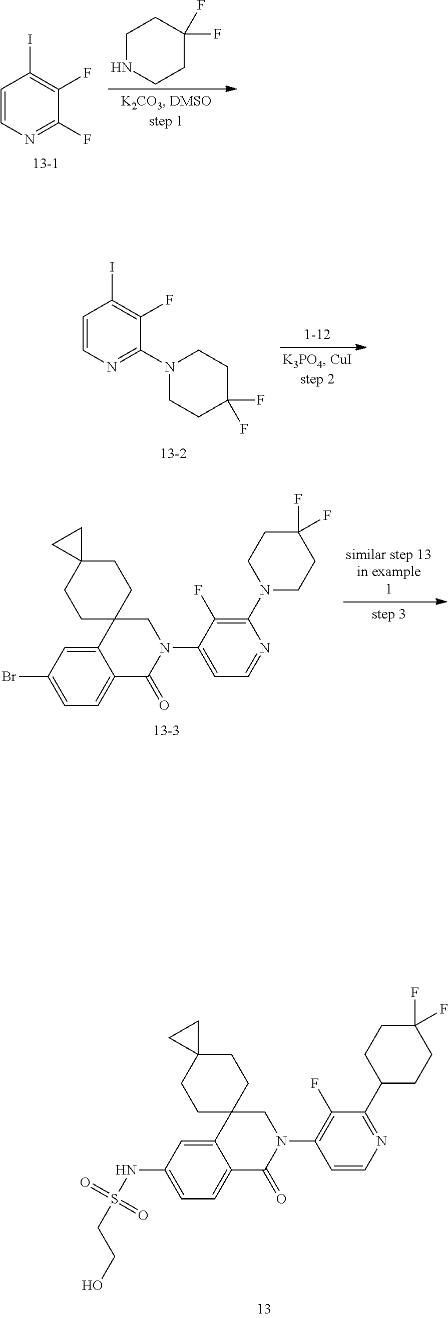

Step 1: A mixture of 13-1 (1.5 g, 6.23 mmol), 4,4-difluorohexahydropyridine (0.75 g, 6.23 mmol) and K₂CO₃ (1.3 g, 9.34 mmol) in DMSO (10 mL) was stirred at 100° C. for 5 h. The mixture was diluted with water, extracted with EtOAc. The combined organic phases were washed with water and dried Na₂SO₄, filtered and concentrated, the residue was purified by column chromatography on silica gel (petroleum ether/ethyl acetate=1/0 to 5/1) to afford 13-2.

Step 2: A mixture of 1-12 (110 mg, 0.34 mmol), 13-2 (176.27 mg, 0.52 mmol), CuI (65.4 mg, 0.34 mmol), K₂CO₃ (189.9 mg, 1.37 mmol) and N,N′-Dimethyl-1,2-ethanediamine (30.3 mg, 0.34 mmol) in dioxane (8 mL) was stirred at 105° C. for 10 h. The mixture was concentrated. The residue was purified by column chromatography on silica gel (petroleum ether/ethyl acetate=1/0 to 3/2) to afford 13-3.

Step 3: Compound 13 was prepared from compound 13-3 following the procedure for the synthesis of compound 1 in example 1. LCMS (ESI, m/z): [M+H]⁺=579.6; ¹H NMR (400 MHz, DMSO-d₆, ppm) δ 8.12-8.03 (m, 1H), 7.94-7.84 (m, 1H), 7.40-7.28 (m, 1H), 7.24-7.16 (m, 1H), 7.04-6.96 (m, 1H), 3.91 (s, 2H), 3.81-3.71 (m, 2H), 3.61-3.46 (m, 4H), 3.33-3.27 (m, 2H), 2.19-2.02 (m, 4H), 1.89-1.74 (m, 6H), 0.96-0.80 (m, 2H), 0.36-0.22 (m, 4H). ¹⁹F NMR (376 MHz, DMSO-d₆, ppm) δ−94.78 (2F), −135.17 (1F).

### Example 9 Synthesis of Compound 14

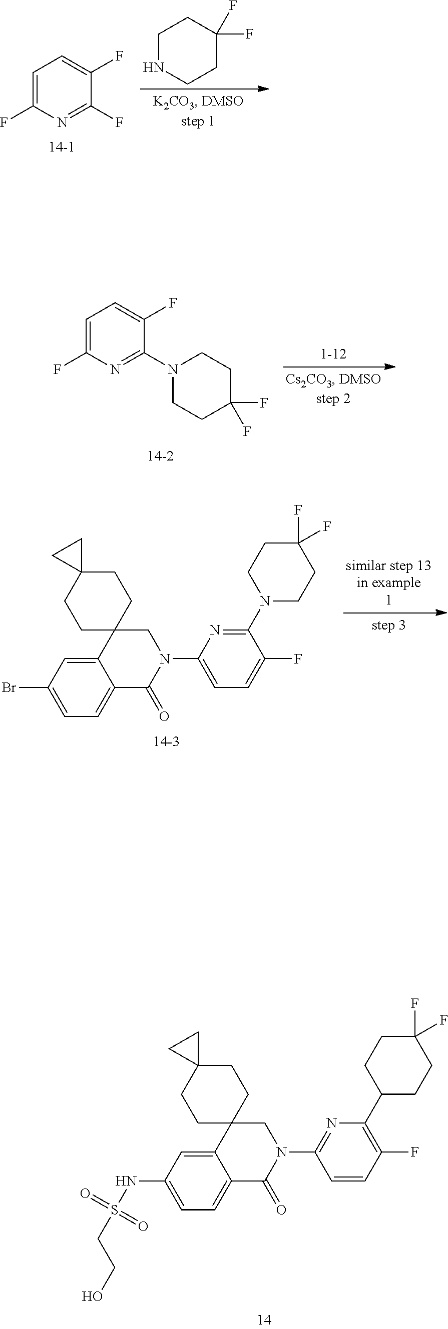

Step 1: A solution of 14-1 (1 g, 7.52 mmol), 4,4-difluorohexahydropyridine (0.91 g, 7.52 mmol) and K₂CO₃ (1.6 g, 11.27 mmol) in DMSO (10 mL) was stirred at 100° C. for 5 h. The mixture was diluted with water, extracted with EtOAc. The combined organic phases were washed with water and dried Na₂SO₄, filtered and concentrated, the residue was purified by column chromatography on silica gel (petroleum ether/ethyl acetate=1/0 to 5/1) to afford 14-2.

Step 2: A solution of 1-12 (80 mg, 0.25 mmol), 14-2 (87.8 mg, 0.38 mmol) and CS₂CO₃ (162.8 mg, 0.50 mmol) in DMSO (6 mL) was stirred at 110° C. for 16 h. The mixture was diluted with water, extracted with EtOAc. The combined organic phases were washed with water and dried Na₂SO₄, filtered and concentrated, the residue was purified by column chromatography on silica gel (petroleum ether/ethyl acetate=1/0 to 2/1) to afford 14-3.

Step 3: Compound 14 was prepared from compound 14-3 following the procedure for the synthesis of compound 1 in example 1. LCMS (ESI, m/z): [M+H]⁺=579.6; ¹H NMR (400 MHz, DMSO-d₆, ppm) δ 10.80-9.60 (m, 1H), 8.04-7.92 (m, 1H), 7.68-7.56 (m, 2H), 7.38-7.31 (m, 1H), 7.28-7.19 (m, 1H), 5.45-4.65 (m, 1H), 4.28 (s, 2H), 3.82-3.72 (m, 2H), 3.70-3.55 (m, 4H), 3.40-3.34 (m, 2H), 2.18-2.04 (m, 4H), 2.03-1.90 (m, 2H), 1.85 1.64 (m, 4H), 0.96-0.80 (m, 2H), 0.40-0.22 (m, 4H). ¹⁹F NMR (376 MHz, DMSO-d₆, ppm) δ−94.52 (2F), −133.68 (1F).

### Example 10 Synthesis of Compound 15

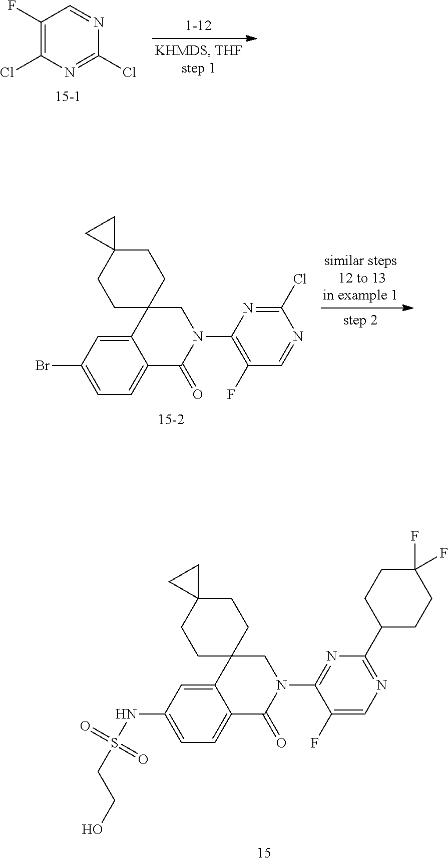 [SMILES: Fc1cnc(Cl)nc1Cl ; O=C1c2ccc(Br)cc2C2(CCC3(CC3)CC2)CN1c1nc(Cl)ncc1F ; O=C1c2ccc(NS(=O)(=O)CCO)cc2C2(CCC3(CC3)CC2)CN1c1nc(C2CCC(F)(F)CC2)ncc1F]

Step 1: To a suspension of 1-12 (77 mg, 0.24 mmol) in THF (5 mL) was added KHMDS (1M, 0.36 mL) at 0° C. After the mixture was stirred at 0° C. for 10 minutes, 15-1 (60.2 mg, 0.36 mmol) was added. Then the mixture was stirred at 60° C. for 2 h. The reaction was quenched with water and extracted with EtOAc. The organic layer was concentrated under vacuo and purified by column chromatography on silica gel (petroleum ether/ethyl acetate=1/0 to 0/1) to afford 15-2.

Step 2: Compound 15 was prepared from compound 15-2 following the procedure for the synthesis of compound 1 in example 1. LCMS (ESI, m/z): [M+H]⁺=580.2; ¹H NMR (400 MHz, DMSO-d₆, ppm) δ 10.29 (s, 1H), 8.49-8.46 (m, 1H), 7.99-7.89 (m, 1H), 7.36 (s, 1H), 7.27-7.18 (m, 1H), 5.00 (s, 1H), 4.04 (s, 2H), 3.89-3.86 (m, 4H), 3.81-3.72 (m, 2H), 2.14-1.94 (m, 5H), 1.94-1.67 (m, 7H), 0.94-0.81 (m, 2H), 0.39-0.21 (m, 4H). ¹⁹F NMR (376 MHz, DMSO-d₆, ppm) δ−95.02 (2F), −149.81 (1F).

### Example 11 Synthesis of Compound 16

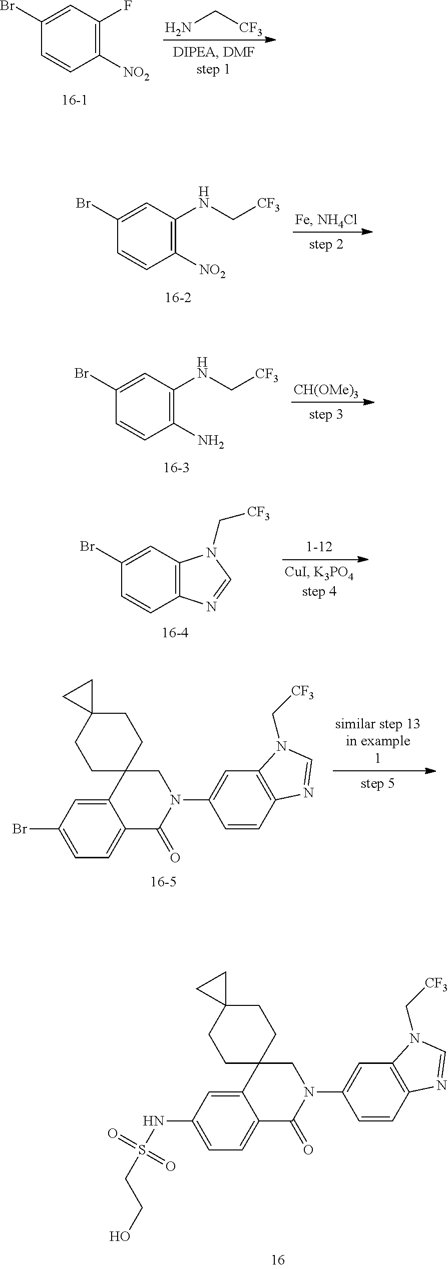

Step 1: To a solution of 16-1 (2 g, 9.09 mmol) in DMF (20 mL) was added 2, 2, 2-trifluoroethan-1-amine (0.99 g, 10.0 mmol) and DIPEA (3.01 mL, 18.18 mmol), the mixture was stirred at 80° C. for 16 h. The mixture was diluted with water, extracted with EtOAc. The combined organic phases were washed with brine and dried Na₂SO₄, filtered and concentrated, the residue was purified by column chromatography on silica gel (petroleum ether/ethyl acetate=1/0 to 1/1) to afford 16-2.

Step 2: To a solution of 16-2 (1 g, 3.34 mmol) in EtOH (10 mL) and H₂O (2 mL) was added Fe (0.9 g, 16.72 mmol) and NH₄Cl (0.9 g, 16.72 mmol), the mixture was stirred at 70° C. for 2 h. The mixture was filtered and the filtrate was concentrated in vacuo to afford 16-3 which was used for the next step directly without further purification.

Step 3: To a solution of 16-3 (400 mg, crude) in DMF (2.5 mL) was added HCl (0.1 mL, 1.5 mmol) and trimethoxymethane (2.5 mL, 1.49 mmol), the mixture was stirred at room temperature for 1 h. The mixture was diluted with water and extracted with EtOAc. The combined organic phases were washed with saturated NaHCO₃ and brine, dried over Na₂SO₄, filtered and concentrated under vacuo, the residue was purified by column chromatography on silica gel (petroleum ether/ethyl acetate=1/0 to 1/1) to afford 16-4.

Step 4: To a solution of 1-12 (100 mg, 0.31 mmol) in DMF (3 mL) were added 16-4 (130.7 mg, 0.47 mmol), CuI (59.5 mg, 0.31 mmol), K₃PO₄ (198.9 mg, 0.94 mmol) and methyl[(1R,2R)-2-(methylamino)cyclohexyl]amine (22.2 mg, 0.16 mmol), the mixture was stirred at 90° C. for 16 h. The mixture was concentrated in vacuo. The residue was purified by column chromatography on silica gel (petroleum ether/ethyl acetate=1/0 to 0/1) to afford 16-5.

Step 5: Compound 16 was prepared from compound 16-5 following the procedure for the synthesis of compound 1 in example 1. LCMS (ESI, m/z): [M+H]⁺=563.2; ¹H NMR (400 MHz, CD₃OD, ppm) δ 8.40-8.25 (m, 1H), 8.02-8.00 (m, 1H), 7.85-7.70 (m, 1H), 7.46-7.45 (m, 1H), 7.36-7.35 (m, 1H), 7.28-7.27 (m, 1H), 7.26-7.25 (m, 1H), 5.25-5.18 (m, 2H), 4.06 (s, 2H), 3.97-3.94 (m, 2H), 3.45-3.30 (m, 2H), 2.14-1.82 (m, 6H), 1.05-0.84 (m, 2H), 0.40-0.25 (m, 4H). ¹⁹F NMR (376 MHz, CD₃OD, ppm) δ−73.00 (3F).

### Example 12 Synthesis of Compound 17

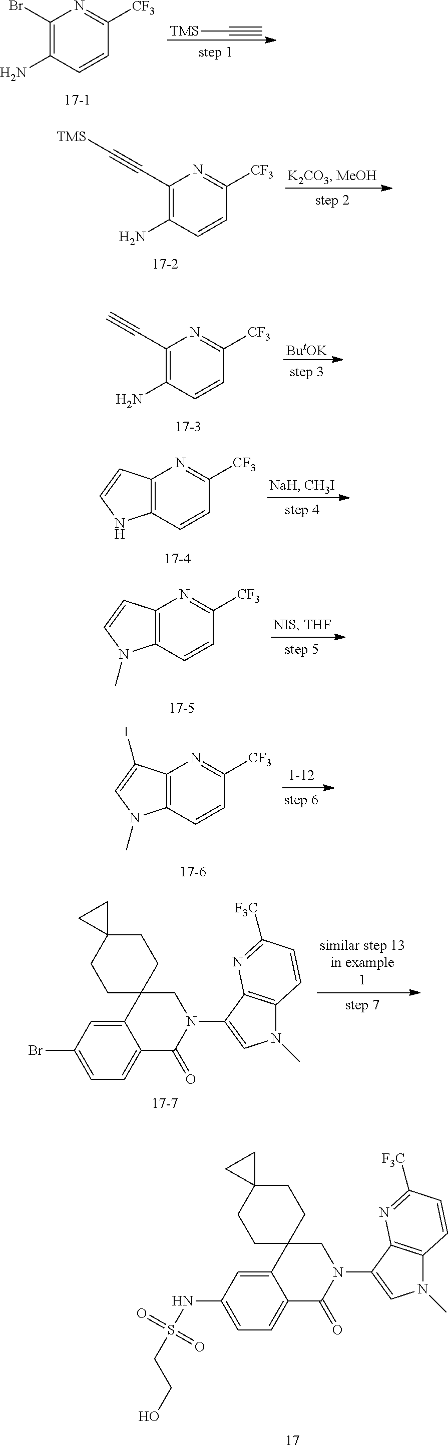

Step 1: To a solution of 17-1 (2 g, 8.3 mmol) and ethynyltrimethylsilane (1.6 g, 16.6 mmol) in THF (20 mL) was added CuI (0.2 g, 0.83 mmol), triethylamine (3.46 mL, 24.9 mmol) and Bis(triphenylphosphine)palladium dichloride (0.6 g, 0.854 mmol). The reaction mixture was stirred at 90° C. for 2 h. The mixture was concentrated under vacuo. The residue was purified by column chromatography on silica gel (petroleum ether/ethyl acetate=1/0 to 1/1) to afford 17-2.

Step 2: To a solution of 17-2 (2 g, 7.74 mmol) in MeOH (20 mL) were added K₂CO₃ (1.6 g, 11.61 mmol), the reaction mixture was stirred at room temperature for 2 h. The mixture was concentrated under vacuo. The residue was purified by column chromatography on silica gel (petroleum ether/ethyl acetate=1/0 to 2/3) to afford 17-3.

Step 3: To a solution of 17-3 (1.1 g, 5.91 mmol) in NMP (10 mL) was added potassium tert-butoxide (1.3 g, 11.82 mmol), the reaction mixture was stirred at room temperature for 16 h. Water was added to the mixture and the mixture was extracted with EtOAc. The combined organic layer was washed with brine, dried over Na₂SO₄, filtered and concentrated under vacuo. The residue was purified by column chromatography on silica gel (petroleum ether/ethyl acetate=1/0 to 0/1) to afford 17-4.

Step 4: To a solution of 17-4 (100 mg, 0.54 mmol) in DMF (2 mL) was added NaH (32.2 mg, 0.81 mmol), the reaction mixture was stirred at 0° C. for 30 minutes, then iodomethane (114.4 mg, 0.81 mmol) was added to the mixture. The mixture was stirred at room temperature for 1 h. The reaction mixture was diluted with EtOAc and ice-water. The combined organic layer was separated and washed with brine, dried over Na₂SO₄, filtered and concentrated under vacuo. The residue was purified by column chromatography on silica gel (petroleum ether/ethyl acetate=1/0 to 1/4) to afford 17-5.

Step 5: To a solution of 17-5 (100 mg, 0.5 mmol) in THF (1 mL) were added NIS (168.6 mg, 0.75 mmol), the reaction mixture was stirred at room temperature for 1 h. The mixture was concentrated in vacuo. The residue was purified by column chromatography on silica gel (petroleum ether/ethyl acetate=1/0 to 2/3) to afford 17-6.

Step 6: To a solution of 1-12 (100 mg, 0.31 mmol) in DMF (3 mL) was added 17-6 (152.7 mg, 0.47 mmol), CuI (59.5 mg, 0.31 mmol), K₃PO₄ (198.9 mg, 0.94 mmol) and methyl[(1R,2R)-2-(methylamino)cyclohexyl]amine (22.2 mg, 0.16 mmol), the reaction mixture was stirred at 90° C. for 3 h. The mixture was concentrated in vacuo. The residue was purified by column chromatography on silica gel (petroleum ether/ethyl acetate=1/0 to 0/1) to afford 17-7.

Step 7: Compound 17 was prepared from compound 17-7 following the procedure for the synthesis of compound 1 in example 1. LCMS (ESI, m/z): [M+H]⁺=563.2; ¹H NMR (400 MHz, CD₃OD, ppm) δ 8.16 (s, 1H), 8.10-8.00 (m, 2H), 7.64-7.62 (m, 1H), 7.44 (s, 1H), 7.28-7.24 (m, 1H), 4.49 (s, 2H), 3.99-3.91 (m, 5H), 3.41-3.32 (m, 2H), 2.36-2.17 (m, 2H), 2.10-1.90 (m, 4H), 0.89-0.86 (m, 2H), 0.32 (s, 4H). ¹⁹F NMR (376 MHz, CD₃OD, ppm) δ−66.84 (3F).

### Example 13 Synthesis of Compound 18

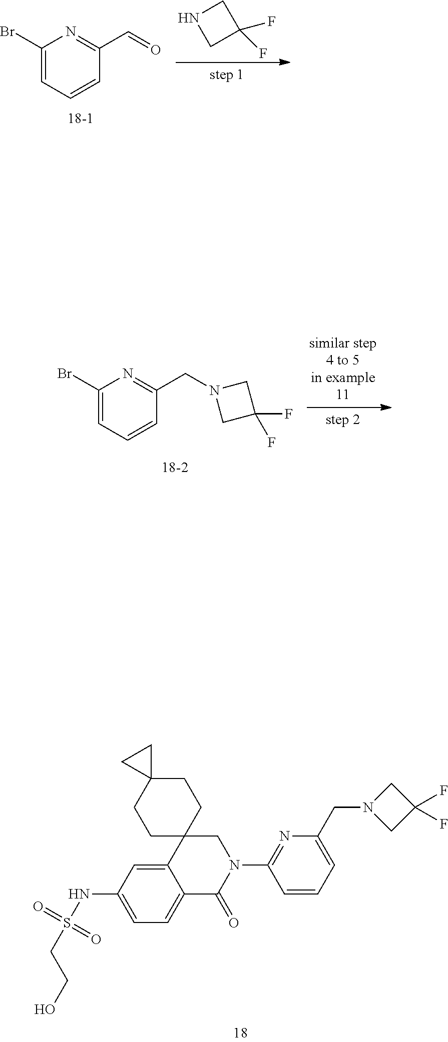 [SMILES: O=Cc1cccc(Br)n1 ; FC1(F)CNC1 ; FC1(F)CN(Cc2cccc(Br)n2)C1 ; O=C1c2ccc(NS(=O)(=O)CCO)cc2C2(CCC3(CC3)CC2)CN1c1cccc(CN2CC(F)(F)C2)n1]

Step 1: To a solution of 18-1 (500 mg, 2.69 mmol) and 3,3-difluoroazetidine (275.2 mg, 2.96 mmol) in DCM (10 mL) was added Sodium triacetoxyborohydride (854.5 mg, 4.03 mmol) at 0° C. Then the reaction mixture was stirred at room temperature for 16 h. The reaction was quenched with water, extracted with EtOAc. The combined organic layers were concentrated in vacuo. The residue was purified by column chromatography on silica gel (petroleum ether/ethyl acetate=1/0 to 2/3) to afford to afford 18-2.

Step 2: Compound 18 was prepared from compound 18-2 following the procedure for the synthesis of compound 16 in example 11. LCMS (ESI, m/z): [M+H]⁺=547.2; ¹H NMR (400 MHz, DMSO-d₆, ppm) δ 10.21 (s, 1H), 8.00-7.93 (m, 2H), 7.82-7.78 (m, 1H), 7.37 (s, 1H), 7.26-7.16 (m, 2H), 5.01-4.92 (m, 1H), 4.30 (s, 2H), 3.85 (s, 2H), 3.79-3.73 (m, 6H), 3.36-3.33 (m, 2H), 2.01-1.69 (m, 6H), 0.89-0.81 (m, 2H), 0.29 (s, 4H). ¹⁹F NMR (376 MHz, DMSO-d₆, ppm) δ−97.75 (2F).

### Example 14 Synthesis of Compound 20

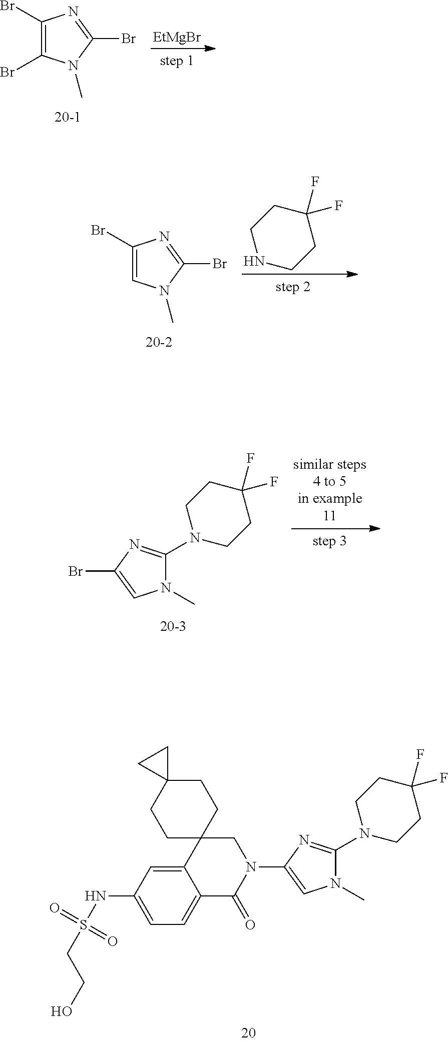

Step 1: To a solution of 20-1 (1 g, 3.14 mmol) in THF (30 mL) was added ethylmagnesium bromide (3.76 mL, 3.76 mmol) at room temperature during 5 minutes under N₂, the reaction was stirred at room temperature for 2 h. The reaction was quenched with NH₄Cl solution, the mixture was concentrated in vacuo. The residue was diluted with EtOAc and water. The organic layer was separated and washed with brine. The organic layer was dried over Na₂SO₄, filtered and concentrated under vacuo. The residue was purified by column chromatography on silica gel (petroleum ether/ethyl acetate=1/0 to 10/1) to afford 20-2.

Step 2: To a solution of 20-2 (400 mg, 1.67 mmol) in toluene (12 mL) was added 4,4-difluorohexahydropyridine (242.4 mg, 2.0 mmol), sodium tert-butoxide (560.8 mg, 5.84 mmol), RuPhos (116.7 mg, 0.25 mmol) and Pd(OAc)₂ (56.2 mg, 0.25 mmol) under N₂, the reaction was stirred at 110° C. for 18 h under N₂. The reaction mixture was concentrated in vacuo. The residue was diluted with EtOAc and water. The organic layer was separated and washed with brine. The organic layer was dried over Na₂SO₄, filtered and concentrated under vacuo. The residue was purified by reverse HPLC (0˜60% MeCN in water) to afford 20-3.

Step 3: Compound 20 was prepared from the compound of 20-3 following the procedure for the synthesis of compound 16 in example 11. LCMS (ESI, m/z): [M+H]⁺=564.2; ¹H NMR (400 MHz, DMSO-d₆, ppm): δ 7.92-7.86 (m, 1H), 7.34-7.27 (m, 2H), 7.23-7.16 (m, 1H), 4.28 (s, 2H), 3.79-3.71 (m, 2H), 3.49 (s, 3H), 3.32-3.27 (m, 2H), 3.19-3.12 (m, 4H), 2.21-2.09 (m, 4H), 2.09-2.00 (m, 2H), 1.82-1.70 (m, 2H), 1.67-1.57 (m, 2H), 0.89-0.78 (m, 2H), 0.38-0.23 (m, 4H). ¹⁹F NMR (376 MHz, DMSO-d₆, ppm) δ−94.77 (2F).

### Example 15 Synthesis of Compound 23

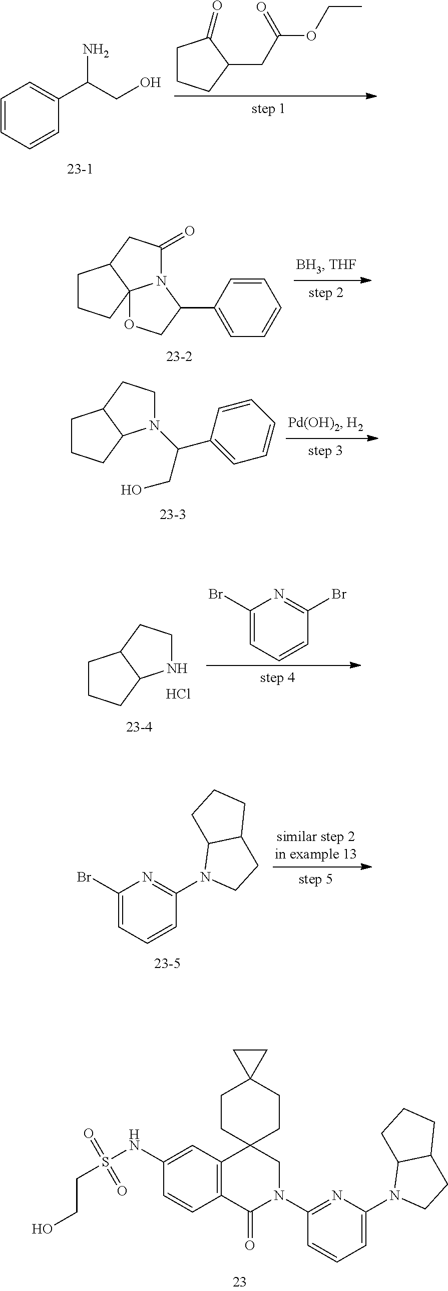

Step 1: A solution of 23-1 (1.81 g, 13.22 mmol) and ethyl (2-oxocyclopentyl)acetate (1.5 g, 8.81 mmol) in toluene (12 mL) was stirred at 110° C. for 16 h. The reaction mixture was concentrated under vacuo. The residue was purified by column chromatography on silica gel (petroleum ether/ethyl acetate=1/0 to 3/2) to afford 23-2.

Step 2: To a solution of 23-2 (1 g, 4.11 mmol) in THF (10 mL) was added BH₃·THF (12.33 mL, 12.33 mmol) at −68° C. The mixture was stirred at −68° C. for 1 h. Then the reaction mixture was stirred at 60° C. for 2 h. HCl (2M) was added to the mixture to quench the reaction at 0° C., then the mixture was stirred at 60° C. for 1 h. NaHCO₃ solution was added to the mixture to adjust pH to 8, the resulting mixture was extracted with EtOAc, the organic layer was dried over Na₂SO₄, filtered and concentrated under vacuo to afford 23-3 which was used for the next step directly without further purification.

Step 3: To a solution of 23-3 (820 mg, crude) in isopropanol (10 mL) was added Pd(OH)₂ (248.9 mg, 1.77 mmol) at 20° C. The mixture was stirred at 55° C. under H₂ balloon for 6 h. The mixture was filtered. HCl (8 mL, 4 M in dioxane) was added to the filtrate, the mixture was concentrated under vacuo to afford 23-4 which was used for the next step directly without further purification.

Step 4: A solution of 2,6-dibromopyridine (200 mg, 0.84 mmol), 23-4 (103.3 mg, crude) and K₃PO₄ (716.8 mg, 3.38 mmol) in dioxane (8 mL) was stirred at 100° C. for 16 h. The mixture was concentrated under vacuo. The residue was purified by column chromatography on silica gel (petroleum ether/ethyl acetate=1/0 to 10/1) to afford 23-5.

Step 5: Compound 23 was prepared from the compound of 23-5 following the procedure for the synthesis of compound 18 in example 13. LCMS (ESI, m/z): [M+H]⁺=551.3; ¹H NMR (400 MHz, DMSO-d₆, ppm): δ 10.65-9.70 (m, 1H), 8.01-7.92 (m, 1H), 7.56-7.46 (m, 1H), 7.42-7.28 (m, 2H), 7.26-7.18 (m, 1H), 6.32-6.22 (m, 1H), 5.25-4.75 (m, 1H), 4.46-4.28 (m, 2H), 4.27-4.19 (m, 1H), 3.82-3.70 (m, 2H), 3.55-3.38 (m, 2H), 3.37-3.34 (m, 2H), 2.83-2.72 (m, 1H), 2.19-1.93 (m, 4H), 1.83-1.43 (m, 1 OH), 0.90-0.78 (m, 2H), 0.35-0.22 (m, 4H).

### Example 16 Synthesis of Compound 24

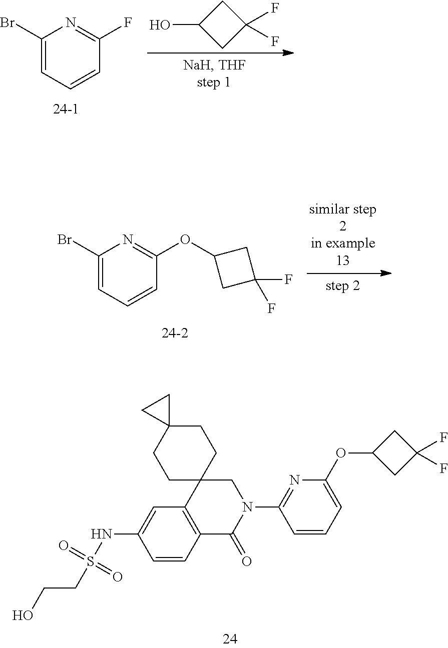

Step 1: To a solution of 3,3-difluorocyclobutan-1-ol (117.9 mg, 1.09 mmol) in THF (2 mL) was added NaH (54.5 mg, 1.36 mmol) at 0° C. After this mixture was stirred at 0° C. for 10 minutes, 24-1 (160 mg, 0.91 mmol) was added to the mixture and the mixture was stirred at room temperature for 1 h. The reaction was quenched with H₂O and extracted with EtOAc. The combined organic layers were dried over Na₂SO₄, filtered and the filtrate was concentrated under vacuo. The residue was purified by column chromatography on silica gel (100% petroleum ether) to afford 24-2.

Step 2: Compound 24 was prepared from compound 24-2 following the procedure for the synthesis of compound 18 in example 13. LCMS (ESI, m/z): [M+H]⁺=548.2; ¹H NMR (400 MHz, DMSO-d₆, ppm): δ 10.38-10.12 (m, 1H), 8.00-7.98 (m, 1H), 7.83-7.76 (m, 2H), 7.36 (s, 1H), 7.27-7.22 (m, 1H), 6.71-6.64 (m, 1H), 5.21-5.10 (m, 1H), 5.07-4.80 (m, 1H), 4.30 (s, 2H), 3.80-3.72 (m, 2H), 3.38-3.35 (m, 2H), 3.26-3.14 (m, 2H), 2.90-2.74 (m, 2H), 2.05-1.92 (m, 2H), 1.86-1.67 (m, 4H), 0.95-0.84 (m, 2H), 0.38-0.22 (m, 4H). ¹⁹F NMR (376 MHz, DMSO-d₆, ppm) 8-83.04 (1F), −94.22 (1F).

### Example 17 Synthesis of Compound 26

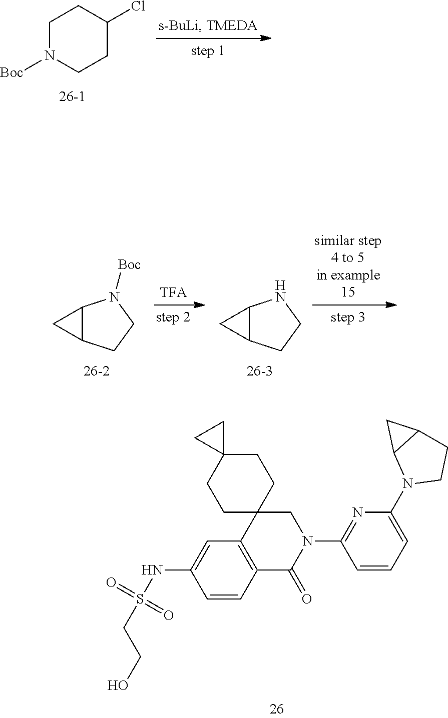 [SMILES: CC(C)(C)OC(=O)N1CCC(Cl)CC1 ; CC(C)(C)OC(=O)N1CCC2CC21 ; C1CC2CC2N1 ; O=C1c2ccc(NS(=O)(=O)CCO)cc2C2(CCC3(CC3)CC2)CN1c1cccc(N2CCC3CC32)n1]

Step 1: To a solution of 26-1 (1 g, 4.55 mmol) in THF (15 mL) was added TMEDA (0.82 mL, 5.46 mmol) and S-BuLi (4.20 mL, 5.46 mmol) at −78° C. The mixture was stirred at −78° C. for 2 h. The mixture was quenched with NH₄Cl solution, and extracted with EtOAc. The organic layer was concentrated. The residue was purified by column chromatography on silica gel (petroleum ether/EtOAc=1/0 to 4/1) to afford 26-2.

Step 2: To a solution of 26-2 (610 mg, 3 mmol) in DCM (8 mL) was added TFA (2 mL, 3.33 mmol) at 20 TC. The mixture was stirred at 20° C. for 2 h. The mixture was concentrated to afford 26-3 as TFA salt.

Step 3: Compound 26 was prepared from compound 26-3 following the procedure for the synthesis of compound 23 in example 15 as TFA salt. LCMS (ESI, M/z): [M+H]⁺=523.2; ¹H NMR (400 MHz, DMSO-d₆, ppm): δ 10.19 (s, 1H), 7.98-7.96 (m, 1H), 7.59-7.51 (m, 1H), 7.49-7.43 (m, 1H), 7.36-7.31 (m, 1H), 7.26-7.20 (m, 1H), 6.53-6.45 (m, 1H), 5.20-4.75 (m, 1H), 4.63-4.54 (m, 1H), 4.24-4.15 (m, 1H), 3.77-3.74 (m, 2H), 3.58-3.53 (m, 2H), 3.38-3.31 (m, 2H), 3.01-2.90 (m, 1H), 2.28-2.17 (m, 1H), 2.13-2.00 (m, 3H), 1.88-1.77 (m, 1H), 1.77-1.63 (m, 4H), 0.89-0.76 (m, 2H), 0.75-0.67 (m, 1H), 0.51-0.45 (m, 1H), 0.34-0.22 (m, 4H). ¹⁹F NMR (376 MHz, DMSO-d₆, ppm): δ −73.74 (3F).

### Example 18 Synthesis of Compound 27

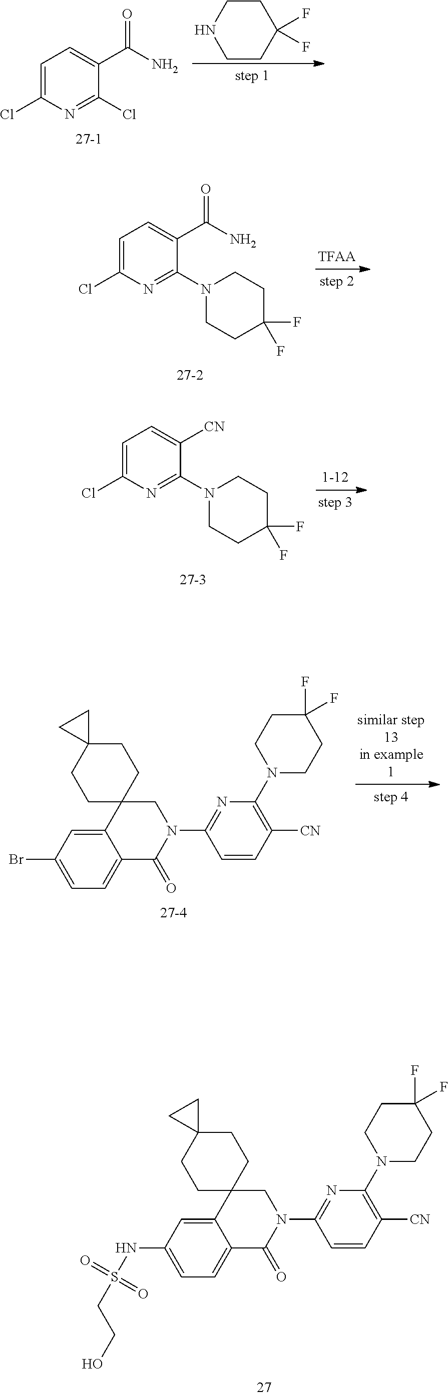

Step 1: To a solution of 27-1 (1 g, 5.24 mmol) in NMP (20 mL) were added 4,4-difluorohexahydropyridine (0.95 g, 7.85 mmol) and DIPEA (2.6 mL, 15.71 mmol) at room temperature. Then the mixture was stirred at 130° C. for 12 h. The reaction was quenched with H₂O and extracted with EtOAc. The combined organic layers were dried over Na₂SO₄, filtered and the filtrate was concentrated under vacuo. The residue was purified by column chromatography on silica gel (petroleum ether/EtOAc=1/0 to 1/1) to afford 27-2.

Step 2: To the reaction mixture of 27-2 (200 mg, 0.73 mmol) and TEA (0.15 mL, 1.09 mmol) in DCM (5 mL) was added trifluoroacetic acid anhydride (228.6 mg, 1.09 mmol) slowly at 0° C. The reaction mixture was stirred at 0° C. for 15 minutes. The reaction was quenched with H₂O and extracted with DCM. The combined organic layers were washed with brine, dried over anhydrous Na₂SO₄, filtered and concentrated under reduced pressure. The residue was purified by column chromatography on silica gel (petroleum ether/EtOAc=1/0 to 1/1) to afford 27-3.

Step 3: To a suspension of 1-12 (50 mg, 0.16 mmol) in DMA (2 mL) was added Cs₂CO₃ (152.6 mg, 0.47 mmol) and 27-3 (80.5 mg, 0.31 mmol). Then the mixture was stirred at 120° C. for 12 h. The mixture was quenched with H₂O and extracted with EtOAc. The combined organic layers were dried over Na₂SO₄, filtered and the filtrate was concentrated under vacuo. The residue was purified by column chromatography on silica gel (petroleum ether/EtOAc=1/0 to 9/1) to afford 27-4.

Step 4: Compound 27 was prepared from compound 27-4 following the procedure for the synthesis of compound 1 in example 1. LCMS (ESI, m/z): [M+H]⁺=586.2; ¹H NMR (400 MHz, DMSO-d₆, ppm): 8 8.10-8.01 (m, 1H), 7.94-7.88 (m, 1H), 7.88-7.82 (m, 1H), 7.19 (s, 1H), 7.13-7.05 (m, 1H), 4.39 (s, 2H), 3.88-3.79 (m, 4H), 3.77-3.69 (m, 2H), 3.22-3.20 (m, 2H), 2.22-2.08 (m, 4H), 2.00-1.86 (m, 2H), 1.85-1.74 (m, 2H), 1.71-1.58 (m, 2H), 0.91-0.77 (m, 2H), 0.29 (s, 4H); ¹⁹F NMR (376 MHz, DMSO-d₆, ppm): 6-94.66 (2F).

### Example 19 Synthesis of Compound 28 and 29

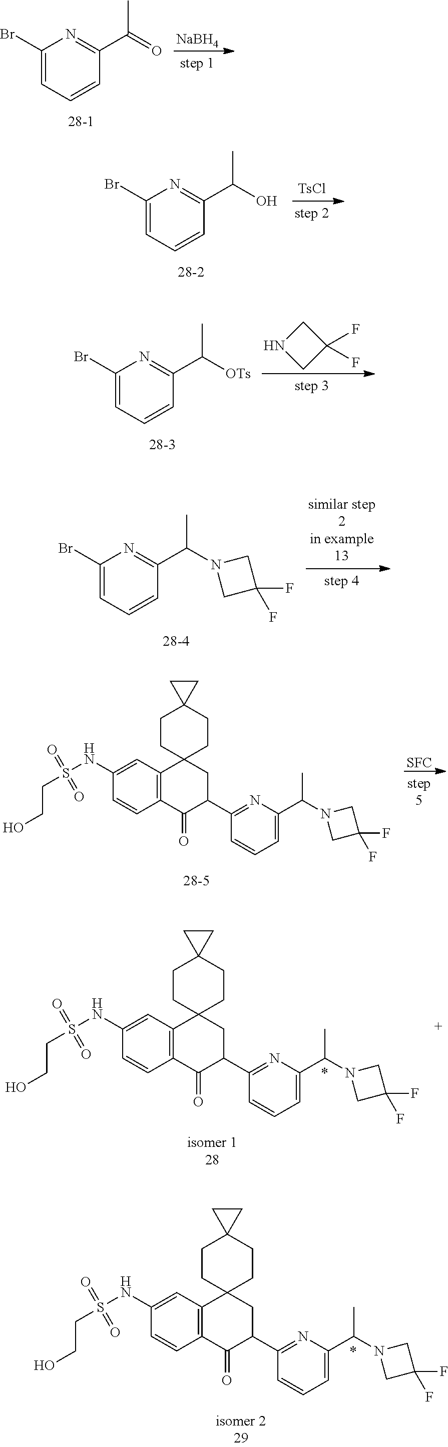

Step 1: To a solution of 28-1 (3 g, 15 mmol) in MeOH (50 mL) was added NaBH₄ (1.1 g, 30 mmol) at 0° C. Then the mixture was stirred at room temperature for 3 h. The reaction was quenched with saturated aqueous NH₄Cl and extracted with EtOAc. The combined organic layers were dried over Na₂SO₄, filtered and the filtrate was concentrated under vacuo to afford 28-2 which was used for the next step directly without further purification.

Step 2: To a solution of 28-2 (3 g, 14.85 mmol) in DCM (50 mL) were added Tosyl chloride (3.68 g, 19.30 mmol) and DMAP (0.2 g, 1.49 mmol). The reaction mixture was stirred at room temperature for 16 h. H₂O was added to the mixture and the mixture was extracted with EtOAc. The combined organic layers were dried over Na₂SO₄, filtered and the filtrate was concentrated under vacuo. The residue was purified by column chromatography on silica gel (petroleum ether/EtOAc=1/0 to 6/1) to afford 28-3.

Step 3: To a solution of 28-3 (1.5 g, 4.21 mmol) in MeCN (10 mL) were added 3,3-difluoroazetidine hydrochloride (0.71 g, 5.47 mmol) and K₂CO₃ (2.9 g, 21.05 mmol). The reaction mixture was stirred at 80° C. for 16 h. The mixture was filtered and the filtrate was concentrated under vacuo. The residue was purified by column chromatography on silica gel (petroleum ether/EtOAc=1/0 to 8/1) to afford 28-4.

Step 4: Compound 28-5 was prepared from compound 28-4 following the procedure for the synthesis of compound 18 in example 13.

Step 5: 28-5 was purified by SFC (column: ChiralCel OJ, 250×30 mm I.D., 10 μm (0.1% NH₃·H₂O in MeOH)/Supercritical CO₂=20/80) to afford 28 and 29 respectively. 28: SFC analysis: 99.66% ee; retention time: 2.636 min; column: ChiralCel OJ, 150×4.6 mm I.D., 5 μm; MeOH (0.05% DEA) in CO₂, 5% to 40%; pressure: 100 bar; flow rate: 2.5 mL/min; LCMS (ESI, m/z): [M+H]⁺=561.3; ¹H NMR (400 MHz, CDCl₃, ppm) δ 8.09-8.07 (m, 1H), 7.94-7.92 (m, 1H), 7.66-7.62 (m, 1H), 7.32 (s, 1H), 7.11-7.09 (m, 2H), 4.32-4.23 (m, 2H), 4.11-4.03 (m, 2H), 3.65-3.48 (m, 5H), 3.32-3.25 (m, 2H), 2.01-1.90 (m, 2H), 1.89-1.78 (m, 2H), 1.75-1.68 (m, 2H), 1.29-1.27 (m, 3H), 0.82-0.74 (m, 2H), 0.30-0.17 (m, 4H). ¹⁹F NMR (376 MHz, CDCl₃, ppm) δ−99.34 (2F). 29: SFC analysis: 97.52% ee; retention time: 2.769 min; column: ChiralCel OJ, 150-4.6 mm I.D., 5 μm; MeOH (0.05% DEA) in CO₂., 5% to 40%; pressure: 100 bar; flow rate: 2.5 mL/min; LCMS (ESI, m/z): [M+H]⁺=561.3; ¹H NMR (400 MHz, CDCl₃, ppm) δ 8.09-8.07 (m, 1H), 7.94-7.92 (m, 1H), 7.66-7.61 (m, 1H), 7.32 (s, 1H), 7.14-7.07 (m, 2H), 4.33-4.23 (m, 2H), 4.11-4.01 (m, 2H), 3.65-3.50 (m, 5H), 3.35-3.25 (m, 2H), 1.99-1.91 (m, 2H), 1.88-1.79 (m, 2H), 1.74-1.69 (m, 2H), 1.29-1.27 (m, 3H), 0.79-0.75 (m, 2H), 0.30-0.17 (m, 4H). ¹⁹F NMR (376 MHz, CDCl₃, ppm) δ−99.34 (2F).

### Example 20 Synthesis of Compound 35

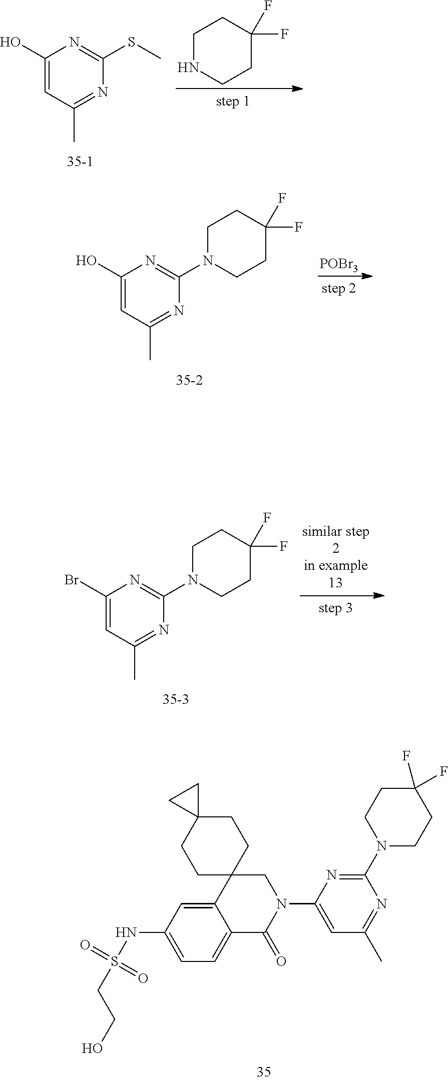

Step 1: A mixture of 35-1 (500 mg, 3.20 mmol), 4,4-difluorohexahydropyridine (387.7 mg, 3.20 mmol) and DIPEA (2.65 mL, 16.01 mmol) was heated at 100° C. for 12 h. The mixture was concentrated under vacuo, the residue was purified by column chromatography on silica gel (DCM/MeOH=l/0 to 10/1) to afford 35-2.

Step 2: To an ice-cold solution of 35-2 (428 mg, 1.87 mmol) in DMF (10 mL) was added POBr₃ (695.8 mg, 2.43 mmol) at 0° C. The mixture was heated at 70° C. for 1 h. Then the mixture was concentrated under vacuo. Saturated NaHCO₃ solution was added to the residue and the mixture was extracted with EtOAc. The combined organic layers were dried over Na₂SO₄, filtered and the filtrate was concentrated under vacuo. The residue was purified by column chromatography on silica gel (petroleum ether/EtOAc=1/0 to 5/1) to afford 35-3.

Step 3: Compound 35 was prepared from compound 35-3 following the procedure for the synthesis of compound 18 in example 13. LCMS (ESI, m/z): [M+H]⁺=576.2; ¹H NMR (400 MHz, DMSO-d₆, ppm): δ 7.69-7.61 (m, 1H), 7.55 (s, 1H), 6.84-6.78 (m, 1H), 6.77-6.70 (m, 1H), 4.35 (s, 2H), 3.98-3.85 (m, 4H), 3.72-3.63 (m, 2H), 2.96-2.83 (m, 2H), 2.28 (s, 3H), 2.01-1.95 (m, 4H), 1.80-1.74 (m, 2H), 1.57-1.53 (m, 4H), 0.83-0.74 (m, 2H), 0.27 (s, 4H); ¹⁹F NMR (376 MHz, DMSO-d₆, ppm): 6-94.60 (2F).

### Example 21 Synthesis of Compound 36

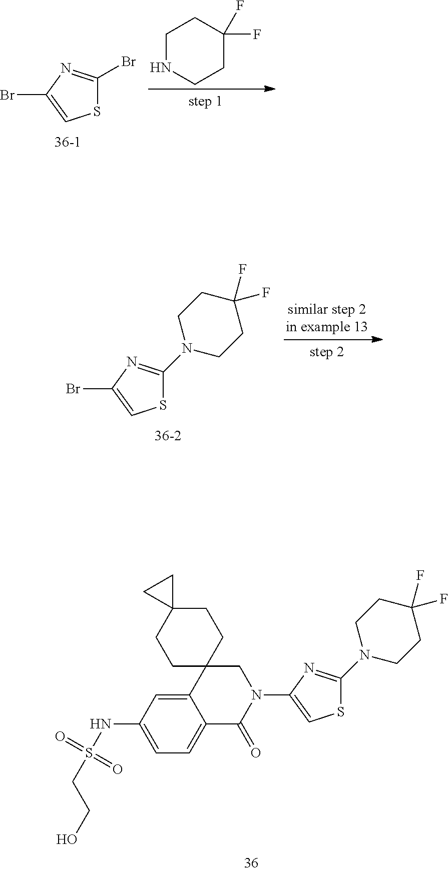

Step 1: A mixture of 36-1 (10 g, 41.17 mmol) and 4,4-difluorohexahydropyridine (2.52 g, 20.79 mmol) in DIPEA (50 mL) was stirred at 55° C. for 16 h. The reaction was quenched with water and extracted with EtOAc. The combined organic layers were concentrated in vacuum. The residue was purified by column chromatography on silica gel (petroleum ether/EtOAc=1/0 to 1/1) to afford 36-2.

Step 2: Compound 36 was prepared from compound 36-2 following the procedure for the synthesis of compound 18 in example 13. LCMS (ESI, m/z): [M+H]⁺=567.2; ¹H NMR (400 MHz, DMSO-d₆, ppm): δ 7.66-7.53 (m, 1H), 7.11 (s, 1H), 6.79 (s, 1H), 6.76-6.69 (m, 1H), 5.09-5.03 (m, 1H), 4.23 (s, 2H), 3.68-3.66 (m, 2H), 3.63-3.55 (m, 4H), 2.94-2.84 (m, 2H), 2.17-2.01 (m, 6H), 1.85-1.70 (m, 2H), 1.65-1.52 (m, 2H), 0.87-0.74 (m, 2H), 0.36-0.23 (m, 4H). ¹⁹F NMR (376 MHz, DMSO-d₆, ppm): 6-95.67 (2F).

### Example 22 Synthesis of Compound 41

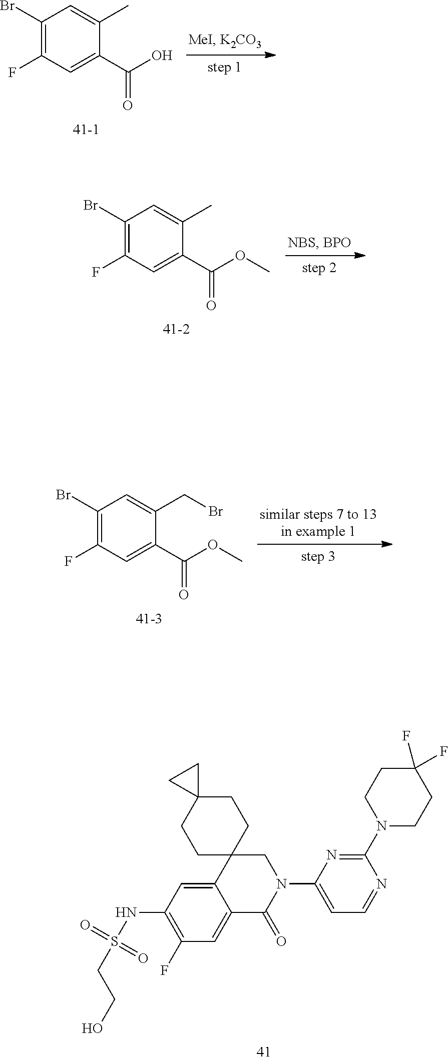 [SMILES: Cc1cc(Br)c(F)cc1C(=O)O ; COC(=O)c1cc(F)c(Br)cc1C ; COC(=O)c1cc(F)c(Br)cc1CBr ; O=C1c2cc(F)c(NS(=O)(=O)CCO)cc2C2(CCC3(CC3)CC2)CN1c1ccnc(N2CCC(F)(F)CC2)n1]

Step 1: To a solution of 41-1 (5.0 g, 21.46 mmol) in DMF (60 mL) were added K₂CO₃ (5.9 g, 42.91 mmol) and CH₃I (2.67 mL, 42.91 mmol) at room temperature. Then the mixture was stirred at room temperature for 1 h. The mixture was filtered and the filtrate was concentrated under vacuo. The residue was purified by column chromatography on silica gel (petroleum ether/EtOAc=1/0 to 5/11) to afford 41-2.

Step 2: To a solution of 41-2 (4.60 g, 18.62 mmol) in CCl₄ (60 mL) was added dibenzoyl peroxide (0.9 g, 3.72 mmol), the reaction mixture was stirred at 70° C. for 15 minutes under N₂, then NBS (4.0 g, 22.34 mmol) was added to the mixture and the mixture was stirred at 85° C. for 16 h under N₂. The reaction was quenched with water and extracted with DCM. The organic layer was dried over Na₂SO₄, filtered and concentrated in vacuo. The residue was purified by column chromatography on silica gel (petroleum ether/EtOAc=1/0 to 2/1) to afford 41-3.

Step 3: Compound 41 was prepared from compound 41-3 following the procedure for the synthesis of compound 1 in example 1. LCMS (ESI, m/z): [M+H]⁺=580.2; ¹H NMR (400 MHz, DMSO-de, ppm): δ 10.54-9.57 (m, 1H), 8.36-8.34 (m, 1H), 7.79-7.76 (m, 1H), 7.65-7.57 (m, 2H), 5.34-4.71 (m, 1H), 4.47 (s, 2H), 3.99-3.89 (m, 4H), 3.83-3.75 (m, 2H), 3.40-3.35 (m, 2H), 2.07-1.94 (m, 6H), 1.85-1.75 (m, 2H), 1.68-1.60 (m, 2H), 0.88-0.82 (m, 2H), 0.30 (s, 4H). ¹⁹F NMR (376 MHz, DMSO-d₆, ppm): 6-94.67 (2F), −126.55 (1F).

### Example 23 Synthesis of Compound 43

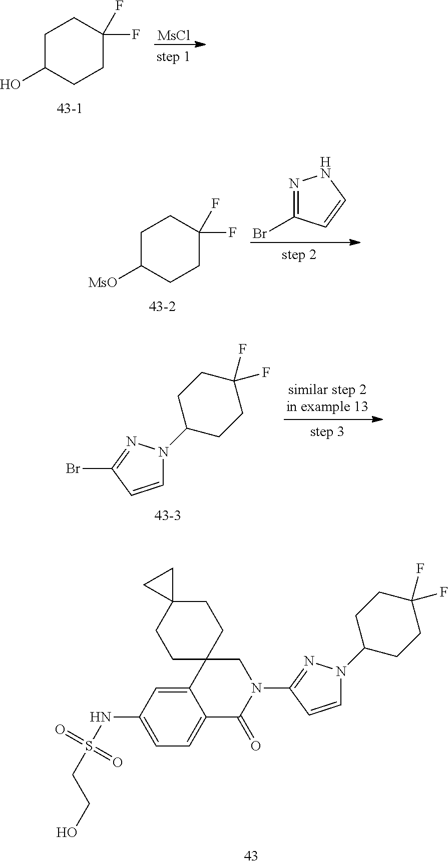

Step 1: To a mixture of 43-1 (2.5 g, 18.36 mmol) in DCM (30 mL) was added TEA (3.31 mL, 23.87 mmol), the mixture was cooled to 0° C., then Mesyl chloride (1.85 mL, 23.87 mmol) was added dropwise to the mixture and the mixture was stirred at room temperature for 2 h. The mixture was diluted with EtOAc and saturated NaHCO₃ solution. The mixture was extracted with EtOAc, the combined organic layer was washed with brine, dried over Na₂SO₄, filtered and concentrated to afford 43-2 which was used for the next step directly without further purification.

Step 2: To a mixture of 43-2 (1.04 g, 4.86 mmol), 3-bromo-1H-pyrazole (650 mg, 4.42 mmol) in DMF (10 mL) was added Cs₂CO₃ (1.87 g, 5.75 mmol). The mixture was then stirred at 100° C. for 5 h. The mixture was filtered and concentrated, the residue was purified by column chromatography on silica gel (petroleum ether/EtOAc=1/0 to 2/3) to afford 43-3.

Step 3: Compound 43 was prepared from compound 43-3 following the procedure for the synthesis of compound 18 in example 13. LCMS (ESI, m/z): [M+H]⁺=549.2; ¹H NMR (400 MHz, DMSO-d₆, ppm): δ 10.15-10.10 (m, 1H), 7.95-7.93 (m, 1H), 7.77 (s, 1H), 7.34-7.33 (m, 1H), 7.23-7.20 (m, 1H), 6.80 (s, 1H), 5.21-4.82 (m, 1H), 4.46-4.33 (m, 1H), 4.25 (s, 2H), 3.77-3.74 (m, 2H), 3.35-3.29 (m, 2H), 2.23-1.88 (m, 10H), 1.84-1.71 (m, 2H), 1.71-1.59 (m, 2H), 0.86 (d, J=13.5 Hz, 2H), 0.40-0.19 (m, 4H). ¹⁹F NMR (376 MHz, DMSO-d₆, ppm) δ −92.79 (1F), −98.40 (1F).

### Example 24 Synthesis of Compound 44

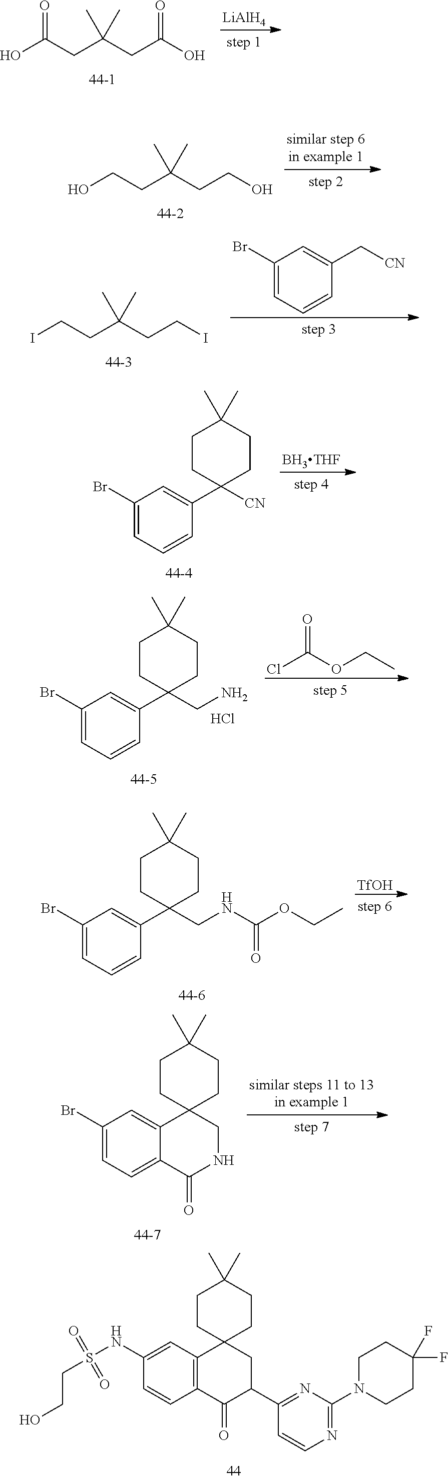

Step 1: To an ice-cold solution of 44-1 (5 g, 31.22 mmol) in THF (50 mL) was added LiAlH₄ (62.43 mL, 62.43 mmol) over 10 min at 0° C. The reaction mixture was stirred at 80° C. for 2 h. The reaction was quenched with water and 10% NaOH. The mixture was filtered and the filtrate was concentrated under vacuo to afford 44-2 which was used for the next step directly without further purification.

Step 2: Compound 44-3 was prepared from the compound of 44-2 following the procedure for the synthesis of compound 1-7 in example 1.

Step 3: To an ice-cold solution of 2-(3-bromophenyl)acetonitrile (1.84 g, 9.38 mmol) and 44-3 (3.0 g, 8.52 mmol) in DMF (50 mL) was added NaH (1.13 g, 28.13 mmol) at 0° C. The reaction mixture was stirred at room temperature for 16 h. The reaction was quenched with water, extracted with EtOAc. The combined organic layer was dried over Na₂SO₄, filtered and concentrated under vacuo, the residue was purified by column chromatography on silica gel (petroleum ether/ethyl acetate=1/0 to 10/1) to afford 44-4.

Step 4: To an ice-cold solution of 44-4 (1.0 g, 3.42 mmol) in THF (20 mL) was added borane tetrahydrofuran (8.55 mL, 8.55 mmol) at 0° C. The reaction mixture was stirred at 75° C. for 2 h. The mixture was cold to 0° C., EtOH was slowly added to quench the reaction, then HCl (4M in MeOH) was added to the mixture and the mixture was stirred at room temperature for another 30 minutes. The mixture was concentrated under vacuo to afford 44-5 as HCl salt which was used for the next step directly without further purification.

Step 5: To an ice-cold solution of 44-5 (1.1 g, crude) and triethyl amine (1.55 mL, 11.14 mmol) in DCM (20 mL) was added ethyl chloromethanoate (483.5 mg, 4.46 mmol) at 0° C. The resulting mixture was stirred at room temperature for 1 h. The reaction was quenched with water, extracted with DCM. The combined organic layer was dried over Na₂SO₄, filtered and concentrated under vacuo to afford 44-6 which was used for the next step directly without further purification.

Step 6: Trifluoromethanesulfonic acid (26.5 g, 176.48 mmol) was slowly added to 44-6 (1.3 g, crude) at room temperature. The resulting mixture was stirred at 70° C. for 16 h. The reaction mixture was poured into ice-water. The resulting mixture was extracted with EtOAc. The combined organic layer was dried over Na₂SO₄, filtered and concentrated under vacuo. the residue was purified by column chromatography on silica gel (petroleum ether/ethyl acetate=1/0 to 0/1) to afford to afford 44-7.

Step 7: Compound 44 was prepared from the compound of 44-7 following the procedure for the synthesis of compound 1 in example 1. LCMS (ESI, m/z): [M+H]⁺=564.3; ¹H NMR (400 MHz, CDCl₃, ppm): δ 8.22-8.21 (m, 1H), 8.11-8.08 (m, 1H), 7.66-7.65 (m, 1H), 7.29-7.28 (m, 1H), 7.13-7.10 (m, 1H), 6.79 (s, 1H), 4.30 (s, 2H), 4.09-4.05 (m, 2H), 3.95-3.92 (m, 4H), 3.29-3.27 (m, 2H), 2.29-2.27 (m, 1H), 2.01-1.91 (m, 4H), 1.88-1.80 (m, 2H), 1.51-1.41 (m, 4H), 1.33-1.29 (m, 2H), 1.19 (s, 3H), 0.95 (s, 3H). ¹⁹F NMR (376 MHz, CDCl₃, ppm): 6-96.91 (2F).

### Example 25 Synthesis of Compound 52

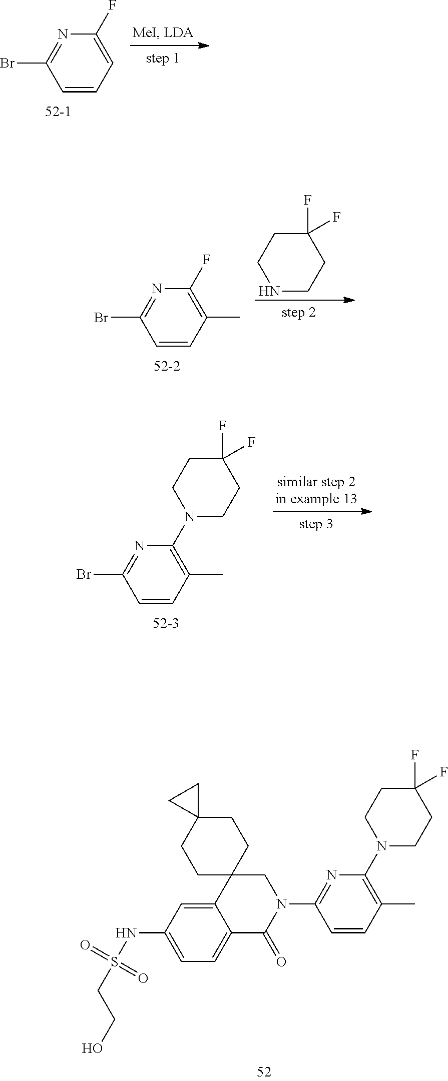

Step 1: A solution of 52-1 (1.0 g, 5.68 mmol) in THF (10 mL) was added dropwise to a solution of LDA (3.41 mL, 6.82 mmol) in THF (10 mL) at −78° C. The mixture was stirred at −78° C. for 4 h. Then iodomethane (0.39 mL, 6.25 mmol) was added to the mixture. The mixture was stirred at room temperature for 16 h. The reaction was quenched with water and extracted with EtOAc. The combined organic layer was dried over Na₂SO₄, filtered and concentrated, the residue was purified by column chromatography on silica gel (petroleum ether/EtOAc=1/0 to 0/1) to afford 52-2.

Step 2: To a solution of 52-2 (200 mg, 0.95 mmol) and 4,4-difluorohexahydropyridine (182.5 mg, 1.51 mmol) in DMSO (3 mL) was added DIPEA (0.52 mL, 3.16 mmol) at room temperature. The mixture was stirred at 130° C. for 16 h. The mixture was concentrated and the residue was purified by column chromatography on silica gel (petroleum ether/EtOAc=1/0 to 1/1) to afford 52-3.

Step 3: Compound 52 was prepared from compound 52-3 following the procedure for the synthesis of compound 18 in example 13. LCMS (ESI, m/z): [M+H]⁺=575.2; ¹H NMR (400 MHz, DMSO-d₆, ppm): δ 10.25-10.11 (m, 1H), 7.97 (s, 1H), 7.75-7.73 (m, 1H), 7.58-7.56 (m, 1H), 7.33-7.23 (m, 2H), 5.09-4.88 (m, 1H), 4.33 (s, 2H), 3.79-3.71 (m, 2H), 3.34-3.28 (m, 6H), 2.25 (s, 3H), 2.17-2.08 (m, 4H), 2.03-1.97 (m, 2H), 1.81-1.75 (m, 2H), 1.69-1.66 (m, 2H), 0.86-0.82 (m, 2H), 0.29 (m, 4H). ¹⁹F NMR (376 MHz, DMSO-d₆, ppm): 6-94.99 (2F).

### Example 26 Synthesis of Compound 55

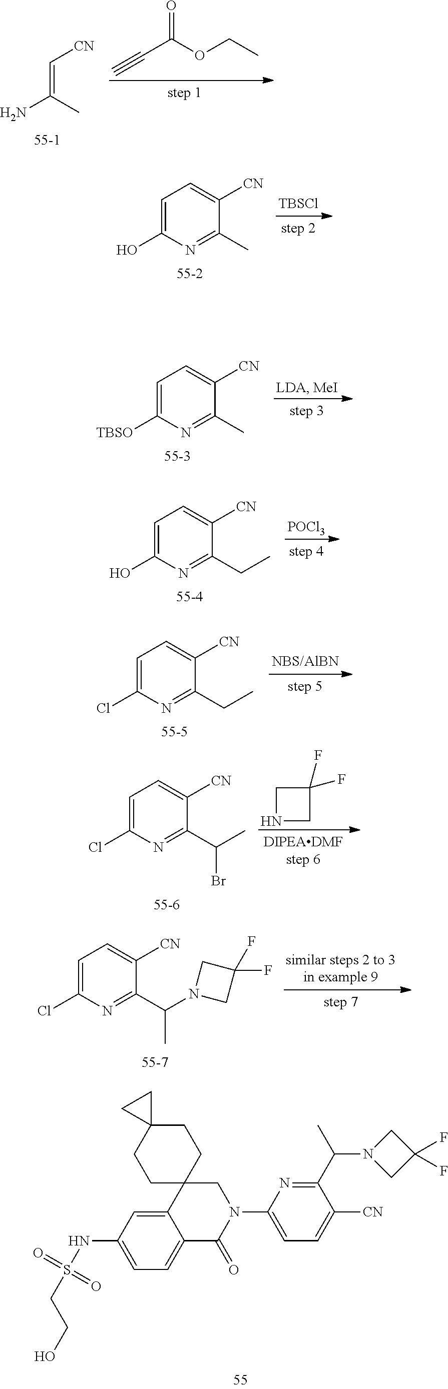

Step 1: A solution of 55-1 (10 g, 121.79 mmol) and ethyl prop-2-ynoate (12.34 mL, 121.79 mmol) in DMF (15 mL) was stirred at 110° C. for 72 h. The mixture was filtered. The cake was washed with MeOH and petroleum ether, dried to afford 55-2.

Step 2: To a solution of 55-2 (2.90 g, 21.62 mmol) and tert-Butyldimethylsilyl chloride (7.2 g, 47.56 mmol) in DMF (45 mL) was added TEA (7.21 mL, 51.89 mmol) at 0° C. The mixture was stirred at 0° C. under N₂ for 16 h. The mixture was diluted with H₂O, extracted with EtOAc. The combined organic phases were washed with H₂O and concentrated. The residue was purified by column chromatography on silica gel (petroleum ether/EtOAc=1/0 to 20/1) to afford 55-3.

Step 3: To a solution of 55-3 (3.19 g, 12.84 mmol) in THF (18 mL) was added LDA (7.71 mL, 15.41 mmol) at −78° C. The mixture was stirred at −78° C. for 25 minutes. Then CH₃I (0.96 mL, 15.41 mmol) was added and the mixture was stirred at −78° C. for 25 minutes. The mixture was quenched with acetic acid (5 mL) and stirred at 50° C. for 1 h. The mixture was concentrated. The residue was purified by column chromatography on silica gel (DCM/MeOH=1/0 to 10/1) to afford 55-4.

Step 4: A solution of 55-4 (1.71 g, 11.54 mmol) in POCl₃ (10 mL) was stirred at 100° C. for 2 h. The mixture was concentrated, then saturated NaHCO₃ solution was added to the residue, the mixture was extracted with EtOAc. The combined organic phases were washed with H₂O and concentrated. The residue was purified by column chromatography on silica gel (petroleum ether/EtOAc=1/0 to 6/1) to afford 55-5.

Step 5: A solution of 55-5 (400 mg, 2.40 mmol), NBS (683.7 mg, 3.84 mmol) and AIBN (157.7 mg, 0.96 mmol) in CCl₄ (6 mL) was stirred at 70° C. for 4 h. The mixture was concentrated. The residue was purified by column chromatography on silica gel (petroleum ether/EtOAc=1/0 to 10/1) to afford 55-6.

Step 6: A solution of 55-6 (200 mg, 0.82 mmol), 3,3-difluoroazetidine hydrochloride (116.1 mg, 0.90 mmol) and DIPEA (0.20 mL, 1.22 mmol) in DMF (6 mL) was stirred at 70° C. for 3 h. The mixture was diluted with H₂O and extracted with EtOAc. The organic phase was washed with H₂O and concentrated. The residue was purified by column chromatography on silica gel (petroleum ether/EtOAc=1/0 to 4/1) to afford 55-7.

Step 7: Compound 55 was prepared from compound 55-7 following the procedure for the synthesis of compound 14 in example 9. LCMS (ESI, m/z): [M+H]⁺=586.2; ¹H NMR (400 MHz, DMSO-d₆, ppm): δ 10.09 (s, 1H), 8.00-7.93 (m, 1H), 7.91-7.85 (m, 1H), 7.29 (s, 1H), 7.21-7.14 (m, 1H), 6.66-6.59 (m, 1H), 6.12-6.06 (m, 1H), 4.94 (s, 1H), 4.62-4.45 (m, 4H), 3.77-3.69 (m, 2H), 3.60-3.44 (m, 2H), 3.32-3.27 (m, 2H), 1.84-1.74 (m, 2H), 1.66-1.59 (m, 1H), 1.56-1.47 (m, 5H), 1.08-0.96 (m, 1H), 0.90-0.81 (m, 1H), 0.57-0.48 (m, 1H), 0.30-0.16 (m, 4H). ¹⁹F NMR (376 MHz, DMSO-d₆, ppm): 6-98.94 (2F).

### Example 27 Synthesis of Compound 56

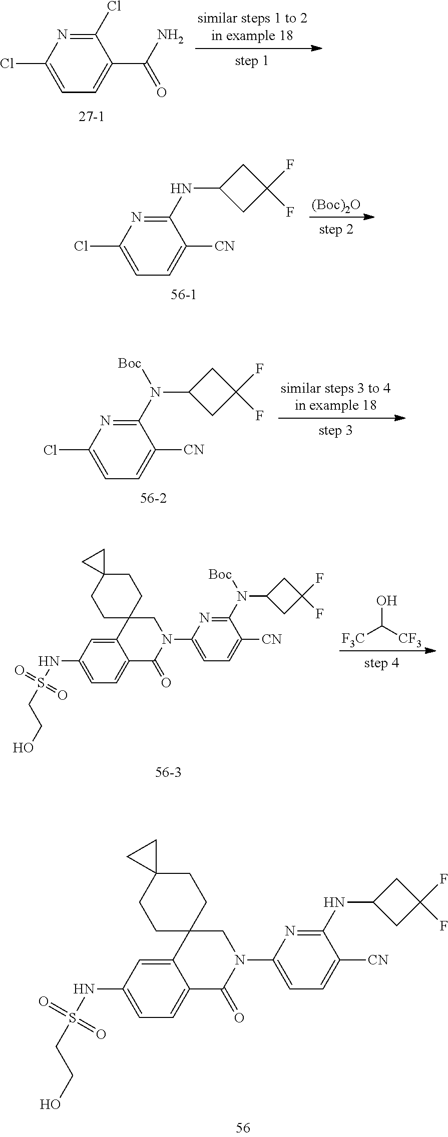

Step 1: Compound 56-1 was prepared from compound 27-1 following the procedure for the synthesis of compound 27-3 in example 18.

Step 2: To a solution of 56-1 (180 mg, 0.74 mmol) in DCM (2 mL) were Di-tert butyl dicarbonate (0.34 mL, 1.45 mmol) and DMAP (9.0 mg, 0.074 mmol), the reaction mixture was stirred at room temperature for 2 h. The mixture was concentrated in vacuo. The residue was purified by column chromatography on silica gel (petroleum ether/EtOAc=1/0 to 4/1) to afford 56-2.

Step 3: Compound 56-3 was prepared from compound 56-2 following the procedure for the synthesis of compound 27 in example 18.

Step 4: A solution of 56-3 (50 mg, 0.074 mmol) in 1,1,1,3,3,3-hexafluoropropan-2-ol (3 mL) was stirred at 80° C. for 72 h. The mixture was concentrated in vacuo. The residue was purified by reverse HPLC (MeCN in H₂O (0˜100%)) to afford 56. LCMS (ESI, m/z): [M+H]⁺=572.2; ¹H NMR (400 MHz, CD₃OD, ppm): δ 8.07-8.05 (m, 1H), 7.83-7.80 (m, 1H), 7.69-7.69 (m, 1H), 7.40 (s, 1H), 7.30-7.20 (m, 1H), 5.35-5.31 (m, 1H), 4.47 (s, 2H), 3.96-3.93 (m, 2H), 3.38-3.30 (m, 2H), 3.12-3.02 (m, 2H), 2.80-2.69 (m, 2H), 2.16-1.99 (m, 4H), 1.77-1.74 (m, 2H), 0.93-0.88 (m, 2H), 0.39-0.32 (m, 4H). ¹⁹F NMR (376 MHz, CD₃OD, ppm): 6-84.88 (1F), −100.21 (1F).

### Example 28 Synthesis of Compound 58

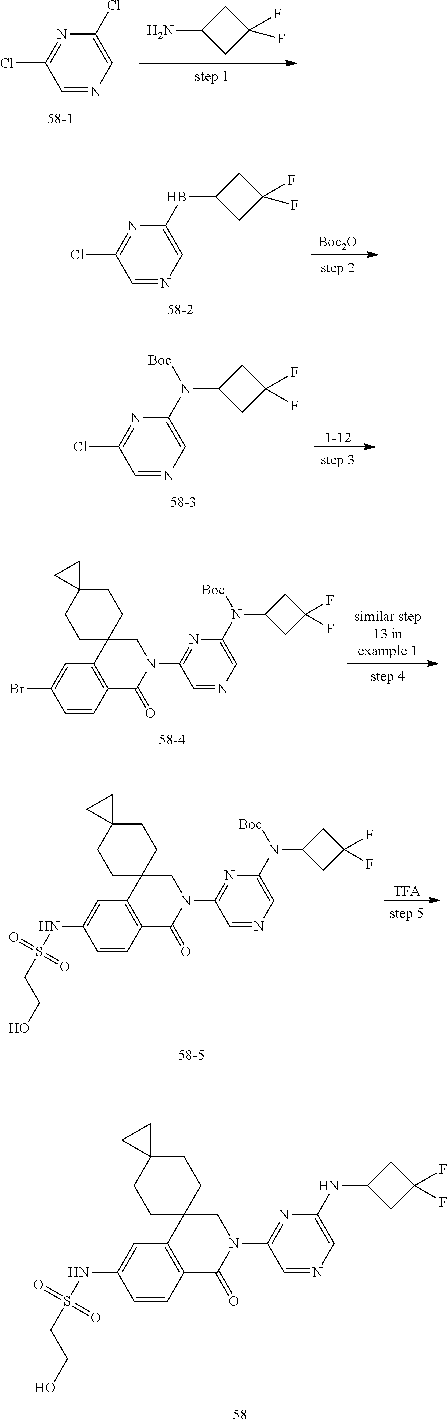

Step 1: To a solution of 58-1 (0.5 g, 3.36 mmol) and 3,3-difluorocyclobutan-1-amine (395.4 mg, 3.69 mmol) in NMP (5 mL) was added DIPEA (1.11 mL, 6.71 mmol). The mixture was stirred at 110° C. for 3 h. Water and EtOAc was added to the mixture and the mixture was extracted with EtOAc, the combined organic layer was dried over Na₂SO₄, filtered and concentrated, the residue was purified by column chromatography on silica gel (petroleum ether/EtOAc=1/0 to 2/1) to afford 58-2.

Step 2: To a solution of 58-2 (627 mg, 2.86 mmol) and Boc₂O (0.72 mL, 3.14 mmol) in DCM (20 mL) were added TEA (0.6 mL, 4.28 mmol) and DMAP (34.9 mg, 0.29 mmol). The mixture was stirred at 35° C. for 16 h. The mixture was concentrated under vacuo. The residue was purified by column chromatography on silica gel (petroleum ether/EtOAc=1/0 to 6/1) to afford 58-3.

Step 3: To a suspension of 1-12 (50 mg, 0.16 mmol) in dioxane (5 mL) was added KHMDS (0.31 mL, 0.31 mmol) at 0° C. After the mixture was stirred at room temperature for 10 minutes, 58-3 (79.88 mg, 0.25 mmol) was added to the mixture and the mixture was stirred at 90° C. for 6 h. The mixture was concentrated. The residue was purified by column chromatography on silica gel (petroleum ether/EtOAc=1/0 to 2/1) to afford 58-4.

Step 4: Compound 58-5 was prepared from compound 58-4 following the procedure for the synthesis of compound 1 in example 1.

Step 5: To the reaction mixture of 58-5 (20 mg, 0.031 mmol) in DCM (2 mL) was added TFA (0.6 mL, 7.84 mmol) slowly at room temperature. The reaction mixture was stirred at 35° C. for 2 h. The reaction mixture was concentrated. The residue was purified by reverse HPLC (MeCN/water (0.05% NH₃·H₂O in H₂O): 5%-35%) to afford 58. LCMS (ESI, m/z): [M+H]⁺=548.2; ¹H NMR (400 MHz, DMSO-d₆, ppm): δ 8.65 (s, 1H), 7.99-7.96 (m, 1H), 7.77-7.68 (m, 2H), 7.33 (s, 1H), 7.25-7.17 (m, 1H), 4.30 (s, 2H), 4.22-4.11 (m, 1H), 3.77-3.74 (m, 2H), 3.33-3.27 (m, 2H), 3.13-2.98 (m, 2H), 2.71-2.53 (m, 2H), 2.10-1.96 (m, 2H), 1.85-1.72 (m, 2H), 1.72-1.59 (m, 2H), 0.87-0.81 (m, 2H), 0.36-0.23 (m, 4H). ¹⁹F NMR (376 MHz, DMSO-d₆, ppm) δ −81.59 (1F), −95.66 (1F).

### Example 29 Synthesis of Compound 59

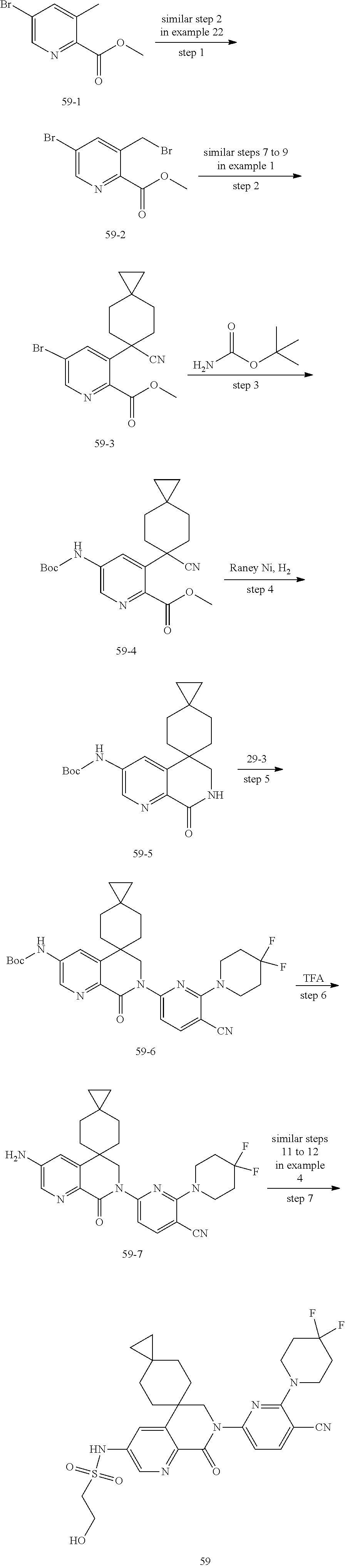

Step 1: Compound 59-2 was prepared from compound 59-1 following the procedure for the synthesis of compound 41-3 in example 22.

Step 2: Compound 59-3 was prepared from compound 59-2 following the procedure for the synthesis of compound 1-11 in example 1.

Step 3: A mixture of 59-3 (2.00 g, 5.73 mmol), tert-butyl carbamate (1.34 g, 11.45 mmol), XantPhos Pd G₂ (381.3 mg, 0.43 mmol) and Cs₂CO₃ (5.6 g, 17.18 mmol) in dioxane (40 mL) was heated at 100° C. for 12 h under N₂. The mixture was filtered and the filtrate was concentrated under vacuo. The residue was purified by column chromatography on silica gel (petroleum ether/EtOAc=1/0 to 3/1) to afford 59-4.

Step 4: To a solution of 59-4 (1.20 g, 3.11 mmol) in MeOH (40 mL) was added Raney Ni (1.0 g) and HCl/dioxane (3 mL), the reaction mixture was warmed to 60° C. and stirred for 72 h under H₂. The mixture was filtered and the filtrate was concentrated under vacuo. The residue was purified by column chromatography on silica gel (DCM/MeOH=1/0 to 5/1) to afford 59-5.

Step 5: To a solution of 59-5 (100 mg, 0.2 mmol) in dioxane (2 mL) were added 29-3 (75.7 mg, 0.29 mmol), Cs₂CO₃ (159.5 mg, 0.49 mmol), Xantphos (22.7 mg, 0.039 mmol) and Pd₂(dba)₃ (35.9 mg, 0.039 mmol). The reaction mixture was stirred at 105° C. for 9 h under N₂. The reaction mixture was concentrated in vacuo. The residue was diluted with EtOAc and water and extracted with EtOAc. The combined organic layer was washed with brine, dried over Na₂SO₄, filtered and concentrated. The residue was purified by column chromatography on silica gel (DCM/MeOH=1/0 to 10/1) to afford 59-6.

Step 6: To a solution of 59-6 (80 mg, 0.14 mmol) in DCM (5 mL) was added TFA (2 mL, 0.14 mmol) and the reaction mixture was stirred at room temperature for 2 h. The mixture was concentrated in vacuo. The residue was diluted with EtOAc and NaHCO₃ solution, extracted with EtOAc. The combined organic layer was washed with brine, dried over Na₂SO₄, filtered and concentrated. The residue was purified by column chromatography on silica gel (DCM/MeOH=1/0 to 20/1) to afford 59-7.

Step 7: Compound 59 was prepared from compound 59-7 following the procedure for the synthesis of compound 6 in example 4. LCMS (ESI, m/z): [M+H]⁺=587.4; ¹H NMR (400 MHz, DMSO-d₆, ppm): δ 8.06-8.00 (m, 1H), 7.94-7.91 (m, 1H), 7.89-7.84 (m, 1H), 7.30 7.23 (m, 1H), 4.38 (s, 2H), 3.85-3.81 (m, 4H), 3.72-3.69 (m, 2H), 2.96-2.93 (m, 2H), 2.20-2.09 (m, 4H), 1.96-1.87 (m, 2H), 1.82-1.72 (m, 2H), 1.64-1.58 (m, 2H), 0.87-0.78 (m, 2H), 0.28 (s, 4H). ¹⁹F NMR (376 MHz, DMSO-d₆, ppm): 6-94.66 (2F).

### Example 30 Synthesis of Compound 60

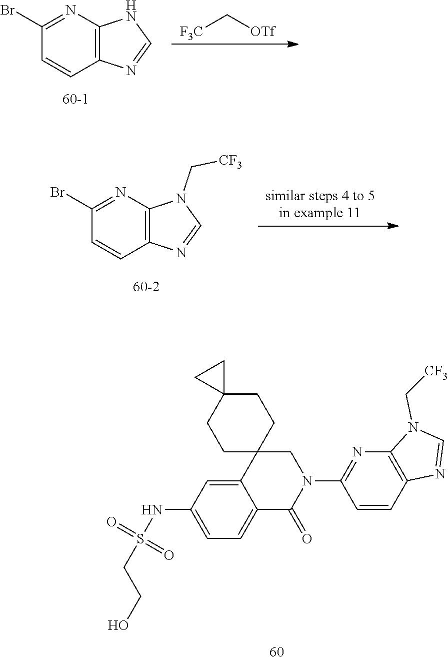 [SMILES: Brc1ccc2nc[nH]c2n1 ; O=S(=O)(OCC(F)(F)F)C(F)(F)F ; FC(F)(F)Cn1cnc2ccc(Br)nc21 ; O=C1c2ccc(NS(=O)(=O)CCO)cc2C2(CCC3(CC3)CC2)CN1c1ccc2ncn(CC(F)(F)F)c2n1]

Step 7: To a solution of 60-1 (500 mg, 2.54 mmol) and Cs₂CO₃ (1.65 g, 5.08 mmol) in DMF (10 mL) was added 2,2,2-trifluoroethyl trifluoromethanesulfonate (647.90 mg, 2.79 mmol). The reaction mixture was stirred at 25° C. for 16 h. The mixture was diluted with water and extracted with EtOAc. The combined organic layer was dried over Na₂SO₄, filtered and concentrated, the residue was purified by column chromatography on silica gel (petroleum ether/EtOAc=1/0 to 0/1) to afford 60-2.

Step 2: Compound 60 was prepared from compound 60-2 following the procedure for the synthesis of compound 16 in example 11. LCMS (ESI, m/z): [M+H]⁺=564.4; ¹H NMR (400 MHz, DMSO-d₆, ppm): δ 8.52-8.51 (m, 1H), 8.20-8.18 (m, 1H), 8.05-7.86 (m, 2H), 7.35-7.22 (m, 1H), 7.22-7.14 (m, 1H), 5.35-5.16 (m, 2H), 4.45-4.26 (m, 2H), 3.63-3.81 (m, 2H), 2.81-2.62 (m, 2H), 2.13-1.94 (m, 2H), 1.91-1.60 (m, 4H), 0.95-0.68 (m, 2H), 0.41-0.18 (m, 4H). ¹⁹F NMR (376 MHz, DMSO-d₆, ppm): 6-70.25 (3F).

### Example 31 Synthesis of Compound 67

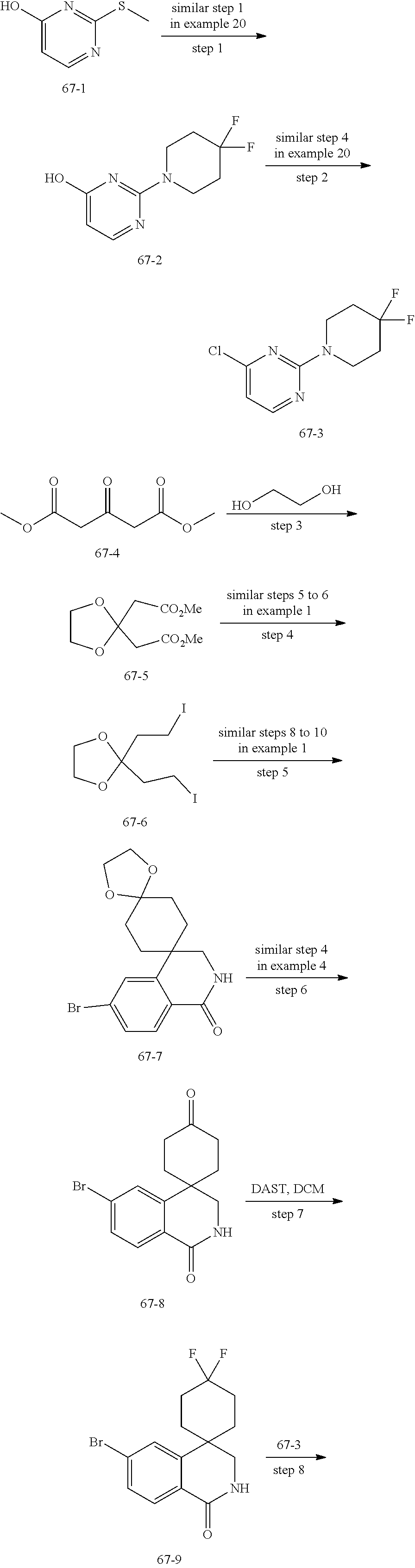

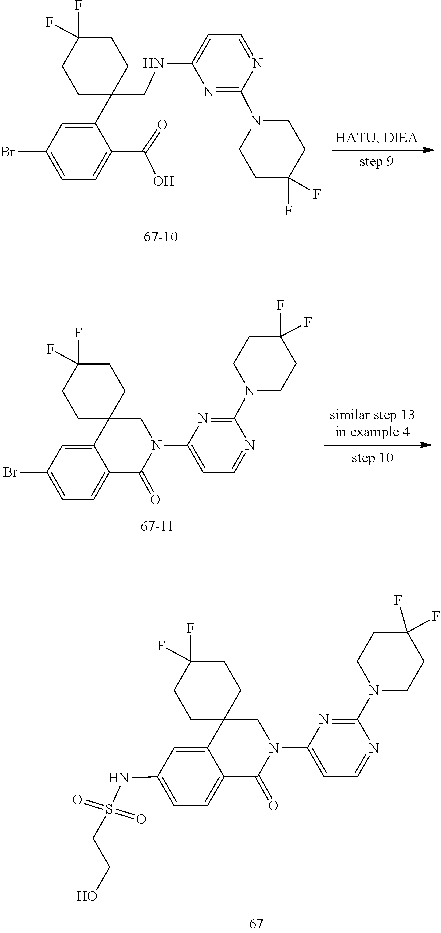 [SMILES: O=C(O)c1ccc(Br)cc1C1(CNc2ccnc(N3CCC(F)(F)CC3)n2)CCC(F)(F)CC1 ; O=C1c2ccc(Br)cc2C2(CCC(F)(F)CC2)CN1c1ccnc(N2CCC(F)(F)CC2)n1 ; O=C1c2ccc(NS(=O)(=O)CCO)cc2C2(CCC(F)(F)CC2)CN1c1ccnc(N2CCC(F)(F)CC2)n1]

Step 1: Compound 67-2 was prepared from compound 67-1 following the procedure for the synthesis of compound 35-2 in example 20.

Step 2: Compound 67-3 was prepared from compound 67-2 following the procedure for the synthesis of compound 55-5 in example 26.

Step 3: To a solution of 67-4 (10 g, 57.42 mmol) in DCM (200 mL) was added ethylene glycol (3.52 mL, 63.16 mmol) and chlorotrimethylsilane (14.56 mL, 114.84 mmol), the reaction mixture was stirred at 50° C. for 16 h. The mixture was diluted with DCM and saturated NaHCO₃. The organic layer was separated, washed with brine, dried over Na₂SO₄, filtered and concentrated under vacuo. The residue was purified by column chromatography on silica gel (petroleum ether/ethyl acetate=1/0 to 6/1) to afford 67-5.

Step 4: Compound 67-6 was prepared from compound 67-5 following the procedure for the synthesis of compound 1-7 in example 1.

Step 5: Compound 67-7 was prepared from compound 67-6 following the procedure for the synthesis of compound 1-12 in example 1.

Step 6: Compound 67-8 was prepared from compound 67-7 following the procedure for the synthesis of compound 6-6 in example 4.

Step 7: To a solution of 67-8 (430 mg, 1.40 mmol) in DCM (10 mL) was added Diethylaminosulfur trifluoride (1.11 mL, 8.37 mmol) at 0° C. Then the mixture was stirred at 0° C. for 3 h. The reaction was quenched with aqueous NH₄Cl and extracted with DCM. The combined organic layers were concentrated and purified by column chromatography on silica gel (DCM/MeOH=1/0 to 10/1) to afford 67-9.

Step 8: To a suspension of 67-9 (100 mg, 0.30 mmol) in DMA (3 mL) was added NaH (36.3 mg, 0.91 mmol) at 0° C. After this mixture was stirred at 0° C. for 10 minutes, 67-3 (92.0 mg, 0.39 mmol) was added. Then the mixture was stirred at 60° C. for 2 h. The reaction was quenched with H₂O and extracted with EtOAc. The combined organic layers were dried over Na₂SO₄, filtered and the filtrate was concentrated under vacuo. The residue was purified by column chromatography on silica gel (DCM/MeOH=1/0 to 10/1) to afford 67-10.

Step 9: To a solution of 67-10 (80 mg, 0.15 mmol) in DMF (3 mL) were added HATU (111.6 mg, 0.29 mmol) and DIPEA (0.24 mL, 1.47 mmol) at room temperature. Then the mixture was heated at 60° C. for 1 h. The mixture was concentrated under vacuo and the residue was purified by column chromatography on silica gel (petroleum ether/EtOAc=1/0 to 10/1) to afford 67-11.

Step 10: Compound 67 was prepared from compound 67-11 following the procedure for the synthesis of compound 1 in example 1. The crude product 67 was purified by column chromatography on silica gel (petroleum ether/EtOAc=1/0 to 2/3) and SFC (column: ChiralCel OJ, 150×4.6 mm I.D., 3 μm (0.05% DEA in MeOH) in Supercritical CO₂=5-40%) to remove the impurity to afford pure 67. LCMS (ESI, m/z): [M+H]⁺=572.3; ¹H NMR (400 MHz, DMSO-d₆, ppm): δ 8.34-8.33 (m, 1H), 7.98-7.96 (m, 1H), 7.68-7.67 (m, 1H), 7.24-7.16 (m, 2H), 4.46 (s, 2H), 3.97-3.84 (m, 4H), 3.76-3.73 (m, 2H), 3.31-3.26 (m, 2H), 2.16-2.10 (m, 2H), 2.10-1.93 (m, 6H), 1.93-1.83 (m, 2H), 1.83-1.73 (m, 2H). ¹⁹F NMR (376 MHz, DMSO-d₆, ppm): 6-89.49 (1F), −94.83 (2F), −99.09 (1F).

### Example 32 Synthesis of Compound 68

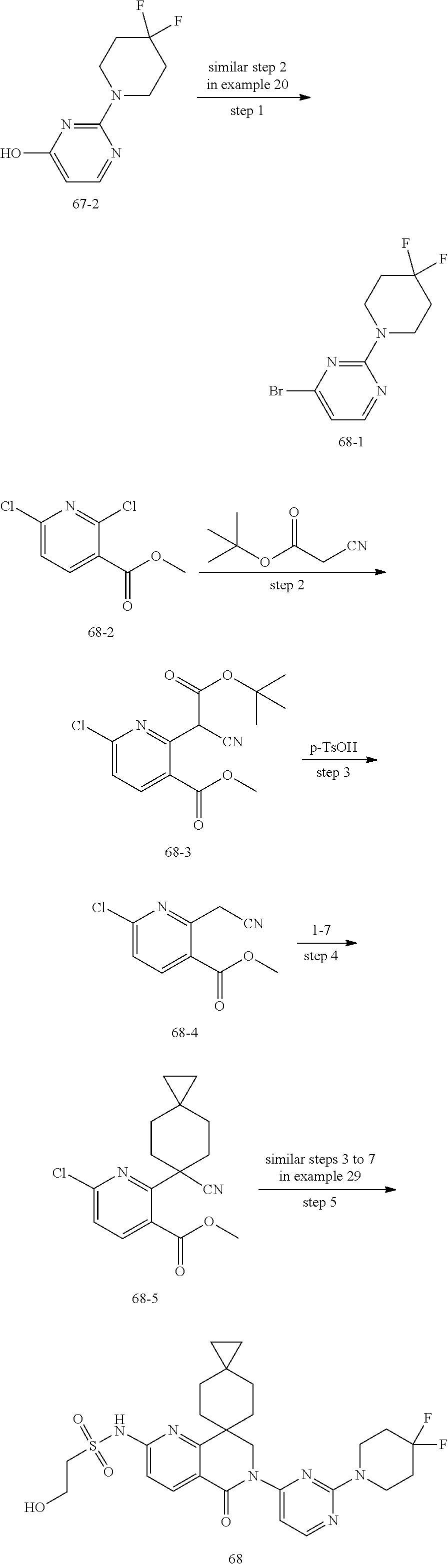

Step 1: Compound 68-1 was prepared from compound 67-2 following the procedure for the synthesis of compound 35-3 in example 20.

Step 2: To a solution of 68-2 (25 g, 121.35 mmol) and 2-methylpropan-2-yl cyanoacetate (20.56 g, 145.62 mmol) in DMF (200 mL) was added K₂CO₃ (16.8 g, 121.35 mmol) at room temperature. The reaction mixture stirred 100° C. for 6 h. The reaction was quenched with H₂O and extracted with EtOAc. The combined organic layers were dried over Na₂SO₄, filtered and the filtrate was concentrated under vacuo. The residue was purified by column chromatography on silica gel (petroleum ether/EtOAc=1/0 to 6/1) to afford 68-3.

Step 3: To a solution of 68-3 (5.20 g, 16.74 mmol) in toluene (50 mL) was added 4-methylbenzenesulfonic acid (0.3 g, 1.67 mmol) at room temperature. The reaction mixture stirred 100 T for 6 h. The reaction mixture was concentrated under vacuo. The residue was purified by column chromatography on silica gel (petroleum ether/EtOAc=1/0 to 2/1) to afford 68-4.

Step 4: To a solution of methyl 68-4 (1.50 g, 7.12 mmol) and 1-7 (2.74 g, 7.83 mmol) in DMA (20 mL) was added K₂CO₃ (3.0 g, 21.37 mmol) at room temperature. The reaction mixture stirred at 100° C. for 30 minutes. This reaction was quenched with H₂O and extracted with EtOAc. The combined organic layers were dried over Na₂SO₄, filtered and the filtrate was concentrated under vacuo to afford 68-5 which was used for the next step directly without further purification.

Step 5: Compound 68 was prepared from compound 68-5 following the procedure for the synthesis of compound 59 in example 29. The product was obtained as 0.3 FA salt. LCMS (ESI, m/z): [M+H]⁺=563.2; ¹H NMR (400 MHz, DMSO-d₆, ppm): δ 8.32-8.31 (m, 1H), 8.21 (s, 0.3H), 8.14-8.12 (m, 1H), 7.58-7.57 (m, 1H), 6.83-6.81 (m, 1H), 4.46 (s, 2H), 3.97-3.90 (m, 4H), 3.83-3.75 (m, 2H), 3.69-3.61 (m, 2H), 2.14-1.94 (m, 6H), 1.94-1.82 (m, 2H), 1.59-1.51 (m, 2H), 0.98-0.89 (m, 2H), 0.34-0.21 (m, 4H). ¹⁹F NMR (376 MHz, DMSO-d₆, ppm): 6-94.69 (2F).

### Example 33 Synthesis of Compound 73

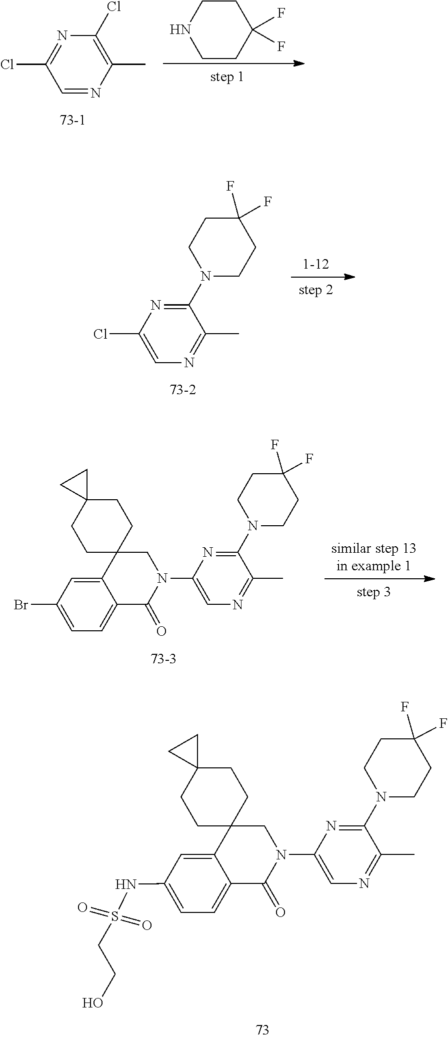

Step 1: To a solution of 73-1 (1 g, 6.14 mmol) in DMF (30 mL) were added 4,4-difluorohexahydropyridine (2.23 g, 18.41 mmol) and Cs₂CO₃ (10.0 g, 30.68 mmol), the reaction mixture was stirred at 100° C. for 6 h. Water was added to the mixture and the mixture was extracted with EtOAc. The combined organic layer was washed with brine and dried over Na₂SO₄, filtered and concentrated. The residue was purified by column chromatography on silica gel (petroleum ether/EtOAc=1/0 to 2/1) to afford 73-2.

Step 2: To a solution of 1-12 (100 mg, 0.31 mmol) in DMA (3 mL) were added 73-2 (154.7 mg, 0.63 mmol) and Cs₂CO₃ (305.2 mg, 0.94 mmol), the reaction mixture was stirred at 150° C. for 18 h. Water was added to the mixture and the mixture was extracted with EtOAc. The combined organic layer was washed with brine and dried over Na₂SO₄, filtered and concentrated. The residue was purified by column chromatography on silica gel (petroleum ether/EtOAc=l/0 to 0/1) to afford 73-3.

Step 3: Compound 73 was prepared from compound 73-3 following the procedure for the synthesis of compound 1 in example 1. LCMS (ESI, m/z): [M+H]⁺=576.2; ¹H NMR (400 MHz, CD₃OD, ppm): δ 8.90 (s, 1H), 7.94-7.92 (m, 1H), 7.27 (s, 1H), 7.14-7.11 (m, 1H), 4.33 (s, 2H), 3.96-3.93 (m, 2H), 3.48-3.42 (m, 4H), 2.53 (s, 3H), 2.18-2.00 (m, 10H), 1.77-1.74 (m, 2H), 0.89-0.80 (m, 2H), 0.33 (s, 4H). ¹⁹F NMR (376 MHz, CD₃OD, ppm): δ −98.55 (2F).

### Example 34 Synthesis of Compound 77

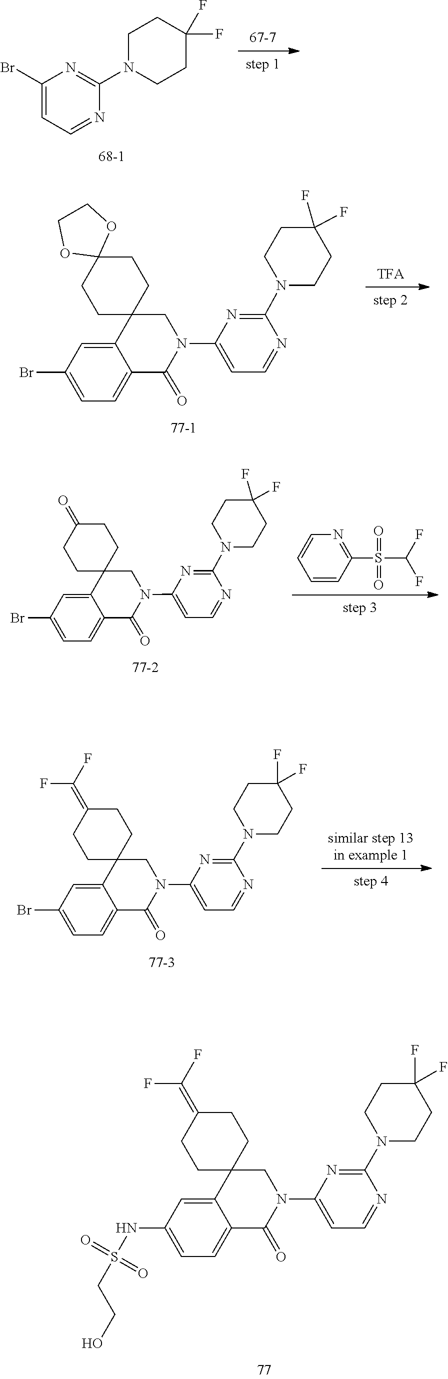

Step 1: A solution of 67-7 (250 mg, 0.71 mmol), 68-1 (296.1 mg, 1.06 mmol), CsF ((323.4 mg, 2.13 mmol), CuI (135.2 mg, 0.71 mmol) and N,N′-Dimethylethylenediamine (31.3 mg, 0.36 mmol) in DMA (2 mL) was heated to stirred at 90° C. for 16 h. The solvent was removed and the residue was purified by column chromatography on silica gel (petroleum ether/ethyl acetate=1/0 to 0/1) to afford 77-1.

Step 2: A solution of 77-1 (130 mg, 0.24 mmol) in TFA (2 mL), THF (1 mL) and water (1.5 mL) was stirred at room temperature for 16 h. The mixture was poured into saturated NaHCO₃ solution and extracted with EtOAc. The combined organic layer was dried and concentrated. The residue was purified by column chromatography on silica gel (petroleum ether/ethyl acetate=1/0 to 0/1) to afford 77-2.

Step 3: To a solution of Bu^{t}OK (31.1 mg, 0.28 mmol) in DMF (0.5 mL) was added a solution of 77-2 (70 mg, 0.14 mmol) and difluoromethyl 2-pyridyl sulfone (29.4 mg, 0.15 mmol) in DMF (0.5 mL) at −50° C. Then the mixture was stirred at 0° C. for 1 h. The reaction was quenched with water. Then the mixture was extracted with EtOAc, the combined organic layer was dried and concentrated. The residue was purified by column chromatography on silica gel (petroleum ether/ethyl acetate=1/0 to 0/1) to afford 77-3.

Step 4: Compound 77 was prepared from compound 77-3 following the procedure for the synthesis of compound 1 in example 1. LCMS (ESI, m/z): [M+H]⁺=584.2; ¹H NMR (400 MHz, DMSO-d₆, ppm): 8 8.32 (d, J=5.7 Hz, 1H), 7.94 (d, J=8.4 Hz, 1H), 7.62 (d, J=5.7 Hz, 1H), 7.20-7.13 (m, 2H), 4.42 (s, 2H), 3.94-3.86 (m, 4H), 3.76-3.73 (m, 2H), 3.30-3.27 (m, 2H), 2.40-2.37 (m, 2H), 2.13-2.08 (m, 2H), 2.03-1.96 (m, 4H), 1.82-1.65 (m, 4H). ¹⁹F NMR (376 MHz, DMSO-d₆, ppm): δ −94.77 (2F), −97.46 (2F).

### Example 35 Synthesis of Compound 78

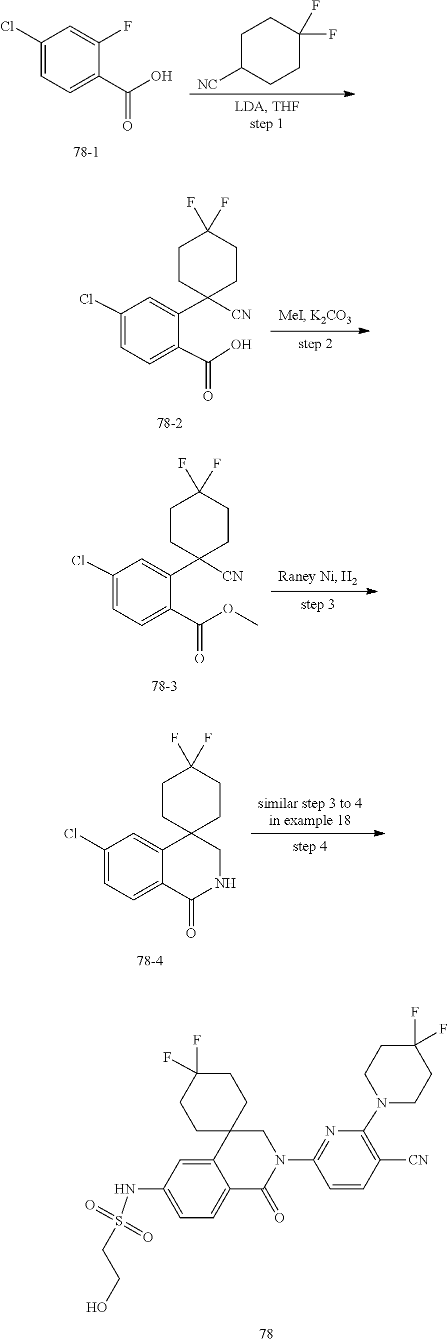 [SMILES: O=C(O)c1ccc(Cl)cc1F ; N#CC1CCC(F)(F)CC1 ; N#CC1(c2cc(Cl)ccc2C(=O)O)CCC(F)(F)CC1 ; COC(=O)c1ccc(Cl)cc1C1(C#N)CCC(F)(F)CC1 ; O=C1NCC2(CCC(F)(F)CC2)c2cc(Cl)ccc21 ; N#Cc1ccc(N2CC3(CCC(F)(F)CC3)c3cc(NS(=O)(=O)CCO)ccc3C2=O)nc1N1CCC(F)(F)CC1]

Step 1: To a solution of 4,4-difluorocyclohexane-1-carbonitrile (2.5 g, 17.22 mmol) in THF (30 mL) was added LDA (21.53 mL, 43.06 mmol) at −78° C. The mixture was stirred at −78° C. for 3 h, then 78-1 (3.01 g, 17.22 mmol) in THF (30 mL) was added dropwise to the mixture at −78° C. The resulting mixture was stirred at room temperature for 16 h. HCl (1N, 200 mL) was added to the mixture and the mixture was extracted with EtOAc. The organic layer was separated and dried Na₂SO₄, filtered and concentrated under vacuum to afford 78-2 which was used for the next step directly without further purification.

Step 2: To a solution of 78-2 (3.9 g, crude) in DMF (50 mL) were added CH₃I (1.62 mL, 26.03 mmol) and K₂CO₃ (5.4 g, 39.04 mmol), the reaction mixture was stirred at room temperature for 16 h. The mixture was filtered and the mixture was concentrated under vacuum. The residue was purified by column chromatography on silica gel (petroleum ether/ethyl acetate=1/0 to 4/1) to afford 78-3.

Step 3: To a solution of 78-3 (4 g, 12.75 mmol) in MeOH (60 mL) was added Raney Ni (3 g) at room temperature. Then the mixture was stirred under H₂ (1 atm, maintained by balloon) at 50° C. for 8 h. This mixture was filtered and the filtrate was concentrated under vacuum, the residue was purified by column chromatography on silica gel (DCM/MeOH=1/0 to 20/1) to afford 78-4.

Step 4: Compound 78 was prepared from compound 78-4 following the procedure for the synthesis of compound 27 in example 18. LCMS (ESI, m/z): [M+H]⁺=596.2; ¹H NMR (400 MHz, DMSO-d₆, ppm): δ 8.08 (d, J=8.6 Hz, 1H), 7.90 (d, J=8.6 Hz, 1H), 7.84 (d, J=8.6 Hz, 1H), 7.16-7.05 (m, 2H), 4.36 (s, 2H), 3.81-3.70 (m, 6H), 3.20 (t, J=6.5 Hz, 2H), 2.20-1.94 (m, 8H), 1.94-1.84 (m, 2H), 1.83-1.74 (m, 2H). ¹⁹F NMR: (376 MHz, DMSO-d₆, ppm): δ −89.95 (1F), −94.96 (2F), −99.24 (1F).

### Example 36 Synthesis of Compound 83

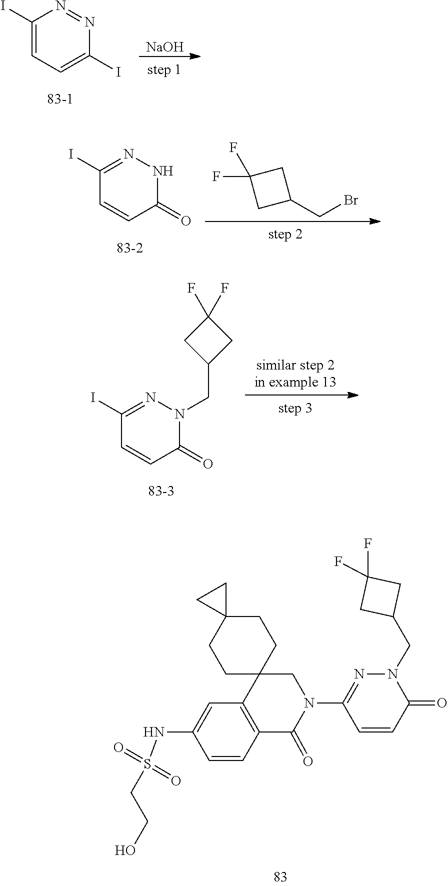

Step 1: A solution of 83-1 (400 mg, 1.21 mmol) and NaOH (48.2 mg, 1.21 mmol) in H₂O (10 mL) was stirred at 110° C. for 1 h by microwave. H₂O was added to the mixture and the mixture was acidified by HCl (2N) to pH=5, then the mixture was extracted with EtOAc. The combined organic layer was washed with brine and dried Na₂SO₄, filtered and concentrated in vacuo. The residue was purified by column chromatography on silica gel (petroleum ether/ethyl acetate=1/0 to 2/3) to afford 83-2.

Step 2: To a solution of 83-2 (300 mg, 1.35 mmol) in DMA (6 mL) was added 3-(bromomethyl)-1,1-difluorocyclobutane (250.0 mg, 1.35 mmol) at 20° C., then K₂CO₃ (373.5 mg, 2.70 mmol) was added and the reaction mixture was stirred at 50° C. for 16 h. H₂O was added to the mixture and the mixture was extracted with EtOAc. The organic fractions were combined, washed with brine, then dried with sodium sulfate, filtered and concentrated in vacuo. The crude mixture was purified by flash chromatography (petroleum ether/ethyl acetate=1/0 to 4/1) to afford 83-3.

Step 3: Compound 83 was prepared from compound 83-3 following the procedure for the synthesis of compound 18 in example 13. LCMS (ESI, m/z): [M+H]⁺=563.2; ¹H NMR (400 MHz, DMSO-d₆, ppm): δ 7.93 (d, J=8.4 Hz, 1H), 7.85 (d, J=9.8 Hz, 1H), 7.34 (s, 1H), 7.22 (d, J=8.6 Hz, 1H), 6.99 (d, J=9.9 Hz, 1H), 4.20 (d, J=6.8 Hz, 2H), 4.04 (s, 2H), 3.75 (t, J=6.4 Hz, 2H), 2.75-2.61 (m, 6H), 2.55-2.51 (m, 1H), 1.98-1.66 (m, 6H), 0.87 (d, J=13.0 Hz, 2H), 0.30 (s, 4H). ¹⁹F NMR (376 MHz, DMSO-d₆, ppm): 8-81.56 (1F), −91.77 (1F).

### Example 37 Synthesis of Compound 85

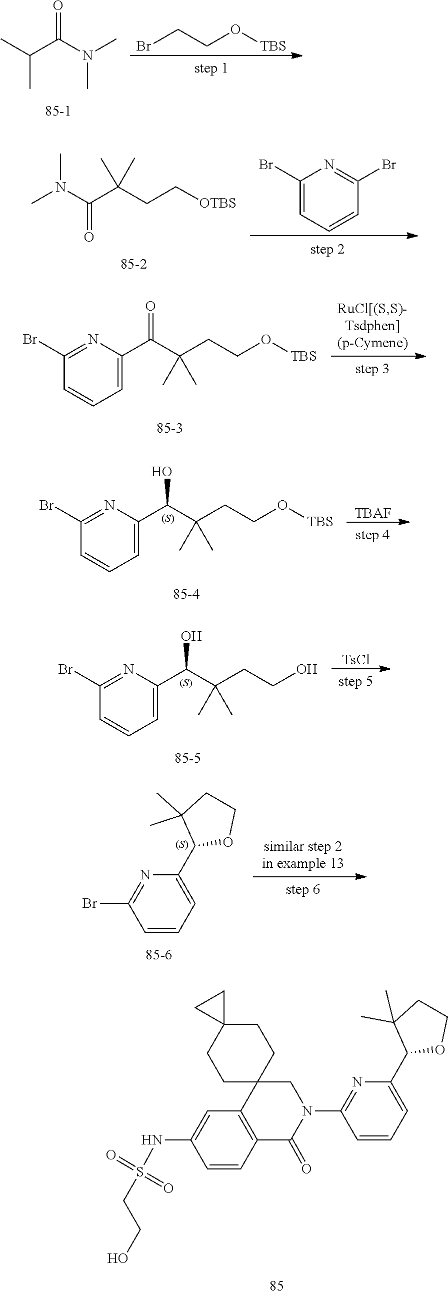

Step 1: To a solution of 85-1 (3.8 g, 32.99 mmol) in THF (40 mL) was added LDA (23.09 mL, 46.19 mmol) at 0° C. After this mixture was stirred at 0° C. for 0.5 h, 1-bromo-4,4,5,5-tetramethyl-3-oxa-4-silahexane (11.84 g, 49.49 mmol) was added to the mixture. Then the mixture was stirred at room temperature for 12 h. The reaction was quenched with H₂O and extracted with EtOAc. The combined organic layers were dried over Na₂SO₄, filtered and the filtrate was concentrated in vacuo. The residue was purified by column chromatography on silica gel (petroleum ether/ethyl acetate=1/0 to 4/1) to afford 85-2.

Step 2: To a solution of 2,6-dibromopyridine (4.1 g, 17.31 mmol) in THF (80 mL) was added n-BuLi (7.62 mL, 19.04 mmol) at −78° C. After this mixture was stirred at −78° C. for 0.5 h, 85-2 (5.21 g, 19.04 mmol) was added to the mixture. Then the mixture was stirred at room temperature for 1 h. The reaction was quenched with H₂O and extracted with EtOAc. The combined organic layers were dried over Na₂SO₄, filtered and the filtrate was concentrated in vacuo. The residue was purified by column chromatography on silica gel (petroleum ether/ethyl acetate=1/0 to 10/1) to afford 85-3.

Step 3: To a mixture of 85-3 (4.2 g, 10.87 mmol), (S,S)—N-(p-Toluenesulfonyl)-1,2-diphenylethanediamine(chloro)(p-cymene)ruthenium(II) (325.1 mg, 0.51 mmol) was added a mixture of TEA (3.78 mL, 27.17 mmol) and formic acid (222.3 mg, 1.16 mmol). The mixture was stirred at 25° C. for 16 h. The reaction was quenched with H₂O and extracted with EtOAc. The combined organic layer was dried over Na₂SO₄, filtered and concentrated. The residue was purified by column chromatography on silica gel (petroleum ether/ethyl acetate=1/0 to 20/1) to afford 85-4.

Step 4: To a solution of 85-4 (1.6 g, 4.12 mmol) in THF (20 mL) was added TBAF (4.53 mL, 4.53 mmol). Then the mixture was stirred at room temperature for 1 h. The mixture was concentrated in vacuo and the residue was purified by column chromatography on silica gel (petroleum ether/ethyl acetate=1/0 to 1/4) to afford 85-5.

Step 5: To a mixture of 85-5 (820 mg, 2.99 mmol) in DCM (40 mL) were added DMAP (401.9 mg, 3.29 mmol), TEA (0.83 mL, 5.98 mmol) and 4-methylbenzenesulfonyl chloride (627.2 mg, 3.29 mmol). The mixture was stirred at room temperature for 1 h. The mixture was concentrated in vacuo and the residue was purified by column chromatography on silica gel (petroleum ether/ethyl acetate=1/0 to 4/1) to afford 85-6.

Step 6: Compound 85 was prepared from compound 85-6 following the procedure for the synthesis of compound 18 in example 13. LCMS (ESI, m/z): [M+H]⁺=540.2; ¹H NMR (400 MHz, DMSO-d₆, ppm): δ 8.02-7.96 (m, 2H), 7.81 (t, J=7.9 Hz, 1H), 7.34 (s, 1H), 7.27-7.18 (m, 2H), 4.51-4.40 (m, 2H), 4.27 (d, J=13.4 Hz, 1H), 4.12-4.08 (m, 1H), 3.99-3.90 (m, 1H), 3.76 (t, J=6.5 Hz, 2H), 2.14-2.03 (m, 1H), 2.02-1.62 (m, 8H), 1.38-1.19 (m, 4H), 0.91-0.73 (m, 2H), 0.61 (s, 3H), 0.34-0.22 (m, 4H).

### Example 38 Synthesis of Compound 91

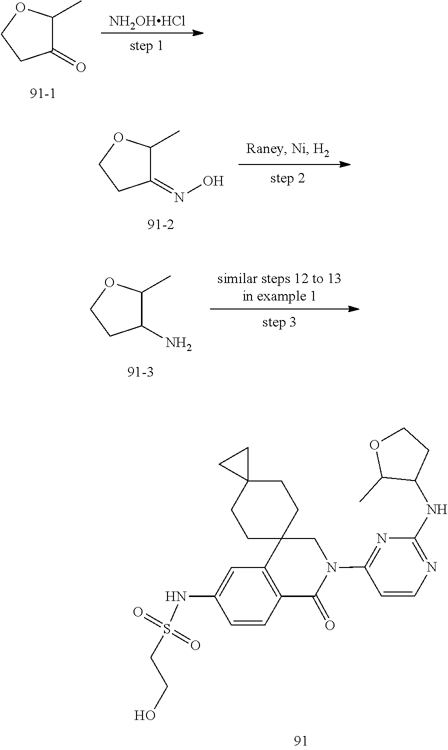

Step 1: To a colorless solution of 91-1 (1.5 g, 14.98 mmol) in saturated Na₂CO₃ (15 mL) was added NH₂OH·HCl (1145.2 mg, 16.48 mmol). The reaction mixture was stirred at 40° C. for 2 h. The mixture was extracted with EtOAc, the organic layer was dried and concentrated to afford 91-2 which was used for the next step directly without further purification.

Step 2: To 91-2 (1.55 g, crude) in MeOH (20 mL) was added Raney Ni (1184.5 mg, 20.18 mmol), the mixture was stirred under H₂ balloon at 60° C. for 16 h. The mixture was filtered and concentrated to afford 91-3 which was used for the next step directly without further purification.

Step 3: Compound 91 was prepared from compound 91-3 following the procedure for the synthesis of compound 1 in example 1. LCMS (ESI, m/z): [M+H]⁺=542.4; ¹H NMR (400 MHz, DMSO-d₆, ppm): δ 8.12 (d, J=5.5 Hz, 1H), 7.71 (d, J=8.6 Hz, 1H), 7.58-7.41 (m, 1H), 7.35-7.15 (m, 1H), 6.93-6.78 (m, 2H), 5.65 (s, 1H), 4.70-4.25 (m, 2H), 4.23-3.98 (m, 1H), 3.95-3.52 (m, 5H), 2.98 (t, J=6.6 Hz, 2H), 2.32-2.17 (m, 1H), 2.08-1.65 (m, 5H), 1.58 (d, J=12.9 Hz, 2H), 1.21 (d, J=6.1 Hz, 2H), 1.01 (d, J=6.2 Hz, 1H), 0.88-0.70 (m, 2H), 0.27 (s, 4H).

### Example 39 Synthesis of Compound 92

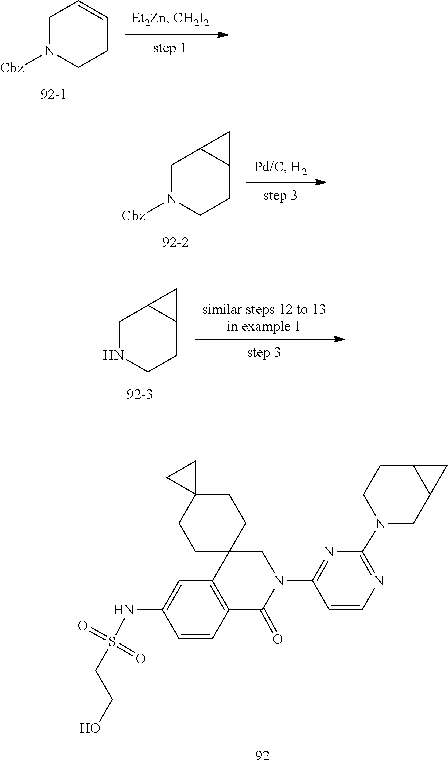 [SMILES: O=C(OCc1ccccc1)N1CC=CCC1 ; O=C(OCc1ccccc1)N1CCC2CC2C1 ; C1CC2CC2CN1 ; O=C1c2ccc(NS(=O)(=O)CCO)cc2C2(CCC3(CC3)CC2)CN1c1ccnc(N2CCC3CC3C2)n1]

Step 1: Diethylzinc (34.52 mL, 34.52 mmol) was added to a solution of 92-1 (1 g, 5.46 mmol) in toluene (50 mL) at −60° C. The mixture was stirred for 15 minutes at this temperature, then diiodomethane (18.5 g, 69.04 mmol) was added dropwise to the mixture over 30 minutes. The mixture was stirred at room temperature for 16 h. The mixture was poured into ice-cooled saturated aqueous NH₄Cl solution, extracted with diethyl ether. The organic phase was washed with water and dried over Na₂SO₄, filtered and concentrated. The residue was purified by column chromatography on silica gel (petroleum ether/ethyl acetate=1/0 to 10/1) to afford 92-2.

Step 2: A mixture of 92-2 (180 mg, 0.78 mmol) and Pd/C (10%, 18 mg) in MeOH (3 mL) was stirred at room temperature for 16 h under H₂. The mixture was filtered, the filtrated was concentrated to afford 92-3 which was used for the next step directly without further purification.

Step 3: Compound 92 was prepared from compound 92-3 following the procedure for the synthesis of compound 1 in example 1. LCMS (ESI, m/z): [M+H]⁺=538.4; ¹H NMR (400 MHz, DMSO-d₆, ppm): δ 8.24 (d, J=5.8 Hz, 1H), 7.95 (d, J=8.6 Hz, 1H), 7.53 (d, J=5.8 Hz, 1H), 7.27-7.17 (m, 2H), 4.47-4.43 (m, 2H), 4.20-4.17 (m, 1H), 3.98-3.88 (m, 1H), 3.77-3.70 (m, 2H), 3.45-3.35 (m, 4H), 2.08-1.99 (m, 4H), 1.79-1.72 (m, 2H), 1.64-1.61 (m, 2H), 1.13-1.02 (m, 2H), 0.85-0.83 (m, 2H), 0.62-0.59 (m, 1H), 0.35-0.28 (m, 4H), 0.15-0.10 (m, 11H).

### Example 40 Synthesis of Compound 93

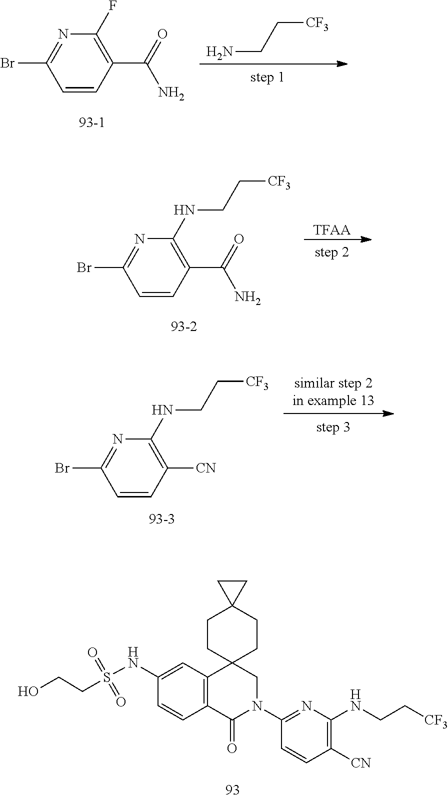

Step 1: A mixture of 93-1 (150 mg, 0.69 mmol), 3,3,3-trifluoropropan-1-amine (116.2 mg, 1.03 mmol) and K₂CO₃ (284.0 mg, 2.06 mmol) in CH₃CN (3 mL) was heated at 80° C. for 1 h. The mixture was filtered and the filtrate was concentrated in vacuo. The residue was purified by column chromatography on silica gel (petroleum ether/ethyl acetate=1/0 to 1/1) to afford 93-2.

Step 2: To a mixture of 93-2 (100 mg, 0.32 mmol) and TEA (0.07 mL, 0.48 mmol) in DCM (3 mL) was added TFAA (100.9 mg, 0.48 mmol). Then the mixture was stirred at room temperature for 15 minutes. The pH of the mixture was adjusted to 8 with TEA. Then the mixture was concentrated in vacuo and the residue was purified by column chromatography on silica gel (petroleum ether/ethyl acetate=1/0 to 4/1) to afford 93-3.

Step 3: Compound 93 was prepared from compound 93-3 following the procedure for the synthesis of compound 18 in example 13. LCMS (ESI, m/z): [M+H]⁺=578.2; ¹H NMR (400 MHz, DMSO-d₆, ppm): δ 7.89-7.85 (m, 2H), 7.60 (d, J=8.6 Hz, 1H), 7.40 (t, J=5.6 Hz, 1H), 6.93-7.18 (m, 2H), 4.36 (s, 2H), 3.75-3.66 (m, 4H), 3.18 (t, J=5.8 Hz, 2H), 2.71-2.59 (m, 2H), 1.92-1.75 (m, 4H), 1.62 (d, J=12.4 Hz, 2H), 0.83 (d, J=13.0 Hz, 2H), 0.27 (s, 4H). ¹⁹F NMR (376 MHz, DMSO-d₆, ppm): δ −63.73 (3F).

### Example 41 Synthesis of Compound 99 and 100

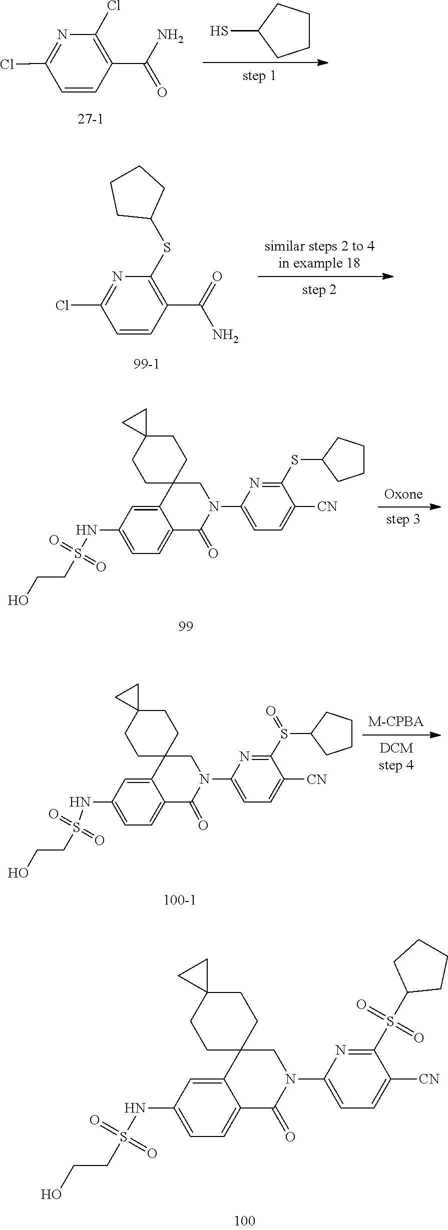 [SMILES: NC(=O)c1ccc(Cl)nc1Cl ; SC1CCCC1 ; NC(=O)c1ccc(Cl)nc1SC1CCCC1 ; N#Cc1ccc(N2CC3(CCC4(CC4)CC3)c3cc(NS(=O)(=O)CCO)ccc3C2=O)nc1SC1CCCC1 ; N#Cc1ccc(N2CC3(CCC4(CC4)CC3)c3cc(NS(=O)(=O)CCO)ccc3C2=O)nc1S(=O)C1CCCC1 ; N#Cc1ccc(N2CC3(CCC4(CC4)CC3)c3cc(NS(=O)(=O)CCO)ccc3C2=O)nc1S(=O)(=O)C1CCCC1]

Step 1: To a solution of 27-1 (300 mg, 1.57 mmol) and K₂CO₃ (325.6 mg, 2.36 mmol) in CH₃CN (6 mL) was added cyclopentanethiol (0.15 mL, 1.41 mmol) at 20° C. The mixture was stirred at 20° C. for 2 h. The mixture was diluted with H₂O and extracted with EtOAc. The organic phase was washed with H₂O and concentrated to afford 99-1 which was used for the next step directly without further purification.

Step 2: Compound 99 was prepared from compound 99-1 following the procedure for the synthesis of compound 27 in example 18. LCMS (ESI, m/z): [M+H]⁺=567.2; ¹H NMR (400 MHz, DMSO-d₆, ppm): δ 8.35 (s, 1H), 8.21-8.07 (m, 2H), 7.93 (d, J=8.6 Hz, 1H), 7.21 (s, 1H), 7.12 (d, J=8.4 Hz, 1H), 4.47 (s, 2H), 4.28-4.15 (m, 1H), 3.74 (t, J=6.6 Hz, 2H), 3.23 (t, J=6.6 Hz, 2H), 2.36-2.20 (m, 2H), 1.96-1.75 (m, 6H), 1.73-1.61 (m, 6H), 0.92-0.81 (m, 2H), 0.33-0.25 (m, 4H).

Step 3: A solution of 99 (110 mg, 0.19 mmol) and oxone (1.19 g, 1.94 mmol) in CH₃CN (18 mL) and H₂O (10 mL) was stirred at 20° C. for 16 h. The mixture was diluted with H₂O and extracted with EtOAc. The combined organic phases were washed with H₂O and concentrated. The residue was purified by column chromatography on silica gel (DCM/MeOH=1/0 to 10/1) to afford 100-1.

Step 4: A solution of 100-1 (51 mg, 0.088 mmol) and 3-chloroperoxybenzoic acid (30.2 mg, 0.18 mmol) in DCM (5 mL) was stirred at 20° C. for 4 h. The mixture was concentrated. The residue was purified by column chromatography on silica gel (DCM/MeOH=1/0 to 10/1) and reverse HPLC (MeCN/in water (0.05% FA)): 5%-70% to afford 100 as 0.3 FA salt. LCMS (ESI, m/z): [M+H]⁺=599.2; ¹H NMR (400 MHz, DMSO-d₆, ppm): δ 8.65-8.55 (m, 2H), 8.28 (s, 0.3H), 7.88 (d, J=8.6 Hz, 1H), 7.27 (s, 1H), 7.18 (d, J=8.5 Hz, 1H), 4.42 (s, 2H), 4.32-4.21 (m, 1H), 3.75 (t, J=6.4 Hz, 2H), 3.29 (t, J=6.4 Hz, 2H), 2.09-2.00 (m, 4H), 1.92-1.60 (m, 10H), 0.90-0.80 (m, 2H), 0.33-0.24 (s, 4H).

### Example 42 Synthesis of Compound 103

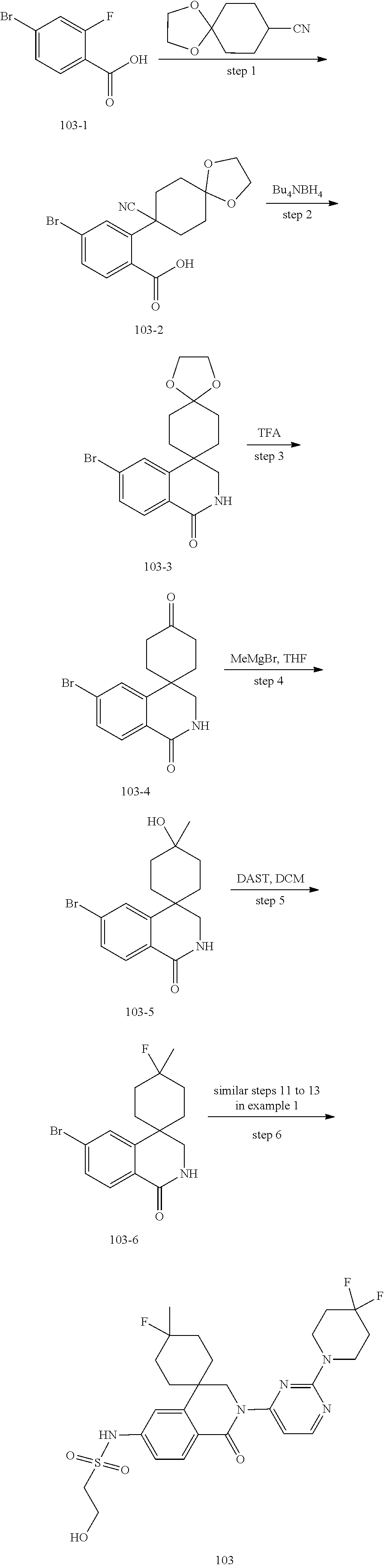

Step 1: To a solution of 1,4-dioxaspiro[4.5]decane-8-carbonitrile (10 g, 59.81 mmol) in THF (80 mL) was added LDA (74.76 mL, 149.51 mmol) at −78° C. for 30 minutes, the reaction mixture was stirred at −78° C. for 1 h. 103-1 (13.10 g, 59.81 mmol) was added dropwise to the mixture at −78° C. The mixture was stirred at room temperature for 16 h. The reaction was quenched with saturated NH₄Cl solution and the mixture was acidized with HCl(2M) to pH=4. The mixture was extracted with EtOAc. The combined organic layer was dried over Na₂SO₄, filtered and concentrated to afford 103-2 which was used for the next step directly without further purification.

Step 2: To a solution of 103-2 (23 g, crude) in DCE (250 mL) was added tetrabutylammonium borohydride (31.8 g, 125.61 mmol) at room temperature. Then the mixture was stirred at 60° C. for 1 h. The reaction was quenched with H₂O and extracted with DCM. The combined organic layers were washed with brine, dried over Na₂SO₄, filtered and the filtrate was concentrated in vacuo. The residue was purified by column chromatography on silica gel (petroleum ether/ethyl acetate=1/0 to 0/1) to afford 103-3.

Step 3: To a solution of 103-3 (2.2 g, 6.25 mmol) in THF (10 mL) was added TFA (20 mL) and water (10 mL), the mixture was stirred at room temperature for 16 h. The mixture was poured into NaHCO₃ solution and extracted with EtOAc. The combined organic layer was dried over Na₂SO₄, filtered and concentrated. The residue was purified by column chromatography on silica gel (DCM/MeOH=1/0 to 10/1) to afford 103-4.

Step 4: To a solution of 103-4 (1.7 g, 5.52 mmol) in THF (50 mL) was added methylmagnesium bromide (5.52 mL, 16.55 mmol) at −78° C., the reaction mixture was stirred at room temperature for 2 h. The reaction was quenched with saturated NH₄Cl solution and extracted with EtOAc, the combined organic layers were washed with brine, dried over Na₂SO₄, filtered and the filtrate was concentrated in vacuo. The residue was purified by column chromatography on silica gel (DCM/MeOH=1/0 to 10/1) to afford 103-5.

Step 5: To a solution of 103-5 (750 mg, 2.31 mmol) in DCM (20 mL) was added DAST (1.12 g, 6.94 mmol) at −78° C., the reaction mixture was stirred at −78° C. for 1 h. The reaction was quenched with H₂O and extracted with EtOAc, the combined organic layers were washed with brine, dried over Na₂SO₄, filtered and the filtrate was concentrated in vacuo. The residue was purified by column chromatography on silica gel (petroleum ether/ethyl acetate=1/0 to 0/1) to afford 103-6.

Step 6: Compound 103 was prepared from compound 103-6 following the procedure for the synthesis of compound 1 in example 1. LCMS (ESI, m/z): [M+H]⁺=568.2; ¹H NMR (400 MHz, DMSO-d₆, ppm): 8 10.28 (s, 1H), 8.33 (d, J=5.7 Hz, 1H), 8.01 (d, J=8.6 Hz, 1H), 7.66 (d, J=5.7 Hz, 1H), 7.32-7.26 (m, 2H), 4.96 (s, 1H), 4.38 (s, 2H), 3.90-3.89 (m, 4H), 3.77-3.74 (m, 2H), 3.38-3.34 (m, 2H), 2.08-1.69 (m, 10H), 1.58-1.55 (m, 2H), 1.35 (d, J=21.1 Hz, 3H). ¹⁹F NMR (376 MHz, DMSO-d₆, ppm): δ −94.91 (2F), −150.36 (1F).

### Example 43 Synthesis of Compound 114

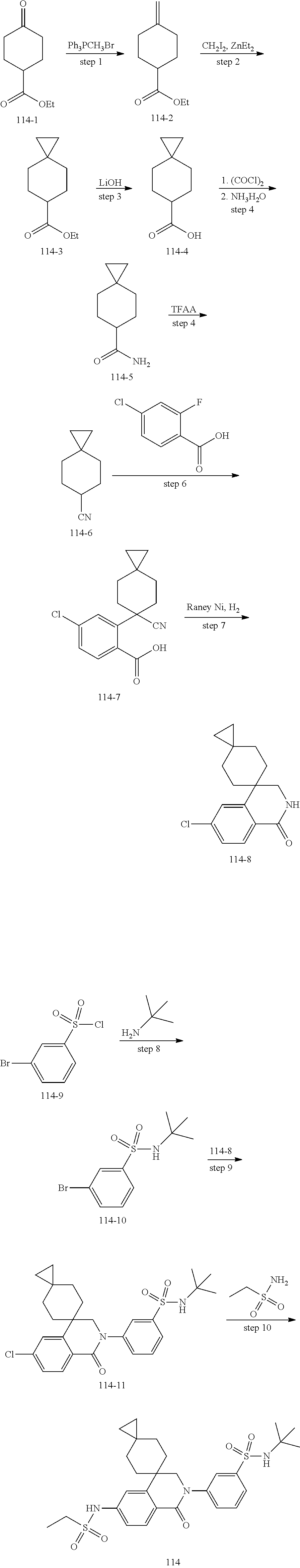

Step 1: To a solution of Methyltriphenylphosphonium bromide (944.4 g, 2643.79 mmol) in THF (7000 mL) was added potassium tert-butoxide (296.7 g, 2643.79 mmol) at −20° C. in portions, then the mixture was stirred for 1 h at 0° C. A solution of 114-1 (300 g, 1762.53 mmol) in THF (500 mL) was added at 0° C., and the mixture was stirred at room temperature for 3 h. Water was added to the mixture with stirring for 10 minutes. The mixture was concentrated and the residue was extracted with tert-Butyl methyl ether/Heptane. The combined organic layer was washed with brine, dried over Na₂SO₄, filtered and concentrated. The residue was precipitated in heptane and filtered with silica gel. The filtrate was concentrated to afford 114-2.

Step 2: To a solution of diethylzinc (1682 mL, 2.0 M, 3364.24 mmol) in DCM (4000 mL) was added dropwise trifluoroacetic acid (383.6 g, 3364.24 mmol) at 0° C., the mixture was stirred for 1 h at 0° C. Then a solution of diiodomethane (901.1 g, 3364.24 mmol) in DCM (600 mL) was added to the mixture at 0° C. The reaction mixture was stirred for 40 minutes. Then a solution of 114-2 (283 g, 1682.12 mmol) in DCM (400 mL) was added and the mixture was stirred for 2 h at 0° C. The mixture was quenched with NH₄Cl solution. Separated and the organic layer was washed with brine, dried over Na₂SO₄, filtered and concentrated to afford 114-3.

Step 3: To a solution of 114-3 (280 g, 1536.27 mmol) in methanol (1400 mL) and water (700 mL) was added lithium hydroxide monohydrate (193.4 g, 4608.80 mmol), and the reaction mixture was stirred for 1 h at 60° C. Then the mixture was concentrated and the residue was washed with tert-Butyl methyl ether. The aqueous layer was adjusted to pH-5 with 2M hydrochloride aqueous and extracted with DCM. The combined organic layer was washed with brine, dried over sodium sulfate, filtered and concentrated to afford 114-4.

Step 4: To a solution of 114-4 (190 g, 1232.09 mmol) in DCM (1500 mL) and DMF (0.5 mL) was added dropwise oxalyl chloride (187.7 g, 1478.50 mmol) at 0° C., and the reaction mixture was stirred for 1 h at room temperature. Then the reaction mixture was concentrated. The residue was dissolved in THF (100 mL) and ammonium hydroxide (920 mL, 25%) was added dropwise at 0° C. The mixture was filtered, the filter cake was dried to afford 114-5.

Step 5: To the mixture of 114-5 (167 g, 1089.94 mmol) and TEA (441.2 g, 4359.74) in THF (1200 mL) was added dropwise a solution of trifluoroacetic anhydride (343.4 g, 1634.90 mmol) in THF (300 mL) at 0° C., and the mixture was stirred for 0.5 h. The reaction mixture was added into 0.5 N hydrochloric acid aqueous (1000 mL) and extracted with tert-Butyl methyl ether. The combined organic layer was washed with 0.5 N hydrochloric acid, aqueous Na₂CO₃ solution and brine, dried over Na₂SO₄, filtered and concentrated. The crude product was purified by distillation under reduced pressure to afford 114-6.

Step 6: To a mixture of 4-chloro-2-fluorobenzoic acid (50 g, 286.43 mmol) and 114-6 (65.84 g, 486.940 mmol) in THF (500 mL) was added dropwise LiHMDS (974 mL, 1.0 M in THF, 974 mmol) at −40° C., and the mixture was stirred at 40° C. for 16 h. The mixture was quenched with water, adjusted to pH˜5 with 2 N hydrochloric acid and extracted with EtOAc. The combined organic layer was washed with brine, dried over Na₂SO₄, filtered and concentrated. The crude product was slurried with acetonitrile and filtered to afford 114-7.

Step 7: To a solution of 114-7 (60 g, 207.07 mmol) in methanol (600 mL) was added ammonium hydroxide (211.2 g, 25%, 3106.02 mmol) and Raney-Ni (120 g), and the mixture was stirred at 30° C. for 24 h under hydrogen atmosphere. Then the mixture was filtered and the filtrate was concentrated. The crude product was slurried with acetonitrile and filtered to afford 114-8.

Step 8: To the solution of 114-9 (1.0 g, 3.91 mmol) in DCM (10 mL) was added dropwise 2-methylpropan-2-amine (1.32 mL, 12.52 mmol) at 0° C., the mixture was stirred at room temperature for 1 h. The reaction mixture was diluted with DCM and washed with water and brine. The collected organic layer was dried over Na₂SO₄, filtered and concentrated to afford 114-10.

Step 9: To a solution of 114-8 (270 mg, 0.98 mmol) in dioxane (4 mL) was added 114-10 (400.5 mg, 1.37 mmol), Cs₂CO₃ (797.5 mg, 2.45 mmol), and XantPhos Pd G₂ (87.0 mg, 0.098 mmol) with N₂ and the reaction mixture was stirred at 85° C. for 2 h. This reaction mixture was filtered and the filtrate was concentrated in vacuo. The residue was purified by column chromatography on silica gel (petroleum ether/ethyl acetate=1/0 to 4/1) to afford 114-11.

Step 10: To a solution of 114-11 (100 mg, 0.21 mmol) in DMSO (2.5 mL) was added ethanesulfonamide (64.2 mg, 0.59 mmol), Copper (I) oxide (14.9 mg, 0.10 mmol), 4-hydroxy-N-(2-methyl-1-naphthyl)pyridine-2-carboxamide (28.6 mg, 0.10 mmol) and potassium tert-butoxide (69.1 mg, 0.62 mmol) under N₂, the reaction mixture was stirred at 130° C. for 16 h. The mixture was filtered and the filtrate was concentrated in vacuo. The residue was purified by reverse HPLC (MeCN in H₂O (0˜100%)) to afford 114. LCMS (ESI, m/z): [M+H]⁺=560.4; ¹H NMR (400 MHz, DMSO-d₆, ppm): δ 10.25 (s, 1H), 7.98-7.87 (m, 2H), 7.71-7.57 (m, 4H), 7.38 (s, 1H), 7.24 (d, J=8.1 Hz, 1H), 3.99 (s, 2H), 3.24-3.19 (m, 2H), 1.80-1.77 (m, 6H), 1.22 (t, J=7.3 Hz, 3H), 1.10 (s, 9H), 0.85 (d, J=10.1 Hz, 2H), 0.26 (s, 4H).

### Example 44 Synthesis of Compound 117

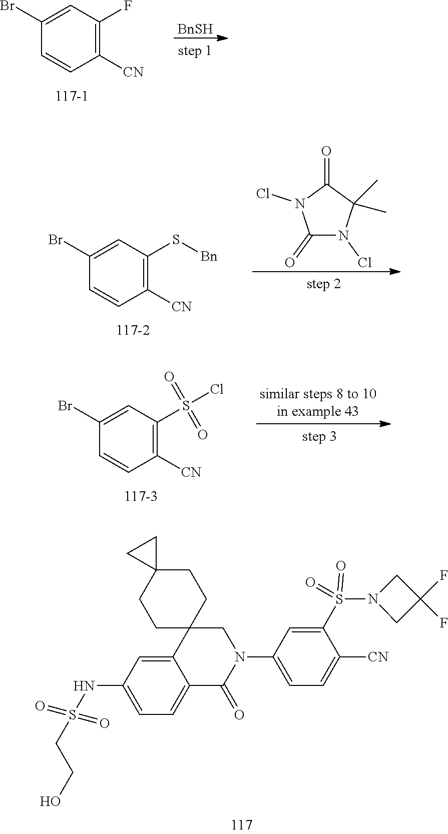

Step 1: To a solution of 117-1 (8 g, 40.0 mmol) in DMF (20 mL) was added phenylmethanethiol (4.97 g, 40.0 mmol) and Cs₂CO₃ (19.5 g, 60.0 mmol), the reaction mixture was stirred at 25° C. for 1.5 h. Water was added to the mixture and stirred for 10 minutes, the mixture was filtered and dried to afford 117-2 which was used for the next step directly without further purification.

Step 2: To a solution of 117-2 (14 g, 36.82 mmol) in MeCN (250 mL) was added AcOH (9.8 mL, 171.19 mmol) and H₂O (5 mL), the mixture was cooled to 0° C., then 1,3-dichloro-5,5-dimethyl-2-oxotetrahydro-1H-imidazol-4-one (14.51 g, 73.64 mmol) was added and the mixture was stirred for 1 h. The mixture was concentrated in vacuo. The residue was diluted with EtOAc and water. The mixture was extracted with EtOAc, the combined organic layers were washed with brine, dried over Na₂SO₄, filtered and the filtrate was concentrated in vacuo. The residue was purified by column chromatography on silica gel (petroleum ether/ethyl acetate=1/0 to 3/1) to afford 117-3.

Step 3: Compound 117 was prepared from compound 117-3 following the procedure for the synthesis of compound 114 in example 43. LCMS (ESI, m/z): [M+H]⁺=621.0; ¹H NMR (400 MHz, DMSO-d₆, ppm): δ 10.28 (s, 1H), 8.29 (d, J=8.5 Hz, 2H), 8.01-7.94 (m, 2H), 7.40 (s, 1H), 7.27-7.24 (m, 1H), 4.99 (s, 1H), 4.50 (t, J=12.5 Hz, 4H), 4.10 (s, 2H), 3.77 (t, J=6.4 Hz, 2H), 3.40-3.36 (m, 2H), 1.84-1.82 (m, 6H), 0.88-0.86 (m, 2H), 0.28 (s, 4H).

### Example 45 Synthesis of Compound 122

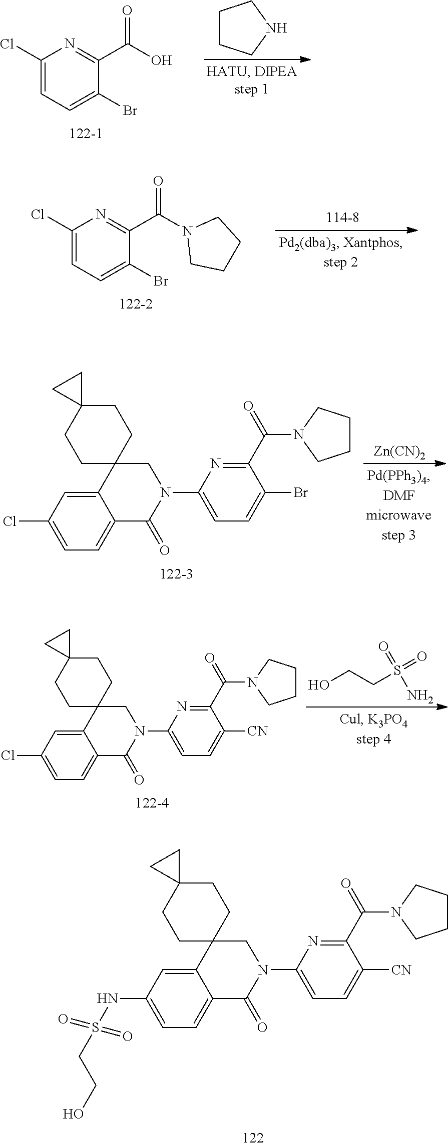

Step 1: A mixture of 122-1 (4 g, 16.92 mmol), tetrahydropyrrole (1.67 mL, 20.30 mmol), HATU (7.7 g, 20.30 mmol) and DIPEA (5.59 mL, 33.83 mmol) in DCM (20 mL) was stirred at room temperature for 2 h. The mixture was concentrated and the residue was purified by column chromatography on silica gel (petroleum ether/ethyl acetate=1/0 to 3/1) to afford 122-2.

Step 2: To a solution of 114-8 (600 mg, 2.17 mmol) in dioxane (5 mL) were added 122-2 (693 mg, 2.39 mmol), Pd₂(dba)₃ (199.2 mg, 0.22 mmol), Cs₂CO₃ (2127.8 mg, 6.53 mmol) and Xantphos (251.8 mg, 0.44 mmol), the reaction mixture was stirred at 90° C. for 2 h under N₂ atmosphere. The mixture was filtered and the filtrate was concentrated in vacuo. The residue was purified by column chromatography on silica gel (petroleum ether/ethyl acetate=1/0 to 3/1) to afford 122-3.

Step 3: A mixture of 122-3 (300 mg, 0.57 mmol), Zn(CN)₂ (66.6 mg, 0.57 mmol) and Pd(PPh₃)₄ (65.5 mg, 0.057 mmol) in DMF (5 mL) was stirred at 100° C. for 3 h under N₂ atmosphere with the microwave condition. The mixture was diluted with water and extracted with EtOAc. The combined organic layers were concentrated and purified by column chromatography on silica gel (petroleum ether/ethyl acetate=1/0 to 3/1) to afford 122-4.

Step 4: To a solution of 122-4 (80 mg, 0.17 mmol) in DMSO (3 mL) was added 2-hydroxyethane-1-sulfonamide (21.08 mg, 0.168 mmol), CuI (32.1 mg, 0.17 mmol), K₃PO₄ (107.2 mg, 0.51 mmol) and methyl[(1R,2R)-2-(methylamino) cyclohexyl]amine (12.0 mg, 0.084 mmol), the mixture was stirred at 130° C. for 2 h under N₂ atmosphere. The mixture was filtered and the filtrate was concentrated, the residue was purified by reverse HPLC (0.05% FA in water/MeCN, 10%-60%) to afford 122 as 0.3FA salt. LCMS (ESI, m/z): [M+H]⁺=564.2; ¹HNMR (400 MHz, DMSO-d₆, ppm): δ 8.40-8.33 (m, 2H), 8.28 (s, 0.3H), 7.98 (d, J=8.5 Hz, 1H), 7.28 (s, 1H), 7.22-7.15 (m, 1H), 4.36 (s, 2H), 3.79-3.71 (m, 2H), 3.64-3.52 (m, 4H), 3.31-3.26 (m, 2H), 1.97-1.63 (m, 10H), 0.88-0.81 (m, 2H), 0.28 (s, 4H).

### Example 46 Synthesis of Compound 125

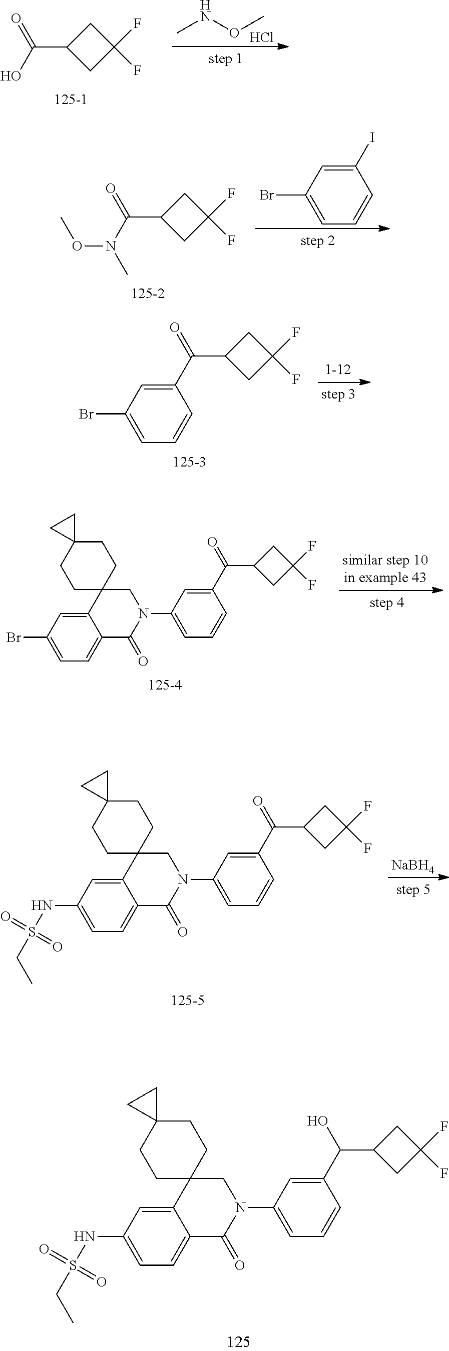

Step 1: A solution of 125-1 (5.0 g, 36.74 mmol), N,O-Dimethylhydroxylamine hydrochloride (4.3 g, 44.09 mmol), TEA (15.32 mL, 110.21 mmol) and HATU (18.16 g, 47.76 mmol) in THF (50 mL) was stirred at 20° C. for 18 h. The mixture was diluted with H₂O and extracted with EtOAc. The combined organic layer was concentrated. The residue was purified by column chromatography on silica gel (DCM/MeOH=1/0 to 10/1) to afford 125-2.

Step 2: To a solution of 1-bromo-3-iodobenzene (500 mg, 1.77 mmol) in THF (20 mL) was added isopropylmagnesium chloride (1.06 mL, 2.12 mmol), the reaction was stirred at −78° C. for 1 h. 125-2 (348.3 mg, 1.94 mmol) was added to the mixture. The mixture was stirred at −78° C. for 2 h. The reaction was quenched with saturated NH₄Cl solution and extracted with EtOAc, the combined organic layers were washed with brine, dried over Na₂SO₄, filtered and the filtrate was concentrated in vacuo. The residue was purified by column chromatography on silica gel (petroleum ether/ethyl acetate=1/0 to 1/1) to afford 125-3.

Step 3: To a solution of 1-12 (180 mg, 0.56 mmol) in dioxane (5 mL) were added 125-3 (231.9 mg, 0.84 mmol), XantPhos Pd G₂ (50.0 mg, 0.056 mmol) and Cs₂CO₃ (549.4 mg, 1.67 mmol), the mixture was stirred at 80° C. for 5 h under N₂. The mixture was concentrated in vacuo. The residue was purified by column chromatography on silica gel (petroleum ether/ethyl acetate=1/0 to 1/1) to afford 125-4.

Step 4: Compound 125-5 was prepared from compound 125-4 following the procedure for the synthesis of compound 114 in example 43.

Step 5: To a solution of 125-5 (60 mg, 0.11 mmol) in THF (2 mL) was added NaBH₄ (8.4 mg, 0.22 mmol), the mixture was stirred at room temperature for 1 h. The crude material was purified by reverse HPLC (H₂O/MeCN=2/1) to afford 125. LCMS (ESI, m/z): [M+H]+=545.1; ¹H NMR (400 MHz, DMSO-d₆, ppm): δ 10.22 (s, 1H), 7.92 (d, J=8.5 Hz, 1H), 7.43-7.33 (m, 3H), 7.20-7.30 (m, 3H), 5.63 (d, J=4.6 Hz, 1H), 4.58 (t, J=4.8 Hz, 1H), 3.91 (s, 2H), 3.24-3.11 (m, 2H), 2.68-2.53 (m, 2H), 2.50-2.30 (m, 3H), 1.90-1.70 (m, 6H), 1.23 (d, J=7.5 Hz, 3H), 0.89 (d, J=8.5 Hz, 2H), 0.28 (s, 4H). ¹⁹F NMR (376 MHz, DMSO-d₆, ppm): δ −80.55 (1F), −94.39 (1F).

### Example 47 Synthesis of Compound 126

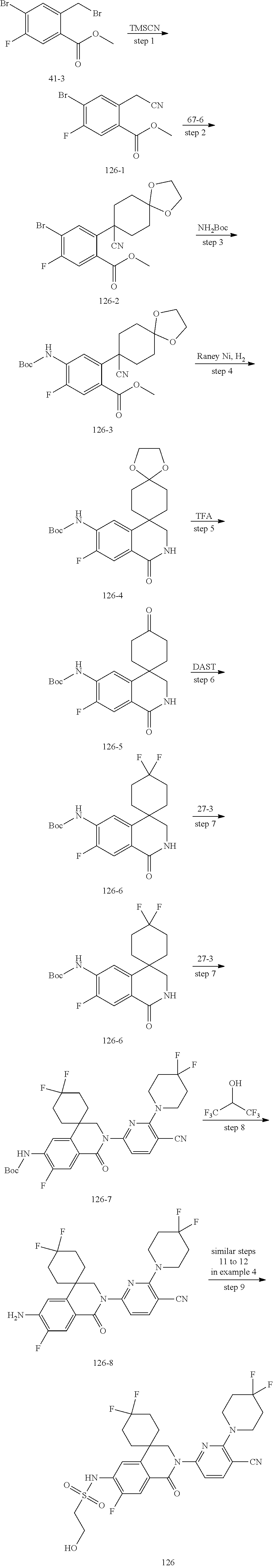

Step 1: To a solution of 41-3 (17 g, 52.15 mmol) and TMSCN (7.18 mL, 57.37 mmol) in THF (50 mL) was added TBAF (57.3 mL, 57.37 mmol) at 0° C. Then the mixture was stirred at 20° C. for 4.5 h. The mixture was diluted with H₂O, extracted with EtOAc. The organic phase was separated, washed with H₂O and concentrated. The residue was purified by column chromatography on silica gel (petroleum ether/ethyl acetate=1/0 to 3/1) to afford 126-1.

Step 2: A solution of methyl 126-1 (5.52 g, 16.23 mmol), 67-6 (6.51 g, 17.04 mmol) and Cs₂CO₃ (18.5 g, 56.81 mmol) in CH₃CN (60 mL) was stirred at 85° C. for 1.5 h. The mixture was concentrated. The residue was purified by column chromatography on silica gel (petroleum ether/ethyl acetate=1/0 to 3/1) to afford 126-2.

Step 3: A solution of 126-2 (2.78 g, 6.98 mmol), tert-butyl carbamate (2.45 g, 20.94 mmol), Cs₂CO₃ (5.7 g, 17.45 mmol) and XantPhos Pd G₂ (0.6 g, 0.69 mmol) in dioxane (30 mL) was stirred at 90° C. for 6 h. The mixture was filtered. The filtrate was concentrated, the residue was purified by column chromatography on silica gel (petroleum ether/ethyl acetate=1/0 to 3/1) to afford 126-3.

Step 4: To a solution of 126-3 (2.37 g, 5.46 mmol) and ammonium hydroxide (4.20 mL, 109.10 mmol) in MeOH (60 mL) was added Raney Ni (1.9 g, 32.73 mmol) at 20° C. The mixture was stirred at 40° C. under H₂ balloon for 20 h. The mixture was filtered, the filtrate was concentrated to afford 126-4.

Step 5: To a solution of 126-4 (500 mg, 1.23 mmol) in THF (4 mL) and H₂O (4 mL) was added TFA (4 mL, 52.24 mmol) at 20° C. and stirred at 20° C. for 2.5 h. The mixture was adjusted pH to 9 by Na₂CO₃ solution, the mixture was extracted with EtOAc. The combined organic phases were concentrated. The residue was purified by column chromatography on silica gel (DCM/MeOH=1/0 to 10/1) to afford 126-5.

Step 6: To a solution of 126-5 (454 mg, 1.13 mmol) in DCM (12 mL) was added DAST (0.89 mL, 6.77 mmol) at 0° C. The mixture was stirred at 0° C. for 3.5 h. The mixture was poured into H₂O, extracted with DCM. The combined organic phases were concentrated. The residue was purified by column chromatography on silica gel (DCM/MeOH=1/0 to 10/1) to afford 126-6.

Step 7: A solution of 126-6 (160 mg, 0.25 mmol), 27-3 (70.79 mg, 0.28 mmol), Xantphos (14.5 mg, 0.025 mmol), Cs₂CO₃ (203.4 mg, 0.62 mmol) and Pd₂(dba)₃ (22.9 mg, 0.025 mmol) in dioxane (6 mL) was stirred at 90° C. for 2 h under N₂. The mixture was concentrated. The residue was purified by column chromatography on silica gel (petroleum ether/ethyl acetate=1/0 to 6/1) to afford 126-7.

Step 8: A solution of 126-7 (120 mg, 0.19 mmol) in 1,1,1,3,3,3-hexafluoro-2-propanol (2 mL) was stirred at 100° C. in microwave for 25 minutes. The mixture was concentrated. The residue was purified by column chromatography on silica gel (petroleum ether/ethyl acetate=1/0 to 2/1) to afford 126-8.

Step 9: Compound 126 was prepared from compound 126-8 following the procedure for the synthesis of compound 6 in example 4. LCMS (ESI, m/z): [M+H]⁺=614.2; ¹H NMR (400 MHz, DMSO-d₆, ppm): δ 10.12 (s, 1H), 8.17-8.09 (m, 1H), 7.85-7.75 (m, 2H), 7.61-7.55 (m, 1H), 5.02 (s, 1H), 4.40 (s, 2H), 3.83-3.75 (m, 6H), 3.43-3.36 (m, 2H), 2.19-2.04 (m, 8H), 1.98-1.76 (m, 4H). ¹⁹F NMR (376 MHz, DMSO-d₆, ppm): δ−89.80 (1F), −94.97 (2F), −98.74 (1F), −125.87 (1F).

### Example 48 Synthesis of Compound 127

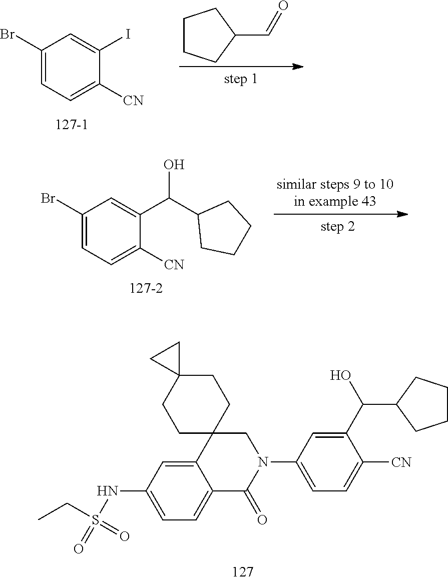 [SMILES: N#Cc1ccc(Br)cc1I ; O=CC1CCCC1 ; N#Cc1ccc(Br)cc1C(O)C1CCCC1 ; CCS(=O)(=O)Nc1ccc2c(c1)C1(CCC3(CC3)CC1)CN(c1ccc(C#N)c(C(O)C3CCCC3)c1)C2=O]

Step 1: A solution of 127-1 (1.0 g, 3.25 mmol) in THF (15 mL) was cooled to −5° C., then isopropyl magnesium chloride (1.95 mL, 3.90 mmol) was added to the solution. After this mixture was stirred at 0° C. for 0.5 h, cyclopentane carbaldehyde (0.42 mL, 3.90 mmol) was added. Then the mixture was stirred below 10° C. for 2 h. Water was added to quench the reaction and the mixture was extracted with EtOAc. The combined organic layer was dried and concentrated, the residue was purified by column chromatography on silica gel (petroleum ether/ethyl acetate=1/0 to 1/1) to afford 127-2.

Step 2: Compound 127 was prepared from compound 127-2 following the procedure for the synthesis of compound 114 in example 43. LCMS (ESI, m/z): [M+H]⁺=548.1; ¹H NMR (400 MHz, DMSO-d₆, ppm): δ 10.31 (s, 1H), 8.22 (d, J=8.4 Hz, 1H), 7.95 (d, J=8.5 Hz, 1H), 7.77-7.67 (m, 2H), 7.41 (d, J=1.8 Hz, 1H), 7.26 (d, J=8.5 Hz, 1H), 5.90 (d, J=5.6 Hz, 1H), 4.03 (s, 2H), 3.26-3.18 (m, 3H), 2.49-2.41 (m, 1H), 1.91-1.76 (m, 7H), 1.69-1.44 (m, 6H), 1.21 (t, J=7.3 Hz, 4H), 0.96-0.77 (m, 2H), 0.27 (s, 4H).

### Example 49 Synthesis of Compound 132

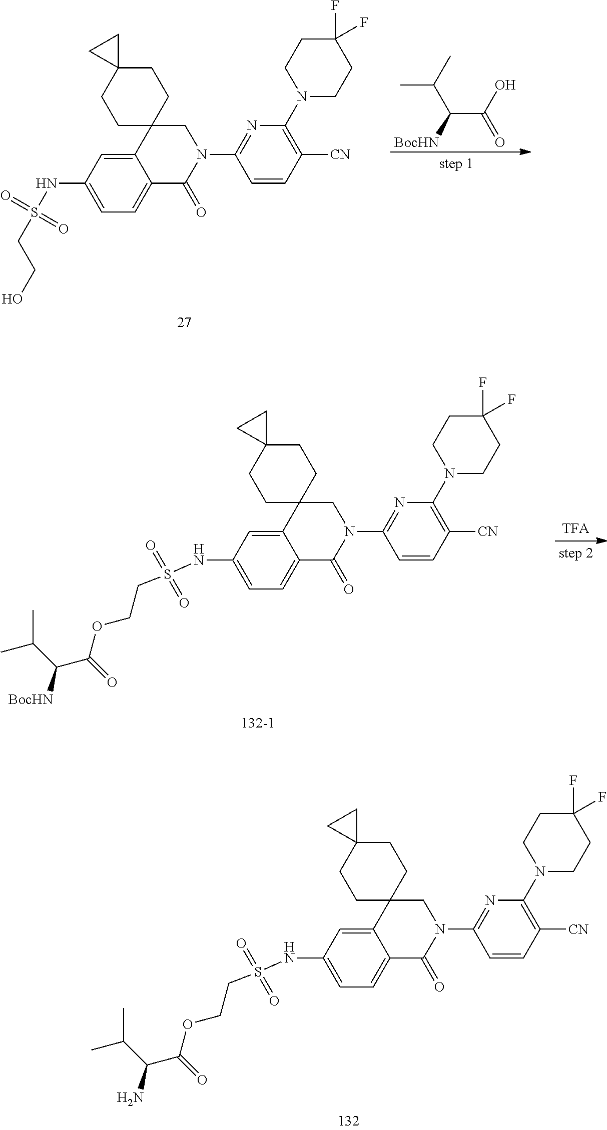 [SMILES: N#Cc1ccc(N2CC3(CCC4(CC4)CC3)c3cc(NS(=O)(=O)CCO)ccc3C2=O)nc1N1CCC(F)(F)CC1 ; CC(C)[C@H](NC(=O)OC(C)(C)C)C(=O)O ; CC(C)[C@H](NC(=O)OC(C)(C)C)C(=O)OCCS(=O)(=O)Nc1ccc2c(c1)C1(CCC3(CC3)CC1)CN(c1ccc(C#N)c(N3CCC(F)(F)CC3)n1)C2=O ; CC(C)[C@H](N)C(=O)OCCS(=O)(=O)Nc1ccc2c(c1)C1(CCC3(CC3)CC1)CN(c1ccc(C#N)c(N3CCC(F)(F)CC3)n1)C2=O]

Step 1: To a solution of 27 (160 mg, 0.27 mmol) in DMF (3 mL) was added N-{[(2-methylprop-2-yl)oxy]carbonyl}-L-valine (89.0 mg, 0.410 mmol), DIPEA (0.14 mL, 0.82 mmol) and HATU (155.8 mg, 0.41 mmol), the mixture was stirred at 25° C. for 20 h. Water was added to the mixture and the mixture was extracted with EtOAc. The combined organic layer was washed with brine and dried over Na₂SO₄, filtered and concentrated. The residue was purified by column chromatography on silica gel (DCM/MeOH=1/0 to 10/1) to afford 132-1.

Step 2: A solution of 132-1 (127 mg, 0.16 mmol) in DCM (0.5 mL) was added TFA (0.2 mL, 0.019 mmol) and the reaction was stirred at 25° C. for 1 h. The reaction was concentrated in vacuo. The residue was diluted with EtOAc and adjusted the pH to 9 with NaHCO₃ solution. the mixture was extracted with EtOAc. The combined organic layer was washed with brine and dried over Na₂SO₄, filtered and concentrated. The residue was purified by column chromatography on silica gel (DCM/MeOH=1/0 to 10/1) and reverse HPLC (MeCN/water: 5%-80%) to afford 132. LCMS (EST, m/z): [M+H]⁺=685.4; ¹H NMR (400 MHz, DMSO-d₆, ppm): 8 8.08 (d, J=8.6 Hz, 1H), 7.98 (d, J=8.6 Hz, 1H), 7.85 (d, J=8.6 Hz, 1H), 7.29 (s, 1H), 7.24-7.19 (m, 1H), 4.44-4.34 (m, 4H), 3.87-3.80 (m, 4H), 3.60-3.57 (m, 2H), 3.05 (d, J=5.2 Hz, 1H), 2.21-2.09 (m, 4H), 1.98-1.88 (m, 2H), 1.85-1.72 (m, 3H), 1.69-1.62 (m, 2H), 0.86 (d, J=13.5 Hz, 2H), 0.80 (d, J=6.8 Hz, 3H), 0.75 (d, J=6.8 Hz, 3H), 0.35-0.23 (m, 4H). ¹⁹F NMR (376 MHz, DMSO-d₆, ppm): δ −94.68 (2F).

### Example 50 Synthesis of Compound 133

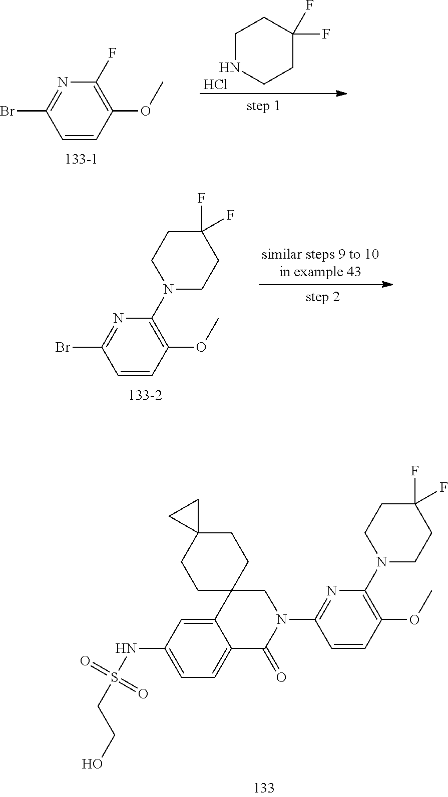

Step 1: To a solution of 133-1 (950 mg, 4.61 mmol) and 4,4-difluoropiperidine hydrochloride (799.4 mg, 5.07 mmol) in DMSO (10 mL) was added DIPEA (2.29 mL, 13.83 mmol) at room temperature. The mixture was stirred at 100° C. for 1 h. The mixture was concentrated in vacuo. The residue was purified by column chromatography on silica gel (petroleum ether/ethyl acetate=1/0 to 1/1) to afford 133-2.

Step 2: Compound 133 was prepared from compound 133-2 following the procedure for the synthesis of compound 114 in example 43. LCMS (ESI, m/z): [M+H]⁺=591.0; ¹H NMR (400 MHz, DMSO-d₆, ppm): δ 10.22 (s, 1H), 7.95 (d, J=8.4 Hz, 1H), 7.59 (d, J=8.5 Hz, 1H), 7.38 (d, J=8.6 Hz, 1H), 7.33 (s, 1H), 7.22 (d, J=8.5 Hz, 1H), 4.99 (s, 1H), 4.24 (s, 2H), 3.84 (s, 3H), 3.76 (t, J=6.5 Hz, 2H), 3.58-3.53 (m, 4H), 3.33-3.31 (m, 2H), 2.10-1.96 (m, 6H), 1.83-1.68 (m, 4H), 0.86 (d, J=13.3 Hz, 2H), 0.29 (s, 4H). ¹⁹F NMR (376 MHz, DMSO-d₆, ppm): δ −94.51 (2F).

### Example 51 Synthesis of Compound 136

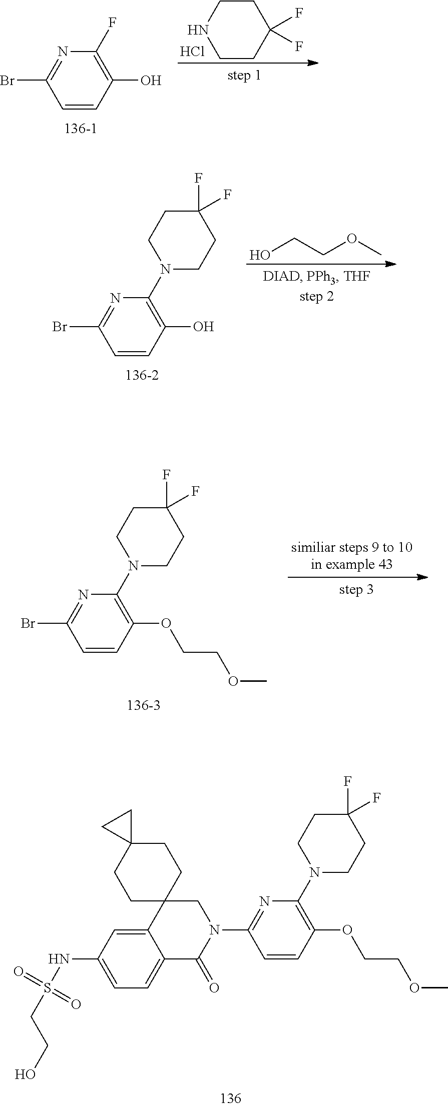

Step 1: To a solution of 136-1 (10 g, 52.09 mmol) and 4,4-Difluoropiperidine hydrochloride (16.42 g, 104.17 mmol) in DMSO (100 mL) was added DIPEA (43.04 mL, 260.43 mmol) at room temperature. The mixture was stirred at 100° C. for 12 h. Water was added to the mixture and the mixture was extracted with EtOAc. The combined organic layer was washed with brine, dried over Na₂SO₄, filtered and concentrated, the residue was purified by column chromatography on silica gel (petroleum ether/ethyl acetate=1/0 to 1/1) to afford 136-2.

Step 2: To a solution of 136-2 (3 g, 10.24 mmol) in THF (60 mL) was added 2-methoxyethan-1-ol (1.21 mL, 15.35 mmol) and PPh₃ (5.4 g, 20.470 mmol), the mixture was stirred at 0° C. for 30 minutes. DIAD (4.1 g, 20.47 mmol) was added, the mixture was stirred at room temperature for 0.5 h. The mixture was concentrated in vacuo. The residue was purified by column chromatography on silica gel (petroleum ether/ethyl acetate=1/0 to 1/1) to afford 136-3.

Step 3: Compound 136 was prepared from compound 136-3 following the procedure for the synthesis of compound 114 in example 43. LCMS (ESI, m/z): [M+H]⁺=635.4; ¹H NMR (400 MHz, DMSO-d₆, ppm): δ 10.17 (s, 1H), 7.96 (d, J=8.5 Hz, 1H), 7.56 (d, J=8.5 Hz, 1H), 7.42-7.32 (m, 2H), 7.23 (d, J=8.6 Hz, 1H), 4.98-4.95 (m, 1H), 4.25 (s, 2H), 4.18-4.13 (m, 2H), 3.77 (t, J=6.4 Hz, 2H), 3.72-3.67 (m, 2H), 3.59 (s, 4H), 3.38-3.31 (m, 5H), 2.14-1.94 (m, 6H), 1.87-1.57 (m, 4H), 0.86 (d, J=13.0 Hz, 2H), 0.30 (s, 4H).

### Example 52 Synthesis of Compound 137

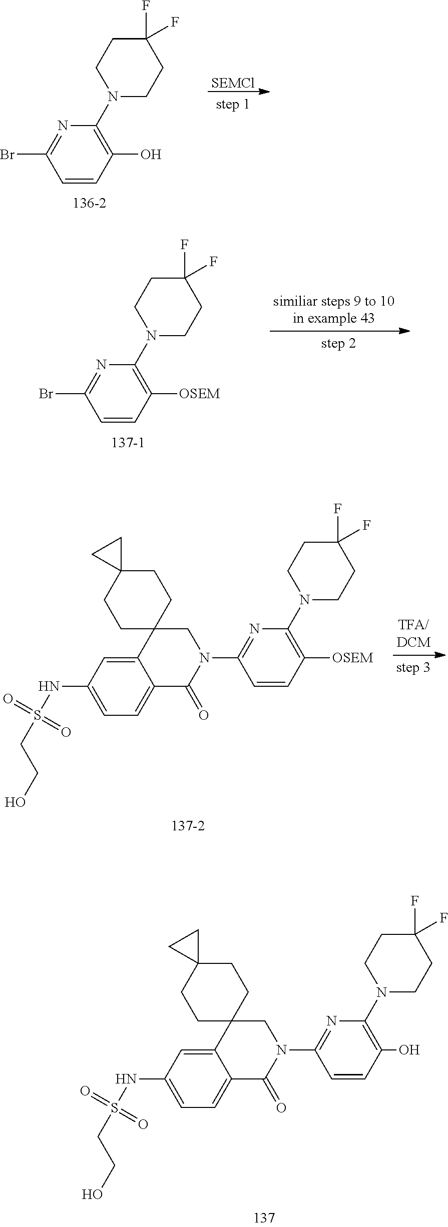

Step 1: To a solution of 136-2 (430 mg, 1.47 mmol) and DIPEA (0.73 mL, 4.40 mmol) in DCM (6.0 mL) was added 2-(Trimethylsilyl)ethoxymethyl chloride (0.52 mL, 2.93 mmol) at 0° C., the mixture was stirred at room temperature for 1 h. The reaction was quenched with NH₄Cl solution and extracted with DCM. The combined organic layer was washed with brine and dried over Na₂SO₄, filtered and concentrated. The residue was purified by column chromatography on silica gel (petroleum ether/ethyl acetate=1/0 to 1/1) to afford 137-1.

Step 2: Compound 137-2 was prepared from compound 137-1 following the procedure for the synthesis of compound 114 in example 43.

Step 3: The solution of 137-2 (60 mg, 0.085 mmol) and TFA (1.0 mL, 13.06 mmol) in DCM (5 mL) was stirred at room temperature for 10 minutes. The mixture was adjusted the pH>7 with NaHCO₃ solution and extracted with DCM. The combined organic phase was dried and concentrated, the residue was purified by reverse HPLC (MeCN/H₂O (5-95%)) to afford 137. LCMS (ESI, m/z): [M+H]⁺=577.4; ¹H NMR (400 MHz, DMSO-d₆, ppm): δ 10.22 (s, 1H), 9.80 (s, 1H), 7.93 (d, J=8.5 Hz, 1H), 7.43 (d, J=8.3 Hz, 1H), 7.33 (s, 1H), 7.23-7.20 (m, 1H), 7.14 (d, J=8.3 Hz, 1H), 4.99 (s, 1H), 4.21 (s, 2H), 3.77-3.74 (m, 2H), 3.58-3.51 (m, 4H), 3.35-3.31 (m, 2H), 2.10-1.97 (m, 6H), 1.80-1.68 (m, 4H), 0.87-0.84 (m, 2H), 0.29 (s, 4H). ¹⁹F NMR (376 MHz, DMSO-d₆, ppm): δ −94.42 (2F).

### Example 53 Synthesis of Compound 138

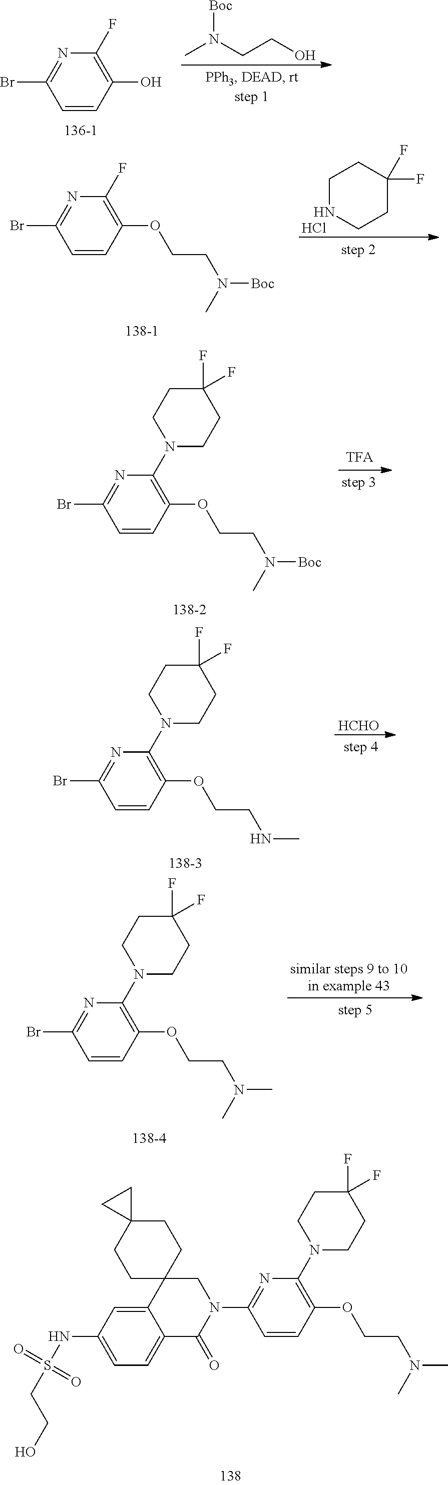

Step 1: The mixture of 136-1 (1.0 g, 5.21 mmol), tert-butyl (2-hydroxyethyl)(methyl)carbamate (2.28 g, 13.02 mmol) and PPh₃ (3.4 g, 13.02 mmol) in THF (15 mL) was stirred at room temperature for 5 minutes under N₂. Diethyl azodicarboxylate (2.05 mL, 13.02 mmol) was added slowly at 0° C., and then the mixture was stirred at room temperature for 1 h. Water was added to the mixture and the mixture was extracted with EtOAc, the combined organic layer was washed with brine and dried over Na₂SO₄, filtered and concentrated. The residue was purified by column chromatography on silica gel (petroleum ether/ethyl acetate=1/0 to 4/1) to afford 138-1.

Step 2: The solution of 138-1 (1.0 g, 2.86 mmol), 4,4-Difluoropiperidine hydrochloride (676.9 mg, 4.30 mmol) and DIPEA (2.37 mL, 14.32 mmol) in DMSO (10 mL) was stirred at 100° C. for 12 h. Water was added to the mixture and the mixture was extracted with EtOAc, the combined organic layer was washed with brine and dried over Na₂SO₄, filtered and concentrated. The residue was purified by column chromatography on silica gel (petroleum ether/ethyl acetate=1/0 to 4/1) to afford 138-2.

Step 3: The solution of 138-2 (410 mg, 1.17 mmol) and TFA (3 mL, 0.22 mmol) in DCM (9 mL) was stirred at room temperature for 0.5 h. NaHCO₃ solution was added to the mixture to adjust the pH>7, the mixture was extracted with DCM. The combined organic layer was dried over Na₂SO₄, filtered and concentrated to afford 138-3 which was used for the next step directly without further purification.

Step 4: To a mixture of 138-3 (310 mg, 0.885 mmol) and paraformaldehyde (53.2 mg, 1.77 mmol) in DCM (6.0 mL) was added DIPEA (0.29 mL, 1.77 mmol) and AcOH (0.005 mL, 0.089 mmol). After this mixture was stirred at room temperature for 1 h, sodium triacetoxyborohydride (562.8 mg, 2.66 mmol) was added. Then the mixture was stirred at room temperature 2 h. The reaction mixture was diluted with DCM and water. The mixture was extracted with DCM. The combined organic phase was dried by Na₂SO₄, filtered and concentrated in vacuo. The residue was purified by column chromatography on silica gel (petroleum ether/ethyl acetate=1/0 to 4/1) to afford 138-4.

Step 5: Compound 138 was prepared from compound 138-4 following the procedure for the synthesis of compound 114 in example 43. LCMS (ESI, m/z): [M+H]⁺=648.2; ¹H NMR (400 MHz, DMSO-d6, ppm): δ 7.94 (d, J=8.5 Hz, 1H), 7.56 (d, J=8.5 Hz, 1H), 7.39 (d, J=8.6 Hz, 1H), 7.32 (s, 1H), 7.21 (d, J=8.5 Hz, 1H), 4.23 (s, 2H), 4.10 (t, J=5.6 Hz, 2H), 3.76 (t, J=6.5 Hz, 2H), 3.60-3.57 (m, 4H), 3.31 (s, 2H), 2.66 (t, J=5.6 Hz, 2H), 2.24 (s, 6H), 2.10-1.96 (m, 6H), 1.82-1.68 (m, 4H), 0.86 (d, J=13.4 Hz, 2H), 0.29 (s, 4H).

### Example 54 Synthesis of Compound 139

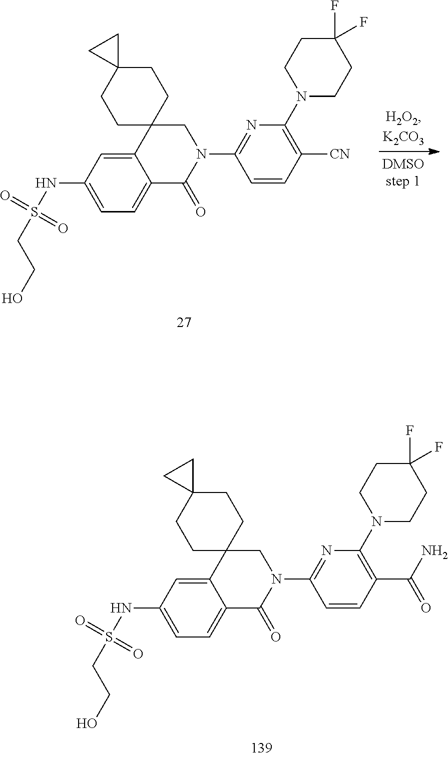 [SMILES: N#Cc1ccc(N2CC3(CCC4(CC4)CC3)c3cc(NS(=O)(=O)CCO)ccc3C2=O)nc1N1CCC(F)(F)CC1 ; NC(=O)c1ccc(N2CC3(CCC4(CC4)CC3)c3cc(NS(=O)(=O)CCO)ccc3C2=O)nc1N1CCC(F)(F)CC1]

Step 1: To a solution of 27 (120 mg, 0.21 mmol) in DMSO (6 mL) was added K₂CO₃ (141.7 mg, 1.03 mmol) and H₂O₂ (348.6 mg, 3.08 mmol) at 0° C. Then the mixture was stirred at room temperature for 12 h. H₂O was added to the mixture. This mixture was stirred at room temperature for 1 h. The mixture was filtered and the solid was collected and dried to afford 139. LCMS (ESI, m/z): [M+H]⁺=604.2; ¹H NMR (400 MHz, DMSO-d₆, ppm): δ 7.90-7.73 (m, 4H), 7.49 (s, 1H), 7.01 (s, 1H), 6.93 (d, J=8.6 Hz, 1H), 4.35 (s, 2H), 3.74-3.69 (m, 2H), 3.47-3.41 (m, 4H), 3.10-3.03 (m, 2H), 2.19-2.06 (m, 4H), 2.02-1.91 (m, 2H), 1.84-1.74 (m, 2H), 1.63 (d, J=12.8 Hz, 2H), 0.83 (d, J=13.4 Hz, 2H), 0.28 (s, 4H).

### Example 55 Synthesis of Compound 140

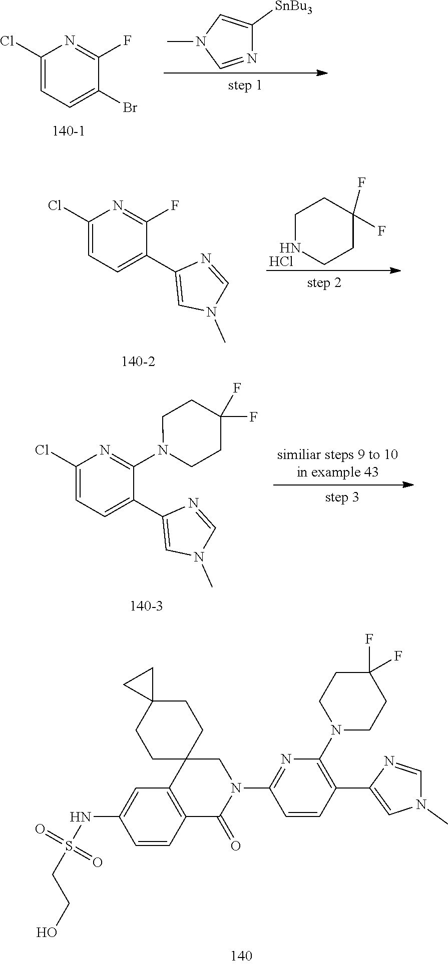

Step 1: A mixture of 140-1 (500 mg, 2.38 mmol), 1-methyl-4-(tributyl-λ4-stannanyl)imidazole (1.06 g, 2.85 mmol) and Pd(PPh₃)₄ (274.6 mg, 0.238 mmol) in DMF (10 mL) was stirred at 100° C. for 16 h under N₂. The mixture was quenched with saturated KF solution and extracted with EtOAc. The combined organic layer was dried and concentrated. The residue was purified by column chromatography on silica gel (petroleum ether/ethyl acetate=1/0 to 0/1) to afford 140-2.

Step 2: To a solution of 140-2 (150 mg, 0.709 mmol) and 4,4-Difluoropiperidine hydrochloride (167.6 mg, 1.06 mmol) in DMSO (0.5 mL) was added DIPEA (458.1 mg, 3.54 mmol) at room temperature. The mixture was stirred at 120° C. for 16 h The mixture was diluted with EtOAc. Washed with saturated NH₄Cl solution. The organic layer was dried and concentrated. The residue was purified by column chromatography on silica gel (petroleum ether/ethyl acetate=1/0 to 0/1) to afford 140-3.

Step 3: Compound 140 was prepared from compound 140-3 following the procedure for the synthesis of compound 114 in example 43. LCMS (ESI, m/z): [M+H]⁺=641.4; ¹H NMR (400 MHz, DMSO-d₆, ppm): δ 10.21 (s, 1H), 8.24 (d, J=8.5 Hz, 1H), 8.00-7.91 (m, 2H), 7.73-7.70 (m, 2H), 7.35 (s, 1H), 7.26-7.23 (m, 1H), 4.98-4.95 (m, 1H), 4.41 (s, 2H), 3.79-3.73 (m, 5H), 3.37-3.25 (m, 6H), 2.18-2.15 (m, 4H), 2.07-2.00 (m, 2H), 1.82-1.68 (m, 4H), 0.86 (d, J=13.4 Hz, 2H), 0.29 (s, 4H).

### Example 56 Synthesis of Compound 141

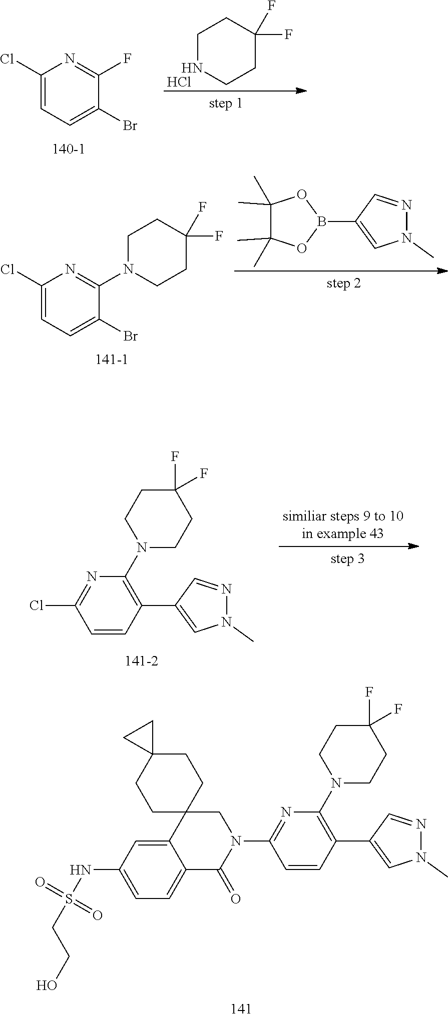

Step 1: To a solution of 140-1 (3 g, 14.26 mmol) in DMSO (50 mL) was 4,4-Difluoropiperidine hydrochloride (3.37 g, 21.39 mmol) and DIPEA (9.46 mL, 57.03 mmol), the mixture was stirred at 120° C. for 3 h. The reaction was diluted with EtOAc and water. The organic layer was separated, washed with brine and concentrated in vacuo. The residue was purified by column chromatography on silica gel (petroleum ether/ethyl acetate=1/0 to 10/1) to afford 141-1.

Step 2: A mixture of 141-1 (500 mg, 1.61 mmol), 1-methyl-4-(4,4,5,5-tetramethyl-1,3,2-dioxaborolan-2-yl)pyrazole (400.7 mg, 1.93 mmol), Pd(dppf)Cl₂·CH₂Cl₂ (117.4 mg, 0.14 mmol) and K₂CO₃ (665.4 mg, 4.82 mmol) in dioxane (8 mL) and H₂O (0.8 mL) was heated at 90° C. for 2 h. The mixture was filtered and the filtrate was concentrated in vacuo. The residue was purified by column chromatography on silica gel (petroleum ether/ethyl acetate=1/0 to 2/1) to afford 141-2.

Step 3: Compound 141 was prepared from compound 141-2 following the procedure for the synthesis of compound 114 in example 43. LCMS (ESI, m/z): [M+H]⁺=641.5; ¹H NMR (400 MHz, DMSO-d₆, ppm): δ 8.20 (s, 1H), 7.99-7.92 (m, 2H), 7.88 (d, J=8.3 Hz, 1H), 7.82 (d, J=8.3 Hz, 1H), 7.33-7.29 (m, 1H), 7.24-7.19 (m, 1H), 4.39 (s, 2H), 3.90 (s, 3H), 3.76 (t, J=6.5 Hz, 2H), 3.33-3.30 (m, 2H), 3.25-3.19 (m, 4H), 2.19-1.97 (m, 6H), 1.85-1.74 (m, 2H), 1.73-1.65 (m, 2H), 0.91-0.82 (m, 2H), 0.35-0.23 (m, 4H).

### Example 57 Synthesis of Compound 143

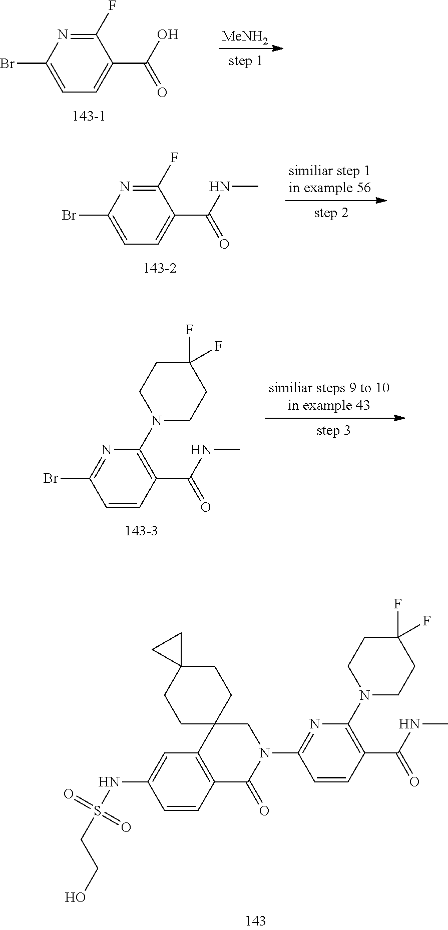 [SMILES: "MeNH2" ; O=C(O)c1ccc(Br)nc1F ; CNC(=O)c1ccc(Br)nc1F ; CNC(=O)c1ccc(Br)nc1N1CCC(F)(F)CC1 ; CNC(=O)c1ccc(N2CC3(CCC4(CC4)CC3)c3cc(NS(=O)(=O)CCO)ccc3C2=O)nc1N1CCC(F)(F)CC1]

Step 1: To a solution of 143-1 (200 mg, 0.91 mmol) in DMF (1.5 mL) were added DIPEA (0.45 mL, 2.73 mmol), HATU (518.5 mg, 1.36 mmol), and methylamine hydrochloride (79.8 mg, 1.18 mmol), the mixture was stirred at 0° C. for 2 h. The reaction diluted with EtOAc and water. The mixture was extracted with EtOAc. The organic layer was washed with brine, dried over Na₂SO₄, filtered and concentrated. The residue was purified by column chromatography on silica gel (petroleum ether/ethyl acetate=1/0 to 3/1) to afford 143-2.

Step 2: Compound 143-3 was prepared from compound 143-2 following the procedure for the synthesis of compound 141-1 in example 56.

Step 3: Compound 143 was prepared from compound 143-3 following the procedure for the synthesis of compound 114 in example 43. LCMS (ESI, m/z): [M+H]⁺=618.4; ¹H NMR (400 MHz, DMSO-d₆, ppm): δ 10.23 (s, 1H), 8.37-8.25 (m, 1H), 7.99 (d, J=8.5 Hz, 1H), 7.84-7.75 (m, 2H), 7.35 (d, J=1.7 Hz, 1H), 7.27-7.20 (m, 1H), 4.97 (s, 1H), 4.40 (s, 2H), 3.76 (t, J=6.5 Hz, 2H), 3.47-3.40 (m, 4H), 3.38-3.36 (m, 2H), 2.83-2.76 (m, 3H), 2.18-2.05 (m, 4H), 2.03-1.92 (m, 2H), 1.83-1.77 (m, 2H), 1.73-1.64 (m, 2H), 0.93-0.80 (m, 2H), 0.30 (s, 4H).

### Example 58 Synthesis of Compound 147

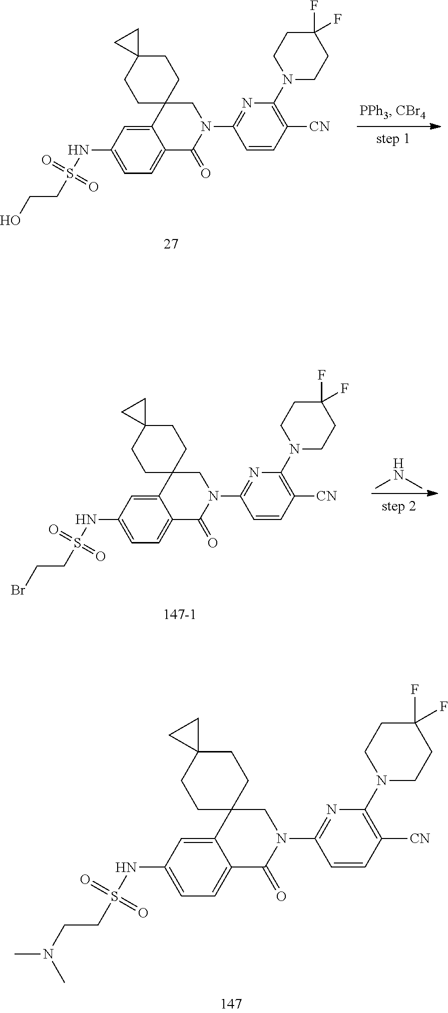

Step 1: To a solution of 27 (30 mg, 0.051 mmol) and tetrabromomethane (33.8 mg, 0.10 mmol) in DCM (3 mL) was added PPh₃ (26.8 mg, 0.10 mmol) at 0° C. The reaction mixture was stirred 0° C. for 2 h. The mixture was quenched with water and extracted with DCM. The combined organic layers were washed with brine, concentrated and dried to afford 147-1 which was used for the next step directly without further purification.

Step 2: A solution of 147-1 (33.1 mg, crude) in MeCN (3 mL) was added dimethylamine (2 M in THF, 0.050 mL, 0.10 mmol) at 0° C., then the mixture was stirred at room temperature for 1 h. The resulting mixture was concentrated and purified by reverse HPLC (0.05% FA in water/MeCN, 10%-50%) to afford 147 as 1.5 FA salts. LCMS (ESI, m/z): [M+H]⁺=613.2; ¹H NMR (400 MHz, DMSO-d₆, ppm): δ 8.35 (s, 1.5H), 8.12-8.00 (m, 1H), 7.96-7.89 (m, 1H), 7.87-7.82 (m, 1H), 7.28-7.18 (m, 1H), 7.17-7.09 (m, 1H), 4.39 (s, 2H), 3.89-3.79 (m, 4H), 3.26-3.20 (m, 2H), 2.66-2.60 (m, 2H), 2.26-2.02 (m, 10H), 1.99-1.87 (m, 2H), 1.85-1.74 (m, 2H), 1.71-1.60 (m, 2H), 0.93-0.79 (m, 2H), 0.39-0.20 (m, 4H).

### Example 59 Synthesis of Compound 149

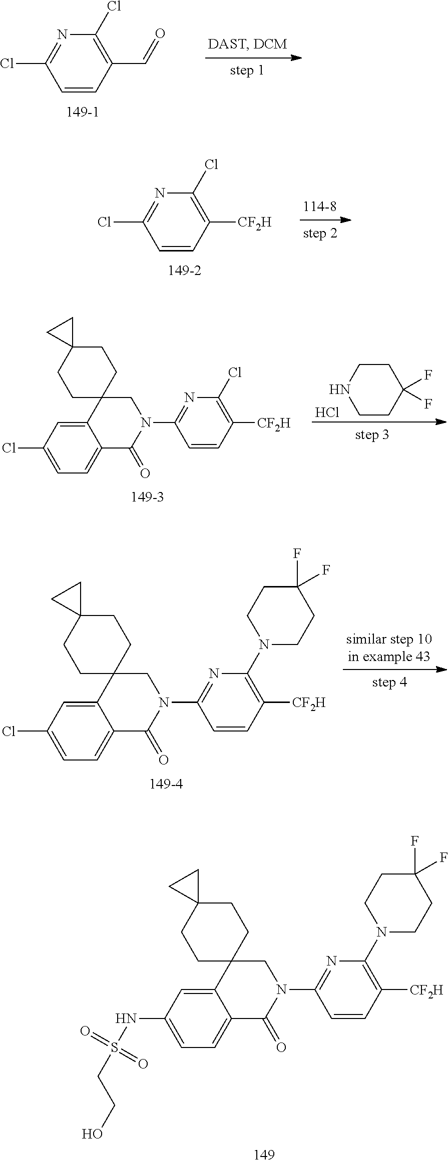

Step 1: To a solution of 149-1 (2 g, 11.36 mmol) in DCM (15 mL) were added DAST (5.5 g, 34.09 mol), the reaction was stirred at 0° C. for 1 h. The mixture was diluted with DCM and saturated NaHCO₃ solution. The organic layer was separated, washed with brine, and concentrated in vacuo. The residue was purified by column chromatography on silica gel (petroleum ether/ethyl acetate=1/0 to 10/1) to afford 149-2.

Step 2: To a solution of 114-8 (100 mg, 0.36 mmol) in dioxane (4 mL) were added 149-2 (71.8 mg, 0.36 mmol), XantPhos Pd G₂ (32.2 mg, 0.036 mmol) and Cs₂CO₃ (354.4 mg, 1.09 mmol), the mixture was stirred at 90° C. for 1 h. The mixture was concentrated in vacuo. The residue was purified by column chromatography on silica gel (petroleum ether/ethyl acetate=1/0 to 1/1) to afford 149-3. 13621 Step 3: To a solution of 149-3 (90 mg, 0.21 mmol) in DMA (2 mL) were added 4,4-difluoropiperidine hydrochloride (648.65 mg, 4.12 mmol) and DIPEA (1.02 mL, 6.17 mmol), the mixture was stirred at 130° C. for 6 h. The mixture was diluted with EtOAc and water. The organic layer was separated, washed with brine and concentrated in vacuo. The residue was purified by column chromatography on silica gel (petroleum ether/ethyl acetate=1/0 to 2/3) to afford 149-4.

Step 4: Compound 149 was prepared from compound 149-4 following the procedure for the synthesis of compound 114 in example 43. LCMS (ESI, m/z): [M+H]⁺=611.2; ¹H NMR (400 MHz, DMSO-d₆, ppm): δ 10.24 (s, 1H), 8.09-7.94 (m, 3H), 7.35 (s, 1H), 7.32-7.22 (m, 1H), 7.18-7.00 (m, 1H), 4.97 (s, 1H), 4.42 (s, 2H), 3.77 (t, J=6.4 Hz, 2H), 3.45-3.33 (m, 6H), 2.26-2.11 (m, 4H), 2.10-1.90 (m, 2H), 1.82-1.77 (m, 2H), 1.70-1.67 (m, 2H), 0.86 (d, J=13.2 Hz, 2H), 0.30 (s, 4H).

### Example 60 Synthesis of Compound 150

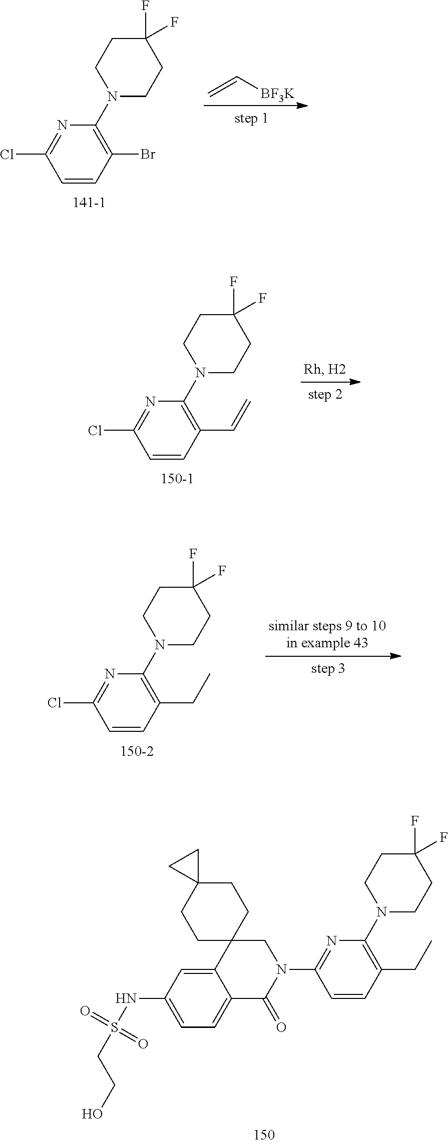

Step 1: To a solution of 141-1 (1.0 g, 3.21 mmol) in dioxane (10 mL) and H₂O (2 mL) was added potassium vinyltrifluoroborate (20.1 mg, 0.18 mmol), Pd(dppf)Cl₂·CH₂Cl₂ (262.1 mg, 0.32 mmol) and Cs₂CO₃ (2.09 g, 6.42 mmol), the mixture was stirred at 100° C. for 2 h under N₂ atmosphere. The mixture was filtered and the filtrate was concentrated in vacuo. The residue was purified by column chromatography on silica gel (petroleum ether/ethyl acetate=1/0 to 3/2) to afford 150-1.

Step 2: A mixture of 150-1 (720 mg, 2.78 mmol) and Rh/C (636.5 mg) in MeOH (50 mL) was stirred at room temperature for 16 h under H₂ atmosphere. The mixture was filtered and the filtrate was concentrated in vacuo to afford 150-2 which was used for the next step directly without further purification.

Step 3: Compound 150 was prepared from compound 150-2 following the procedure for the synthesis of compound 114 in example 43. LCMS (ESI, m/z): [M+H]⁺=589.4; ¹H NMR (400 MHz, DMSO-d₆, ppm): 8 10.19 (s, 1H), 7.97 (d, J=8.5 Hz, 1H), 7.83 (d, J=8.2 Hz, 1H), 7.67 (d, J=8.3 Hz, 1H), 7.34 (s, 1H), 7.27-7.19 (m, 1H), 4.97 (s, 1H), 4.35 (s, 2H), 3.87-3.66 (m, 2H), 3.33 (s, 2H), 3.28-3.19 (m, 4H), 2.66-2.60 (m, 2H), 2.21-2.08 (m, 4H), 2.07-1.96 (m, 2H), 1.85-1.73 (m, 2H), 1.73-1.63 (m, 2H), 1.25-1.18 (m, 3H), 0.92-0.75 (m, 2H), 0.29 (s, 4H).

### Example 61 Synthesis of Compound 152

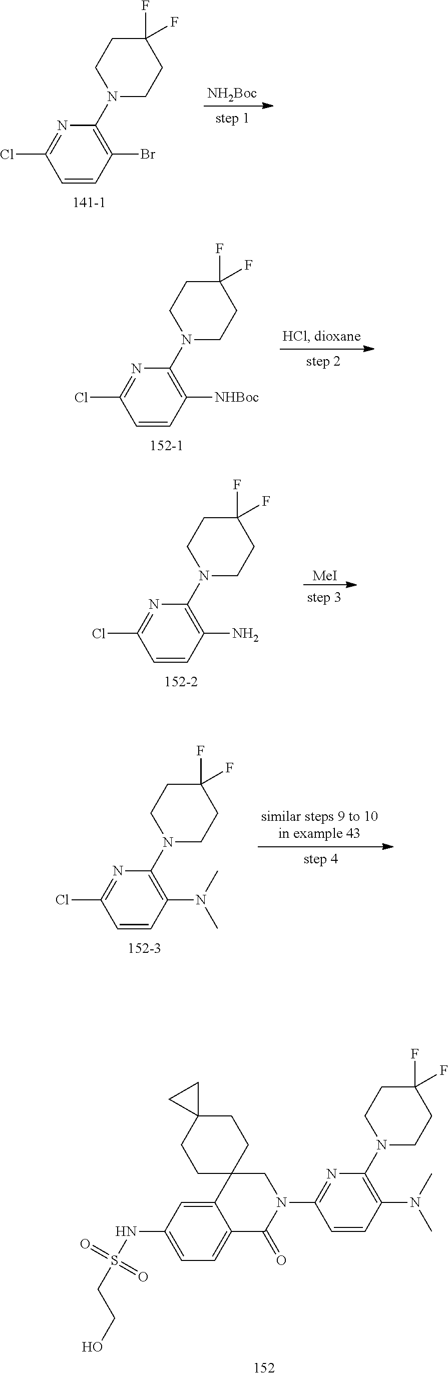

Step 1: To a solution of 141-1 (500 mg, 1.61 mmol) in dioxane (20 mL) was added tert-butyl carbamate (188.0 mg, 1.61 mmol), Cs₂CO₃ (1568.7 mg, 4.82 mmol) and XantPhos Pd G₂ (142.6 mg, 0.16 mmol), the mixture was stirred at 80° C. for 12 h under N₂ atmosphere. The mixture was concentrated in vacuo. The residue was purified by column chromatography on silica gel (petroleum ether/ethyl acetate=1/0 to 4/1) to afford 152-1.

Step 2: A mixture of 152-1 (550 mg, 1.58 mmol) in HCl/dioxane (5 mL)/dioxane (15 mL) was stirred at room temperature for 16 h. The mixture was concentrated in vacuo to afford 152-2 which was use for the next step directly without further purification.

Step 3: To an ice-cold solution of 152-2 (400 mg, crude) in DMSO (10 mL) was added NaH (323.0 mg, 8.08 mmol). The mixture was stirred at room temperature for 1 h. Then CH₃I (0.50 mL, 8.08 mmol) was added to the mixture at 0° C. The mixture was stirred at room temperature for 16 h. The reaction was quenched with saturated NH₄Cl solution and extracted with EtOAc. The combined organic layer was dried and concentrated. The residue was purified by column chromatography on silica gel (petroleum ether/ethyl acetate=1/0 to 10/1) to afford 152-3.

Step 4: Compound 152 was prepared from compound 152-3 following the procedure for the synthesis of compound 114 in example 43. LCMS (ESI, m/z): [M+H]⁺=604.4; ¹H NMR (400 MHz, DMSO-d₆, ppm): δ 9.78 (s, 1H), 7.95 (d, J=8.5 Hz, 1H), 7.62 (d, J=8.4 Hz, 1H), 7.31 (dd, J=7.6, 5.3 Hz, 2H), 7.21 (dd, J=8.5, 1.9 Hz, 1H), 4.28 (s, 2H), 3.76 (t, J=6.5 Hz, 2H), 3.68-3.51 (m, 4H), 3.33-3.30 (m, 3H), 2.73 (s, 6H), 2.19-2.06 (m, 4H), 2.06-1.93 (m, 2H), 1.84-1.73 (m, 2H), 1.73-1.65 (m, 2H), 0.93-0.77 (m, 2H), 0.37-0.20 (m, 4H).

### Example 62 Synthesis of Compound 157

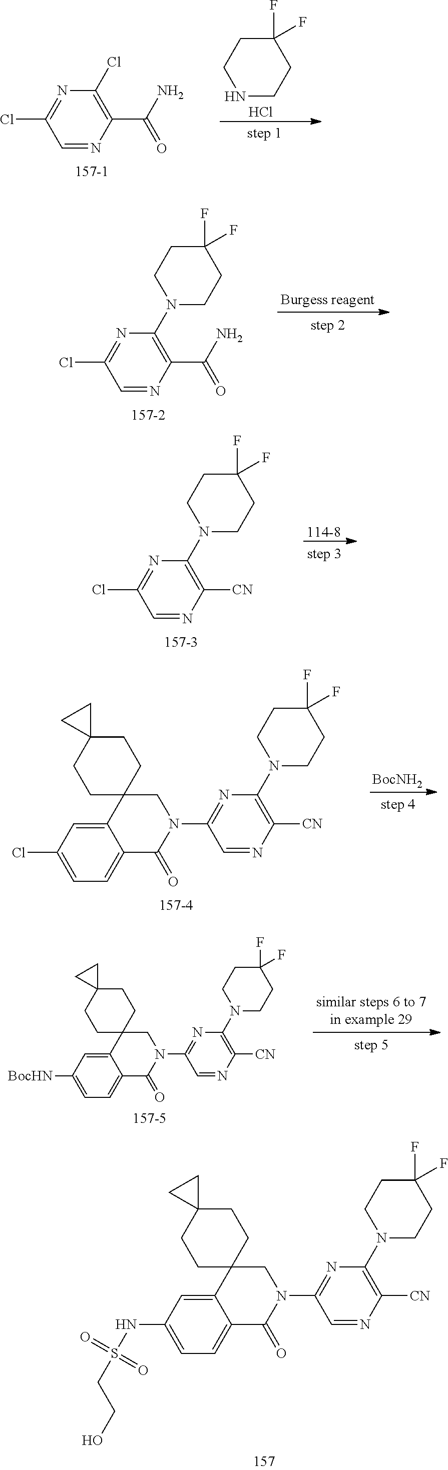

Step 1: The mixture of 157-1 (1.50 g, 7.81 mmol), 4,4-difluoropiperidine hydrochloride (1.29 g, 8.20 mmol) and DIPEA (3.87 mL, 23.44 mmol) in NMP (15 mL) was stirred at 90° C. for 2 h. The mixture was extracted with EtOAc and the combined organic layer was washed with brine. The organic layer was dried over Na₂SO₄, filtered and concentrated. The residue was purified by column chromatography on silica gel (petroleum ether/ethyl acetate=1/0 to 3/1) to afford 157-2.

Step 2: To a solution of 157-2 (800 mg, 2.9 mmol) in DCM (10 mL) was added (Methoxycarbonylsulfamoyl)triethylammonium Hydroxide Inner Salt (2.07 g, 8.68 mmol), the mixture was stirred at room temperature for 1 h. H₂O was added to the mixture and the mixture was extracted with DCM. The combined organic layer was concentrated in vacuo. The residue was purified by column chromatography on silica gel (petroleum ether/ethyl acetate=1/0 to 6/1) to afford 157-3.

Step 3: To a solution of 114-8 (200 mg, 0.73 mmol) in dioxane (2.0 mL) was added 157-3 (197 mg, 0.76 mmol), Xantphos Pd G₂ (64.5 mg, 0.073 mmol) and Cs₂CO₃ (708.9 mg, 2.18 mmol) at room temperature, the mixture was stirred at 100° C. for 3 h. The mixture was filtered and the filtrate was concentrated under vacuo. The residue was purified by column chromatography on silica gel (petroleum ether/ethyl acetate=1/0 to 1/1) to afford 157-4.

Step 4: To a solution of 157-4 (160 mg, 0.32 mmol) in dioxane (2.0 mL) was added tert-butyl carbamate (75.3 mg, 0.64 mmol), Cs₂CO₃ (261.7 mg, 0.80 mmol) and Xantphos Pd G₂ (28.6 mg, 0.032 mmol), the mixture was stirred at 100° C. for 2 h. The mixture was filtered and the filtrate was concentrated in vacuo. The residue was purified by column chromatography on silica gel (petroleum ether/ethyl acetate=1/0 to 4/1) to afford 157-5.

Step 5: Compound 157 was prepared from compound 157-5 following the procedure for the synthesis of compound 59 in example 29. LCMS (ESI, m/z): [M+H]⁺=587.4; ¹H NMR (400 MHz, DMSO-d₆, ppm): δ 9.04 (s, 1H), 8.02 (d, J=8.6 Hz, 1H), 7.36 (s, 1H), 7.26 (d, J=8.5 Hz, 1H), 4.36 (s, 2H), 3.96-3.89 (m, 4H), 3.76 (t, J=6.3 Hz, 2H), 3.39-3.31 (m, 2H), 2.19-2.14 (m, 4H), 1.92 (t, J=12.0 Hz, 2H), 1.80 (t, J=11.8 Hz, 2H), 1.69 (d, J=12.2 Hz, 2H), 0.87 (d, J=13.2 Hz, 2H), 0.30 (s, 4H). ¹⁹F NMR (376 MHz, DMSO-d6, ppm): δ −94.67 (2F).

### Example 63 Synthesis of Compound 158

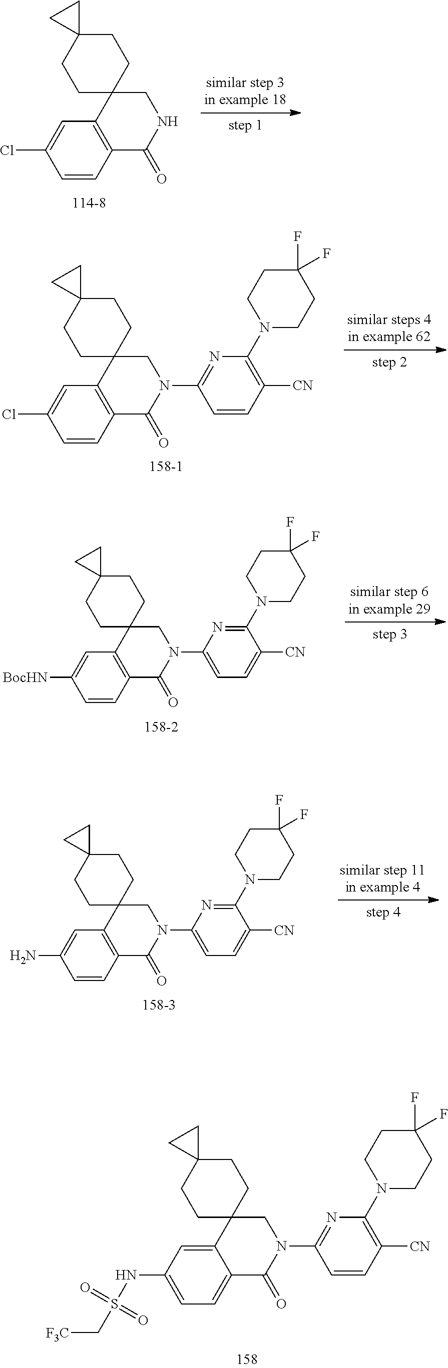 [SMILES: O=C1NCC2(CCC3(CC3)CC2)c2cc(Cl)ccc21 ; N#Cc1ccc(N2CC3(CCC4(CC4)CC3)c3cc(Cl)ccc3C2=O)nc1N1CCC(F)(F)CC1 ; CC(C)(C)OC(=O)Nc1ccc2c(c1)C1(CCC3(CC3)CC1)CN(c1ccc(C#N)c(N3CCC(F)(F)CC3)n1)C2=O ; N#Cc1ccc(N2CC3(CCC4(CC4)CC3)c3cc(N)ccc3C2=O)nc1N1CCC(F)(F)CC1 ; N#Cc1ccc(N2CC3(CCC4(CC4)CC3)c3cc(NS(=O)(=O)CC(F)(F)F)ccc3C2=O)nc1N1CCC(F)(F)CC1]

Step 1: Compound 158-1 was prepared from compound 114-8 following the procedure for the synthesis of compound 27-4 in example 18.

Step 2: Compound 158-2 was prepared from compound 158-1 following the procedure for the synthesis of compound 157-5 in example 62.

Step 3: Compound 158-3 was prepared from compound 158-2 following the procedure for the synthesis of compound 59-7 in example 29.

Step 4: Compound 158 was prepared from compound 158-3 following the procedure for the synthesis of compound 6-15 in example 4. LCMS (ESI, m/z): [M+H]⁺=624.2; ¹H NMR (400 MHz, DMSO-d₆, ppm): δ 8.08 (d, J=8.3 Hz, 1H), 7.99 (d, J=8.2 Hz, 1H), 7.85 (d, J=8.4 Hz, 1H), 7.30 (s, 1H), 7.24 (d, J=8.1 Hz, 1H), 4.66-4.51 (m, 2H), 4.41 (s, 2H), 3.88-3.81 (m, 4H), 2.21-2.08 (m, 4H), 2.00-1.87 (m, 2H), 1.86-1.74 (m, 2H), 1.71-1.60 (m, 2H), 0.86 (d, J=11.7 Hz, 2H), 0.29 (s, 4H). ¹⁹F NMR (376 MHz, DMSO-d6, ppm): δ −60.89 (3F), −94.69 (2F).

### Example 64 Synthesis of Compound 169

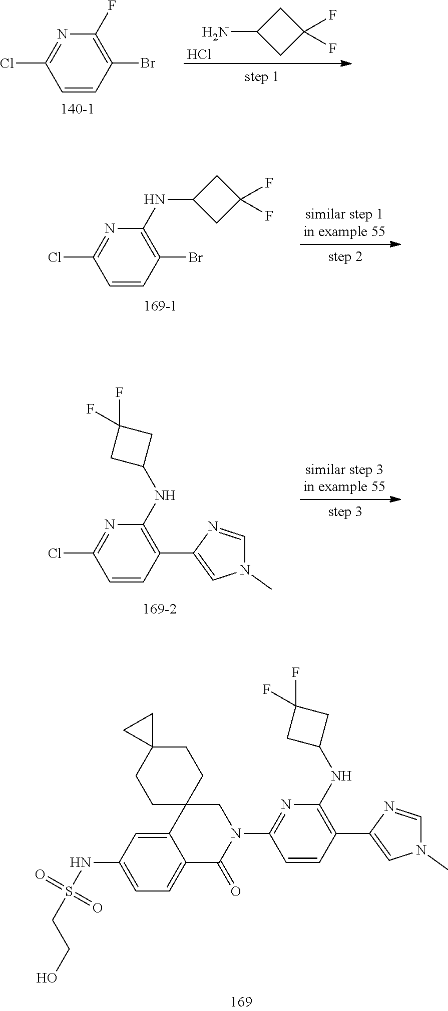

Step 1: To a solution of 140-1 (3 g, 14.257 mmol) in DMSO (30 mL) was added 3,3-difluorocyclobutan-1-amine hydrochloride (3.07 g, 21.39 mmol) and DIPEA (9.46 mL, 57.03 mmol), the reaction was stirred at 120° C. for 3 h. The mixture was diluted with EtOAc and water. The organic layer was separated, washed with brine and concentrated in vacuo. The residue was purified by column chromatography on silica gel (petroleum ether/ethyl acetate=1/0 to 10/1) to afford 169-1.

Step 2: Compound 169-2 was prepared from compound 169-1 following the procedure for the synthesis of compound 140-2 in example 55.

Step 3: Compound 169 was prepared from compound 169-2 following the procedure for the synthesis of compound 140 in example 55. LCMS (ESI, m/z): [M+H]⁺=627.2; ¹H NMR (400 MHz, DMSO-d₆, ppm): δ 10.19 (s, 1H), 9.09 (d, J=5.4 Hz, 1H), 7.98 (d, J=8.5 Hz, 1H), 7.83-7.80 (m, 2H), 7.67 (s, 1H), 7.51 (d, J=8.2 Hz, 1H), 7.35 (d, J=1.8 Hz, 1H), 7.22 (m, 1H), 4.97 (t, J=5.6 Hz, 1H), 4.39 (s, 3H), 3.81-3.70 (m, 5H), 3.39-3.32 (m, 2H), 3.19-3.08 (m, 2H), 2.69-2.55 (m, 2H), 2.09-2.01 (m, 2H), 1.84-1.68 (m, 4H), 0.88 (d, J=13.5 Hz, 2H), 0.31 (d, J=4.6 Hz, 4H). ¹⁹F NMR (376 MHz, DMSO-d6, ppm): δ −81.68 (1F), −95.19 (1F).

### Example 65 Synthesis of Compound 171

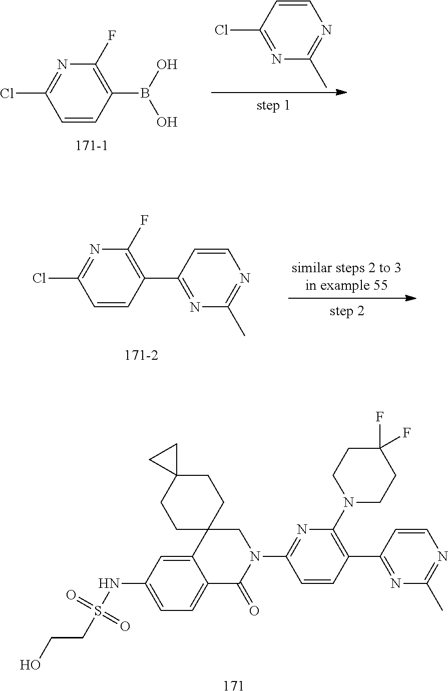

Step 1: A solution of 171-1 (358.0 mg, 2.04 mmol), 4-chloro-2-methylpyrimidine (250 mg, 1.95 mmol), K₂CO₃ (806.2 mg, 5.83 mmol) and Pd(dppf)Cl₂·CH₂Cl₂ (158.8 mg, 0.19 mmol) in dioxane (8 mL) was stirred at 80° C. for 2 h. The mixture was concentrated in vacuo. The residue was purified by column chromatography on silica gel (petroleum ether/ethyl acetate=1/0 to 1/1) to afford 171-2.

Step 2: Compound 171 was prepared from compound 171-2 following the procedure for the synthesis of compound 140 in example 55. LCMS (ESI, m/z): [M+H]⁺=653.2; ¹H NMR (400 MHz, DMSO-d₆, ppm): δ 10.24 (s, 1H), 8.71 (d, J=5.3 Hz, 1H), 8.13 (d, J=8.4 Hz, 1H), 8.04-7.95 (m, 2H), 7.90 (d, J=5.3 Hz, 1H), 7.36 (d, J=1.9 Hz, 1H), 7.29-7.22 (m, 1H), 4.97 (s, 1H), 4.46 (s, 2H), 3.77 (t, J=6.4 Hz, 2H), 3.36-3.25 (m, 6H), 2.68 (s, 3H), 2.15-1.95 (m, 6H), 1.87-1.66 (m, 4H), 0.89 (d, J=13.5 Hz, 2H), 0.33 (s, 4H). ¹⁹F NMR (376 MHz, DMSO-d6, ppm): δ −95.27 (2F).

### Example 66 Synthesis of Compound 173

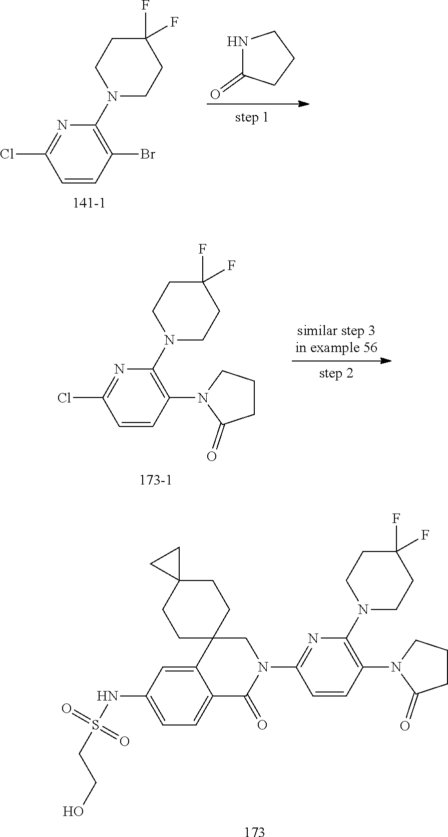 [SMILES: FC1(F)CCN(c2nc(Cl)ccc2Br)CC1 ; O=C1CCCN1 ; O=C1CCCN1c1ccc(Cl)nc1N1CCC(F)(F)CC1 ; O=C1c2ccc(NS(=O)(=O)CCO)cc2C2(CCC3(CC3)CC2)CN1c1ccc(N2CCCC2=O)c(N2CCC(F)(F)CC2)n1 ; "173"]

Step 1: A mixture of 141-1 (450 mg, 1.44 mmol), tetrahydropyrrol-2-one (200 mg, 2.35 mmol), CuI (275.1 mg, 1.44 mmol), K₃PO₄ (919.8 mg, 4.33 mmol) and methyl[(1R,2R)-2-(methylamino)cyclohexyl]amine (102.7 mg, 0.72 mmol) in DMF (5 mL) was heated at 90° C. for 6 h. The mixture was filtered and the filtrate was concentrated under vacuo. The residue was purified by column chromatography on silica gel (petroleum ether/ethyl acetate=1/0 to 1/1) to afford 173-1.

Step 2: Compound 173 was prepared from compound 173-1 following the procedure for the synthesis of compound 141 in example 56. LCMS (ESI, m/z): [M+H]⁺=644.2; ¹H NMR (400 MHz, DMSO-d₆, ppm): δ 7.80 (d, J=8.5 Hz, 1H), 7.71 (d, J=8.6 Hz, 1H), 7.55 (d, J=8.5 Hz, 1H), 6.91 (s, 1H), 6.84 (d, J=8.6 Hz, 1H), 4.29 (s, 2H), 3.73-3.65 (m, 4H), 3.41-3.35 (m, 4H), 2.98 (t, J=6.6 Hz, 2H), 2.43 (t, J=7.9 Hz, 2H), 2.16-2.04 (m, 6H), 2.02-1.92 (m, 2H), 1.85-1.74 (m, 2H), 1.63 (d, J=12.8 Hz, 2H), 0.82 (d, J=13.3 Hz, 2H), 0.28 (s, 4H). ¹⁹F NMR (376 MHz, DMSO-d6, ppm): δ −95.04 (2F).

### Example 67 Synthesis of Compound 177

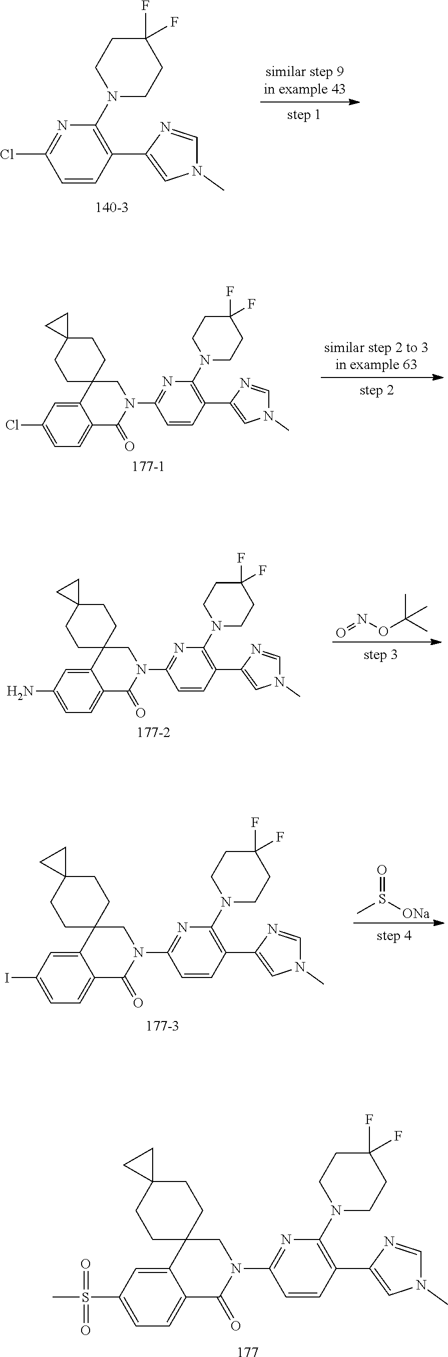

Step 1: Compound 177-1 was prepared from compound 140-3 following the procedure for the synthesis of compound 114-11 in example 43.

Step 2: Compound 177-2 was prepared from compound 177-1 following the procedure for the synthesis of compound 158-3 in example 63.

Step 3: To a solution of 177-2 (300 mg, 0.56 mmol) and tert-Butyl nitrite (100 μL, 0.85 mmol) in MeCN (5 mL) was added CuI (160.9 mg, 0.85 mmol) at room temperature. The mixture was stirred at 80° C. for 1 h. The mixture was concentrated, the residue was purified by column chromatography on silica gel (DCM/MeOH=1/0 to 20/1) to afford 177-3.

Step 4: A solution of 177-3 (100 mg, 0.16 mmol), sodium methanesulfinate (31.7 mg, 0.31 mmol), CuI (29.6 mg, 0.16 mmol), K₃PO₄ (99.0 mg, 0.47 mmol) and methyl[(1R,2R)-2-(methylamino)cyclohexyl]amine (11.1 mg, 0.078 mmol) in DMF (2 mL) was stirred at 100° C. for 0.5 h. The mixture was filtered and the filtrated was purified by reverse HPLC (0-100% of MeCN in water) to afford 177. LCMS (ESI, m/z): [M+H]⁺=596.2; ¹H NMR (400 MHz, DMSO-d₆, ppm): δ 8.30-8.26 (m, 2H), 8.06 (s, 1H), 7.99 (d, J=9.0 Hz, 1H), 7.95 (d, J=8.4 Hz, 1H), 7.76 (s, 1H), 7.69 (s, 1H), 4.50 (s, 2H), 3.74 (s, 3H), 3.35-3.31 (m, 3H), 3.28-3.22 (m, 4H), 2.24-2.05 (m, 6H), 2.00-1.95 (m, 2H), 1.74 (d, J=12.5 Hz, 2H), 0.87 (d, J=13.0 Hz, 2H), 0.32 (s, 4H). ¹⁹F NMR (376 MHz, DMSO-d₆, ppm): 8-95.44 (2F).

### Example 68 Synthesis of Compound 181

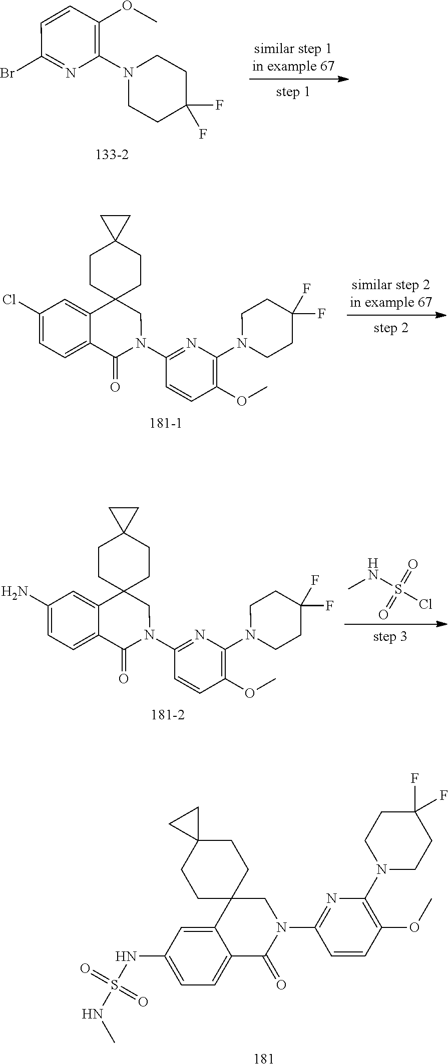

Step 1: Compound 181-1 was prepared from compound 133-2 following the procedure for the synthesis of compound 177-1 in example 67.

Step 2: Compound 181-2 was prepared from compound 181-1 following the procedure for the synthesis of compound 177-2 in example 67.

Step 3: To a solution of 181-2 (100 mg, 0.21 mmol), TEA (0.06 mL, 0.41 mmol) and DMAP (12.7 mg, 0.10 mmol) in THF (5 mL) was added methylsulfamoyl chloride (40.3 mg, 0.31 mmol), the mixture was stirred at 40° C. for 2 h. The reaction was quenched with MeOH and the mixture was concentrated in vacuo. The residue was purified by reverse HPLC (0˜68% MeCN in H₂O) to afford 181. LCMS (ESI, m/z): [M+H]⁺=576.5; ¹H NMR (400 MHz, DMSO-d₆, ppm): 8 10.11 (s, 1H), 7.91 (d, J=8.5 Hz, 1H), 7.62-7.55 (m, 2H), 7.37 (d, J=8.7 Hz, 1H), 7.26 (d, J=1.6 Hz, 1H), 7.18 (d, J=8.5, 1.8 Hz, 1H), 4.23 (s, 2H), 3.83 (s, 3H), 3.57-3.53 (m, 4H), 2.49-2.45 (m, 3H), 2.14-1.94 (m, 6H), 1.86-1.72 (m, 2H), 1.73-1.59 (m, 2H), 0.83 (d, J=13.3 Hz, 2H), 0.28 (s, 4H). 19F NMR (376 MHz, DMSO-d₆, ppm): δ −94.53 (2F).

### Example 69 Synthesis of Compound 182

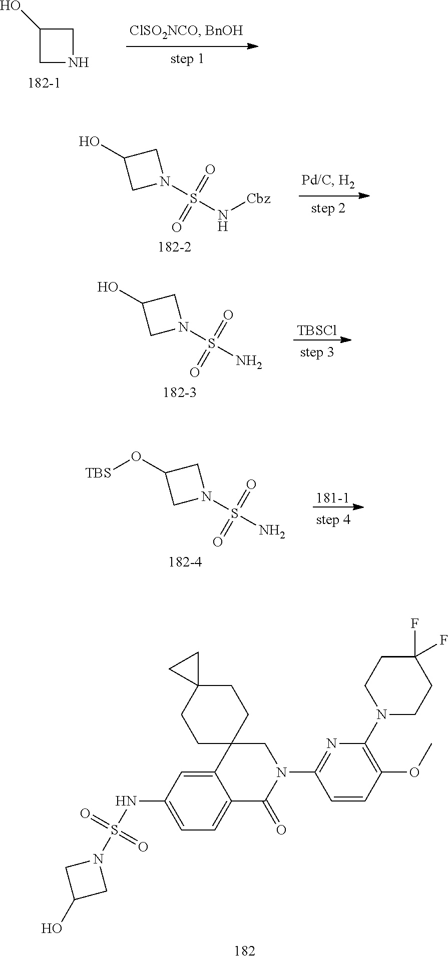

Step 1: To a solution of (oxomethylidene)azanesulfonyl chloride (0.31 mL, 3.53 mmol) in DCM (10 mL) was added benzyl alcohol (0.39 mL, 3.75 mmol) below −10° C. and stirred at −10° C. for 30 minutes. Then TEA (0.74 mL, 5.30 mmol) and 182-1 (335.7 mg, 4.59 mmol) in DMF (5 mL) were added and the mixture was stirred at −10° C. for 30 minutes. The mixture was stirred at 20° C. for 16 h. The mixture was diluted with H₂O and adjusted pH to 4 by HCl (1 M), extracted with DCM. The combined organic phase was washed with H₂O and concentrated. The residue was purified by column chromatography on silica gel (DCM/MeOH=1/0 to 20/1) to afford 182-2.

Step 2: A solution of 182-2 (454 mg, 1.59 mmol) and Pd/C (10%, 100 mg) in propan-2-ol (12 mL) was stirred at 20° C. under H₂ balloon for 2.5 h. The mixture was filtered and the filtrate was concentrated to afford 182-3 which was used for the next step directly without further purification.

Step 3: A solution of 182-3 (242 mg, 0.95 mmol), TEA (0.20 mL, 1.43 mmol), DMAP (58.3 mg, 0.48 mmol) and tert-butyldimethylsilyl chloride (215.7 mg, 1.43 mmol) in DMF (8 mL) was stirred at 20° C. for 2 h. The mixture was concentrated. The residue was purified by column chromatography on silica gel (petroleum ether/EtOAc=1/0 to 0/1) to afford 182-4.

Step 4: A solution of 181-1 (80 mg, 0.16 mmol), 182-4 (84.9 mg, 0.32 mmol), Cu₂O (23.1 mg, 0.16 mmol), potassium 2-methylpropan-2-olate (53.6 mg, 0.48 mmol) and 4-hydroxy-N-(2-methyl-1-naphthyl)pyridine-2-carboxamide (22.2 mg, 0.080 mmol) in DMSO (8 mL) was stirred at 130° C. for 16 h. The mixture was concentrated. The residue was purified by column chromatography on silica gel (DCM/MeOH=1/0 to 10/1) and reverse HPLC (MeCN/in water: 5%-65%) to afford 182. LCMS (ESI, m/z): [M+H]⁺=618.2; ¹H NMR (400 MHz, DMSO-d₆, ppm): δ 10.37 (s, 1H), 7.93 (d, J=8.5 Hz, 1H), 7.59 (d, J=8.5 Hz, 1H), 7.38 (d, J=8.6 Hz, 1H), 7.32 (s, 1H), 7.25-7.19 (m, 1H), 5.87-5.78 (m, 1H), 4.40-4.31 (m, 1H), 4.23 (s, 2H), 3.94-3.87 (m, 2H), 3.84 (s, 3H), 3.69-3.62 (m, 2H), 3.59-3.50 (m, 4H), 2.15-1.90 (m, 6H), 1.83-1.64 (m, 4H), 0.92-0.81 (m, 2H), 0.35-0.23 (m, 4H). ¹⁹F NMR (376 MHz, DMSO-d₆, ppm): δ −94.53 (2F).

Table 1 below shows characterization of some exemplary compounds of the present disclosure.

TABLE 1
Characterization of some exemplary compounds of the present disclosure

| Com- | | | |
|---|---|---|---|
| pound | | [M + | |
| No. | Structure | H]⁺ | NMR |
| 8 | 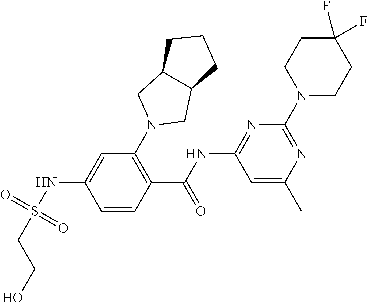 [SMILES: Cc1cc(NC(=O)c2ccc(NS(=O)(=O)CCO)cc2N2C[C@H]3CCC[C@H]3C2)nc(N2CCC(F)(F)CC2)n1] | 565.4 | ¹H NMR (400 MHz, CDCl₃, ppm) δ 11.10-11.07 (m, 1H), 8.05-7.94 (m, 1H), 7.52 (s, 1H), 7.15 (s, 1H), 7.05-6.87 (m, 2H), 4.21-4.10 (m, 2H), 4.02-3.91 (m, 4H), 3.41-3.26 (m, 4H), 2.91-2.76 (m, 4H), 2.57-2.30 (m, 4H), 2.07- 1.93 (m, 4H), 1.83-1.56 (m, 6H). ¹⁹F NMR (376 MHz, CDCl₃, ppm) δ −96.92 (2F). |
| 10 | 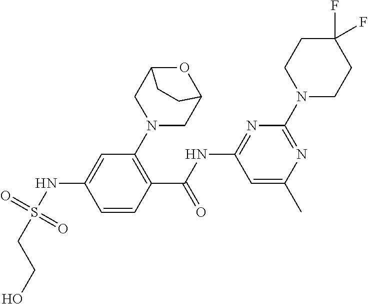 [SMILES: Cc1cc(NC(=O)c2ccc(NS(=O)(=O)CCO)cc2N2CC3CCC(C2)O3)nc(N2CCC(F)(F)CC2)n1] | 567.2 | ¹H NMR ((400 MHz, CDCl₃, ppm) δ 10.67 (s, 1H), 8.04-8.02 (m, 1H), 7.55 (s, 1H), 7.21 (s, 1H), 7.09-7.06 (m, 1H), 4.53- 4.39 (m, 2H), 4.23-4.08 (m, 2H), 4.02-3.88 (m, 4H), 3.46- 3.26 (m, 2H), 3.25-3.15 (m, 2H), 2.98-2.94 (m, 2H), 2.72- 2.55 (m, 1H), 2.39 (s, 3H), 2.36-2.29 (m, 2H), 2.08-1.88 (m, 6H). ¹⁹F NMR (376 MHz, CDCl₃, ppm) δ −96.81 (2F). |
| 12 | 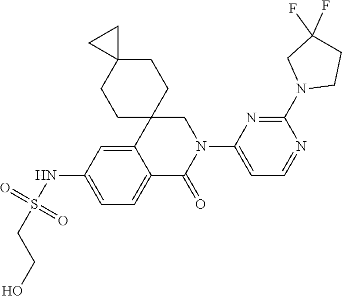 [SMILES: O=C1c2ccc(NS(=O)(=O)CCO)cc2C2(CCC3(CC3)CC2)CN1c1ccnc(N2CCC(F)(F)C2)n1] | 548.2 | ¹H NMR ((400 MHz, CDCl₃, ppm) δ 8.22-8.20 (m, 1H), 8.08- 8.06 (m, 1H), 7.64-7.63 (m, 1H), 7.32-7.31 (m, 1H), 7.21- 7.19 (m, 1H), 7.13-7.11 (m, 1H), 4.37 (s, 2H), 4.11-4.03 (m, 2H), 3.93-3.87 (m, 2H), 3.81-3.77 (m, 2H), 3.33-3.26 (m, 2H), 2.89-2.59 (m, 1H), 2.52-2.36 (m, 2H), 2.04-1.94 (m, 2H), 1.87-1.78 (m, 2H), 1.67-1.64 (m, 2H), 0.80-0.77 (m, 2H), 0.32-0.19 (m, 4H). ¹⁹F NMR (376 MHz, CDCl₃, ppm) δ −100.82 (2F). |
| 19 | 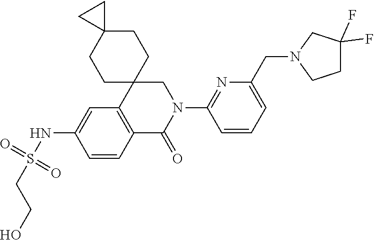 [SMILES: O=C1c2ccc(NS(=O)(=O)CCO)cc2C2(CCC3(CC3)CC2)CN1c1cccc(CN2CCC(F)(F)C2)n1] | 561.5 | ¹H NMR (400 MHz, DMSO-d₆, ppm) δ 10.21 (s, 1H), 8.00- 7.94 (m, 2H), 7.84-7.80 (m, 1H), 7.37 (s, 1H), 7.26-7.20 (m, 2H), 4.99-4.91 (m, 1H), 4.32 (s, 2H), 3.79-3.72 (m, 4H), 3.37- 3.33 (m, 2H), 3.04-2.97 (m, 2H), 2.85-2.82 (m, 2H), 2.32-2.23 (M, 2H), 1.96-1.90 (m, 2H), 1.82-1.69 (m, 4H), 0.85-0.82 (m, 2H), 0.28 (s, 4H). ¹⁹F NMR (376 MHz, DMSO-d₆, ppm) δ −90.99 (2F). |
| 21 | 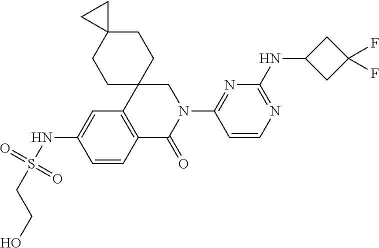 [SMILES: O=C1c2ccc(NS(=O)(=O)CCO)cc2C2(CCC3(CC3)CC2)CN1c1ccnc(NC2CC(F)(F)C2)n1] | 548.3 | ¹H NMR (400 MHz, DMSO-d₆, ppm) δ 8.23-8.22 (m, 1H), 7.97-7.95 (m, 1H), 7.74-7.69 (m, 1H), 7.62-7.58 (m, 1H), 7.29 (s, 1H), 7.20-7.18 (m, 1H), 4.45-4.37 (m, 2H), 4.25- 4.22 (m, 1H), 3.77-3.74 (m, 2H), 3.33-3.28 (m, 4H), 3.02- 2.96 (m, 2H), 2.76-2.62 (m, 2H), 2.03-1.98 (m, 2H), 1.82- 1.76 (m, 2H), 1.66-1.62 (m, 2H), 0.88-0.85 (m, 2H), 0.31- 0.30 (m, 4H). ¹⁹F NMR (376 MHz, DMSO-d₆, ppm) δ −81.55 (1F), −82.07 (1F). |
| 22 | 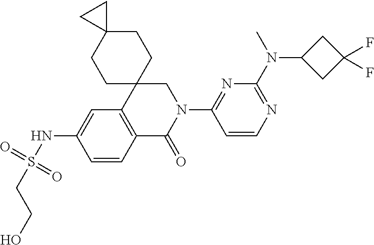 [SMILES: CN(c1nccc(N2CC3(CCC4(CC4)CC3)c3cc(NS(=O)(=O)CCO)ccc3C2=O)n1)C1CC(F)(F)C1] | 562.2 | ¹H NMR (400 MHz, DMSO-d₆, ppm) δ 10.28 (s, 1H), 8.35- 8.29 (m, 1H), 8.03-7.95 (m, 1H), 7.70-7.64 (m, 1H), 7.33 (s, 1H), 7.26-7.20 (m, 1H), 5.11-5.00 (m, 1H), 4.99-4.78 (m, 1H), 4.48 (s, 2H), 3.83-3.70 (m, 2H), 3.36-3.31 (m, 2H), 3.13 (s, 3H), 3.03-2.82 (m, 4H), 2.07-1.93 (m, 2H), 1.86- 1.74 (m, 2H), 1.72-1.61 (m, 2H), 0.95-0.81 (m, 2H), 0.39-0.24 (m, 4H). ¹⁹F NMR (376 MHz, DMSO-d₆, ppm) δ −82.48 (1F), −98.89 (1F). |
| 25 | 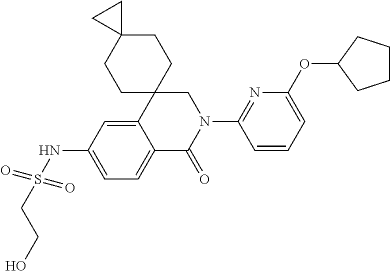 [SMILES: O=C1c2ccc(NS(=O)(=O)CCO)cc2C2(CCC3(CC3)CC2)CN1c1cccc(OC2CCCC2)n1] | 526.4 | ¹H NMR (400 MHz, CD₃OD, ppm): δ 7.99-7.97 (m, 1H), 7.71-7.56 (m, 1H), 7.51-7.43 (m, 1H), 7.33 (s, 1H), 7.20- 7.15 (m, 1H), 6.54-6.52 (m, 1H), 5.45-5.38 (m, 1H), 4.36 (s, 2H), 3.96-3.93 (m, 2H), 3.40-3.35 (m, 2H), 2.23-1.60 (m, 14H), 0.97-0.84 (m, 2H), 0.32 (s, 4H). |
| 30 | 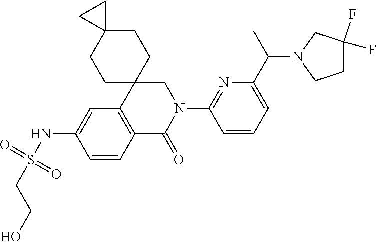 [SMILES: CC(c1cccc(N2CC3(CCC4(CC4)CC3)c3cc(NS(=O)(=O)CCO)ccc3C2=O)n1)N1CCC(F)(F)C1] | 575.3 | ¹H NMR (400 MHz, CDCl₃, ppm): δ 7.94-7.92 (m, 1H), 7.81- 7.79 (m, 1H), 7.51-7.47 (m, 1H), 7.16 (s, 1H), 6.97-6.94 (m, 2H), 6.87-6.73 (m, 1H), 4.23-4.07 (m, 2H), 3.96-3.86 (m, 2H), 3.60-3.27 (m, 1H), 3.18-3.06 (m, 2H), 2.83- 2.64 (m, 2H), 2.57-2.24 (m, 2H), 2.18-1.95 (m, 2H), 1.87- 1.75 (m, 2H), 1.73-1.62 (m, 2H), 1.61-1.52 (m, 2H), 1.28-1.22 (m, 3H), 0.62-0.59 (m, 2H), 0.24-−0.04 (m, 4H); ¹⁹F NMR (376 MHz, CDCl₃, ppm): δ −92.42 (2F). |
| 31 | 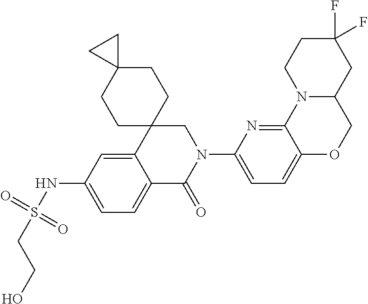 [SMILES: O=C1c2ccc(NS(=O)(=O)CCO)cc2C2(CCC3(CC3)CC2)CN1c1ccc2c(n1)N1CCC(F)(F)CC1CO2] | 589.2 | ¹H NMR (400 MHz, DMSO-d₆, ppm): δ 10.17 (s, 1H), 7.95- 7.93 (m, 1H), 7.34 (s, 1H), 7.29-7.27 (m, 1H), 7.24-7.19 (m, 1H), 7.13-7.11 (m, 1H), 4.96 (s, 1H), 4.72-4.59 (m, 1H), 4.38-4.30 (m, 1H), 4.27-4.18 (m, 1H), 4.16-4.09 (m, 1H), 4.08-4.00 (m, 1H), 3.80-3.71 (m, 2H), 3.60-3.50 (m, 1H), 3.37-3.34 (m, 2H), 2.97-2.84 (m, 1H), 2.30-2.09 (m, 2H), 2.03-1.66 (m, 8H), 0.94-0.78 (m, 2H), 0.36-0.23 (m, 4H). ¹⁹F NMR (376 MHz, DMSO-d₆, ppm): δ −87.53 (1F), −100.68 (1F). |
| 32 | 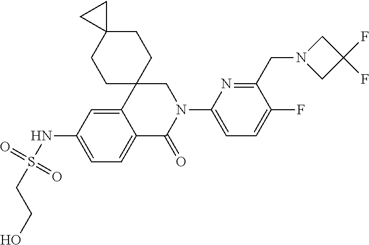 [SMILES: O=C1c2ccc(NS(=O)(=O)CCO)cc2C2(CCC3(CC3)CC2)CN1c1ccc(F)c(CN2CC(F)(F)C2)n1] | 565.2 | ¹H NMR (400 MHz, DMSO-d₆, ppm): δ 10.18 (s, 1H), 8.05- 7.94 (m, 2H), 7.79-7.75 (m, 1H), 7.34 (s, 1H), 7.24-7.22 (m, 1H), 5.21-4.80 (m, 1H), 4.27 (s, 2H), 3.90 (s, 2H), 3.85-3.72 (m, 6H), 3.33-3.28 (m, 2H), 1.98-1.87 (m, 2H), 1.86-1.68 (m, 4H), 0.92-0.80 (m, 2H), 0.29 (s, 4H). ¹⁹F NMR (376 MHz, DMSO-d₆, ppm): δ −97.91 (2F), −131.55 (1F). |
| 33 | 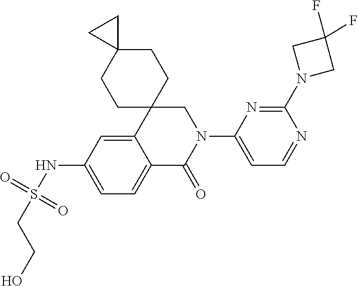 [SMILES: O=C1c2ccc(NS(=O)(=O)CCO)cc2C2(CCC3(CC3)CC2)CN1c1ccnc(N2CC(F)(F)C2)n1] | 534.2 | 0.37 FA salt. ¹H NMR (400 MHz, DMSO-d₆, ppm): δ 8.36- 8.34 (m, 1H), 8.28 (s, 0.37H), 7.96-7.94 (m, 1H), 7.74-7.73 (m, 1H), 7.28 (s, 1H), 7.19-7.17 (m, 1H), 4.53-4.50 (m, 4H), 4.41 (s, 2H), 3.80-3.72 (m, 2H), 3.32-3.27 (m, 2H), 2.08- 1.95 (m, 2H), 1.86-1.74 (m, 2H), 1.70-1.57 (m, 2H), 0.93-0.76 (m, 2H), 0.42-0.21 (m, 4H). ¹⁹F NMR (376 MHz, DMSO-d₆, ppm) δ −99.05 (2F). |
| 34 | 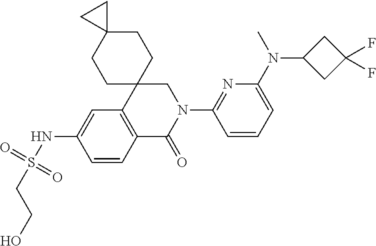 [SMILES: CN(c1cccc(N2CC3(CCC4(CC4)CC3)c3cc(NS(=O)(=O)CCO)ccc3C2=O)n1)C1CC(F)(F)C1] | 561.2 | ¹H-NMR (400 MHz, DMSO-d₆, ppm): δ 10.19 (s, 1H), 7.98- 7.96 (m, 1H), 7.62-7.58 (m, 1H), 7.51-7.49 (m, 1H), 7.35 (s, 1H), 7.25-7.22 (m, 1H), 6.53-6.51 (m, 1H), 4.96-4.84 (m, 2H), 4.35 (s, 2H), 3.78-3.74 (m, 2H), 3.36-3.35 (m, 2H), 2.97 (s, 3H), 2.93-2.87 (m, 4H), 2.03-1.97 (m, 2H), 1.82-1.68 (m, 4H), 0.88-0.85 (m, 2H), 0.30-0.29 (m, 4H). ¹⁹F NMR (376 MHz, DMSO-d₆, ppm): δ −82.04 (1F), −98.22 (1F). |
| 37 | 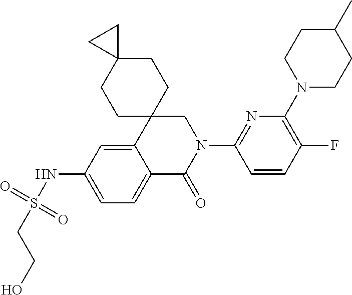 [SMILES: CC1CCN(c2nc(N3CC4(CCC5(CC5)CC4)c4cc(NS(=O)(=O)CCO)ccc4C3=O)ccc2F)CC1] | 557.2 | ¹H NMR (400 MHz, DMSO-d₆, ppm): δ 7.66-7.60 (m, 1H), 7.60-7.53 (m, 1H), 7.50-7.41 (m, 1H), 6.81 (s, 1H), 6.77- 6.69 (m, 1H), 5.08 (s, 1H), 4.23 (s, 2H), 4.09-3.98 (m, 2H), 3.74-3.63 (m, 2H), 2.98-2.85 (m, 4H), 2.05-1.91 (m, 2H), 1.84-1.73 (m, 2H), 1.72-1.56 (m, 5H), 1.33- 1.18 (m, 2H), 0.97-0.89 (m, 3H), 0.85-0.73 (m, 2H), 0.27 (s, 4H). ¹⁹F NMR (376 MHz, DMSO-d₆, ppm): δ −135.37 (1F). |
| 38 | 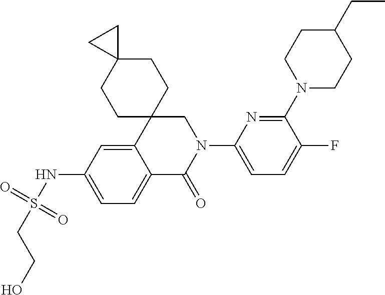 [SMILES: CCC1CCN(c2nc(N3CC4(CCC5(CC5)CC4)c4cc(NS(=O)(=O)CCO)ccc4C3=O)ccc2F)CC1] | 571.4 | ¹H NMR (400 MHz, CD₃OD, ppm): δ 8.02-8.00 (m, 1H), 7.42-7.35 (m, 3H), 7.28-7.18 (m, 1H), 4.32 (s, 2H), 4.18- 4.15 (m, 2H), 3.96-3.92 (m, 2H), 3.37-3.30 (m, 2H), 2.97- 2.90 (m, 2H), 2.17-2.07 (m, 2H), 2.04-1.90 (m, 2H), 1.80- 1.77 (m, 4H), 1.48-1.39 (m, 1H), 1.36-1.29 (m, 4H), 0.99- 0.84 (m, 5H), 0.33 (s, 4H). ¹⁹F NMR (376 MHz, CD₃OD, ppm): δ −134.56 (1F). |
| 39 | 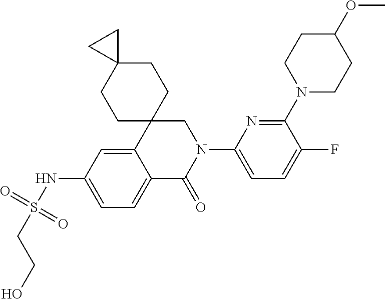 [SMILES: COC1CCN(c2nc(N3CC4(CCC5(CC5)CC4)c4cc(NS(=O)(=O)CCO)ccc4C3=O)ccc2F)CC1] | 573.3 | ¹H NMR (400 MHz, DMSO-d₆, ppm): δ 10.21 (s, 1H), 7.98- 7.95 (m, 1H), 7.58-7.51 (m, 2H), 7.33 (s, 1H), 7.25-7.20 (m, 1H), 4.98 (s, 1H), 4.30 (s, 2H), 3.87-3.72 (m, 4H), 3.47- 3.43 (m, 1H), 3.34-3.31 (m, 2H), 3.28 (s, 3H), 3.24-3.16 (m, 2H), 2.07-1.90 (m, 4H), 1.84-1.65 (m, 4H), 1.58-1.47 (m, 2H), 0.90-0.81 (m, 2H), 0.37-0.21 (m, 4H). ¹⁹F NMR (376 MHz, DMSO-d₆, ppm): δ −133.64 (1F). |
| 40 | 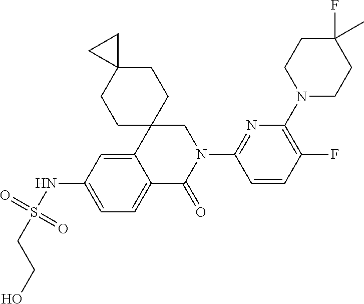 [SMILES: CC1(F)CCN(c2nc(N3CC4(CCC5(CC5)CC4)c4cc(NS(=O)(=O)CCO)ccc4C3=O)ccc2F)CC1] | 575.3 | 0.6 TFA salt. ¹H NMR (400 MHz, DMSO-d₆, ppm): δ 10.20 (s, 1H), 8.02-7.92 (m, 1H), 7.62-7.52 (m, 2H), 7.34 (s, 1H), 7.27-7.20 (m, 1H), 4.96 (s, 1H), 4.31 (s, 2H), 3.91-3.81 (m, 2H), 3.80-3.71 (m, 2H), 3.39-3.34 (m, 2H), 3.31-3.22 (m, 2H), 2.05-1.93 (m, 2H), 1.89-1.74 (m, 6H), 1.73-1.64 (m, 2H), 1.40-1.35 (d, J = 21.6 Hz, 3H), 0.92-0.81 (m, 2H), 0.30 (s, 4H). ¹⁹F NMR (376 MHz, DMSO-d₆, ppm): δ −73.47 (1.8F), −133.86 (1F), −148.06 (1F). |
| 42 | 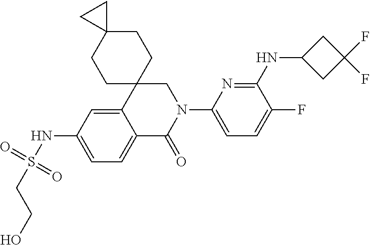 [SMILES: O=C1c2ccc(NS(=O)(=O)CCO)cc2C2(CCC3(CC3)CC2)CN1c1ccc(F)c(NC2CC(F)(F)C2)n1] | 565.2 | ¹H NMR (400 MHz, DMSO-d₆, ppm): δ 10.19 (s, 1H), 8.02- 7.91 (m, 1H), 7.54-7.41 (m, 1H), 7.38-7.26 (m, 3H), 7.25- 7.19 (m, 1H), 4.98 (s, 1H), 4.36-4.23 (m, 3H), 3.80-3.71 (m, 2H), 3.35-3.31 (m, 2H), 3.07-2.91 (m, 2H), 2.85-2.66 (m, 2H), 2.12-2.00 (m, 2H), 1.86-1.62 (m, 4H), 0.93-0.81 (m, 2H), 0.38-0.23 (m, 4H). ¹⁹F NMR (376 MHz, DMSO- d₆, ppm): δ −81.27 (1F), −96.80 (1F), −144.92 (1F). |
| 45 | 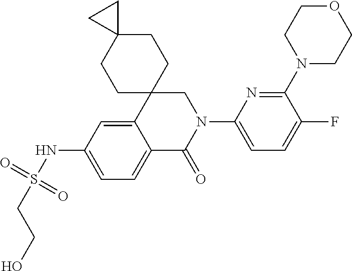 [SMILES: O=C1c2ccc(NS(=O)(=O)CCO)cc2C2(CCC3(CC3)CC2)CN1c1ccc(F)c(N2CCOCC2)n1] | 545.2 | ¹H NMR (400 MHz, DMSO-d₆, ppm): δ 7.97-7.95 (m, 1H), 7.47-7.73 (m, 2H), 7.34 (s, 1H), 7.24-7.22 (m, 1H), 4.27 (s, 2H), 3.76-3.75 (m, 6H), 3.43-3.45 (m, 4H), 3.31-3.32 (m, 2H), 2.02-1.96 (m, 2H), 1.82-1.68 (m, 4H), 0.88-0.84 (m, 2H), 0.30 (s, 4H). ¹⁹F NMR (376 MHz, DMSO-d₆, ppm): δ −134.54 (1F). |
| 46 | 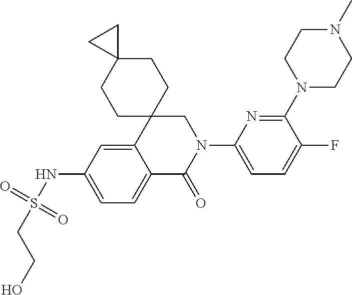 [SMILES: CN1CCN(c2nc(N3CC4(CCC5(CC5)CC4)c4cc(NS(=O)(=O)CCO)ccc4C3=O)ccc2F)CC1] | 558.3 | ¹H NMR (400 MHz, DMSO-d₆, ppm): δ 7.97-7.95 (m, 1H), 7.48-7.61 (m, 2H), 7.33 (s, 1H), 7.23-7.21 (m, 1H), 4.29 (s, 2H), 3.76-3.74 (m, 2H), 3.49-3.47 (m, 4H), 3.36-3.31 (m, 2H), 2.34-2.49 (m, 4H), 2.22 (s, 3H), 2.03-1.97 (m, 2H), 1.79-1.67 (m, 4H), 0.87-0.84 (m, 2H), 0.30 (s, 4H). ¹⁹F NMR (376 MHz, DMSO-d₆, ppm): δ −134.14 (1F). |
| 47 | 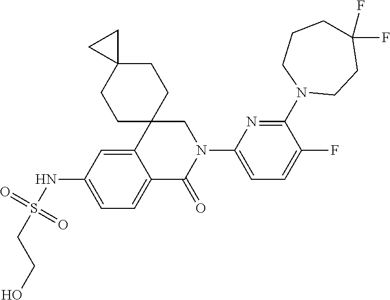 [SMILES: O=C1c2ccc(NS(=O)(=O)CCO)cc2C2(CCC3(CC3)CC2)CN1c1ccc(F)c(N2CCCC(F)(F)CC2)n1] | 593.2 | ¹H NMR (400 MHz, DMSO-d₆, ppm): δ 7.64-7.61 (m, 1H), 7.46-7.42 (m, 2H), 6.80 (s, 1H), 6.75-6.72 (m, 1H), 5.09 (s, 1H), 4.19 (s, 2H), 3.59-3.81 (m, 6H), 2.91-2.88 (m, 2H), 2.39-2.28 (m, 2H), 2.22-2.04 (m, 2H), 1.92-1.89 (m, 4H), 1.80-1.75 (m, 2H), 1.63-1.60 (m, 2H), 0.81-0.78 (m, 2H), 0.26 (s, 4H). ¹⁹F NMR (376 MHz, DMSO-d₆, ppm): δ −87.18 (2F), −139.67 (1F). |
| 48 | 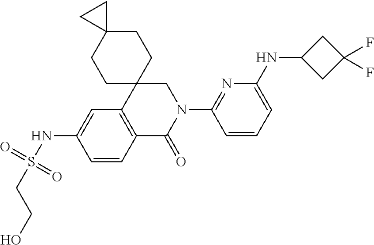 [SMILES: O=C1c2ccc(NS(=O)(=O)CCO)cc2C2(CCC3(CC3)CC2)CN1c1cccc(NC2CC(F)(F)C2)n1] | 547.2 | ¹H NMR (400 MHz, CD₃OD, ppm): δ 8.03-7.01 (m, 1H), 7.48-7.44 (m, 1H), 7.41 (s, 1H), 7.27-7.24 (m, 1H), 7.21-7.19 (m, 1H), 6.36-6.34 (m, 1H), 4.30-4.26 (m, 3H), 3.96-3.93 (m, 2H), 3.38-3.35 (m, 2H), 3.04-3.00 (m, 2H), 2.56-2.50 (m, 2H), 2.18-2.11 (m, 2H), 1.99-1.92 (m, 2H), 1.81-1.78 (m, 2H), 0.92-0.88 (m, 2H), 0.36-0.34 (m, 4H). ¹⁹F NMR (376 MHz, CD₃OD, ppm): δ −84.54 (1F), −98.96 (1F). |
| 49 | 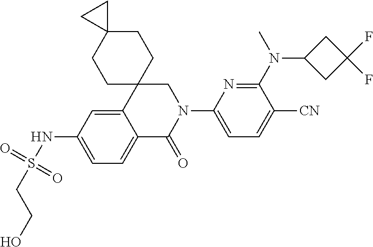 [SMILES: CN(c1nc(N2CC3(CCC4(CC4)CC3)c3cc(NS(=O)(=O)CCO)ccc3C2=O)ccc1C#N)C1CC(F)(F)C1] | 586.2 | ¹H NMR (400 MHz, CDCl₃, ppm): δ 8.21-8.18 (m, 1H), 7.92- 7.75 (m, 2H), 7.40 (s, 1H), 7.22-7.15 (m, 1H), 6.75 (s, 1H), 4.62-4.60 (m, 1H), 4.39 (s, 2H), 4.24-4.13 (m, 2H), 3.43- 3.31 (m, 2H), 3.26 (s, 3H), 3.09-2.91 (m, 2H), 2.84-2.68 (m, 2H), 2.13-1.90 (m, 4H), 1.86-1.70 (m, 2H), 0.93-0.90 (m, 2H), 0.45-0.28 (m, 4H). ¹⁹F NMR (376 MHz, CDCl₃, ppm): δ −83.68 (1F), −99.15 (1F). |
| 50 | 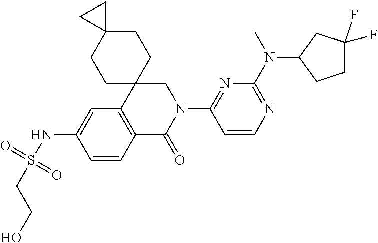 [SMILES: CN(c1nccc(N2CC3(CCC4(CC4)CC3)c3cc(NS(=O)(=O)CCO)ccc3C2=O)n1)C1CCC(F)(F)C1] | 576.3 | ¹H NMR (400 MHz, DMSO-d₆, ppm): δ 10.27 (s, 1H), 8.31- 8.30 (m, 1H), 8.01-7.99 (m, 1H), 7.67-7.65 (m, 1H), 7.35 (s, 1H), 7.27-7.22 (m, 1H), 5.50-5.34 (m, 1H), 4.97 (s, 1H), 4.60-4.40 (m, 2H), 3.80-3.71 (m, 2H), 3.39-3.34 (m, 2H), 3.05 (s, 3H), 2.42-2.25 (m, 3H), 2.16-1.94 (m, 5H), 1.86- 1.72 (m, 2H), 1.69-1.59 (m, 2H), 0.91-0.79 (m, 2H), 0.39- 0.21 (m, 4H). ¹⁹F NMR (376 MHz, DMSO-d₆, ppm): δ −87.42 (1F), −89.13 (1F). |
| 51 | 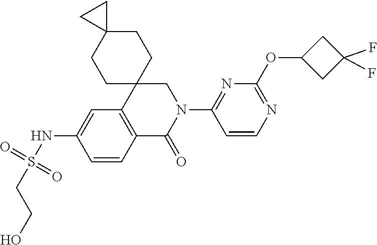 [SMILES: O=C1c2ccc(NS(=O)(=O)CCO)cc2C2(CCC3(CC3)CC2)CN1c1ccnc(OC2CC(F)(F)C2)n1] | 549.2 | ¹H NMR (400 MHz, DMSO-d₆, ppm): δ 10.31 (s, 1H), 8.52- 8.51 (m, 1H), 8.06-7.95 (m, 2H), 7.37 (s, 1H), 7.27-7.22 (m, 1H), 5.19-5.09 (m, 1H), 5.03-4.89 (m, 1H), 4.41 (s, 2H), 3.80-3.72 (m, 2H), 3.40-3.35 (m, 2H), 3.25-3.14 (m, 2H), 2.95-2.80 (m, 2H), 1.98-1.87 (m, 2H), 1.86-1.74 (m, 2H), 1.72-1.63 (m, 2H), 0.94-0.82 (m, 2H), 0.38-0.24 (m, 4H). ¹⁹F NMR (376 MHz, DMSO-d₆, ppm): δ −83.80 (1F), −94.26 (1F). |
| 53 | 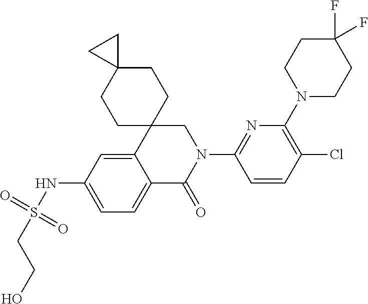 [SMILES: O=C1c2ccc(NS(=O)(=O)CCO)cc2C2(CCC3(CC3)CC2)CN1c1ccc(Cl)c(N2CCC(F)(F)CC2)n1] | 595.2 | ¹H NMR (400 MHz, DMSO-d₆, ppm): δ 7.99-7.97 (m, 1H), 7.85 (s, 2H), 7.34 (s, 1H), 7.24-7.22 (m, 1H), 4.35 (s, 2H), 3.78-3.74 (m, 2H), 3.52-3.47 (m, 4H), 3.35-3.31 (m, 2H), 2.19-2.08 (m, 4H), 2.00-1.95 (m, 2H), 1.82-1.77 (m, 2H), 1.70-1.67 (m, 2H), 0.88-0.85 (m, 2H), 0.29 (s, 4H). ¹⁹F NMR (376 MHz, DMSO-d₆, ppm): δ −95.04 (2F). |
| 54 | 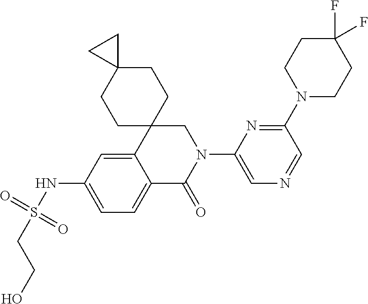 [SMILES: O=C1c2ccc(NS(=O)(=O)CCO)cc2C2(CCC3(CC3)CC2)CN1c1cncc(N2CCC(F)(F)CC2)n1] | 562.2 | ¹H NMR (400 MHz, DMSO-d₆, ppm): δ 8.72 (s, 1H), 8.20 (s, 1H), 8.00-7.98 (m, 1H), 7.34 (s, 1H), 7.25-7.23 (m, 1H), 4.29 (s, 2H), 3.86-3.72 (m, 6H), 3.33-3.23 (m, 2H), 2.12-1.89 (m, 6H), 1.85-1.61 (m, 4H), 0.88-0.85 (m, 2H), 0.30 (s, 4H). ¹⁹F NMR (376 MHz, DMSO-d₆, ppm): δ −94.52 (2F). |
| 57 | 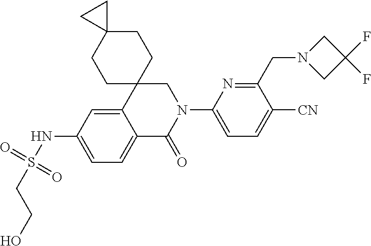 [SMILES: N#Cc1ccc(N2CC3(CCC4(CC4)CC3)c3cc(NS(=O)(=O)CCO)ccc3C2=O)nc1CN1CC(F)(F)C1] | 572.2 | ¹H NMR (400 MHz, DMSO-d₆, ppm): δ 7.99-7.97 (m, 1H), 7.83-7.81 (m, 1H), 7.29 (s, 1H), 7.17-7.15 (m, 1H), 6.57-6.55 (m, 1H), 4.86 (s, 2H), 4.42-4.36 (m, 4H), 3.76-3.72 (m, 4H), 3.32-3.26 (m, 2H), 1.78-1.70 (m, 6H), 0.79-0.75 (m, 2H), 0.28-0.26 (m, 4H). ¹⁹F NMR (376 MHz, DMSO-d₆, ppm): δ −99.13 (2F). |
| 61 | 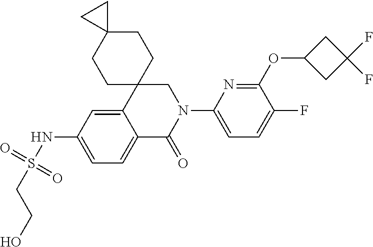 [SMILES: O=C1c2ccc(NS(=O)(=O)CCO)cc2C2(CCC3(CC3)CC2)CN1c1ccc(F)c(OC2CC(F)(F)C2)n1] | 566.2 | ¹H NMR (400 MHz, CD₃OD, ppm): δ 8.04-8.02 (m, 1H), 7.65-7.57 (m, 2H), 7.40 (s, 1H), 7.28-7.16 (m, 1H), 5.34-5.21 (m, 1H), 4.47 (s, 2H), 3.96-3.93 (m, 2H), 3.38-3.30 (m, 2H), 3.22-3.10 (m, 2H), 2.80-2.70 (m, 2H), 2.16-1.99 (m, 4H), 1.76-1.72 (m, 2H), 0.98-0.82 (m, 2H), 0.34 (s, 4H). ¹⁹F NMR (376 MHz, CD₃OD, ppm): δ −86.03 (1F), −97.89 (1F), −146.18 (1F). |
| 62 | 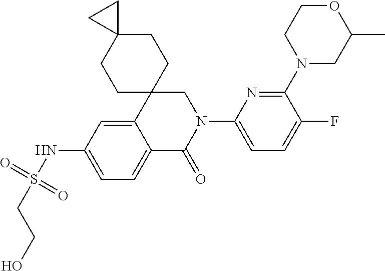 [SMILES: CC1CN(c2nc(N3CC4(CCC5(CC5)CC4)c4cc(NS(=O)(=O)CCO)ccc4C3=O)ccc2F)CCO1] | 559.2 | ¹H NMR (400 MHz, DMSO-d₆, ppm): δ 10.21 (s, 1H), 7.97- 7.93 (m, 1H), 7.70-7.54 (m, 2H), 7.33 (s, 1H), 7.26-7.17 (m, 1H), 5.19-4.88 (m, 1H), 4.39-4.18 (m, 2H), 3.94-3.79 (m, 3H), 3.78-3.72 (m, 2H), 3.72-3.61 (m, 2H), 3.37-3.35 (m, 1H), 3.33-3.30 (m, 1H), 3.04-2.94 (m, 1H), 2.74-2.63 (m, 1H), 2.13-1.99 (m, 2H), 1.85-1.73 (m, 2H), 1.73-1.63 (m, 2H), 1.16 (d, J = 6.2 Hz, 3H), 0.88-0.85 (m, 2H), 0.30 (s, 4H). ¹⁹F NMR (376 MHz, DMSO-d₆, ppm): δ −134.54 (1F). |
| 63 | 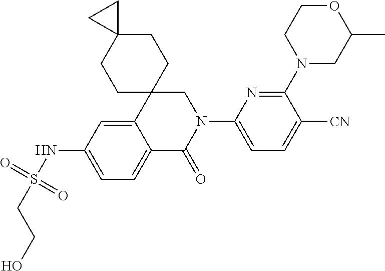 [SMILES: CC1CN(c2nc(N3CC4(CCC5(CC5)CC4)c4cc(NS(=O)(=O)CCO)ccc4C3=O)ccc2C#N)CCO1] | 566.4 | ¹H NMR (400 MHz, DMSO-d₆, ppm): δ 8.08-7.93 (m, 2H), 7.90-7.88 (m, 1H), 7.31 (s, 1H), 7.24-7.21 (m, 1H), 4.56-4.33 (m, 2H), 4.26-4.08 (m, 2H), 3.96-3.93 (m, 1H), 3.81-3.71 (m, 2H), 3.71-3.55 (m, 2H), 3.36-3.34 (m, 1H), 3.32-3.27 (m, 1H), 3.21-3.12 (m, 1H), 2.87-2.78 (m, 1H), 2.09-1.87 (m, 2H), 1.87-1.70 (m, 2H), 1.69-1.53 (m, 2H), 1.17 (d, J = 6.2 Hz, 3H), 0.87-0.83 (m, 2H), 0.31 (s, 4H). |
| 64 | 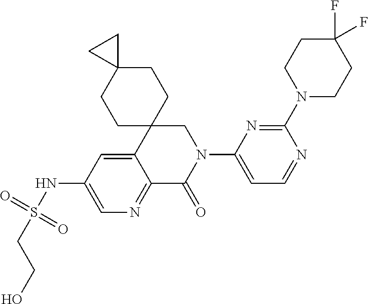 [SMILES: O=C1c2ncc(NS(=O)(=O)CCO)cc2C2(CCC3(CC3)CC2)CN1c1ccnc(N2CCC(F)(F)CC2)n1] | 563.2 | ¹H NMR (400 MHz, CD₃OD, ppm): δ 8.48-8.43 (m, 1H), 8.31 (s, 1H), 7.84 (s, 1H), 7.76-7.62 (m, 1H), 4.53 (s, 2H), 4.06-3.92 (m, 6H), 3.41-3.34 (m, 2H), 2.17-1.88 (m, 8H), 1.80-1.68 (m, 2H), 0.98-0.85 (m, 2H), 0.36 (s, 4H). ¹⁹F NMR (376 MHz, CD₃OD, ppm): δ −98.21 (2F). |
| 65 | 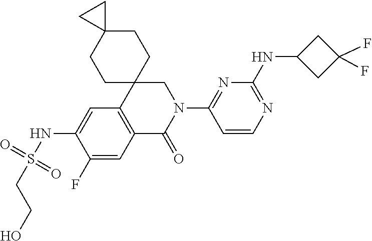 [SMILES: O=C1c2cc(F)c(NS(=O)(=O)CCO)cc2C2(CCC3(CC3)CC2)CN1c1ccnc(NC2CC(F)(F)C2)n1] | 566.2 | ¹H NMR (400 MHz, DMSO-d₆, ppm): δ 8.25-8.17 (m, 1H), 7.73 (s, 1H), 7.65-7.52 (m, 2H), 7.52-7.46 (m, 1H), 4.46- 4.40 (m, 2H), 4.29-4.17 (m, 1H), 3.81-3.70 (m, 2H), 3.24- 3.12 (m, 2H), 3.05-2.90 (m, 2H), 2.76-2.61 (m, 2H), 2.07-1.92 (m, 2H), 1.82-1.72 (m, 2H), 1.66-1.56 (m, 2H), 0.89-0.80 (m, 2H), 0.30 (s, 4H). ¹⁹F NMR (376 MHz, DMSO-d₆, ppm): δ −81.80 (1F), −96.69 (1F), −129.21 (1F). |
| 66 | 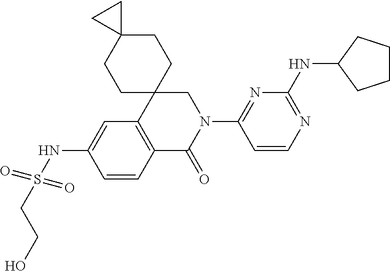 [SMILES: O=C1c2ccc(NS(=O)(=O)CCO)cc2C2(CCC3(CC3)CC2)CN1c1ccnc(NC2CCCC2)n1] | 526.2 | ¹H NMR (400 MHz, DMSO-d₆, ppm): δ 8.12-8.11 (m, 1H), 7.76-7.73 (m, 1H), 7.63-7.37 (m, 1H), 7.18-7.03 (m, 1H), 6.95 (s, 1H), 6.91-6.83 (m, 1H), 4.60-4.27 (m, 2H), 4.27-4.11 (m, 1H), 3.74-3.66 (m, 2H), 3.07-2.96 (m, 2H), 2.15-1.92 (m, 4H), 1.84-1.67 (m, 4H), 1.63-1.47 (m, 6H), 0.2-0.79 (m, 2H), 0.28 (s, 4H). |
| 69 | 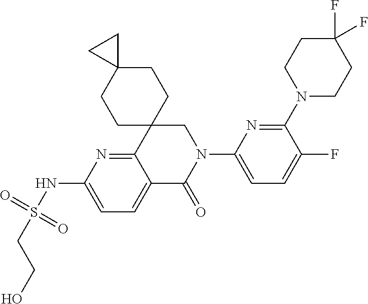 [SMILES: O=C1c2ccc(NS(=O)(=O)CCO)nc2C2(CCC3(CC3)CC2)CN1c1ccc(F)c(N2CCC(F)(F)CC2)n1] | 580.4 | 3.0 TFA salt. ¹H NMR (400 MHz, DMSO-d₆, ppm): δ 11.06 (s, 1H), 8.23-8.21 (m, 1H), 7.68-7.58 (m, 1H), 7.57-7.49 (m, 1H), 6.95-6.88 (m, 1H), 5.01-4.96 (m, 1H), 4.30 (s, 2H), 3.83-3.74 (m, 4H), 3.66-3.59 (m, 4H), 2.18-2.02 (m, 6H), 1.91-1.80 (m, 2H), 1.67-1.60 (m, 2H), 1.01- 0.94 (m, 2H), 0.32-0.21 (m, 4H). ¹⁹F NMR (376 MHz, DMSO-d₆, ppm): δ −73.53 (9F), −94.52 (2 F), −133.40 (1F). |
| 70 | 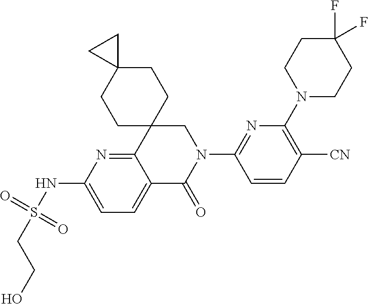 [SMILES: N#Cc1ccc(N2CC3(CCC4(CC4)CC3)c3nc(NS(=O)(=O)CCO)ccc3C2=O)nc1N1CCC(F)(F)CC1] | 587.2 | ¹H NMR (400 MHz, DMSO-d₆, ppm): δ 11.19 (s, 1H), 8.22- 8.20 (m, 1H), 8.12-8.06 (m, 1H), 7.81-7.74 (m, 1H), 6.90- 6.88 (m, 1H), 4.99-4.95 (m, 1H), 4.43 (s, 2H), 3.87-3.77 (m, 6H), 3.77-3.71 (m, 2H), 2.22-2.02 (m, 6H), 1.88- 1.77 (m, 2H), 1.63-1.55 (m, 2H), 1.00-0.93 (m, 2H), 0.32- 0.21 (m, 4H). ¹⁹F NMR (376 MHz, DMSO-d₆, ppm): δ −94.65 (2F). |
| 71 | 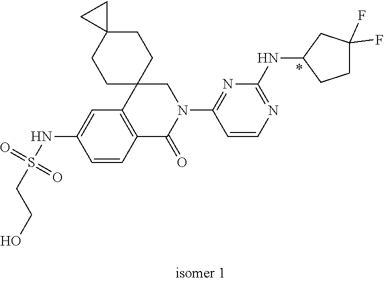 [SMILES: O=C1c2ccc(NS(=O)(=O)CCO)cc2C2(CCC3(CC3)CC2)CN1c1ccnc(NC2CCC(F)(F)C2)n1] | 562.3 | SFC analysis: 100% ee; retention time: 1.803 min; column: ChiralPak IC, 100 × 4.6 mm I.D., 3 μm, MeOH (0.05% of DEA) in CO₂, 40%; pressure: 100 bar; flow rate: 2.5 mL/min. ¹H NMR (400 MHz, DMSO-d₆, ppm): δ 10.25 (s, 1H), 8.23- 8.21 (m, 1H), 8.01-7.99 (m, 1H), 7.68-7.42 (m, 2H), 7.35 (s, 1H), 7.26-7.24 (m, 1H), 4.96 (s, 1H), 4.64-4.28 (m, 3H), 3.80-3.73 (m, 2H), 3.39-3.34 (m, 2H), 2.64-2.53 (m, 1H), 2.37-2.09 (m, 4H), 2.05-1.90 (m, 2H), 1.88-1.71 (m, 3H), 1.69-1.59 (m, 2H), 0.91-0.81 (m, 2H), 0.37-0.23 (m, 4H). ¹⁹F NMR (376 MHz, DMSO-d₆, ppm): δ −86.49 (1F), −88.14 (1F). |
| 72 | 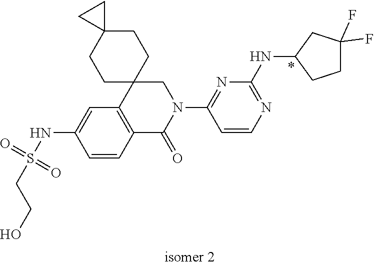 [SMILES: O=C1c2ccc(NS(=O)(=O)CCO)cc2C2(CCC3(CC3)CC2)CN1c1ccnc(NC2CCC(F)(F)C2)n1] | 562.3 | SFC analysis: 99.34% ee; retention time: 2.203 min; column: ChiralPak IC, 100 × 4.6 mm I.D., 3 μm, MeOH (0.05% of DEA) in CO2, 40%; pressure: 100 bar; flow rate: 2.5 mL/min. ¹H NMR (400 MHz, DMSO-d₆, ppm): δ 10.26 (s, 1H), 8.23- 8.21 (m, 1H), 8.01-7.99 (m, 1H), 7.68-7.42 (m, 2H), 7.35 (s, 1H), 7.27-7.22 (m, 1H), 4.96 (s, 1H), 4.64-4.28 (m, 3H), 3.80-3.72 (m, 2H), 3.39-3.34 (m, 2H), 2.65-2.53 (m, 1H), 2.37-2.09 (m, 4H), 2.06-1.92 (m, 2H), 1.90-1.71 (m, 3H), 1.68-1.60 (m, 2H), 0.91-0.81 (m, 2H), 0.36-0.23 (m, 4H). ¹⁹F NMR (376 MHz, DMSO-d₆, ppm): δ −86.50 (1F), −88.14 (1F). |
| 74 | 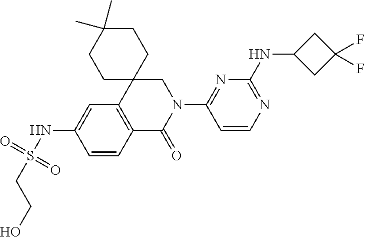 [SMILES: CC1(C)CCC2(CC1)CN(c1ccnc(NC3CC(F)(F)C3)n1)C(=O)c1ccc(NS(=O)(=O)CCO)cc12] | 550.2 | ¹H NMR (400 MHz, DMSO-d₆, ppm): δ 8.21-8.19 (m, 1H), 7.90-7.88 (m, 1H), 7.77-7.62 (m, 1H), 7.62-7.46 (m, 1H), 7.23 (s, 1H), 7.11-7.09 (m, 1H), 4.48-4.11 (m, 3H), 3.76-3.73 (m, 2H), 3.27-3.15 (m, 2H), 3.06-2.86 (m, 2H), 2.80-2.58 (m, 2H), 1.90-1.71 (m, 2H), 1.56-1.24 (m, 6H), 0.97 (d, J = 6.3 Hz, 6H). ¹⁹F NMR (376 MHz, DMSO-d₆, ppm): δ −81.65 (1F), −96.53 (1F). |
| 75 | 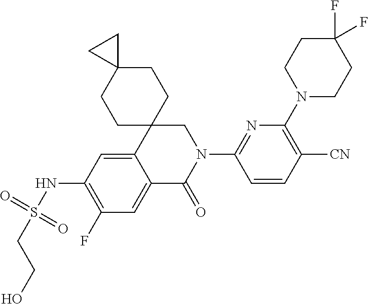 [SMILES: N#Cc1ccc(N2CC3(CCC4(CC4)CC3)c3cc(NS(=O)(=O)CCO)c(F)cc3C2=O)nc1N1CCC(F)(F)CC1] | 604.2 | ¹H NMR (400 MHz, DMSO-d₆, ppm): δ 10.50-9.69 (m, 1H), 8.14-8.06 (m, 1H), 7.86-7.81 (m, 1H), 7.79-7.72 (m, 1H), 7.62-7.55 (m, 1H), 5.34-4.79 (m, 1H), 4.42 (s, 2H), 3.87-3.81 (m, 4H), 3.81-3.76 (m, 2H), 3.37-3.31 (m, 2H), 2.20-2.09 (m, 4H), 1.98-1.88 (m, 2H), 1.85-1.75 (m, 2H), 1.70-1.63 (m, 2H), 0.89-0.82 (m, 2H), 0.29 (s, 4H). ¹⁹F NMR (376 MHz, DMSO-d₆, ppm): δ −73.43 (1F), −94.68 (2F). |
| 76 | 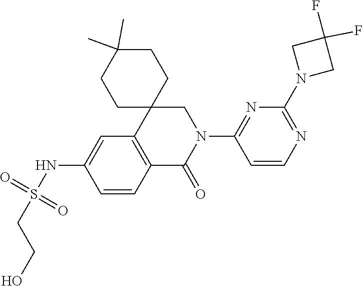 [SMILES: CC1(C)CCC2(CC1)CN(c1ccnc(N3CC(F)(F)C3)n1)C(=O)c1ccc(NS(=O)(=O)CCO)cc12] | 536.4 | ¹HNMR (400 MHz, CDCl₃, ppm): δ 8.25-8.21 (m, 1H), 8.15- 8.08 (m, 1H), 7.79-7.77 (m, 1H), 7.21-6.93 (m, 2H), 4.40- 4.10 (m, 8H), 3.43-3.32 (m, 2H), 1.93-1.81 (m, 2H), 1.52- 1.45 (m, 2H), 1.29-1.08 (m, 4H), 0.93 (s, 6H). ¹⁹F NMR (376 MHz, CDCl₃, ppm): δ −99.97 (2F). |
| 79 | 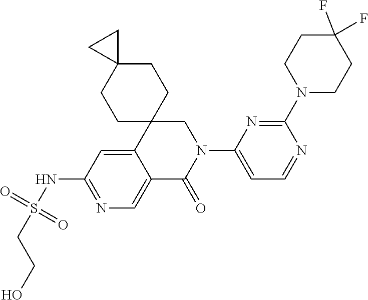 [SMILES: O=C1c2cnc(NS(=O)(=O)CCO)cc2C2(CCC3(CC3)CC2)CN1c1ccnc(N2CCC(F)(F)CC2)n1] | 563.2 | ¹HNMR (400 MHz, DMSO-d6, ppm): δ 8.61 (s, 1H), 8.29- 8.28 (m, 2H), 7.63 (d, J = 5.8 Hz, 1H), 6.60 (s, 1H), 4.38 (s, 2H), 3.99-3.84 (m, 4H), 3.76-3.52 (m, 4H), 2.09-1.89 (m, 6H), 1.81-1.69 (m, 2H), 1.64-1.54 (m, 2H), 0.88- 0.80 (m, 2H), 0.39-0.17 (m, 4H). ¹⁹F NMR (376 MHz, DMSO-d6, ppm): δ−94.23 (2 F). |
| 80 | 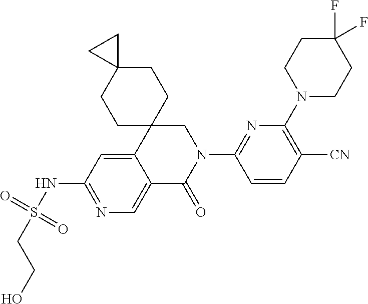 [SMILES: N#Cc1ccc(N2CC3(CCC4(CC4)CC3)c3cc(NS(=O)(=O)CCO)ncc3C2=O)nc1N1CCC(F)(F)CC1] | 587.4 | ¹HNMR (400 MHz, DMSO-d6, ppm): δ 8.67 (s, 1H), 8.10- 8.03 (m, 1H), 7.88-7.81 (m, 1H), 6.83-6.68 (m, 1H), 4.36 (s, 2H), 3.90-3.68 (m, 6H), 3.52-3.43 (m, 2H), 2.21-2.08 (m, 4H), 1.96-1.85 (m, 2H), 1.82-1.71 (m, 2H), 1.69- 1.61 (m, 2H), 0.90-0.79 (m, 2H), 0.38-0.18 (m, 4H). ¹⁹F NMR (376 MHz, DMSO-d6, ppm) δ −94.68 (2F). |
| 81 | 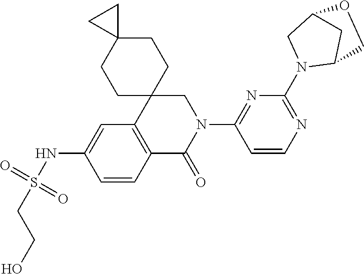 [SMILES: O=C1c2ccc(NS(=O)(=O)CCO)cc2C2(CCC3(CC3)CC2)CN1c1ccnc(N2C[C@H]3C[C@@H]2CO3)n1] | 540.2 | ¹H NMR (400 MHz, DMSO-d6, ppm): δ 8.26 (d, J = 5.7 Hz, 1H), 7.95 (d, J = 8.5 Hz, 1H), 7.61 (d, J = 5.6 Hz, 1H), 7.28 (s, 1H), 7.18 (d, J = 8.3 Hz, 1H), 4.93 (s, 1H), 4.70 (s, 1H), 3.87-3.68 (m, 4H), 3.56-3.41 (m, 2H), 3.30-3.20 (m, 2H), 2.01-1.91 (m, 6H), 1.64-1.61 (m, 2H), 0.87-0.84 (m, 4H), 0.31-0.28 (m, 4H). |
| 82 | 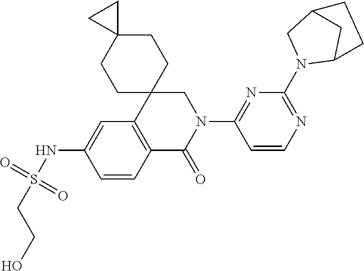 [SMILES: O=C1c2ccc(NS(=O)(=O)CCO)cc2C2(CCC3(CC3)CC2)CN1c1ccnc(N2CC3CCC2C3)n1] | 538.4 | ¹H NMR (400 MHz, DMSO-d6, ppm): δ 8.20 (d, J = 5.7 Hz, 1H), 7.93 (d, J = 8.3 Hz, 1H), 7.53 (s, 1H), 7.25 (s, 1H), 7.16 (d, J = 8.5 Hz, 1H), 4.63 (s, 1H), 3.75 (t, J = 6.5 Hz, 2H), 3.43 (s, 1H), 3.30-3.26 (m, 2H), 3.18 (s, 1H), 2.67-2.64 (m, 1H), 2.14-1.92 (m, 4H), 1.71 (s, 4H), 1.62 (d, J = 9.8 Hz, 4H), 0.85 (t, J = 6.6 Hz, 4H), 0.29 (d, J = 10.6 Hz, 4H). |
| 84 | 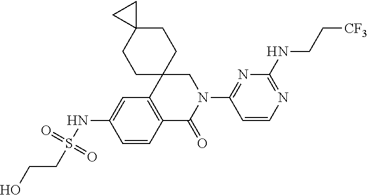 [SMILES: O=C1c2ccc(NS(=O)(=O)CCO)cc2C2(CCC3(CC3)CC2)CN1c1ccnc(NCCC(F)(F)F)n1] | 554.2 | ¹H NMR (400 MHz, DMSO-d6, ppm): δ 10.28 (s, 1H), 8.22 (d, J = 5.6 Hz, 1H), 8.00 (d, J = 8.6 Hz, 1H), 7.56 (s, 1H), 7.35 (s, 2H), 7.24 (dd, J = 8.4, 1.8 Hz, 1H), 4.97 (s, 1H), 4.43 (s, 2H), 3.76 (t, J = 6.4 Hz, 2H), 3.59-3.51 (m, 2H), 3.35- 3.31 (m, 2H), 2.73-2.53 (m, 2H), 1.98-1.92 (m, 2H), 1.79 (t, J = 11.0 Hz, 2H), 1.64 (d, J = 13.2 Hz, 2H), 0.86 (d, J = 13.2 Hz, 2H), 0.28 (s, 4H). ¹⁹F NMR (376 MHz, DMSO-d6, ppm): δ −63.75 (3F). |
| 86 | 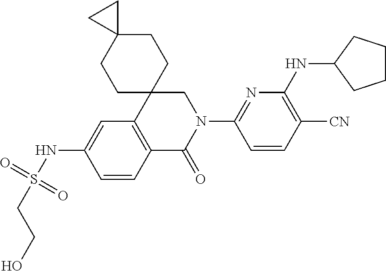 [SMILES: N#Cc1ccc(N2CC3(CCC4(CC4)CC3)c3cc(NS(=O)(=O)CCO)ccc3C2=O)nc1NC1CCCC1] | 550.2 | ¹H NMR (400 MHz, DMSO-d6, ppm): δ 7.95 (d, J = 8.6 Hz, 1H), 7.86 (d, J = 8.6 Hz, 1H), 7.67 (d, J = 8.6 Hz, 1H), 7.26 (d, J = 1.5 Hz, 1H), 7.20-7.13 (m, 1H), 7.01 (d, J = 7.0 Hz, 1H), 4.48 (s, 2H), 4.43-4.33 (m, 1H), 3.75 (t, J = 6.6 Hz, 2H), 3.29 (t, J = 6.6 Hz, 2H), 2.09-1.95 (m, 4H), 1.85-1.69 (m, 4H), 1.68-1.52 (m, 6H), 0.89-0.79 (m, 2H), 0.29 (s, 4H). |
| 87 | 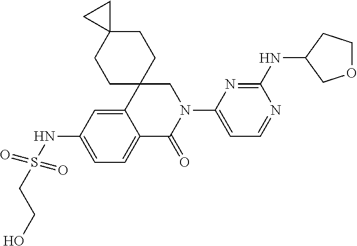 [SMILES: O=C1c2ccc(NS(=O)(=O)CCO)cc2C2(CCC3(CC3)CC2)CN1c1ccnc(NC2CCOC2)n1] | 528.4 | ¹H NMR (400 MHz, DMSO-d6, ppm): δ 11.35-9.22 (m, 1H), 8.21 (d, J = 5.7 Hz, 1H), 7.98 (d, J = 8.6 Hz, 1H), 7.65- 7.38 (m, 2H), 7.33 (s, 1H), 7.23 (d, J = 8.4 Hz, 1H), 4.55- 4.25 (m, 3H), 3.99-3.81 (m, 2H), 3.80-3.69 (m, 3H), 3.67- 3.58 (m, 1H), 3.40-3.29 (m, 3H), 2.24-2.12 (m, 1H), 2.05-1.87 (m, 3H), 1.86-1.70 (m, 2H), 1.69-1.59 (m, 2H), 0.91-0.77 (m, 2H), 0.39-0.20 (m, 4H). |
| 88 | 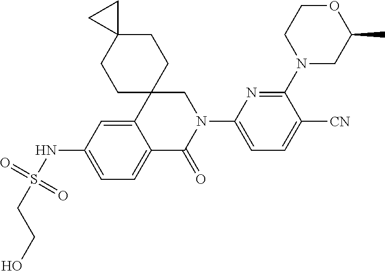 [SMILES: C[C@H]1CN(c2nc(N3CC4(CCC5(CC5)CC4)c4cc(NS(=O)(=O)CCO)ccc4C3=O)ccc2C#N)CCO1] | 556.2 | ¹H NMR (400 MHz, DMSO-d6, ppm): δ 8.00-7.84 (m, 2H), 7.68 (d, J = 8.6 Hz, 1H), 6.87-6.71 (m, 2H), 5.09 (s, 1H), 4.42 (d, J = 13.4 Hz, 1H), 4.33 (d, J = 13.4 Hz, 1H), 4.18 (t, J = 12.4 Hz, 2H), 4.00-3.86 (m, 1H), 3.71-3.60 (m, 4H), 3.25-3.12 (m, 1H), 2.93-2.86 (m, 2H), 2.84-2.81 (m, 1H), 2.10-1.88 (m, 2H), 1.86-1.72 (m, 2H), 1.58 (d, J = 12.6 Hz, 2H), 1.18 (d, J = 6.2 Hz, 3H), 0.80 (d, J = 12.0 Hz, 2H), 0.29 (s, 4H). |
| 89 | 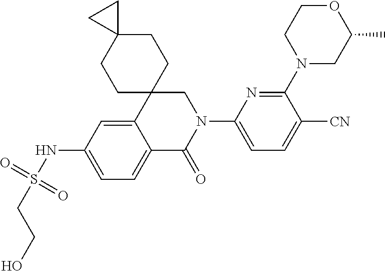 [SMILES: C[C@@H]1CN(c2nc(N3CC4(CCC5(CC5)CC4)c4cc(NS(=O)(=O)CCO)ccc4C3=O)ccc2C#N)CCO1] | 556.2 | ¹H NMR (400 MHz, DMSO-d6, ppm): δ 8.00-7.84 (m, 2H), 7.77 (d, J = 8.6 Hz, 1H), 6.95-6.82 (m, 2H), 4.42 (d, J = 13.4 Hz, 1H), 4.33 (d, J = 13.4 Hz, 1H), 4.18 (t, J = 12.4 Hz, 2H), 4.00-3.86 (m, 1H), 3.75-3.55 (m, 4H), 3.25-3.12 (m, 1H), 3.09-2.95 (m, 2H), 2.90-2.78 (m, 1H), 2.10-1.88 (m, 2H), 1.86-1.72 (m, 2H), 1.58 (d, J = 12.6 Hz, 2H), 1.18 (d, J = 6.2 Hz, 3H), 0.80 (d, J = 12.0 Hz, 2H), 0.29 (s, 4H). |
| 90 | 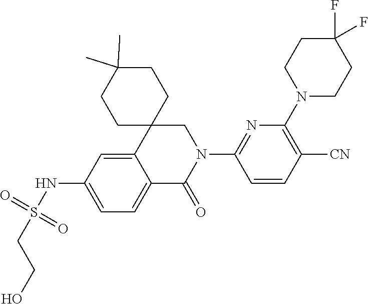 [SMILES: CC1(C)CCC2(CC1)CN(c1ccc(C#N)c(N3CCC(F)(F)CC3)n1)C(=O)c1ccc(NS(=O)(=O)CCO)cc12] | 588.2 | ¹H NMR (400 MHz, DMSO-d6, ppm): δ 8.36 (s, 1H), 8.10- 8.02 (m, 1H), 7.93-7.81 (m, 2H), 7.24-7.18 (m, 1H), 7.12- 7.06 (m, 1H), 4.30 (s, 2H), 3.82-3.70 (m, 6H), 3.24-3.19 (m, 2H), 2.24-2.09 (m, 4H), 1.88-1.75 (m, 2H), 1.55-1.46 (m, 2H), 1.44-1.26 (m, 4H), 1.01-0.90 (m, 6H). ¹⁹F NMR (376 MHz, DMSO-d6, ppm): δ −94.82 (2F). |
| 94 | 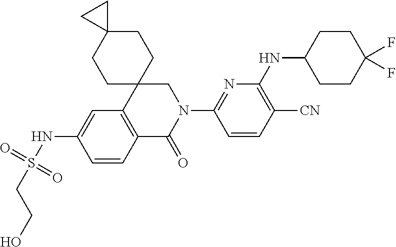 [SMILES: N#Cc1ccc(N2CC3(CCC4(CC4)CC3)c3cc(NS(=O)(=O)CCO)ccc3C2=O)nc1NC1CCC(F)(F)CC1] | 600.6 | ¹H NMR (400 MHz, DMSO-d6, ppm): δ 7.91-7.87 (m, 2H), 7.68 (d, J = 8.6 Hz, 1H), 7.19 (s, 1H), 7.12-7.05 (m, 2H), 4.44 (s, 2H), 4.16 (s, 1H), 3.74 (t, J = 6.4 Hz, 2H), 3.22 (t, J = 6.3 Hz, 2H), 2.15-2.08 (m, 2H), 1.99-1.89 (m, 6H), 1.85-1.76 (m, 4H), 1.64 (d, J = 12.5 Hz, 2H), 0.86 (d, J = 13.0 Hz, 2H), 0.30 (s, 4H). ¹⁹F NMR (376 MHz, DMSO-d6, ppm): δ −91.21 (1F), −99.59 (1F). |
| 95 | 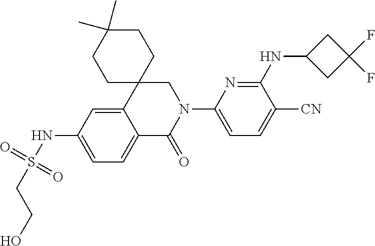 [SMILES: CC1(C)CCC2(CC1)CN(c1ccc(C#N)c(NC3CC(F)(F)C3)n1)C(=O)c1ccc(NS(=O)(=O)CCO)cc12] | 574.5 | ¹H NMR (400 MHz, DMSO-d6, ppm): δ 7.99-7.93 (m, 2H), 7.74 (d, J = 6.0 Hz, 1H), 7.63 (d, J = 8.6 Hz, 1H), 7.38 (s, 1H), 7.24-7.21 (m, 1H), 4.33-4.31 (m, 3H), 3.76 (t, J = 6.5 Hz, 2H), 3.37-3.34 (m, 2H), 3.06-2.79 (m, 4H), 1.82-1.78 (m, 2H), 1.54 (d, J = 13.3 Hz, 2H), 1.39-1.32 (m, 4H), 0.97 (d, J = 14.0 Hz, 6H). ¹⁹F NMR (376 MHz, DMSO-d6, ppm): δ −81.56 (1F), −96.74 (1F). |
| 96 | 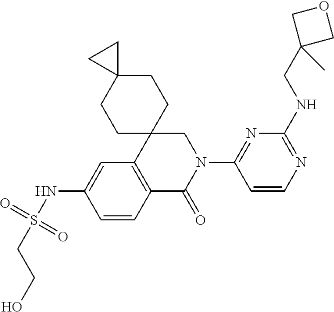 [SMILES: CC1(CNc2nccc(N3CC4(CCC5(CC5)CC4)c4cc(NS(=O)(=O)CCO)ccc4C3=O)n2)COC1] | 542.4 | ¹H NMR (400 MHz, DMSO-d6, ppm): δ 8.20 (d, J = 5.7 Hz, 1H), 8.00-7.93 (m, 1H), 7.62-7.35 (m, 2H), 7.30 (s, 1H), 7.23-7.16 (m, 1H), 4.49 (d, J = 5.7 Hz, 2H), 4.46-4.33 (m, 2H), 4.22 (d, J = 5.6 Hz, 2H), 3.75 (t, J = 6.5 Hz, 2H), 3.55 (d, J = 6.4 Hz, 2H), 3.31 (d, J = 6.5 Hz, 2H), 2.06-1.87 (m, 2H), 1.85-1.71 (m, 2H), 1.69-1.59 (m, 2H), 1.31 (s, 3H), 0.91-0.77 (m, 2H), 0.38-0.21 (m, 4H). |
| 97 | 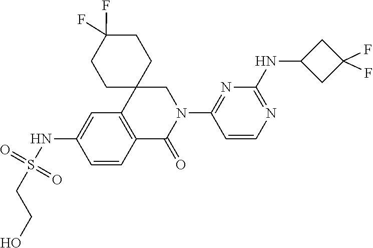 [SMILES: O=C1c2ccc(NS(=O)(=O)CCO)cc2C2(CCC(F)(F)CC2)CN1c1ccnc(NC2CC(F)(F)C2)n1] | 558.2 | ¹H NMR (400 MHz, DMSO-d6, ppm): δ 10.30 (s, 1H), 8.26 (d, J = 5.7 Hz, 1H), 8.02 (d, J = 8.5 Hz, 1H), 7.86-7.68 (m, 1H), 7.68-7.56 (m, 1H), 7.33-7.25 (m, 2H), 4.97 (s, 1H), 4.44 (s, 2H), 4.26-4.12 (m, 1H), 3.84-3.66 (m, 2H), 3.39- 3.34 (m, 2H), 3.06-2.88 (m, 2H), 2.77-2.59 (m, 2H), 2.21-2.07 (m, 3H), 2.06-1.94 (m, 1H), 1.95-1.73 (m, 4H). ¹⁹F NMR (376 MHz, DMSO-d6, ppm) δ −81.92 (1F), −89.93 (1F), −96.33 (1F), −99.59 (1F). |
| 98 | 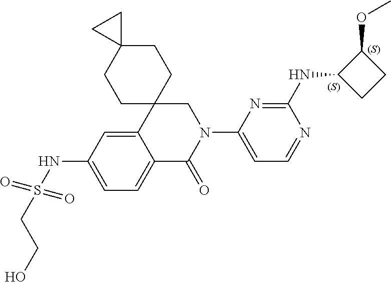 | 542.2 | ¹H NMR (400 MHz, DMSO-d6, ppm): δ 10.22 (s, 1H), 8.19 (s, 1H), 7.99 (d, J = 8.5 Hz, 1H), 7.59 (d, J = 46.5 Hz, 2H), 7.35 (s, 1H), 7.24 (d, J = 8.5 Hz, 1H), 5.82-4.78 (m, 1H), 4.79-4.51 (m, 1H), 4.40-4.21 (m, 2H), 3.91-3.81 (m, 1H), 3.76 (t, J = 6.5 Hz, 2H), 3.42-3.35 (m, 2H), 3.21 (s, 3H), 2.19- 1.94 (m, 4H), 1.88-1.71 (m, 2H), 1.70-1.58 (m, 2H), 1.50- 1.35 (m, 2H), 0.93-0.77 (m, 2H), 0.39-0.21 (m, 4H). |
| 101 | 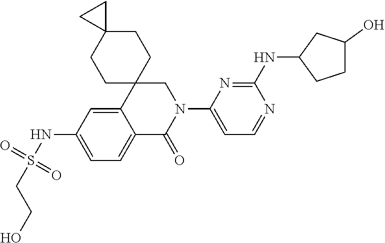 [SMILES: O=C1c2ccc(NS(=O)(=O)CCO)cc2C2(CCC3(CC3)CC2)CN1c1ccnc(NC2CCC(O)C2)n1] | 542.0 | ¹H NMR (400 MHz, DMSO-d6, ppm): δ 10.39-10.00 (m, 1H), 8.18-8.17 (m, 1H), 8.00-7.98 (d, J = 8.6 Hz, 1H), 7.50- 7.53 (m, 1H), 7.34 (s, 1H), 7.25-7.23 (m, 2H), 4.72-4.67 (m, 1H), 4.50 (s, 1H), 4.41-4.36 (m, 1H), 4.31-4.14 (m, 2H), 3.78- 3.75 (m, 2H), 3.37-3.35 (m, 1H), 2.08 (m, 2H), 1.92-1.63 (m, 10H), 1.51-1.46 (m, 2H), 0.88-0.85 (m, 2H), 0.32-0.29 (m, 4H). |
| 102 | 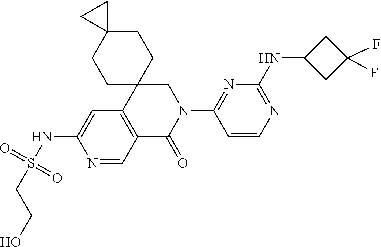 [SMILES: O=C1c2cnc(NS(=O)(=O)CCO)cc2C2(CCC3(CC3)CC2)CN1c1ccnc(NC2CC(F)(F)C2)n1] | 549.2 | ¹H NMR (400 MHz, DMSO-d6, ppm): δ 8.62 (d, J = 6.6 Hz, 1H), 8.27 (s, 1H), 8.19 (d, J = 6.6 Hz, 1H), 7.68 (s, 1H), 7.59 (s, 1H), 6.67 (d, J = 17.4 Hz, 1H), 4.37 (s, 2H), 4.27-4.19 (m, 1H), 3.76-3.68 (m, 2H), 3.10-2.86 (m, 4H), 2.76-2.60 (m, 2H), 1.98-1.93 (m, 2H), 1.81-1.69 (m, 2H), 1.61 (d, J = 13 Hz, 2H), 0.85 (d, J = 13.5 Hz, 2H), 0.32-0.23 (m, 4H). ¹⁹F NMR (376 MHz, DMSO-d6, ppm) δ −81.41 (1 F), −96.66 (1F). |
| 104 | 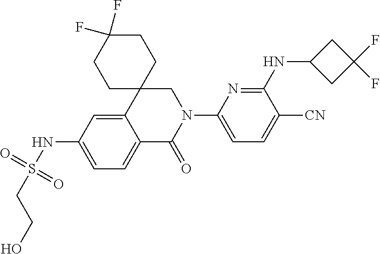 [SMILES: N#Cc1ccc(N2CC3(CCC(F)(F)CC3)c3cc(NS(=O)(=O)CCO)ccc3C2=O)nc1NC1CC(F)(F)C1] | 582.0 | ¹H NMR (400 MHz, DMSO-d6, ppm): δ 10.29 (s, 1H), 8.03- 7.95 (m, 2H), 7.72 (d, J = 6.4 Hz, 1H), 7.65 (d, J = 8.6 Hz, 1H), 7.31-7.26 (m, 2H), 4.96 (s, 1H), 4.40 (s, 2H), 4.36-4.32 (m, 1H), 3.77-3.74 (m, 2H), 3.38-3.33 (m, 2H), 2.95-2.84 (m, 4H), 2.13-2.08 (m, 4H), 1.93-1.86 (m, 4H). ¹⁹F NMR (376 MHz, DMSO-d6, ppm): δ −81.84 (1F), −89.79 (1F), −97.01 (1F), −99.13 (1F). |
| 105 | 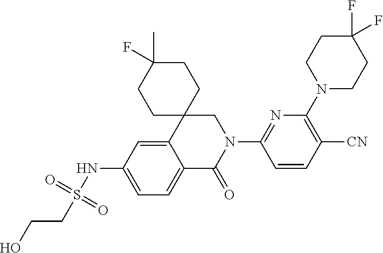 [SMILES: CC1(F)CCC2(CC1)CN(c1ccc(C#N)c(N3CCC(F)(F)CC3)n1)C(=O)c1ccc(NS(=O)(=O)CCO)cc12] | 592.0 | ¹H NMR (400 MHz, DMSO-d6, ppm): δ 8.35 (s, 1H), 8.10- 8.02 (m, 1H), 7.92 (d, J = 8.5 Hz, 1H), 7.85-7.77 (m, 1H), 7.22 (s, 1H), 7.15 (d, J = 8.5 Hz, 1H), 4.32 (s, 2H), 3.76-3.71 (m, 6H), 3.27-3.24 (m, 2H), 2.22-2.05 (m, 4H), 2.00-1.77 (m, 4H), 1.76-1.53 (m, 4H), 1.32 (d, J = 21.2 Hz, 3H). ¹⁹F NMR (376 MHz, DMSO-d6, ppm): δ −94.85 (2F), −150.02 (1F). |
| 106 | 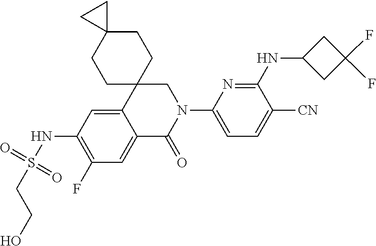 [SMILES: N#Cc1ccc(N2CC3(CCC4(CC4)CC3)c3cc(NS(=O)(=O)CCO)c(F)cc3C2=O)nc1NC1CC(F)(F)C1] | 590.2 | ¹H NMR (400 MHz, DMSO-d6, ppm): δ 7.96 (d, J = 8.6 Hz, 1H), 7.76-7.69 (m, 3H), 7.58 (d, J = 7.5 Hz, 1H), 4.50-4.36 (m, 3H), 3.79 (t, J = 6.4 Hz, 2H), 3.38-3.34 (m, 2H), 3.05- 2.79 (m, 4H), 2.04-1.92 (m, 2H), 1.86-1.74 (m, 2H), 1.71- 1.61 (m, 2H), 0.86 (d, J = 13.2 Hz, 2H), 0.30 (s, 4H). ¹⁹F NMR (376 MHz, DMSO-d6, ppm): δ −81.76 (1F), −97.51 (1F), −126.95 (1F). |
| 107 | 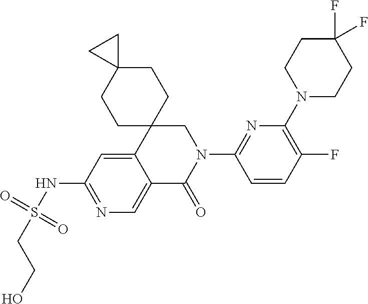 [SMILES: O=C1c2cnc(NS(=O)(=O)CCO)cc2C2(CCC3(CC3)CC2)CN1c1ccc(F)c(N2CCC(F)(F)CC2)n1] | 580.4 | 1.0 FA salt. ¹HNMR (400 MHz, DMSO-d6, ppm): δ 8.56 (s, 1H), 8.35 (s, 2H), 7.64-7.53 (m, 2H), 6.64-6.54 (m, 1H), 4.20 (s, 2H), 3.74-3.68 (m, 2H), 3.65-3.54 (m, 6H), 2.17- 2.04 (m, 4H), 1.97-1.87 (m, 2H), 1.82-1.71 (m, 2H), 1.68-1.61 (m, 2H), 0.89-0.75 (m, 2H), 0.28 (s, 4H). ¹⁹F NMR (376 MHz, DMSO-d6, ppm): δ −94.51 (2 F), −134.32 (1F). |
| 108 | 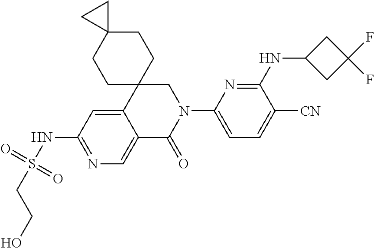 [SMILES: N#Cc1ccc(N2CC3(CCC4(CC4)CC3)c3cc(NS(=O)(=O)CCO)ncc3C2=O)nc1NC1CC(F)(F)C1] | 573.4 | 2.0 TFA salt. ¹H NMR (400 MHz, DMSO-d6, ppm): δ 11.00 (s, 1H), 8.81 (s, 1H), 7.98 (d, J = 8.5 Hz, 1H), 7.77 (d, J = 6.5 Hz, 1H), 7.71 (d, J = 8.5 Hz, 1H), 7.06-7.02 (m, 1H), 4.99- 4.94 (m, 1H), 4.47-4.34 (m, 3H), 3.82-3.65 (m, 4H), 3.10-2.75 (m, 4H), 2.01-1.89 (m, 2H), 1.81-1.64 (m, 4H), 0.94-0.86 (m, 2H), 0.36-0.25 (m, 4H). ¹⁹F NMR (376 MHz, DMSO-d6, ppm): δ −73.54 (6F), −82.03 (1 F), −97.79 (1F). |
| 109 | 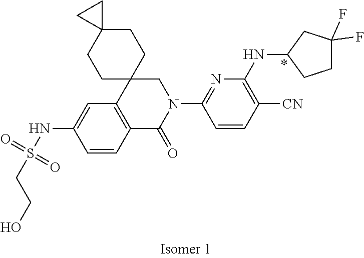 [SMILES: N#Cc1ccc(N2CC3(CCC4(CC4)CC3)c3cc(NS(=O)(=O)CCO)ccc3C2=O)nc1NC1CCC(F)(F)C1] | 586.2 | SFC analysis: 97% ee; retention time: 3.344 min; column: (S, S) Whelk O1, 100 × 4.6 mm I.D., 3 μm, 40% of methanol (0.05% DEA) in CO₂; pressure: 100 bar; flow rate: 2.5 mL/min. ¹H NMR (400 MHz, DMSO-d6, ppm): δ 10.70- 9.60 (m, 1H), 8.00 (d, J = 8.5 Hz, 1H), 7.92 (d, J = 8.6 Hz, 1H), 7.71 (d, J = 8.6 Hz, 1H), 7.42-7.31 (m, 2H), 7.24 (d, J = 8.7 Hz, 1H), 5.10-4.78 (m, 1H), 4.71-4.55 (m, 1H), 4.54- 4.38 (m, 2H), 3.76 (t, J = 6.4 Hz, 2H), 3.35 (t, J = 6.6 Hz, 2H), 2.62-2.52 (m, 1H), 2.40-2.19 (m, 3H), 2.12-1.88 (m, 4H), 1.85-1.72 (m, 2H), 1.70-1.61 (m, 2H), 0.86 (d, J = 12.9 Hz, 2H), 0.29 (s, 4H). ¹⁹F NMR (376 MHz, DMSO- d6, ppm): δ −86.50 (1F), −88.82 (1F). |
| 110 | 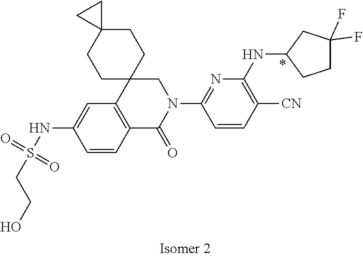 [SMILES: N#Cc1ccc(N2CC3(CCC4(CC4)CC3)c3cc(NS(=O)(=O)CCO)ccc3C2=O)nc1NC1CCC(F)(F)C1] | 586.2 | SFC analysis: 99.08% ee; retention time: 3.624 min; column: (S, S) Whelk O1, 100 × 4.6 mm I.D., 3 μm, 40% of methanol (0.05% DEA) in CO2; pressure: 100 bar; flow rate: 2.5 mL/min. ¹H NMR (400 MHz, DMSO-d6, ppm): δ 10.90- 9.40 (m, 1H), 8.00 (d, J = 8.5 Hz, 1H), 7.92 (d, J = 8.6 Hz, 1H), 7.71 (d, J = 8.6 Hz, 1H), 7.39 (d, J = 7.3 Hz, 1H), 7.34 (s, 1H), 7.24 (d, J = 8.5 Hz, 1H), 5.20-4.75 (m, 1H), 4.69- 4.58 (m, 1H), 4.52-4.40 (m, 2H), 3.76 (t, J = 6.4 Hz, 2H), 3.36 (t, J = 6.4 Hz, 2H), 2.62-2.52 (m, 1H), 2.40-2.18 (m, 3H), 2.10-1.90 (m, 4H), 1.85-1.74 (m, 2H), 1.70-1.61 (m, 2H), 0.86 (d, J = 13.4 Hz, 2H), 0.29 (s, 4H). 19F NMR (376 MHz, DMSO-d6, ppm): δ −86.50 (1F), −88.82 (1F). |
| 111 | 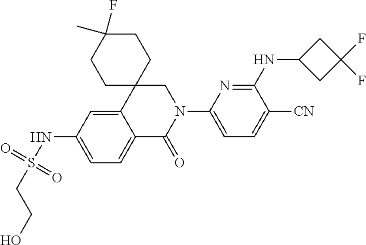 [SMILES: CC1(F)CCC2(CC1)CN(c1ccc(C#N)c(NC3CC(F)(F)C3)n1)C(=O)c1ccc(NS(=O)(=O)CCO)cc12] | 578.2 | ¹H NMR (400 MHz, DMSO-d6, ppm): δ 8.00-7.89 (m, 2H), 7.73 (d, J = 6.1 Hz, 1H), 7.61 (d, J = 8.6 Hz, 1H), 7.30-7.16 (m, 2H), 4.35-4.22 (m, 3H), 3.76-3.73 (m, 2H), 3.30 (d, J = 5.6 Hz, 2H), 2.98-2.80 (m, 4H), 2.03-1.84 (m, 4H), 1.80- 1.55 (m, 4H), 1.35 (d, J = 21.3 Hz, 3H). ¹⁹F NMR (376 MHz, DMSO-d6, ppm): δ −81.65 (1F), −96.79 (1F), −150.29 (1F). |
| 112 | 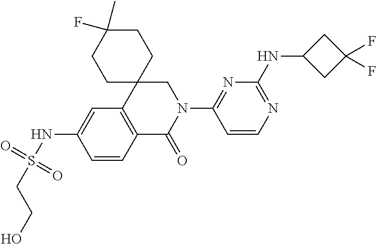 [SMILES: CC1(F)CCC2(CC1)CN(c1ccnc(NC3CC(F)(F)C3)n1)C(=O)c1ccc(NS(=O)(=O)CCO)cc12] | 554.2 | ¹H NMR (400 MHz, DMSO-d6, ppm): δ 8.24 (d, J = 5.7 Hz, 1H), 7.98 (d, J = 8.5 Hz, 1H), 7.73 (s, 1H), 7.59 (d, J = 4.7 Hz, 1H), 7.31-7.17 (m, 2H), 4.35 (s, 2H), 4.19-4.12 (m, 1H), 3.77-3.74 (m, 2H), 3.38-3.30 (m, 2H), 3.04-2.88 (m, 2H), 2.75-2.55 (m, 2H), 1.99-1.78 (m, 4H), 1.76-1.52 (m, 4H), 1.39 (d, J = 21.3 Hz, 3H). ¹⁹F NMR (376 MHz, DMSO-d6, ppm): δ −81.50 (1F), −95.28 (1F), −149.53 (1F). |
| 113 | 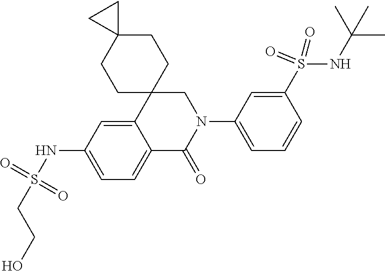 [SMILES: CC(C)(C)NS(=O)(=O)c1cccc(N2CC3(CCC4(CC4)CC3)c3cc(NS(=O)(=O)CCO)ccc3C2=O)c1] | 576.5 | ¹H NMR (400 MHz, DMSO-d6, ppm): δ 10.22 (s, 1H), 7.96- 7.88 (m, 2H), 7.70-7.61 (m, 4H), 7.37 (s, 1H), 7.24 (d, J = 8.5 Hz, 1H), 4.97 (s, 1H), 3.99 (s, 2H), 3.77 (t, J = 6.5 Hz, 2H), 3.36 (d, J = 6.5 Hz, 2H), 1.88-1.72 (m, 6H), 1.10 (s, 9H), 0.86 (d, J = 10.4 Hz, 2H), 0.26 (s, 4H). |
| 115 | 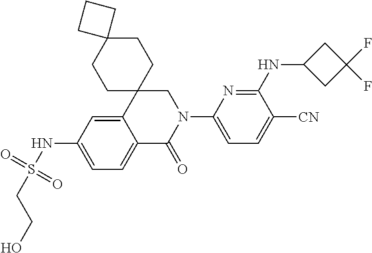 [SMILES: N#Cc1ccc(N2CC3(CCC4(CCC4)CC3)c3cc(NS(=O)(=O)CCO)ccc3C2=O)nc1NC1CC(F)(F)C1] | 586.3 | ¹HNMR (400 MHz, DMSO-d6, ppm): δ 10.26-10.14 (m, 1H), 8.00-7.93 (m, 2H), 7.72-7.70 (m, 2H), 7.29-7.23 (m, 2H), 4.97-4.83 (m, 1H), 4.36-4.28 (m, 3H), 3.77-3.74 (m, 2H), 3.36-3.35 (m, 2H), 2.98-2.78 (m, 4H), 1.87-1.85 (m, 4H), 1.82-1.62 (m, 6H), 1.55-1.54 (m, 4H). ¹⁹F NMR (376 MHz, DMSO-d6, ppm): δ −81.70 (1F), −97.07 (1F). |
| 116 | 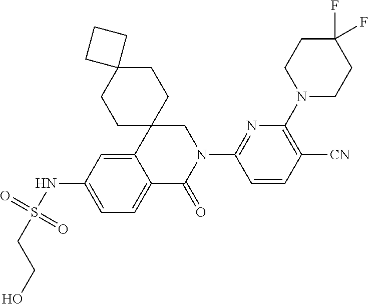 [SMILES: N#Cc1ccc(N2CC3(CCC4(CCC4)CC3)c3cc(NS(=O)(=O)CCO)ccc3C2=O)nc1N1CCC(F)(F)CC1] | 600.3 | ¹HNMR (400 MHz, DMSO-d6, ppm): δ 8.36 (s, 1H), 8.07- 8.05 (m, 1H), 7.90-7.87 (m, 2H), 7.10-7.08 (m, 2H), 4.34 (s, 2H), 3.80-3.72 (m, 6H), 3.21-3.18 (m, 2H), 2.17-2.08 (m, 4H), 1.86-1.80 (m, 4H), 1.70-1.63 (m, 6H), 1.52-1.49 (m, 4H). ¹⁹F NMR (376 MHz, DMSO-d6, ppm): δ −94.76 (2F). |
| 118 | 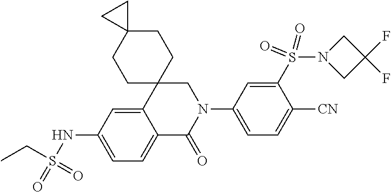 [SMILES: CCS(=O)(=O)Nc1ccc2c(c1)C1(CCC3(CC3)CC1)CN(c1ccc(C#N)c(S(=O)(=O)N3CC(F)(F)C3)c1)C2=O] | 605.4 | ¹HNMR (400 MHz, DMSO-d6, ppm): δ 10.31 (s, 1H), 8.33- 8.24 (m, 2H), 8.01-7.96 (m, 2H), 7.41 (d, J = 1.8 Hz, 1H), 7.26 (dd, J = 8.5, 1.9 Hz, 1H), 4.53-4.46 (m, 4H), 4.09 (s, 2H), 3.26-3.20 (m, 2H), 1.87-1.79 (m, 6H), 1.22 (t, J = 6.8 Hz, 3H), 0.89-0.86 (m, 2H), 0.27 (s, 4H). ¹⁹F NMR (376 MHz, DMSO-d6, ppm): δ −98.87 (2F). |
| 119 | 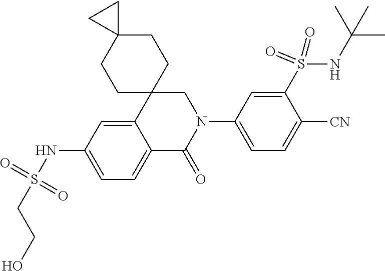 [SMILES: CC(C)(C)NS(=O)(=O)c1cc(N2CC3(CCC4(CC4)CC3)c3cc(NS(=O)(=O)CCO)ccc3C2=O)ccc1C#N] | 601.2 | ¹H NMR (400 MHz, DMSO-d6, ppm): δ 11.10-9.33 (m, 1H), 8.22 (s, 1H), 8.12 (d, J = 8.4 Hz, 1H), 8.07 (s, 1H), 7.97 (d, J = 8.5 Hz, 1H), 7.89-7.83 (m, 1H), 7.37 (s, 1H), 7.29- 7.19 (m, 1H), 5.91-4.41 (m, 1H), 4.06 (s, 2H), 3.77 (t, J = 6.5 Hz, 2H), 3.39-3.35 (m, 2H), 1.88-1.71 (m, 6H), 1.17 (s, 9H), 0.90-0.78 (m, 2H), 0.26 (s, 4H). |
| 120 | 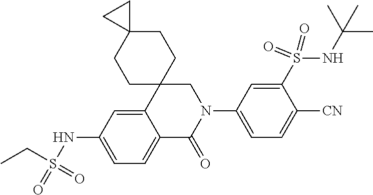 [SMILES: CCS(=O)(=O)Nc1ccc2c(c1)C1(CCC3(CC3)CC1)CN(c1ccc(C#N)c(S(=O)(=O)NC(C)(C)C)c1)C2=O] | 585.2 | ¹H NMR (400 MHz, DMSO-d6, ppm): δ 10.32 (s, 1H), 8.22 (d, J = 2.1 Hz, 1H), 8.12 (d, J = 8.4 Hz, 1H), 8.07 (s, 1H), 7.97 (d, J = 8.5 Hz, 1H), 7.87-7.84 (m, 1H), 7.39 (d, J = 1.8 Hz, 1H), 7.27-7.25 (m, 1H), 4.07 (s, 2H), 3.26-3.20 (m, 2H), 1.86-1.70 (m, 6H), 1.21 (t, J = 7.3 Hz, 3H), 1.17 (s, 9H), 0.91-0.79 (m, 2H), 0.26 (s, 4H). |
| 121 | 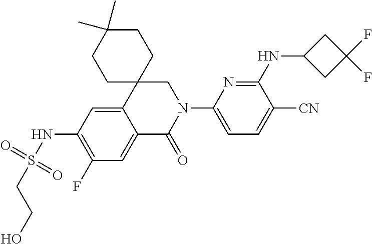 [SMILES: CC1(C)CCC2(CC1)CN(c1ccc(C#N)c(NC3CC(F)(F)C3)n1)C(=O)c1cc(F)c(NS(=O)(=O)CCO)cc12] | 592.6 | ¹H NMR (400 MHz, DMSO-d6, ppm): δ 10.10 (s, 1H), 7.96 (d, J = 8.6 Hz, 1H), 7.80-7.74 (m, 2H), 7.66 (d, J = 7.5 Hz, 1H), 7.61 (d, J = 8.6 Hz, 1H), 5.02 (s, 1H), 4.34 (d, J = 8.2 Hz, 3H), 3.80 (t, J = 6.4 Hz, 2H), 3.39 (t, J = 6.4 Hz, 2H), 3.04-2.81 (m, 4H), 1.90-1.76 (m, 2H), 1.55 (d, J = 13.8 Hz, 2H), 1.38 (d, J = 13.7 Hz, 4H), 0.97 (d, J = 14.9 Hz, 6H). ¹⁹F NMR (376 MHz, DMSO-d6, ppm): δ −81.56 (1F), −96.71 (1F), −126.34 (1F). |
| 123 | 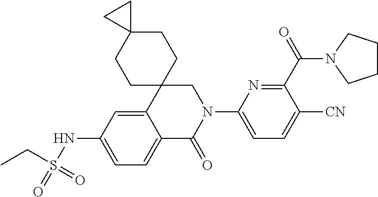 [SMILES: CCS(=O)(=O)Nc1ccc2c(c1)C1(CCC3(CC3)CC1)CN(c1ccc(C#N)c(C(=O)N3CCCC3)n1)C2=O] | 548.4 | ¹H NMR (400 MHz, DMSO-d6, ppm): δ 8.37 (s, 2H), 7.95 (d, J = 8.6 Hz, 1H), 7.26 (s, 1H), 7.16 (d, J = 8.2 Hz, 1H), 4.35 (s, 2H), 3.64-3.52 (m, 4H), 3.14-3.08 (m, 2H), 2.01-1.62 (m, 10H), 1.19 (t, J = 7.3 Hz, 3H), 0.87-0.80 (m, 2H), 0.28 (s, 4H). |
| 124 | 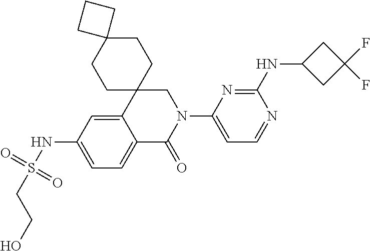 [SMILES: O=C1c2ccc(NS(=O)(=O)CCO)cc2C2(CCC3(CCC3)CC2)CN1c1ccnc(NC2CC(F)(F)C2)n1] | 562.4 | ¹H NMR (400 MHz, DMSO-d6, ppm): δ 10.58-10.01 (m, 1H), 8.23 (d, J = 5.8 Hz, 1H), 7.98 (d, J = 8.5 Hz, 1H), 7.73- 7.62 (m, 2H), 7.28-7.22 (m, 2H), 5.22-4.97 (m, 1H), 4.73- 4.37 (m, 2H), 4.18-4.15 (m, 1H), 3.77-3.74 (m, 2H), 3.36- 3.33 (m, 2H), 3.00-2.89 (m, 2H), 2.73-2.60 (m, 2H), 1.87- 1.80 (m, 4H), 1.74-1.50 (m, 10H) |
| 128 | 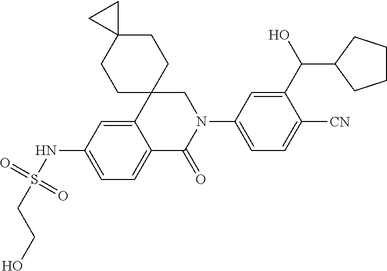 [SMILES: N#Cc1ccc(N2CC3(CCC4(CC4)CC3)c3cc(NS(=O)(=O)CCO)ccc3C2=O)cc1C(O)C1CCCC1] | 564.1 | ¹H NMR (400 MHz, DMSO-d6, ppm): δ 10.28 (s, 1H), 8.51- 8.24 (m, 1H), 8.01-7.91 (m, 1H), 7.88-7.67 (m, 2H), 7.45- 7.32 (m, 1H), 7.26 (d, J = 8.5 Hz, 1H), 6.05 (s, 1H), 5.02 (s, 1H), 4.06 (s, 2H), 3.81-3.68 (m, 2H), 3.38-3.37 (m, 3H), 2.59-2.51 (m, 1H), 2.00-1.70 (m, 7H), 1.69-1.42 (m, 6H), 1.28-1.12 (m, 1H), 0.86 (d, J = 9.6 Hz, 2H), 0.27 (s, 4H). |
| 129 | 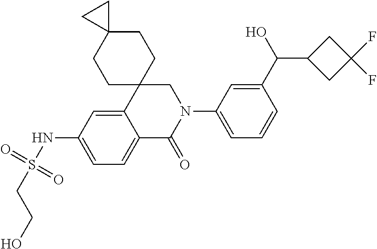 [SMILES: O=C1c2ccc(NS(=O)(=O)CCO)cc2C2(CCC3(CC3)CC2)CN1c1cccc(C(O)C2CC(F)(F)C2)c1] | 561.2 | ¹H NMR (400 MHz, DMSO-d6, ppm): δ 7.58 (d, J = 8.5 Hz, 1H), 7.37-7.26 (m, 2H), 7.25-7.16 (m, 2H), 6.84 (s, 1H), 6.76 (d, J = 8.6 Hz, 1H), 5.61 (s, 1H), 5.11 (s, 1H), 4.56 (s, 1H), 3.82 (s, 2H), 3.70 (t, J = 6.6 Hz, 2H), 2.91 (t, J = 6.6 Hz, 2H), 2.68-2.52 (m, 2H), 2.47-2.35 (m, 3H), 1.86-1.71 (m, 6H), 0.82 (d, J = 10.7 Hz, 2H), 0.26 (s, 4H). ¹⁹F NMR (376 MHz, DMSO-d6, ppm): δ −80.52 (1F), −94.34 (1F). |
| 130 | 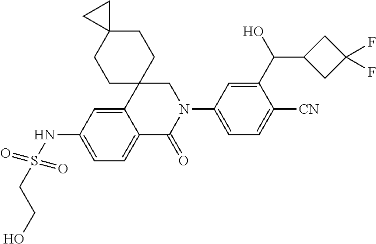 [SMILES: N#Cc1ccc(N2CC3(CCC4(CC4)CC3)c3cc(NS(=O)(=O)CCO)ccc3C2=O)cc1C(O)C1CC(F)(F)C1] | 586.0 | ¹H NMR (400 MHz, DMSO-d6, ppm): δ 8.23-7.96 (m, 1H), 7.92 (d, J = 8.5 Hz, 1H), 7.63-7.51 (m, 2H), 7.36 (s, 1H), 7.22 (d, J = 8.5 Hz, 1H), 5.64 (d, J = 4.1 Hz, 1H), 3.98 (s, 2H), 3.81-3.72 (m, 2H), 3.33-3.22 (m, 3H), 2.85-2.63 (m, 3H), 2.46-2.34 (m, 1H), 2.34-2.18 (m, 1H), 1.92-1.69 (m, 6H), 0.97-0.78 (m, 2H), 0.27 (d, J = 2.3 Hz, 4H). ¹⁹F NMR (376 MHz, DMSO-d6, ppm): δ −80.70 (1F), −94.8 (1F). |
| 131 | 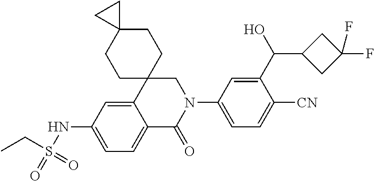 [SMILES: CCS(=O)(=O)Nc1ccc2c(c1)C1(CCC3(CC3)CC1)CN(c1ccc(C#N)c(C(O)C3CC(F)(F)C3)c1)C2=O] | 570.0 | ¹H NMR (400 MHz, DMSO-d6, ppm): δ 10.22 (s, 1H), 8.21- 7.98 (m, 1H), 7.92 (d, J = 8.5 Hz, 1H), 7.57 (d, J = 8.8 Hz, 2H), 7.39 (s, 1H), 7.24 (d, J = 8.5 Hz, 1H), 5.63 (s, 1H), 4.07- 3.94 (m, 2H), 3.34-3.34 (m, 1H), 3.25-3.14 (m, 2H), 2.80- 2.64 (m, 3H), 2.47-2.17 (m, 2H), 1.91-1.69 (m, 6H), 1.22- 1.20 (m, 3H), 0.94-0.79 (m, 2H), 0.34-0.20 (m, 4H). ¹⁹F NMR (376 MHz, DMSO-d6, ppm): δ −80.69 (1F), −95.00 (1F). |
| 134 | 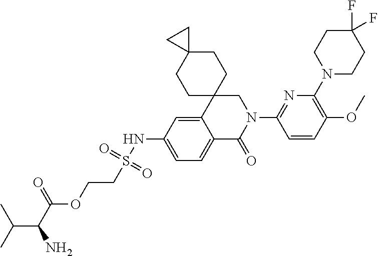 [SMILES: COc1ccc(N2CC3(CCC4(CC4)CC3)c3cc(NS(=O)(=O)CCOC(=O)[C@@H](N)C(C)C)ccc3C2=O)nc1N1CCC(F)(F)CC1] | 690.6 | ¹H NMR (400 MHz, DMSO-d6, ppm): δ 7.95 (d, J = 8.5 Hz, 1H), 7.59 (d, J = 8.5 Hz, 1H), 7.38 (d, J = 8.6 Hz, 1H), 7.30 (s, 1H), 7.21 (d, J = 8.5 Hz, 1H), 4.38 (t, J = 5.7 Hz, 2H), 4.23 (s, 2H), 3.84 (s, 3H), 3.62-3.55 (m, 6H), 3.04 (d, J = 5.2 Hz, 1H), 2.11-1.96 (m, 6H), 1.82-1.68 (m, 5H), 0.87-0.76 (m, 8H), 0.29 (s, 4H). ¹⁹F NMR (376 MHz, DMSO-d6, ppm): δ −94.51 (2F). |
| 135 | 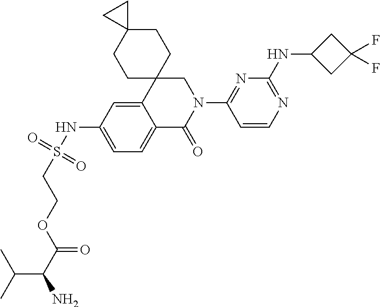 [SMILES: CC(C)[C@H](N)C(=O)OCCS(=O)(=O)Nc1ccc2c(c1)C1(CCC3(CC3)CC1)CN(c1ccnc(NC3CC(F)(F)C3)n1)C2=O] | 647.4 | ¹H NMR (400 MHz, DMSO-d6, ppm): δ 8.23 (d, J = 5.7 Hz, 1H), 7.97 (d, J = 8.6 Hz, 1H), 7.73 (s, 1H), 7.61 (s, 1H), 7.29 (s, 1H), 7.24-7.17 (m, 1H), 4.50-4.19 (m, 5H), 3.60-3.56 (m, 2H), 3.06-3.03 (m, 1H), 3.02-2.91 (m, 2H), 2.76- 2.61 (m, 2H), 2.06-1.99 (m, 2H), 1.85-1.72 (m, 3H), 1.68- 1.59 (m, 2H), 0.88-0.74 (m, 8H), 0.38-0.22 (m, 4H). ¹⁹F NMR (376 MHz, DMSO-d6, ppm): δ −81.82 (1F), −96.71 (1F). |
| 142 | 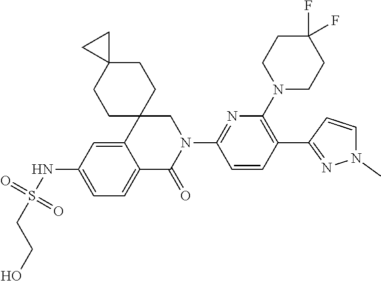 [SMILES: Cn1ccc(-c2ccc(N3CC4(CCC5(CC5)CC4)c4cc(NS(=O)(=O)CCO)ccc4C3=O)nc2N2CCC(F)(F)CC2)n1] | 641.4 | ¹H NMR (400 MHz, DMSO-d6, ppm): δ 10.21 (s, 1H), 8.00- 7.97 (m, 2H), 7.89 (d, J = 8.4 Hz, 1H), 7.77 (s, 1H), 7.35 (s, 1H), 7.26-7.23 (m, 1H), 6.77 (s, 1H), 4.96 (s, 1H), 4.42 (s, 2H), 3.90 (s, 3H), 3.78-3.75 (m, 2H), 3.37-3.24 (m, 6H), 2.12- 2.00 (m, 6H), 1.82-1.68 (m, 4H), 0.86 (d, J = 13.4 Hz, 2H), 0.29 (s, 4H). |
| 144 | 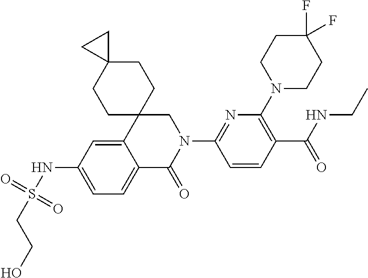 [SMILES: CCNC(=O)c1ccc(N2CC3(CCC4(CC4)CC3)c3cc(NS(=O)(=O)CCO)ccc3C2=O)nc1N1CCC(F)(F)CC1] | 632.5 | ¹H NMR (400 MHz, DMSO-d6, ppm): δ 10.23 (s, 1H), 8.41- 8.38 (m, 1H), 7.99 (d, J = 8.5 Hz, 1H), 7.81-7.76 (m, 2H), 7.35 (s, 1H), 7.26-7.33 (m, 1H), 5.06 (s, 1H), 4.40 (s, 2H), 3.78- 3.75 (m, 2H), 3.46-3.44 (m, 4H), 3.37-3.25 (m, 4H), 2.14- 2.06 (m, 4H), 2.00-1.95 (m, 2H), 1.83-1.67 (m, 4H), 1.14- 1.11 (m, 3H), 0.84 (d, J = 13.2 Hz, 2H), 0.29 (s, 4H). |
| 145 | 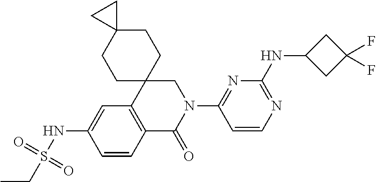 [SMILES: CCS(=O)(=O)Nc1ccc2c(c1)C1(CCC3(CC3)CC1)CN(c1ccnc(NC3CC(F)(F)C3)n1)C2=O] | 532.4 | 1H NMR (400 MHz, DMSO-d6, ppm): δ 10.29 (s, 1H), 8.24 (d, J = 5.7 Hz, 1H), 7.99 (d, J = 8.6 Hz, 1H), 7.74 (s, 1H), 7.61 (s, 1H), 7.36 (s, 1H), 7.25 (d, J = 8.6, 1H), 4.44 (s, 2H), 4.29-4.09 (m, 1H), 3.26-3.15 (m, 2H), 3.06-2.90 (m, 2H), 2.79-2.60 (m, 2H), 2.07-1.88 (m, 2H), 1.87-1.72 (m, 2H), 1.70-1.59 (m, 2H), 1.21 (t, J = 7.3 Hz, 3H), 0.87 (d, J = 13.2 Hz, 2H), 0.37-0.22 (m, 4H). |
| 146 | 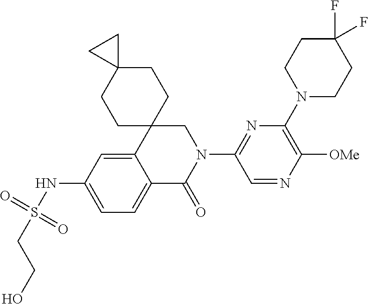 [SMILES: COc1ncc(N2CC3(CCC4(CC4)CC3)c3cc(NS(=O)(=O)CCO)ccc3C2=O)nc1N1CCC(F)(F)CC1] | 592.2 | ¹H NMR (400 MHz, DMSO-d6, ppm): δ 10.19 (s, 1H), 8.28 (s, 1H), 7.96 (d, J = 8.4 Hz, 1H), 7.35 (s, 1H), 7.23 (d, J = 8.4 Hz, 1H), 4.97 (s, 1H), 4.18 (s, 2H), 3.95 (s, 3H), 3.76-3.71 (m, 6H), 2.18-1.83 (m, 8H), 1.82-1.70 (m, 4H), 0.87 (d, J = 12.5 Hz, 2H), 0.30 (s, 4H). |
| 148 | 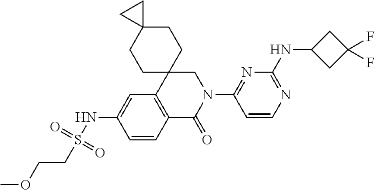 [SMILES: COCCS(=O)(=O)Nc1ccc2c(c1)C1(CCC3(CC3)CC1)CN(c1ccnc(NC3CC(F)(F)C3)n1)C2=O] | 562.4 | ¹H NMR (400 MHz, DMSO-d6, ppm): δ 8.57-8.09 (m, 2H), 7.96 (d, J = 8.6 Hz, 1H), 7.73 (s, 1H), 7.60 (s, 1H), 7.30 (s, 1H), 7.20 (d, J = 8.9 Hz, 1H), 4.59-4.31 (m, 2H), 4.29-4.17 (m, 1H), 3.67 (t, J = 5.9 Hz, 2H), 3.44-3.42 (m, 2H), 3.17 (s, 3H), 3.03-2.94 (m, 2H), 2.75-2.63 (m, 2H), 2.07-1.90 (m, 2H), 1.84-1.72 (m, 2H), 1.68-1.57 (m, 2H), 0.92- 0.81 (m, 2H), 0.28 (s, 4H). |
| 151 | 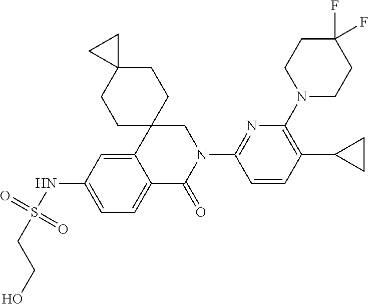 [SMILES: O=C1c2ccc(NS(=O)(=O)CCO)cc2C2(CCC3(CC3)CC2)CN1c1ccc(C2CC2)c(N2CCC(F)(F)CC2)n1] | 601.4 | ¹H NMR (400 MHz, DMSO-d6, ppm): δ 10.20 (s, 1H), 7.96 (d, J = 8.4 Hz, 1H), 7.73 (d, J = 8.3 Hz, 1H), 7.34-7.21 (m, 3H), 4.97 (s, 1H), 4.34 (s, 2H), 3.83-3.66 (m, 2H), 3.45- 3.39 (m, 4H), 3.35-3.32 (m, 2H), 2.24-2.10 (m, 4H), 2.08- 1.93 (m, 3H), 1.86-1.73 (m, 2H), 1.72-1.62 (m, 2H), 1.06-0.96 (m, 2H), 0.90-0.80 (m, 2H), 0.79-0.70 (m, 2H), 0.29 (s, 4H). |
| 153 | 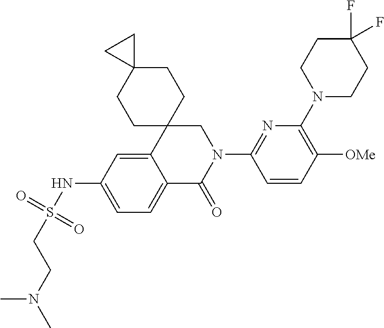 [SMILES: COc1ccc(N2CC3(CCC4(CC4)CC3)c3cc(NS(=O)(=O)CCN(C)C)ccc3C2=O)nc1N1CCC(F)(F)CC1] | 618.2 | ¹H NMR (400 MHz, DMSO-d6, ppm): δ 7.96 (d, J = 8.4 Hz, 1H), 7.58 (d, J = 8.4 Hz, 1H), 7.29-7.40 (m, 2H), 7.25-7.22 (m, 1H), 4.23 (s, 2H), 3.84 (s, 3H), 3.59-3.52 (m, 6H), 3.34- 3.30 (m, 2H), 2.61-2.69 (m, 2H), 2.09 (s, 6H), 1.97-2.04 (m, 2H), 1.72-1.79 (m, 4H), 1.25-1.21 (m, 2H), 0.87 (d, J = 11.1 Hz, 2H), 0.29 (d, J = 4.0 Hz, 4H). |
| 154 | 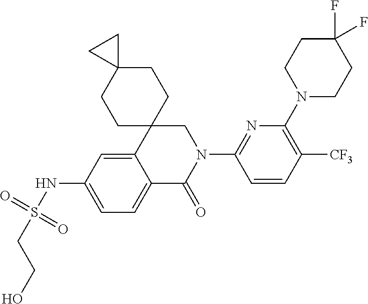 [SMILES: O=C1c2ccc(NS(=O)(=O)CCO)cc2C2(CCC3(CC3)CC2)CN1c1ccc(C(F)(F)F)c(N2CCC(F)(F)CC2)n1] | 628.9 | ¹H NMR (400 MHz, DMSO-d6, ppm): δ 10.29 (brs, 1H), 8.13- 8.04 (m, 2H), 8.01 (d, J = 8.5 Hz, 1H), 7.35 (s, 1H), 7.28- 7.25 (m, 1H), 4.97 (brs, 1H), 4.43 (s, 2H), 3.76 (t, J = 6.4 Hz, 2H), 3.45-3.39 (m, 4H), 3.35-3.33 (m, 2H), 2.21-2.06 (m, 4H), 2.04-1.89 (m, 2H), 1.79 (t, J = 11.6 Hz, 2H), 1.67 (d, J = 12.6 Hz, 2H), 0.86 (d, J = 13.3 Hz, 2H), 0.35-0.23 (m, 4H). |
| 155 | 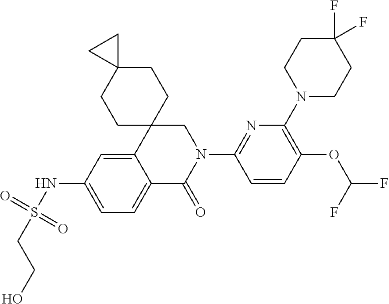 [SMILES: O=C1c2ccc(NS(=O)(=O)CCO)cc2C2(CCC3(CC3)CC2)CN1c1ccc(OC(F)F)c(N2CCC(F)(F)CC2)n1] | 627.4 | ¹H NMR (400 MHz, DMSO-d6, ppm): δ 10.20 (s, 1H), 7.97 (d, J = 8.5 Hz, 1H), 7.72 (d, J = 8.6 Hz, 1H), 7.58 (d, J = 8.5 Hz, 1H), 7.34 (s, 1H), 7.25-7.20 (m, 1H), 7.32-6.93 (t, J = 73.92 Hz, 1H), 4.95 (s, 1H), 4.31 (s, 2H), 3.77-3.74 (m, 2H), 3.62-3.53 (m, 4H), 3.37-3.33 (m 2H), 2.17-2.04 (m, 4H), 2.03-1.92 (m, 2H), 1.85-1.75 (m, 2H), 1.74-1.65 (m, 2H), 0.91-0.83 (m, 2H), 0.35-0.24 (m, 4H). ¹⁹F NMR (376 MHz, DMSO-d6, ppm): δ −80.95 (2F), −94.76 (2F). |
| 156 | 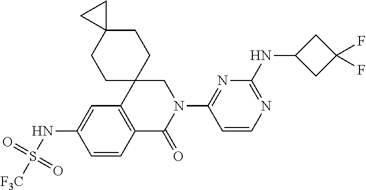 [SMILES: O=C1c2ccc(NS(=O)(=O)C(F)(F)F)cc2C2(CCC3(CC3)CC2)CN1c1ccnc(NC2CC(F)(F)C2)n1] | 572.3 | ¹H NMR (400 MHz, DMSO-d6, ppm): δ 8.21 (d, J = 5.7 Hz, 1H), 7.88 (d, J = 8.5 Hz, 1H), 7.78 (s, 1H), 7.61 (s, 1H), 7.21 (s, 1H), 7.11 (d, J = 8.6 Hz, 1H), 4.42 (s, 2H), 4.29-4.16 (m, 1H), 3.08-2.87 (m, 2H), 2.80-2.60 (m, 2H), 2.07-1.89 (m, 2H), 1.86-1.73 (m, 2H), 1.66-1.56 (m, 2H), 0.84 (d, J = 13.2 Hz, 2H), 0.37-0.22 (m, 4H). ¹⁹F NMR (376 MHz, DMSO-d6, ppm): δ −76.02 (3F), −81.85 (1F), −96.67 (1F). |
| 159 | 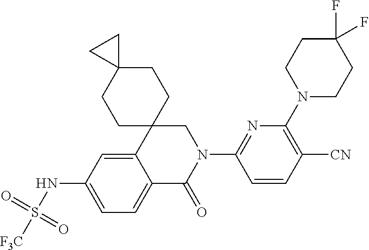 [SMILES: N#Cc1ccc(N2CC3(CCC4(CC4)CC3)c3cc(NS(=O)(=O)C(F)(F)F)ccc3C2=O)nc1N1CCC(F)(F)CC1] | 610.2 | ¹H NMR (400 MHz, DMSO-d6, ppm): δ 8.09 (d, J = 8.6 Hz, 1H), 8.01 (d, J = 8.4 Hz, 1H), 7.85 (d, J = 8.6 Hz, 1H), 7.33 (s, 1H), 7.24 (d, J = 8.4 Hz, 1H), 4.42 (s, 2H), 3.87-3.79 (m, 4H), 2.20-2.09 (m, 4H), 1.98-1.88 (m, 2H), 1.84-1.74 (m, 2H), 1.70-1.63 (m, 2H), 0.90-0.81 (m, 2H), 0.29 (s, 4H). ¹⁹F NMR (376 MHz, DMSO-d6, ppm): δ −75.91 (3F), −94.68 (2F). |
| 160 | 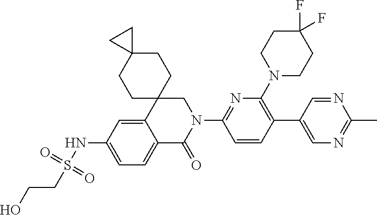 [SMILES: Cc1ncc(-c2ccc(N3CC4(CCC5(CC5)CC4)c4cc(NS(=O)(=O)CCO)ccc4C3=O)nc2N2CCC(F)(F)CC2)cn1] | 653.6 | ¹H NMR (400 MHz, DMSO-d6, ppm): δ 9.00 (s, 2H), 7.99- 7.96 (m, 2H), 7.80 (d, J = 8.3 Hz, 1H), 7.33 (s, 1H), 7.22 (d, J = 8.9 Hz, 1H), 4.43 (s, 2H), 3.78-3.75 (m, 2H), 3.49-3.40 (m, 2H), 3.22-3.15 (m, 4H), 2.67 (s, 3H), 2.06-1.95 (m, 6H), 1.84-1.81 (m, 2H), 1.78-1.69 (m, 2H), 0.90-0.86 (m, 2H), 0.31 (s, 4H). ¹⁹F NMR (376 MHz, DMSO-d6, ppm): δ −95.22 (2F). |
| 161 | 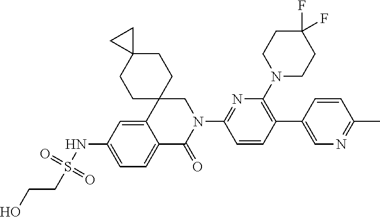 [SMILES: Cc1ccc(-c2ccc(N3CC4(CCC5(CC5)CC4)c4cc(NS(=O)(=O)CCO)ccc4C3=O)nc2N2CCC(F)(F)CC2)cn1] | 652.4 | ¹H NMR (400 MHz, DMSO-d6, ppm): δ 8.70 (s, 1H), 7.99 (d, J = 8.3 Hz, 2H), 7.91 (d, J = 8.2 Hz, 1H), 7.71 (d, J = 8.2 Hz, 1H), 7.35 (d, J = 6.2 Hz, 2H), 7.23 (d, J = 8.3 Hz, 1H), 4.42 (s, 2H), 3.80-3.71 (m, 2H), 3.59-3.50 (m, 2H), 3.24- 3.31 (m, 4H), 2.51 (s, 3H), 2.09-1.88 (m, 6H), 1.84-1.75 (m, 2H), 1.75-1.66 (m, 2H), 0.88 (d, J = 13.4 Hz, 2H), 0.34- 0.25 (m, 4H). ¹⁹F NMR (376 MHz, DMSO-d6, ppm): δ −94.17 (2F). |
| 162 | 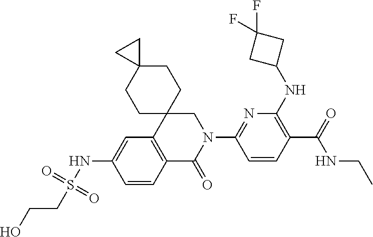 [SMILES: CCNC(=O)c1ccc(N2CC3(CCC4(CC4)CC3)c3cc(NS(=O)(=O)CCO)ccc3C2=O)nc1NC1CC(F)(F)C1] | 618.4 | ¹H NMR (400 MHz, DMSO-d6, ppm): δ 8.94 (d, J = 6.0 Hz, 1H), 8.42 (t, J = 5.2 Hz, 1H), 8.00 (d, J = 8.7 Hz, 1H), 7.85 (d, J = 8.3 Hz, 1H), 7.60 (d, J = 8.6 Hz, 1H), 7.18-6.99 (m, 2H), 4.46-4.32 (m, 3H), 3.74 (t, J = 6.5 Hz, 2H), 3.28-3.23 (m, 2H), 3.22-3.14 (m, 2H), 3.12-3.01 (m, 2H), 2.70-2.56 (m, 2H), 2.05-1.93 (m, 2H), 1.85-1.73 (m, 2H), 1.68-1.59 (m, 2H), 1.12 (t, J = 7.2 Hz, 3H), 0.89-0.79 (m, 2H), 0.29 (s, 4H). ¹⁹F NMR (376 MHz, DMSO-d6, ppm): δ −82.46 (1F), −95.71 (1F) |
| 163 | 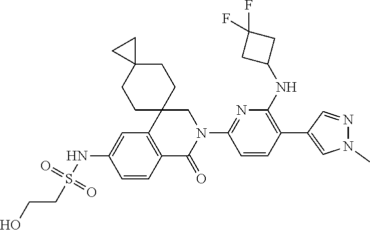 [SMILES: Cn1cc(-c2ccc(N3CC4(CCC5(CC5)CC4)c4cc(NS(=O)(=O)CCO)ccc4C3=O)nc2NC2CC(F)(F)C2)cn1] | 627.2 | ¹H NMR (400 MHz, DMSO-d6, ppm): δ 10.18 (s, 1H), 7.82- 8.10 (m, 2H), 7.70 (s, 1H), 7.56-7.50 (m, 2H), 7.35 (s, 1H), 7.23 (d, J = 8.4 Hz, 1H), 6.28 (d, J = 6.8 Hz, 1H), 4.96 (s, 1H), 4.38 (s, 3H), 3.90 (s, 3H), 3.78-3.75 (m, 2H), 3.39-3.32 (m, 2H), 2.95-2.97 (m, 2H), 2.67-2.78 (m, 2H), 2.08-2.02 (m, 2H), 1.69-1.83 (m, 4H), 0.89 (d, J = 13.2 Hz, 2H), 0.30 (s, 4H). ¹⁹F NMR (376 MHz, DMSO-d6, ppm): δ −81.81 (1F), δ −97.67 (1F). |
| 164 | 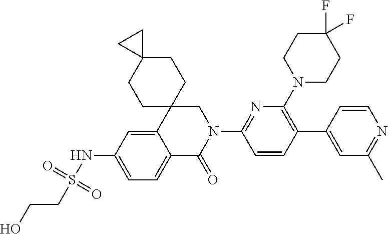 [SMILES: Cc1cc(-c2ccc(N3CC4(CCC5(CC5)CC4)c4cc(NS(=O)(=O)CCO)ccc4C3=O)nc2N2CCC(F)(F)CC2)ccn1] | 652.2 | ¹H NMR (400 MHz, DMSO-d6, ppm): δ 8.49 (d, J = 5.1 Hz, 1H), 7.95 (d, J = 8.4 Hz, 1H), 7.69 (t, J = 8.5 Hz, 2H), 7.56- 7.49 (m, 2H), 6.86 (s, 1H), 6.78 (d, J = 8.6 Hz, 1H), 5.29 (s, 1H), 4.36 (s, 2H), 3.70 (t, J = 6.6 Hz, 2H), 3.27-3.19 (m, 4H), 2.93 (t, J = 6.6 Hz, 2H), 2.52 (s, 3H), 2.06-1.93 (m, 6H), 1.87-1.76 (m, 2H), 1.64 (d, J = 12.7 Hz, 2H), 0.82 (d, J = 13.4 Hz, 2H), 0.28 (s, 4H). ¹⁹F NMR (376 MHz, DMSO- d6, ppm): δ −95.15 (2F) |
| 165 | 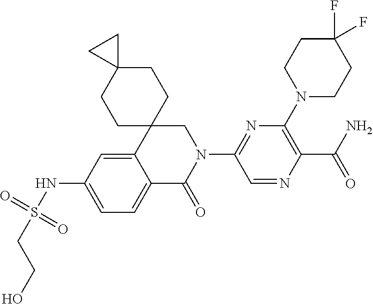 [SMILES: NC(=O)c1ncc(N2CC3(CCC4(CC4)CC3)c3cc(NS(=O)(=O)CCO)ccc3C2=O)nc1N1CCC(F)(F)CC1] | 605.4 | ¹H NMR (400 MHz, DMSO-d6, ppm): δ 8.89 (s, 1H), 7.98 (s, 1H), 7.78 (d, J = 8.6 Hz, 1H), 7.47 (s, 1H), 6.99 (s, 1H), 6.92 (d, J = 8.6 Hz, 1H), 4.29 (s, 2H), 3.72 (t, J = 6.6 Hz, 2H), 3.62-3.58 (m, 4H), 3.05 (t, J = 6.6 Hz, 2H), 2.13-2.07 (m, 4H), 1.97-1.91 (m, 2H), 1.83-1.78 (m, 2H), 1.66-1.63 (m, 2H), 0.83 (d, J = 13.3 Hz, 2H), 0.29 (s, 4H). ¹⁹F NMR (376 MHz, DMSO-d6, ppm): δ −94.71 (2F) |
| 166 | 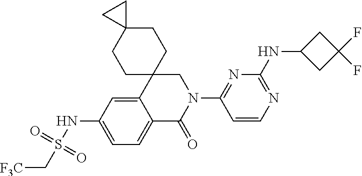 [SMILES: O=C1c2ccc(NS(=O)(=O)CC(F)(F)F)cc2C2(CCC3(CC3)CC2)CN1c1ccnc(NC2CC(F)(F)C2)n1] | 586.2 | ¹H NMR (400 MHz, DMSO-d6, ppm): δ 10.93 (s, 1H), 8.24 (d, J = 5.7 Hz, 1H), 8.01 (d, J = 8.6 Hz, 1H), 7.75 (s, 1H), 7.61 (s, 1H), 7.35 (s, 1H), 7.30-7.25 (m, 1H), 4.77-4.61 (m, 2H), 4.44 (s, 2H), 4.30-4.13 (m, 1H), 3.08-2.91 (m, 2H), 2.79-2.60 (m, 2H), 2.10-1.89 (m, 2H), 1.86-1.74 (m, 2H), 1.69-1.59 (m, 2H), 0.87 (d, J = 13.5 Hz, 2H), 0.37- 0.24 (m, 4H). ¹⁹F NMR (376 MHz, DMSO-d6, ppm): δ −60.92 (3F), −81.82 (1F), −96.73 (1F). |
| 167 | 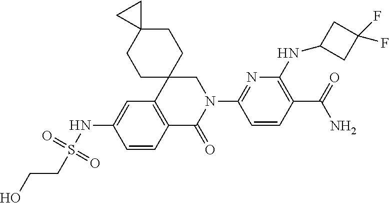 [SMILES: NC(=O)c1ccc(N2CC3(CCC4(CC4)CC3)c3cc(NS(=O)(=O)CCO)ccc3C2=O)nc1NC1CC(F)(F)C1] | 590.2 | ¹H NMR (400 MHz, DMSO-d6, ppm): δ 9.05 (d, J = 5.9 Hz, 1H), 8.05 (d, J = 8.6 Hz, 1H), 7.93 (d, J = 8.5 Hz, 2H), 7.58 (d, J = 8.6 Hz, 1H), 7.33 (s, 1H), 7.24 (s, 1H), 7.14 (d, J = 8.1 Hz, 1H), 4.45-4.30 (m, 3H), 3.75 (t, J = 6.5 Hz, 2H), 3.26- 3.22 (m, 2H), 3.13-3.05 (m, 2H), 2.68-2.60 (m, 2H), 2.10- 1.92 (m, 2H), 1.85-1.75 (m, 2H), 1.70-1.60 (m, 2H), 0.90- 0.83 (m, 2H), 0.45-0.25 (m, 4H). ¹⁹F NMR (376 MHz, DMSO-d6, ppm): δ −82.18 (1F), −95.54 (1F). |
| 168 | 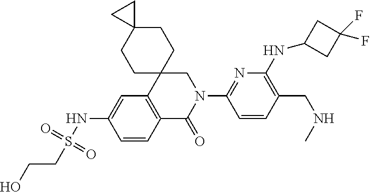 [SMILES: CNCc1ccc(N2CC3(CCC4(CC4)CC3)c3cc(NS(=O)(=O)CCO)ccc3C2=O)nc1NC1CC(F)(F)C1] | 604.2 | ¹H NMR (400 MHz, DMSO-d6, ppm): δ 8.92 (d, J = 5.9 Hz, 1H), 8.43 (d, J = 4.5 Hz, 1H), 7.98 (t, J = 8.1 Hz, 2H), 7.60 (d, J = 8.5 Hz, 1H), 7.31 (s, 1H), 7.21 (d, J = 8.6 Hz, 1H), 4.43-4.38 (m, 3H), 3.78-3.70 (m, 2H), 3.32-3.29 (m, 2H), 3.15-3.03 (m, 2H), 2.76 (d, J = 4.4 Hz, 3H), 2.63-2.45 (m, 2H), 2.12-1.94 (m, 2H), 1.84-1.72 (m, 2H), 1.68-1.52 (m, 2H), 0.92-0.78 (m, 2H), 0.40-0.30 (m, 4H). ¹⁹F NMR (376 MHz, DMSO-d6, ppm): δ −82.25 (1F), −95.44 (1F). |
| 170 | 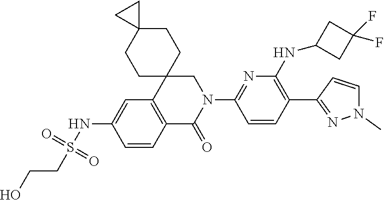 [SMILES: Cn1ccc(-c2ccc(N3CC4(CCC5(CC5)CC4)c4cc(NS(=O)(=O)CCO)ccc4C3=O)nc2NC2CC(F)(F)C2)n1] | 627.2 | ¹H NMR (400 MHz, DMSO-d6, ppm): δ 10.20 (s, 1H), 8.55 (d, J = 5.6 Hz, 1H), 8.00-7.97 (m, 2H), 7.81 (d, J = 2.4 Hz, 1H), 7.60 (d, J = 8.4 Hz, 1H), 7.35 (d, J = 1.8 Hz, 1H), 7.25- 7.23 (m, 1H), 6.80 (d, J = 2.4 Hz, 1H), 4.96 (s, 1H), 4.42 (s, 3H), 3.95 (s, 3H), 3.77 (t, J = 6.2 Hz, 2H), 3.42-3.38 (m, 2H), 3.14-3.21 (m, 2H), 2.67-2.74 (m, 2H), 2.07 (t, J = 11.8 Hz, 2H), 1.69-1.83 (m, 4H), 0.89 (d, J = 13.4 Hz, 2H), 0.32-0.28 (m, 4H). ¹⁹F NMR (376 MHz, DMSO-d6, ppm): δ −81.99 (1F), δ −95.52 (1F). |
| 172 | 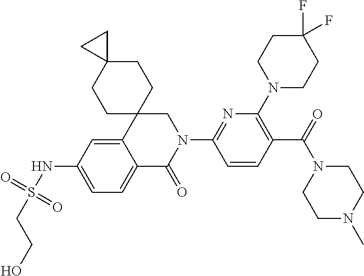 [SMILES: CN1CCN(C(=O)c2ccc(N3CC4(CCC5(CC5)CC4)c4cc(NS(=O)(=O)CCO)ccc4C3=O)nc2N2CCC(F)(F)CC2)CC1] | 687.2 | ¹H NMR (400 MHz, DMSO-d6, ppm): δ 10.20 (s, 1H), 7.99 (d, J = 8.5 Hz, 1H), 7.76 (d, J = 8.2 Hz, 1H), 7.58 (d, J = 8.2 Hz, 1H), 7.35 (s, 1H), 7.27-7.21 (m, 1H), 4.99 (s, 1H), 4.50 (d, J = 13.4 Hz, 1H), 4.27 (d, J = 13.4 Hz, 1H), 3.76 (t, J = 6.5 Hz, 2H), 3.66-3.48 (m, 6H), 3.39-3.35 (m, 2H), 3.32- 3.16 (m, 2H), 2.43-2.30 (m, 2H), 2.29-2.23 (m, 2H), 2.20 (s, 3H), 2.14-1.93 (m, 6H), 1.87-1.68 (m, 4H), 0.94-0.80 (m, 2H), 0.30 (s, 4H). ¹⁹F NMR (376 MHz, DMSO-d6, ppm): δ −94.48 (2F). |
| 174 | 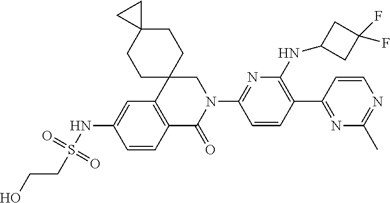 [SMILES: Cc1nccc(-c2ccc(N3CC4(CCC5(CC5)CC4)c4cc(NS(=O)(=O)CCO)ccc4C3=O)nc2NC2CC(F)(F)C2)n1] | 639.2 | ¹H NMR (400 MHz, DMSO-d6, ppm): δ 10.25 (s, 1H), 10.14 (d, J = 5.7 Hz, 1H), 8.69 (d, J = 5.7 Hz, 1H), 8.39 (d, J = 8.9 Hz, 1H), 8.01 (d, J = 8.5 Hz, 1H), 7.90 (d, J = 5.7 Hz, 1H), 7.75 (d, J = 8.7 Hz, 1H), 7.35 (d, J = 1.8 Hz, 1H), 7.28-7.22 (m, 1H), 4.97 (s, 1H), 4.55-4.42 (m, 3H), 3.77 (t, J = 6.4 Hz, 2H), 3.38-3.33 (m, 2H), 3.25-3.12 (m, 2H), 2.78-2.62 (m, 5H), 2.07-1.97 (m, 2H), 1.87-1.75 (m, 2H), 1.74-1.64 (m, 2H), 0.93-0.83 (m, 2H), 0.38-0.24 (m, 4H). ¹⁹F NMR (376 MHz, DMSO-d6, ppm): δ −82.69 (1F), −94.37 (1F). |
| 175 | 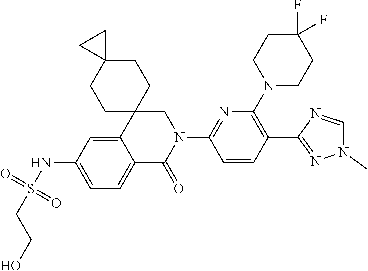 [SMILES: Cn1cnc(-c2ccc(N3CC4(CCC5(CC5)CC4)c4cc(NS(=O)(=O)CCO)ccc4C3=O)nc2N2CCC(F)(F)CC2)n1] | 642.2 | ¹H NMR (400 MHz, DMSO-d6, ppm): δ 10.22 (s, 1H), 8.57 (s, 1H), 8.13 (d, J = 8.4 Hz, 1H), 8.00 (d, J = 8.5 Hz, 1H), 7.88 (d, J = 8.4 Hz, 1H), 7.35 (s, 1H), 7.26-7.24 (m, 1H), 4.97 (s, 1H), 4.44 (s, 2H), 3.94 (s, 3H), 3.77 (t, J = 6.5 Hz, 2H), 3.40-3.34 (m, 6H), 2.16-2.00 (m, 6H), 1.84-1.78 (m, 2H), 1.70 (d, J = 12.8 Hz, 2H), 0.88 (d, J = 13.5 Hz, 2H), 0.31 (s, 4H). ¹⁹F NMR (376 MHz, DMSO-d6, ppm): δ −94.79 (2F). |
| 176 | 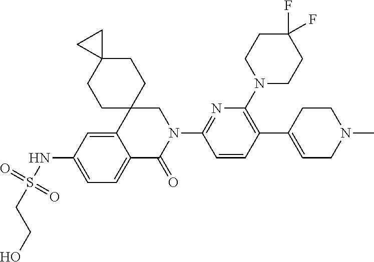 [SMILES: CN1CC=C(c2ccc(N3CC4(CCC5(CC5)CC4)c4cc(NS(=O)(=O)CCO)ccc4C3=O)nc2N2CCC(F)(F)CC2)CC1] | 656.3 | ¹H NMR (400 MHz, DMSO-d6, ppm): δ 7.75 (d, J = 8.2 Hz, 1H), 7.64 (d, J = 8.6 Hz, 1H), 7.41 (d, J = 8.2 Hz, 1H), 6.81 (d, J = 1.9 Hz, 1H), 6.76-6.72 (m, 1H), 5.88 (s, 1H), 5.07 (s, 1H), 4.28 (s, 2H), 3.69 (t, J = 6.6 Hz, 2H), 3.47-3.40 (m, 4H), 3.04-2.98 (m, 2H), 2.90 (t, J = 6.6 Hz, 2H), 2.58-2.52 (m, 2H), 2.47-2.42 (m, 2H), 2.28 (s, 3H), 2.12-1.93 (m, 6H), 1.84-1.73 (m, 2H), 1.61 (d, J = 12.8 Hz, 2H), 0.80 (d, J = 13.2 Hz, 2H), 0.27 (s, 4H). ¹⁹F NMR (376 MHz, DMSO- d6, ppm): δ −94.84 (2F). |
| 178 | 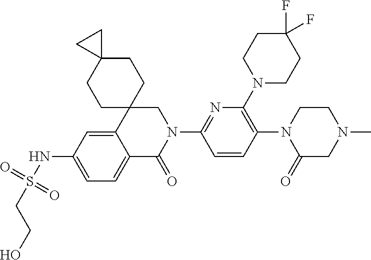 [SMILES: CN1CCN(c2ccc(N3CC4(CCC5(CC5)CC4)c4cc(NS(=O)(=O)CCO)ccc4C3=O)nc2N2CCC(F)(F)CC2)C(=O)C1] | 673.2 | ¹H NMR (400 MHz, DMSO-d6, ppm): δ 7.82 (d, J = 8.5 Hz, 1H), 7.69 (d, J = 8.6 Hz, 1H), 7.47 (d, J = 8.5 Hz, 1H), 6.88 (s, 1H), 6.81 (d, J = 8.6 Hz, 1H), 4.54-4.30 (m, 1H), 4.25- 4.07 (m, 1H), 3.80-3.66 (m, 3H), 3.48-3.35 (m, 4H), 3.28- 3.19 (m, 2H), 3.03-2.90 (m, 3H), 2.88-2.76 (m, 1H), 2.67- 2.55 (m, 1H), 2.30 (s, 3H), 2.22-1.90 (m, 6H), 1.88-1.70 (m, 2H), 1.63 (d, J = 11.7 Hz, 2H), 0.82 (d, J = 12.9 Hz, 2H), 0.28 (s, 4H). ¹⁹F NMR (376 MHz, DMSO-d6, ppm): δ −94.89 (2F). |
| 179 | 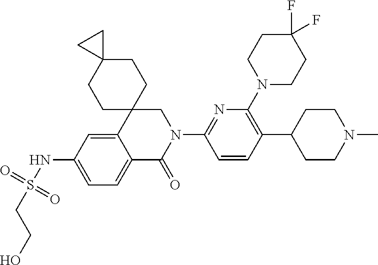 [SMILES: CN1CCC(c2ccc(N3CC4(CCC5(CC5)CC4)c4cc(NS(=O)(=O)CCO)ccc4C3=O)nc2N2CCC(F)(F)CC2)CC1] | 658.4 | ¹H NMR (400 MHz, DMSO-d6, ppm): δ 7.95 (d, J = 8.5 Hz, 1H), 7.89 (d, J = 8.4 Hz, 1H), 7.70 (d, J = 8.4 Hz, 1H), 7.31 (s, 1H), 7.23-7.18 (m, 1H), 4.36 (s, 2H), 3.75 (t, J = 6.5 Hz, 2H), 3.31-3.29 (m, 2H), 3.22-3.14 (m, 4H), 2.91 (d, J = 11.1 Hz, 2H), 2.77-2.66 (m, 1H), 2.23 (s, 3H), 2.21-2.09 (m, 4H), 2.08-1.97 (m, 4H), 1.82-1.74 (m, 2H), 1.71-1.64 (m, 6H), 0.88-0.81 (m, 2H), 0.29 (s, 4H). ¹⁹F NMR (376 MHz, DMSO-d6, ppm): δ −95.16 (2F). |
| 180 | 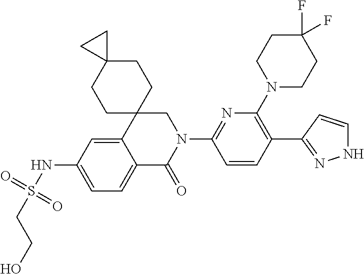 [SMILES: O=C1c2ccc(NS(=O)(=O)CCO)cc2C2(CCC3(CC3)CC2)CN1c1ccc(-c2cc[nH]n2)c(N2CCC(F)(F)CC2)n1] | 627.4 | ¹H NMR (400 MHz, DMSO-d6, ppm): δ 13.08-12.95 (m, 1H), 8.02-7.58 (m, 4H), 7.17 (s, 1H), 7.08 (d, J = 8.4 Hz, 1H), 6.80-6.59 (m, 1H), 4.39 (s, 2H), 3.74 (t, J = 6.5 Hz, 2H), 3.25-3.19 (m, 6H), 2.14-1.98 (m, 6H), 1.85-1.64 (m, 4H), 0.85 (d, J = 13.3 Hz, 2H), 0.29 (s, 4H). ¹⁹F NMR (376 MHz, DMSO-d6, ppm): δ −95.21 (2F) |
| 183 | 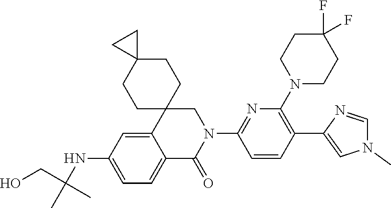 [SMILES: Cn1cnc(-c2ccc(N3CC4(CCC5(CC5)CC4)c4cc(NC(C)(C)CO)ccc4C3=O)nc2N2CCC(F)(F)CC2)c1] | 605.4 | ¹H NMR (400 MHz, CD₃OD, ppm): δ 8.05 (d, J = 8.7 Hz, 1H), 7.99 (d, J = 8.3 Hz, 1H), 7.77 (d, J = 8.3 Hz, 1H), 7.70 (s, 1H), 7.60 (s, 1H), 7.08 (s, 1H), 6.98 (d, J = 8.7 Hz, 1H), 4.41 (s, 2H), 3.87 (s, 2H), 3.80 (s, 3H), 3.37-3.31 (m, 4H), 2.15- 2.08 (m, 4H), 2.04-1.95 (m, 2H), 1.78 (d, J = 12.6 Hz, 2H), 1.39-1.20 (m, 8H), 0.88 (d, J = 14.1 Hz, 2H), 0.34 (s, 4H). ¹⁹F NMR (376 MHz, CD₃OD, ppm): δ −98.53 (2F). |

### Biological Example A: OVCAR3 Cell Viability Assay

OVCAR3 Cells (ATCC, Cat #HTB-161) were seeded at a density of 2000 cells/well in a 96-well clear bottom plate (Greiner, Cat #655098) in 100 μL of complete media (RPMI1640+20% FBS+0.01 mg/mL human insulin). One hundred microliter of complete media was added into the blank well (column 1) for low control. Cells were allowed to adhere to the plate overnight in the incubator at 37° C., 5% CO₂. On the following day, 0.5 μL of serially-diluted compounds were added to the cells (columns 2-10) and incubated for 6 days at 37 C, 5% CO₂ (final 0.5% DMSO concentration); 0.5 μL of DMSO solution was added into the wells (column 11) for high control. Cell viability was detected according to the CellTiter-Glo Luminescent Cell Viability Assay Kit (Promega, Cat #G7573). Luminescence was recorded on the Tecan Spark plate reader. Inhibition rate (IR) of the tested compounds was determined by the following formula: IR (%)=(1−(RLU_{compound}−RLU_{low control})/(RLU_{high control}−RLU_{low control}))×100%. The IC₅₀ value was calculated using the non-linear regression equation: Y=Bottom+(Top−Bottom)/(1+10\hat{ }((Log IC₅₀−X)×HillSlope)), where X is Log of compound concentration, Y is percent inhibition (IR (%)), Top and Bottom are plateaus in same units as Y.

Table 2 below shows IC₅₀ values measured and/or calculated according to this biological example.

TABLE 2
OVCAR3 CTG (IC₅₀) (A: <200 nM; B: 200 nM-2 uM; C: >2 uM)

| Compound No. | OVCAR3 CTG IC₅₀ |
|---|---|
| 1 | A |
| 2 | A |
| 3 | C |
| 4 | A |
| 5 | A |
| 6 | A |
| 8 | A |
| 10 | B |
| 11 | A |
| 12 | B |
| 14 | A |
| 15 | C |
| 16 | C |
| 17 | B |
| 18 | A |
| 19 | A |
| 20 | B |
| 21 | A |
| 24 | B |
| 26 | B |
| 27 | A |
| 28 | A |
| 30 | B |
| 31 | B |
| 33 | B |
| 34 | A |
| 35 | B |
| 36 | C |
| 37 | B |
| 40 | A |
| 41 | B |
| 42 | A |
| 44 | A |
| 46 | B |
| 47 | A |
| 48 | A |
| 49 | A |
| 50 | B |
| 52 | A |
| 54 | A |
| 55 | C |
| 56 | A |
| 57 | C |
| 58 | B |
| 59 | B |
| 60 | B |
| 61 | B |
| 63 | A |
| 64 | B |
| 65 | B |
| 66 | A |
| 67 | B |
| 68 | C |
| 70 | B |
| 71 | A |
| 73 | A |
| 74 | A |
| 77 | B |
| 78 | A |
| 80 | A |
| 82 | B |
| 83 | B |
| 84 | A |
| 88 | B |
| 89 | A |
| 90 | A |
| 93 | A |
| 95 | A |
| 96 | B |
| 97 | B |
| 100 | A |
| 103 | B |
| 104 | A |
| 105 | A |
| 107 | A |
| 111 | B |
| 112 | B |
| 113 | A |
| 114 | A |
| 115 | A |
| 116 | A |
| 117 | B |
| 118 | A |
| 119 | A |
| 120 | A |
| 121 | A |
| 122 | B |
| 124 | A |
| 126 | A |
| 132 | A |
| 133 | A |
| 134 | A |
| 135 | A |
| 136 | A |
| 137 | A |
| 138 | B |
| 139 | A |
| 140 | A |
| 141 | A |
| 142 | A |
| 143 | A |
| 144 | A |
| 145 | A |
| 146 | A |
| 147 | B |
| 148 | B |
| 149 | A |
| 150 | A |
| 151 | A |
| 152 | A |
| 153 | B |
| 154 | A |
| 155 | A |
| 156 | C |
| 157 | A |
| 158 | A |
| 159 | B |
| 160 | A |
| 161 | A |
| 162 | A |
| 163 | A |
| 164 | A |
| 165 | A |
| 166 | C |
| 167 | A |
| 168 | A |
| 169 | A |
| 170 | B |
| 171 | A |
| 172 | B |
| 173 | A |
| 174 | A |
| 175 | A |
| 176 | B |
| 177 | A |
| 178 | A |
| 179 | B |
| 180 | A |
| 181 | A |
| 182 | A |
| Compound A | B |

Compound A: CN115594664A, Example 05.

### Biological Example B: HCC1806 Cell Viability Assay

HCC1806 Cells (ATCC, Cat #CRL-2335) were seeded at a density of 500 cells/well in a 96-well clear bottom plate (Greiner, Cat #655098) in 100 μL of complete media (RPMI1640+10% FBS). One hundred microliter of complete media was added into the blank wells (column 1) for low control. Cells were allowed to adhere to the plate overnight in the incubator at 37° C., 5% CO₂. On the following day, 0.5 μL of serially-diluted compounds were added to the cells (columns 2-10) and incubated for 6 days at 37° C., 5% CO₂ (final 0.5% DMSO concentration); 0.5 μL of DMSO solution was added into the wells (column 11) for high control. Cell viability was detected according to the CellTiter-Glo Luminescent Cell Viability Assay Kit (Promega, Cat #G7573). Luminescence was recorded on the Tecan Spark plate reader. Inhibition rate (IR) of the tested compounds was determined by the following formula: IR (%)=(1−(RLU_{compound}−RLU_{low control})/(RLU_{high control}−RLU_{low control}))×100%. The IC₅₀ value was calculated using the non-linear regression equation: Y=Bottom+(Top−Bottom)/(1+10\hat{ }((Log IC₅₀−X)×HillSlope)), where X is Log of compound concentration, Y is percent inhibition (IR (%)), Top and Bottom are plateaus in same units as Y.

Table 3 below shows IC₅₀ values measured and/or calculated according to this biological example.

TABLE 3
HCC1806 CTG (IC₅₀) (A: <200 nM; B: 200 nM-2 uM; C: >2 uM)

| Compound No. | HCC1806 CTG IC₅₀ |
|---|---|
| 1 | A |
| 3 | C |
| 4 | A |
| 5 | A |
| 6 | A |
| 7 | C |
| 9 | A |
| 11 | B |
| 12 | B |
| 13 | C |
| 14 | A |
| 15 | C |
| 16 | C |
| 26 | B |
| 27 | A |
| 29 | B |
| 31 | B |
| 34 | A |

### Biological Example C: Human Microsomal Clearance Assay

This study aimed to assess the metabolic stability of a compound in human liver microsomes using a microsomal clearance assay.

A mixture containing 100 mM potassium phosphate, pH 7.4, 0.5 mg/mL liver microsomes, 2 mM NADPH, and 1 μM compound were prepared and added to 96-well plate. The plates were then incubated at 37° C. for different time points (0, 5, 15, 30, 45 minutes) and the reaction was stopped with acetonitrile solution containing an internal standard. The samples were then analyzed by LC/MS/MS to determine how much of the compound remained at each time point. The elimination rate constant and half-life were calculated from the data as follows: Elimination rate constant (k)=−slope; Half-life (T_{1/2})=0.693/k.

The in vitro intrinsic clearance, Clint, was calculated from the T_{1/2} as follows: Clᵢₙₜ=(0.693/T_{1/2})×(1/(microsomal protein concentration (0.5 mg/mL)))×Physiological Scaling Factor.

Table 4 below shows in vitro intrinsic clearance values of representative compounds.

TABLE 4
In vitro intrinsic clearance values of representative compounds.

| Compound No. | Clᵢₙₜ (mL/min/kg) | T_{1/2} (min) |
|---|---|---|
| 56 | <12.4 | 142 |
| 95 | 14.6 | 119 |
| 110 | <12.4 | 151 |
| Compound B | 123 | 14.2 |

Compound B: WO2023028564A1, Example 134.

### Biological Example D: Human Plasma Protein Binding Assay

The plasma protein binding of compounds in human plasma was determined using a dialysis method. The dialysis membrane strips were prepared by soaking them in ultra-pure water for about 1 hr at room temperature, followed by separation and soaking in ethanol:water (20:80 v:v) for about 20 min, and a final rinse with ultra-pure water. Prior to use, the membranes were rinsed and soaked for another 20 min in ultra-pure water.

The blank plasma samples were thawed, centrifuged and verified its pH values. Only the plasma with pH between 7.0-8.0 was used in the experiment. The final concentration of compound in the spiked plasma is 1 μM, with final DMSO<=1%. All samples were prepared in triplicates. The time zero (T₀) samples was used for determining the recovery of the compound of interest after dialysis. It was prepared in the same way as other dialysis samples except it was stored at 2-8° C. before LC-MS/MS analysis.

The other spiked plasma samples were loaded onto the dialysis device and incubated at and 37±1° C. with 5% CO₂ for 6 hr. At the end of the dialysis, aliquots of samples from the plasma and buffer sides of the dialysis device were taken and processed for LC-MS/MS analysis.

The % Unbound, % Bound, and % Recovery of the compounds were calculated from the peak area ratios of the analyte and internal standard in the plasma and buffer samples as shown in the following equations:

$$\% Unbound = 100 \times F / T$$

$$\% Bound = 100 - \% Unbound$$

$$\% Recovery = 100 * ( F + T ) / {T}_{0}$$

where [F] is the peak area ratio of analyte/internal standard on the buffer (receiver) side of the membrane; [T] is the peak area ratio of analyte/internal standard on the plasma (donor) side of the membrane; [T₀] is the peak area ratio of analyte/internal standard in the plasma sample at time zero.
   Table 5 below shows human plasma protein binding values of representative compounds.

TABLE 5
Human plasma protein binding values
of representative compounds.

| Compound No. | % Unbound |
|---|---|
| 21 | 0.20 |
| 71 | 0.14 |
| 133 | 0.10 |
| 114 | 1.02 |
| 120 | 1.22 |
| Compound C | 0.012 |

Compound C: WO2021211549A1, Example C14.

### Biological Example E: Mouse PK Assay

This study measured pharmacokinetic profiles of compounds following a single oral dose in male BALB/c mouse. Each tested compound was prepared at 0.1 mg/ml in the formulation (clear solution), and administered at a dose of 5 mg/kg to 3 male mice with body weight ˜18 g (Vital River Laboratory Animal Technology Co., Ltd). Blood samples (0.02 mL) were collected at 0.083, 0.25, 0.5, 1, 2, 4, 6 and 24 h after compound administration.

The collected blood samples were centrifuged to prepare plasma samples, which were then frozen at −70° C. until analysis. The plasma samples were mixed with ACN solution containing internal standards and vortexed for 5 min. The supernatant of the mixture obtained by centrifuging at 14000 rpm at 4° C. for 10 min were injected to LC-MS/MS for plasma concentration determination.

The pharmacokinetic parameters were calculated using standard noncompartmental methods with Phoenix WinNonLin Professional Version 8.1. The calculated parameters included terminal half-life (T_{1/2}), area under the concentration-time curve (AUC), Tₘₐₓ, Cₘₐₓ, and other parameters.

Table 6 below shows mouse PK data of representative compounds.

TABLE 6
Mouse PK data of representative compounds.

| Compound No. | IV MRT (h) | PO T_{1/2} (h) |
|---|---|---|
| 1 | 7.5 | 4.8 |
| 27 | 6.2 | 3.7 |
| 75 | 8 | 6.3 |
| 116 | 6.9 | 4.6 |
| Compound B | 1.9 | 2.3 |

### Biological Example F: In Vitro Cytochrome P450 Inhibition Study

CYP inhibition was evaluated by monitoring corresponding metabolite decrease after incubation with known industry-accepted CYP450 substrates, test articles and microsomes (HLM).

Eight reaction wells with 30 μL of a solution containing 100 mM potassium phosphate, pH 7.4 and 1:3 serial dilutions of the test compound were prepared along with eight wells of 1:3 serial dilutions of Sulfaphenazole (a positive control inhibitor). The assay plates were pre-warmed after 15 μL of probe substrate was added into appropriate wells. Then the reactions were initiated by adding 15 μL of a pre-warmed 8 mM NADPH solution resulting in a final NADPH concentration of 2 mM. The concentrations of test compound ranged from 0.137 μM to 10 μM. A zero time-point control reaction was prepared by adding 135 μL acetonitrile (ACN) containing tolbutamide (200 ng/mL) as internal standard (IS) to 30 μL. of the reaction solution to inactivate the enzymes before adding the probe substrate. A control reaction with no inhibitor was also prepared. After a suitable incubation at 37° C., 10 min, the reactions were terminated by the addition of 135 μL ACN containing IS. The reactions were prepared and analyzed for the metabolite forms of the probe substrate using LC-MS/MS.

Table 7 below shows P450 2C9 inhibition data of representative compounds.

TABLE 7
P450 2C9 inhibition data of representative compounds.

| Compound No. | 2C9, IC₅₀ (uM) |
|---|---|
| 80 | >10 |
| 90 | >10 |
| 116 | 6.7 |
| 133 | >10 |
| 136 | >10 |
| Compound C | 2.5 |

### Biological Example G: In Vitro PXR Activation Study

Prepared the culture medium for DPX2 cells using DPX2 culturing medium supplemented with 10% FBS. Cultivated DPX2 cells in T-75 flasks in a cell culture incubator set at 37 C, 5% CO₂, 95% relative humidity. Allowed cells to reach 80-90% confluence before detaching and splitting. Rinsed cultivated cells in T-75 flasks with 5 mL PBS. Aspirate off, add 1.5 mL trypsin, and incubated at 37° C. for approximately 5 minutes or until the cells detach and float. Inactivated trypsin by adding excess serum containing medium. Removed cell suspension to a conical tube and pellet cells by centrifugation at 150×g for 5 minutes. Resuspended cells in seeding medium at a density of 3.2×10⁵ cells/mL. Transferred 25 μL to each well of 384-well cell culture plate. Placed plate(s) in incubator and incubate at 37° C. for 24 hours.

Prepared stock solutions of test compounds and inducers in DMSO. Final concentration of DMSO in the treatment group should be 0.1%. Removed the plates from the incubator and directly add 25 μL of the negative control, inducers, or test article solutions. Returned plates to the incubator for 48 hours. Check cell morphology and monolayer integrity prior to initiating the experiment with the substrates to ensure that the monolayers are of acceptable quality for the study. After 48 h of treatment, the cultures were ready for the determination of quantitation of PXR activation.

The fold-activation mRNA level was determined by the equation:

$$Fold of activation = \frac{{RLU}_{test} / {RFU}_{test}}{{RLU}_{vehicle} / {RFU}_{vehicle}}$$

Table 8 below shows PXR data of representative compounds.

TABLE 8
PXR data of representative compounds.

| Compound # | Concentration (uM) | Induction Fold |
|---|---|---|
| 27 | 30 | 1.8x |
| 133 | 30 | 1.7x |
| Compound C | 30 | 5.8x |
| Compound B | 30 | 5.7x |

### Biological Example H: OVCAR3 In Vivo Mouse Xenograft Model Study

In vivo efficacy studies were performed in human ovarian cancer cell OVCAR3 (TP53^{MUT}, CCNE1^{AMP}) mouse xenograft model to evaluate anti-tumor activity of KIF18A inhibitors. Female BALB/c Nude mice (6-8 weeks) were purchased from Beijing Vital River Laboratory Animal Technology Co., Ltd. The OVCAR3 cells growing in an exponential growth phase were harvested for tumor inoculation. OVCAR3 cells (1×10⁷ cells mixed 1:1 with Matrigel) were subcutaneously implanted into the right flanks of BALB/c nude mice. After tumors size reached 100-200 mm, mice were treated orally once per day (PO, QD) with KIF18A inhibitors or vehicle control. Tumor volume and body weight of mice were recorded twice per week. Tumor sizes were measured in two dimensions using a caliper and expressed in mm³ using the formula: Volume=0.5 a×b² where a and b are the longest and shortest diameters of the tumor, respectively. Tumor growth inhibition (TGI) was calculated according to the following equation: TGI (%)=(1-(TV_{Treatment/Dn}−TV_{Treatment/D0})/(TV_{Control/Dn}−TV_{Control/D0}))×100%, where the Dn is the final tumor volume and DO is starting tumor volume prior to treatment. Tumor regression was calculated with the following equation: Regression (%)=−(TV_{Treatment/Dn}−TV_{Treatment/D0})/TV_{Treatment/D0}×100%.

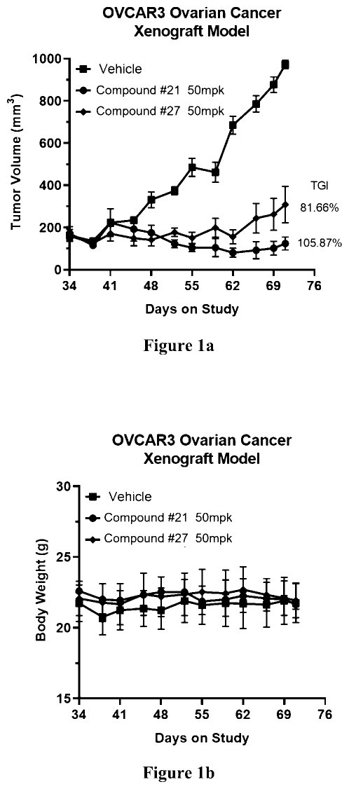

Exemplary compound has been tested in this model and were found to inhibit tumor growth (see FIGS. 1a and 1b).

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 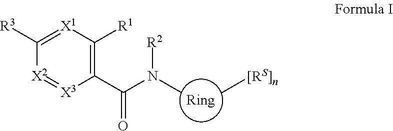
wherein:
R¹ are an optionally substituted C₃₋₁₀ carbocyclyl, an optionally substituted 4-10 membered heterocyclyl, an optionally substituted aryl, or an optionally substituted heteroaryl;
R² is hydrogen, an optionally substituted C₁₋₆ alkyl, an optionally substituted C₁₋₆ heteroalkyl, or a nitrogen protecting group;
or
R¹ and R², together with the intervening C, C, C, and N atoms, are joined to form a 5-14 membered heterocyclyl, which is optionally substituted;
R³ is R^{A}, OR^{A}, SR^{A}, S(O)R^{A}, S(O)₂R^{A}, COR^{A}, COOR^{A}, CN, NHR^{A}, CONHR^{A}, S(O)₂NHR^{A}, S(O)(NH)R^{A}, NHCOR^{A}, NHS(O)₂R^{A}, or NO₂, wherein R^{A} is independently hydrogen, halogen, CN, OH, an optionally substituted C₁₋₆ alkyl, an optionally substituted C₂₋₆ alkenyl, an optionally substituted C₂₋₆ alkynyl, an optionally substituted C₁₋₆ heteroalkyl, an optionally substituted C₁₋₆ haloalkyl, an optionally substituted C₃0.10 carbocyclyl, an optionally substituted 4-10 membered heterocyclyl, an optionally substituted aryl, or an optionally substituted heteroaryl;
X¹ is N or CR⁴, wherein R⁴ is H, F, Cl, OH, NH₂, CN, CD₃, CF₃, an optionally substituted C₁₋₄ alkyl, an optionally substituted C₁₋₄ heteroalkyl, or an optionally substituted C₃₋₆ cycloalkyl;
X² is N or CR⁵, wherein R⁵ is H, F, Cl, OH, NH₂, CN, CD₃, CF₃, an optionally substituted C₁₋₄ alkyl, an optionally substituted C₁₋₄ heteroalkyl, or an optionally substituted C₃₋₆ cycloalkyl;
X³ is N or CR⁶, wherein R⁶ is H, F, Cl, OH, NH₂, CN, CD₃, CF₃, an optionally substituted C₁₋₄ alkyl, an optionally substituted C₁₋₄ heteroalkyl, or an optionally substituted C₃₋₆ cycloalkyl;
or
R⁵ and R⁶, together with the intervening C and C atoms, are joined to form a 5-8 membered heteroaryl, which is optionally substituted; 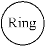
is an optionally substituted C₃₋₁₀ carbocyclic ring, an optionally substituted 4-10 membered heterocyclic ring, an optionally substituted aromatic ring containing 0, 1, 2, or 3 heteroatoms independently selected from N, O, and S;
R^{S} at each occurrence is independently R^{T}, OR^{T}, SR^{T}, NHR^{T}, COR^{T}, COOR^{T}, CONHR^{T}, NHCOR^{T}, CN, or NO₂, wherein R^{T} is independently hydrogen, halogen (e.g. F, Cl, or Br), CN, OH, an optionally substituted C₁₋₆ alkyl, an optionally substituted C₂₋₆ alkenyl, an optionally substituted C₂₋₆ alkynyl, an optionally substituted C₁₋₆ heteroalkyl, an optionally substituted C₁₋₆ haloalkyl, an optionally substituted C₃₋₁₀ carbocyclyl, an optionally substituted 4-10 membered heterocyclyl, an optionally substituted aryl, or an optionally substituted heteroaryl; and
n is 0, 1, 2, or 3.

2. The compound of claim 1, or a pharmaceutically acceptable salt thereof, characterized by having Formula I-1 or I-2: 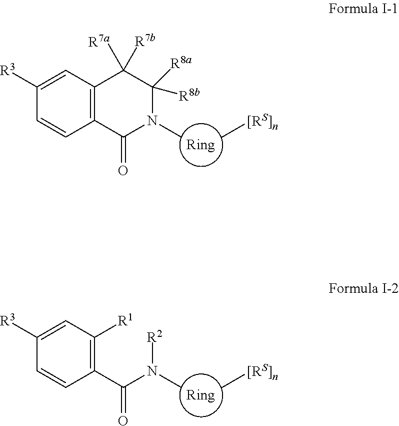
wherein R^{7a} and R^{7b} in Formula I-1 are each independently hydrogen, halogen, CN, OH, an optionally substituted C₁₋₆ alkyl, an optionally substituted C₂₋₆ alkenyl, an optionally substituted C₂₋₆ alkynyl, an optionally substituted C₁₋₆ heteroalkyl, or an optionally substituted C₁₋₆ haloalkyl; or
R^{7a} and R^{7b}, together with the intervening C atom, are joined to form an optionally substituted C₃₋₁₀ carbocyclyl, or an optionally substituted 4-10 membered heterocyclyl;
wherein R^{8a} and R^{8b} in Formula I-1 are each independently H, F, Cl, CN, OH, C₁₋₄ alkyl (e.g., methyl, ethyl, n-propyl, isopropyl), C₁₋₄haloalkyl (e.g., fluoromethyl, difluoromethyl, trifluoromethyl, etc.), C₁₋₄ alkoxy (e.g., methoxy, ethoxy, isopropoxy, etc.), or C₁₋₄ haloalkoxy (e.g., CF₃O—, CF₃CH₂O—, etc.);
wherein R¹ and R² in Formula I-2 are not joined to form a 5-14 membered heterocyclyl, which is optionally substituted.

3. The compound of any one of claims 1-2, or a pharmaceutically acceptable salt thereof, characterized by having Formula I-1-A: 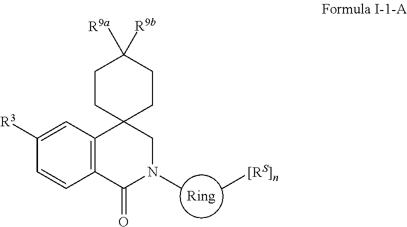
wherein R^{9a} and R^{9b} are each independently hydrogen, halogen, an optionally substituted C₁₋₆ alkyl, or an optionally substituted C₁₋₆ heteroalkyl; or
R^{9a} and R^{9b}, together with the intervening C atom, are joined to form an optionally substituted C₃₋₆ carbocyclyl, or an optionally substituted 4-6 membered heterocyclyl, or R^{9a} and R^{9b} are joined to form ═CF₂, ═CCl₂, or ═C(CH₃)₂.

4. The compound of any one of claims 1-3, or a pharmaceutically acceptable salt thereof, characterized by having Formula I-1-A-a, I-1-A-b, I-1-A-c, I-1-A-d, I-1-A-e, I-1-A-f, I-1-A-g, or I-1-A-h: 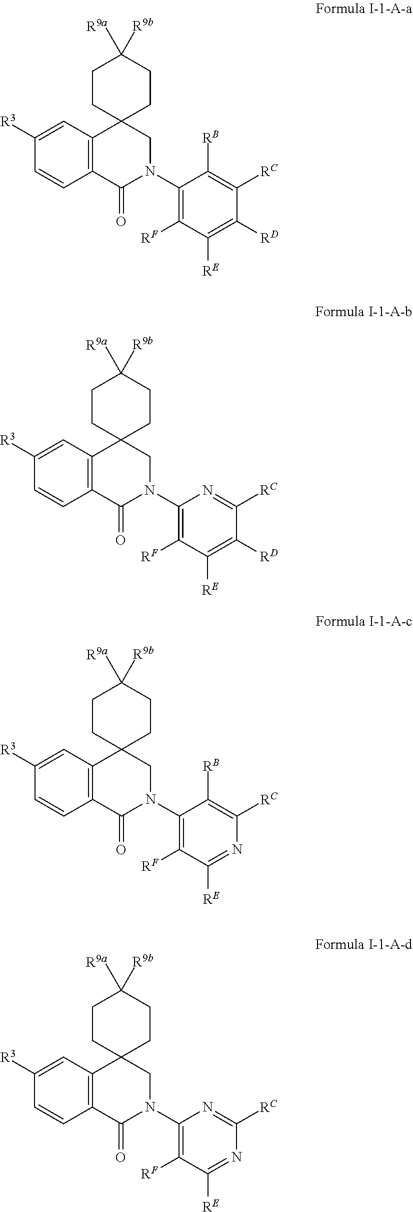 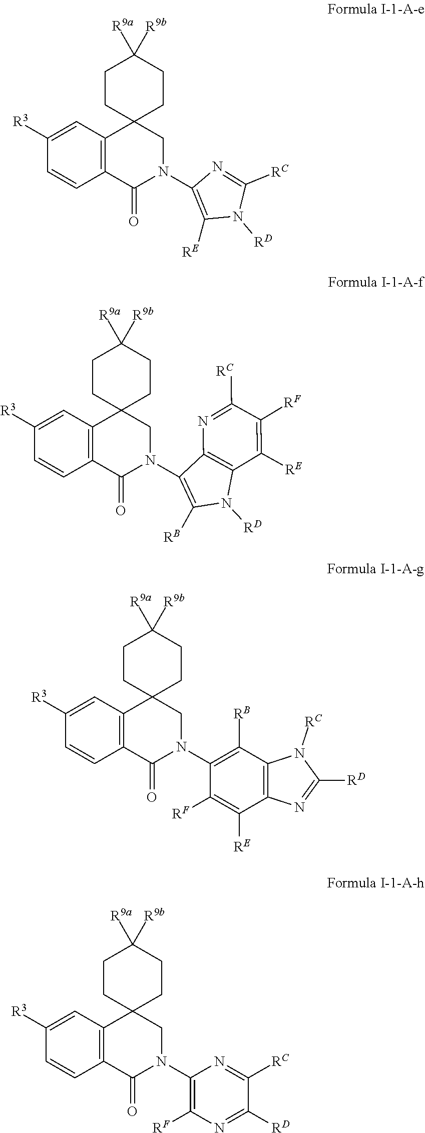
wherein:
R^{B}, R^{E}, and R^{F} are each independently hydrogen, halogen, CN, OH, NH₂, an optionally substituted C₁₋₆ alkyl, an optionally substituted C₁₋₄ heteroalkyl, or an optionally substituted C₃₋₆ cycloalkyl;
R^{C} is -L₁-L₂, wherein Li is null, —O—, —NH—, —N(C₁₋₆ alkyl)-, —CH₂—, —CH(C₁₋₆ alkyl)-, —CH(OH)—, —C(O)—, —S—, —S(O)₂—, or —S(O)₂NH—, and L₂ is an optionally substituted C₁₋₆ alkyl, an optionally substituted C₁₋₆ heteroalkyl, an optionally substituted C₁₋₆ haloalkyl, an optionally substituted C₃₋₁₀ carbocyclyl, or an optionally substituted 4-10 membered heterocyclyl, preferably L₂ is C₄₋₈ carbocyclyl, which is monocyclic, or includes a spiro, bridged, and/or fused ring, and is unsubstituted or substituted with one or more groups independently selected from F, OH, and C₁₋₆ alkyl, or C₄₋₁₀ heterocyclyl, which is monocyclic, or includes a spiro, bridged, and/or fused ring, and is unsubstituted or substituted with one or more groups independently selected from F, OH, and C₁₋₆ alkyl and containing one, two or three heteroatoms independently selected from N, O and S; R^{D} is hydrogen, halogen, CN, OH, NH₂, C₁₋₆ alkyl, O(C₁₋₆ alkyl), O(CH₂)₁₋₃O(C₁₋₆ alkyl), O(CH₂)₁₋₃NH(C₁₋₆ alkyl), O(CH₂)₁₋₃N(C₁₋₆ alkyl)(C₁₋₆ alkyl), NH(C₁₋₆ alkyl), N(C₁₋₆)alkyl)(C₁₋₆ alkyl), C(O)NH₂, C(O)NH(C₁₋₆ alkyl), C(O)N(C₁₋₆ alkyl)(C₁₋₆ alkyl), an optionally substituted C₃₋₆ cycloalkyl, an optionally substituted 4-6 membered heterocyclyl, an optionally substituted phenyl, or an optionally substituted 5-6 membered heteroaryl, wherein C₁₋₆ alkyl is optionally substituted; or
R^{D} is C(O)-(5-6 membered heterocyclyl), wherein the heterocyclyl is optionally substituted.

5. The compound of any one of claims 1-4, or a pharmaceutically acceptable salt thereof, wherein, in Formula I, I-1, I-2, I-1-A, I-1-A-a, I-1-A-b, I-1-A-c, I-1-A-d, I-1-A-e, I-1-A-f, I-1-A-g, or I-1-A-h, R³ is —CONHR^{A}, —S(O)₂NHR^{A}, —NHCOR^{A}, or —NHS(O)₂R^{A}, wherein R^{A} is a C₂₋₄ alkyl optionally substituted with F, OH, or NH₂; preferably, R³ is 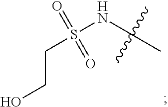 [SMILES: *NS(=O)(=O)CCO]
or
R³ is —NHS(O)₂R^{A}, wherein R^{A} is —CH₂CH₃, —CH₂CH₂OCH₃, —CH₂CH₂N(CH₃)₂, or CH₂CH₂OC(O)CH(NH₂)CH(CH₃)₂; or
R³ is —NHS(O)₂R^{A}, wherein R^{A} is a C₁₋₄ alkyl optionally substituted with F, such as CF₃ or CH₂CF₃; preferably, R³ is 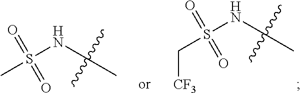
or
R³ is —NHS(O)₂R^{A}, wherein R^{A} is NH—C₁₋₄ alkyl or 4-membered heterocyclyl, which is optionally substituted with F and/or OH, such as NH—CH₃ or 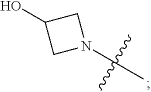 [SMILES: *N1CC(O)C1]
preferably, R³ is 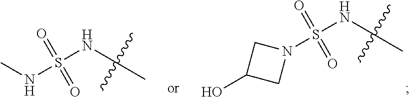
or
R³ is S(O)₂R^{A}, wherein R^{A} is C₁₋₄ alkyl optionally substituted with F and/or OH, such as CH₃; preferably, R³ is 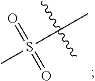 [SMILES: *S(C)(=O)=O]
or
R³ is NHR^{A}, wherein R^{A} is C₁₋₆ alkyl optionally substituted with F and/or OH, such as C(CH₃)₂CH₂OH; preferably, R³ is

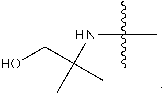 [SMILES: *NC(C)(C)CO]

6. The compound of any one of claims 1-4, or a pharmaceutically acceptable salt thereof, wherein in Formula I, I-1, I-2, I-1-A, I-1-A-a, I-1-A-b, I-1-A-c, I-1-A-d, I-1-A-e, I-1-A-f, I-1-A-g, or I-1-A-h, R³ is —NHS(O)₂R^{A}, wherein R^{A} is —CH₂CH₂OH, —CH₂CH₃, or —CH₂CH₂N(CH₃)₂.

7. The compound of any one of claims 1-4, or a pharmaceutically acceptable salt thereof, wherein in Formula I, I-1, I-2, I-1-A, I-1-A-a, I-1-A-b, I-1-A-c, I-1-A-d, I-1-A-e, I-1-A-f, I-1-A-g, or I-1-A-h, R³ is —NHS(O)₂R^{A}, wherein R^{A} is —CH₂CH₂OC(O)R″, wherein R″ is an optionally substituted C₁₋₆ alkyl, an optionally substituted C₂₋₆ alkenyl, an optionally substituted C₂₋₆ alkynyl, an optionally substituted C₁₋₆ heteroalkyl, or an optionally substituted C₃₋₆ cycloalkyl, for example, R^{A} is —CH₂CH₂OC(O)CH(NH₂)CH(CH₃)₂.

8. The compound of any one of claims 3-7, or a pharmaceutically acceptable salt thereof, wherein in Formula I-1-A, I-1-A-a, I-1-A-b, I-1-A-c, I-1-A-d, I-1-A-e, I-1-A-f, I-1-A-g, or 1-1-A-h, both R^{9a} and R^{9b} are F or methyl; or R^{9a} is F and R^{9b} is methyl; or R^{9a} and R^{9b}, together with the intervening C atom, are joined to form an optionally substituted cyclopropyl or cyclobutyl; or R^{9a} and R^{9b} are joined to form ═CF₂.

9. The compound of any one of claims 4-8, or a pharmaceutically acceptable salt thereof, wherein in Formula I-1-A-a, I-1-A-c, I-1-A-f, or I-1-A-g, R^{B} is selected from H, halogen, CN, C₁₋₃ alkyl, or C₁₋₃ alkoxy; preferably H, F, Cl, CN, CH₃, or OCH₃.

10. The compound of any one of claims 4-8, or a pharmaceutically acceptable salt thereof, wherein in Formula I-1-A-a, I-1-A-c, I-1-A-f, or I-1-A-g, R^{B} is H, or R^{B} is F.

11. The compound of any one of claims 4-10, or a pharmaceutically acceptable salt thereof, wherein in Formula I-1-A-a, I-1-A-b, I-1-A-c, I-1-A-d, I-1-A-e, I-1-A-f, or I-1-A-g, R^{E} is H.

12. The compound of any one of claims 4-11, or a pharmaceutically acceptable salt thereof, wherein in Formula I-1-A-a, I-1-A-b, I-1-A-c, I-1-A-d, I-1-A-f, I-1-A-g, or I-1-A-h, R^{F} is H.

13. The compound of any one of claims 4-12, or a pharmaceutically acceptable salt thereof, wherein in Formula I-1-A-a, I-1-A-b, I-1-A-c, I-1-A-d, I-1-A-e, I-1-A-f, I-1-A-g, or I-1-A-h, R^{C} is an optionally substituted C₁₋₆ alkyl, an optionally substituted C₁₋₆ heteroalkyl, or an optionally substituted C₁₋₆ haloalkyl; or
R^{C} is selected from the following groups which are further optionally substituted:

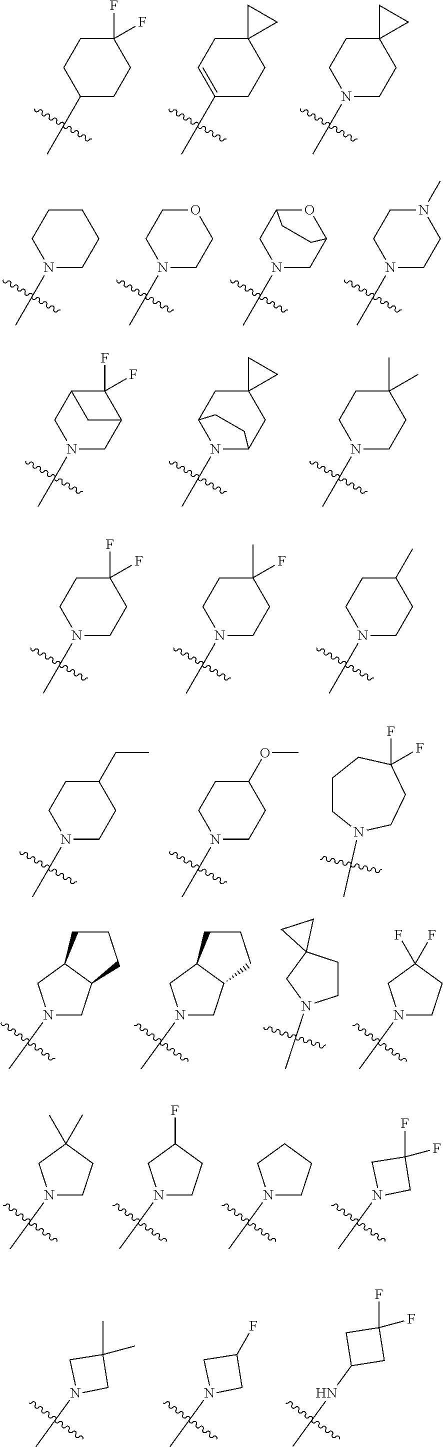

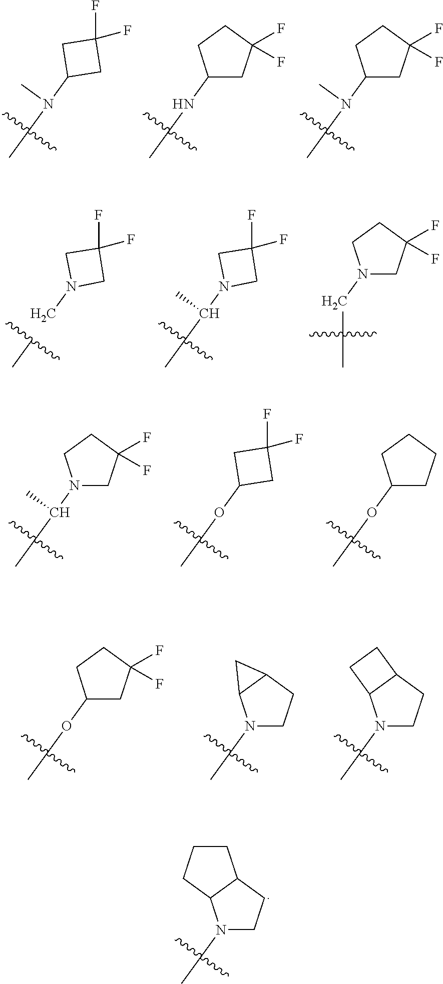

14. The compound of any one of claims 4-12, or a pharmaceutically acceptable salt thereof, wherein in Formula I-1-A-a, I-1-A-b, I-1-A-c, I-1-A-d, I-1-A-e, I-1-A-f, I-1-A-g, or I-1-A-h, R^{C} is selected from the following groups which are further optionally substituted:

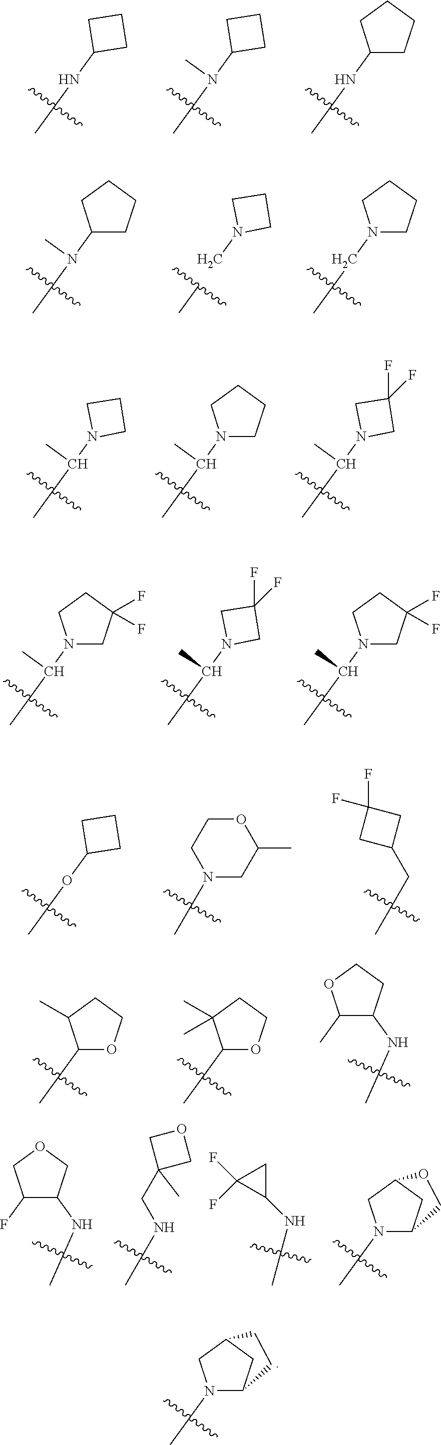

15. The compound of any one of claims 4-12, or a pharmaceutically acceptable salt thereof, wherein in Formula I-1-A-a, I-1-A-b, I-1-A-c, I-1-A-d, I-1-A-e, I-1-A-f, I-1-A-g, or I-1-A-h, R^{C} is selected from the following groups which are further optionally substituted:

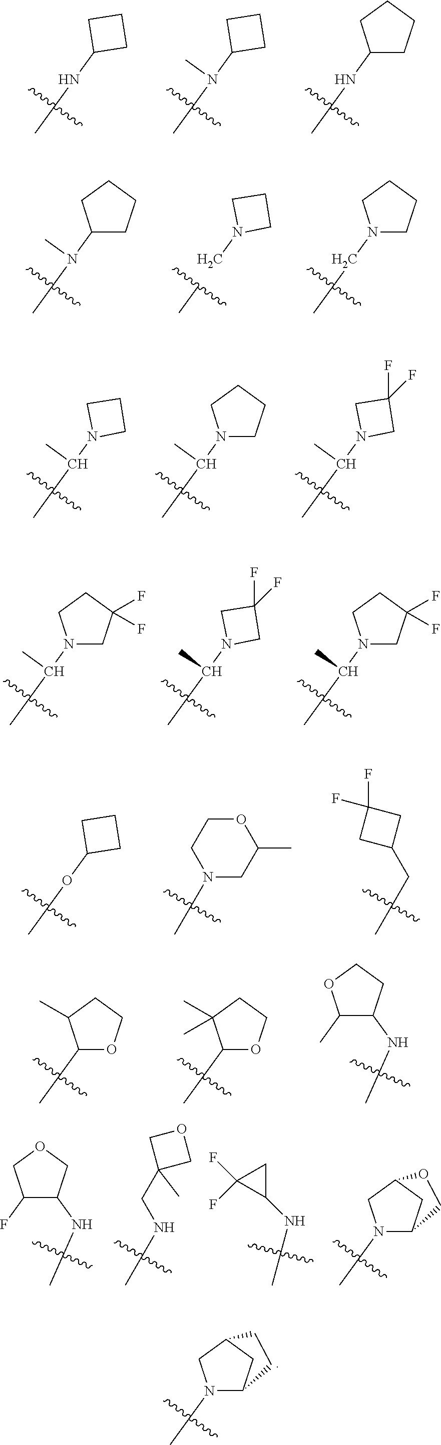

16. The compound of any one of claims 4-12, or a pharmaceutically acceptable salt thereof, wherein in Formula I-1-A-a, I-1-A-b, I-1-A-c, I-1-A-d, I-1-A-e, I-1-A-f, I-1-A-g, or I-1-A-h, R^{C} is CH₃, CH₂CF₃, CH₂CH₂CF₃, OCF₃, OCH₂CF₃, OCH₂CH₂CF₃, or CF₃; or R^{C} is NHCH₂CH₂CF₃.

17. The compound of any one of claims 4-12, or a pharmaceutically acceptable salt thereof, wherein in Formula I-1-A-a, I-1-A-b, I-1-A-c, I-1-A-d, I-1-A-e, I-1-A-f, I-1-A-g, or I-1-A-h, R^{C} is selected from the following groups which are further optionally substituted:

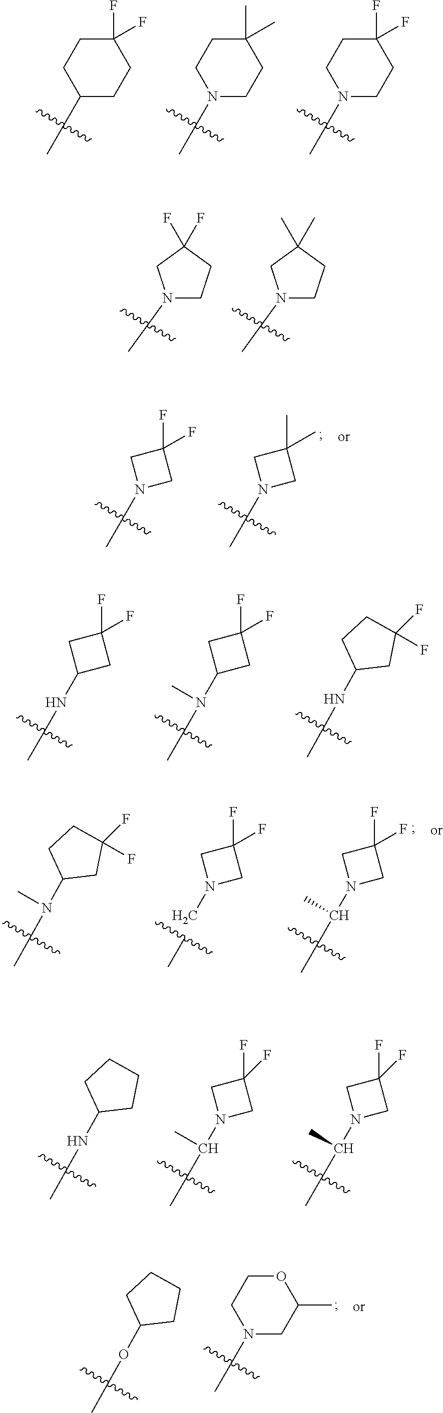

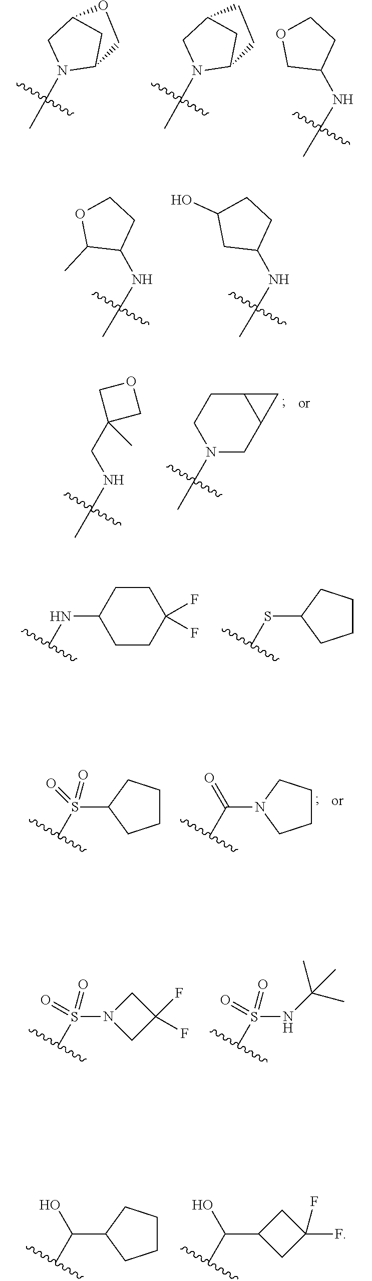

18. The compound of any one of claims 4-17, or a pharmaceutically acceptable salt thereof, wherein, in Formula I-1-A-a, I-1-A-b, I-1-A-e, I-1-A-f, I-1-A-g, or I-1-A-h, R^{D} is hydrogen, F, Cl, CN, OH, NH₂, C₁₋₆ alkyl, O(C₁₋₆ alkyl), O(CH₂)₁₋₃O(C₁₋₆ alkyl), O(CH₂)₁₋₃NH(C₁₋₆ alkyl), O(CH₂)₁₋₃N(C₁₋₆ alkyl)(C₁₋₆ alkyl), NH(C₁₋₆ alkyl), N(C₁₋₆ alkyl)(C₁₋₆ alkyl), C(O)NH₂, C(O)NH(C₁₋₆ alkyl), C(O)N(C₁₋₆ alkyl)(C₁₋₆ alkyl), C₃₋₅ cycloalkyl, 4-6 membered heterocyclyl, phenyl, or 5-6 membered heteroaryl, wherein cycloalkyl, heterocyclyl, phenyl, or heteroaryl is unsubstituted or substituted with one or more groups independently selected from halogen (such as F or Cl) and C₁₋₆ alkyl, and C₁₋₆ alkyl is unsubstituted or substituted with F.

19. The compound of any one of claims 4-17, or a pharmaceutically acceptable salt thereof, wherein, in Formula I-1-A-a, I-1-A-b, I-1-A-e, I-1-A-f, I-1-A-g, or I-1-A-h, R^{D} is hydrogen, F, Cl, CN, OH, NH₂, C₁₋₆ alkyl, O(C₁₋₆ alkyl), O(CH₂)₁₋₃O(C₁₋₆ alkyl), O(CH₂)₁₋₃NH(C₁₋₆ alkyl), O(CH₂)₁₋₃N(C₁₋₆ alkyl)(C₁₋₆ alkyl), NH(C₁₋₆ alkyl), N(C₁₋₆ alkyl)(C₁₋₆)alkyl), C(O)NH₂, C(O)NH(C₁₋₆ alkyl), C(O)N(C₁₋₆ alkyl)(C₁₋₆ alkyl), C₃₋₅ cycloalkyl, or 5-6 membered heteroaryl containing one, two or three ring nitrogen atoms, wherein cycloalkyl or heteroaryl is unsubstituted or substituted with one or more groups independently selected from halogen (such as F or Cl) and C₁₋₆ alkyl, and C₁₋₆ alkyl is unsubstituted or substituted with F.

20. The compound of any one of claims 4-17, or a pharmaceutically acceptable salt thereof, wherein, in Formula I-1-A-a, I-1-A-b, I-1-A-e, I-1-A-f, I-1-A-g, or I-1-A-h, R^{D} is selected from H, halogen, CN, C₁₋₃ alkyl, or C₁₋₃ alkoxy; preferably H, F, Cl, CN, CH₃, or OCH₃;
or
R^{D} is H, F, Cl, OH, CN, CH₃, OCH₃, CHF₂, CF₃, OCHF₂, CH₂CH₃, N(CH₃)₂, C(O)NH₂, C(O)NHCH₃, C(O)NHCH₂CH₃, OCH₂CH₂OCH₃, OCH₂CH₂N(CH₃)₂, or cyclopropyl; or
R^{D} is 5-membered heteroaryl containing one, two or three ring nitrogen atoms and optionally substituted with C₁₋₂ alkyl, such as pyrazolyl optionally substituted with methyl, imidazolyl optionally substituted with methyl, or triazolyl optionally substituted with methyl; preferably, R^{D} is selected from 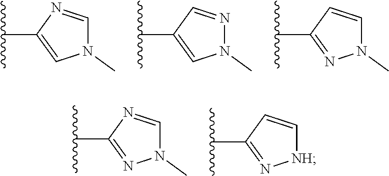
or
R^{D} is 6-membered heteroaryl containing one or two ring nitrogen atoms and optionally substituted with C₁₋₂ alkyl, such as pyridinyl optionally substituted with methyl or pyrimidinyl optionally substituted with methyl; preferably, R^{D} is selected from 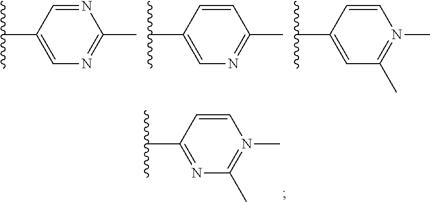
or
R^{D} is 5-6 membered heterocyclyl containing one or two ring nitrogen atoms and optionally substituted with oxo and/or C₁₋₂ alkyl, such as pyrrolidinyl optionally substituted with oxo, piperidinyl optionally substituted with methyl, piperazinyl optionally substituted with oxo and methyl, or tetrahydropyridinyl optionally substituted with methyl; preferably, R^{D} is selected from 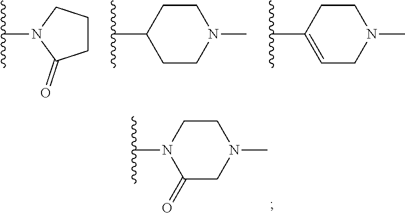
or R^{D} is C(O)-(5-6 membered heterocyclyl), wherein the heterocyclyl contains one or two ring nitrogen atoms and optionally substituted with C₁₋₂ alkyl, such as C(O)-piperazinyl optionally substituted with methyl; preferably, R^{D} is

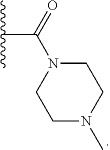 [SMILES: *C(=O)N1CCN(C)CC1]

21. A compound selected from those as shown in Table A, or a pharmaceutically acceptable salt thereof.

22. A prodrug (e.g., an ester prodrug or an amino ester prodrug) of the compound of any one of claims 1-21 or a pharmaceutically acceptable salt thereof.

23. A pharmaceutical composition comprising the compound of any one of claims 1-21 or a pharmaceutically acceptable salt thereof or the prodrug of claim 22, and a pharmaceutically acceptable excipient.

24. A method of inhibiting KIF18A protein in a cell, the method comprising contacting the cell with the compound of any one of claims 1-21 or a pharmaceutically acceptable salt thereof.

25. A method of treating a cancer in a subject, the method comprising administering to the subject a therapeutically effective amount of the compound of any one of claims 1-21 or a pharmaceutically acceptable salt thereof, or the pharmaceutical composition of claim 23.

26. The method of claim 25, wherein the cancer is selected from breast, bladder, colon, cervix, lung, pancreas, prostate, and/or ovarian cancers.

27. The method of claim 25 or 26, further comprising treating the subject with an additional therapy, such as a chemotherapeutic agent, therapeutic antibody, radiation, cell therapy, or immunotherapy.

28. The method of any one of claims 25-27, wherein the subject has a cancer associated with KIF18A protein.
